(19) United States
(12) Patent Application Publication
(10) Pub. No.: US 2012/0202806 A1
(43) Pub. Date: Aug. 9, 2012

(54) Novel Pyrimidine- And Triazine-Hepcidine Antagonists

(75) Inventors: Franz Dürrenberger, Dornach (CH); Susanna Burckhardt, Zurich (CH); Peter O. Geisser, St. Gallen (CH); Wilm Buhr, Konstanz (DE); Felix Funk, Winterthur (CH); Julia M. Bainbridge, Didcot Oxon Oxfordshire (GB); Vincent A. Corden, Stanford in the Vale (GB); Stephen M. Courtney, Stanford in the Vale (GB); Tara Davenport, Abingdon (GB); Stefan Jaeger, Hamburg (DE); Mark P. Ridgill, Horsham (GB); Mark Slack, Hamburg (DE); Christopher J. Yarnold, Didcot Oxon (GB); Wei Tsung Yau, Didcot Oxon (GB)

(73) Assignee: VIFOR (INTERNATIONAL) AG, St. Gallen (CH)

(21) Appl. No.: 13/391,712

(22) PCT Filed: Aug. 31, 2010
(86) PCT No.: PCT/EP2010/062708

§ 371 Date: Mar. 12, 2012

(30) Foreign Application Priority Data
Sep. 2, 2009 (EP) 09169286.3

(51) Int. Cl. A61K 31/5377; A61P 3/02; A61P 7/06; A61K 31/506

(52) U.S. Cl. 514/232.2; 514/256; 514/235.8

(57) ABSTRACT

The present invention relates to new hepcidin antagonists, pharmaceutical compositions containing them and the use thereof as a drug, in particular for the treatment of iron metabolism disorders such as, in particular, iron deficiency diseases and anaemia, in particular anaemia associated with chronic inflammatory disease (ACD: anaemia of chronic disease and AI: anaemia of inflammation).

### INTRODUCTION

The invention relates to novel hepcidin antagonists of general formula (I), pharmaceutical compositions comprising them and the use thereof for the treatment of iron metabolism disorders, in particular of anaemia related to chronic inflammatory disease (anaemia of chronic disease (ACD) and anaemia of inflammation (AI)) or of iron deficiency disorders and iron deficiency anaemia.

### BACKGROUND

Iron is an essential trace element for almost all organisms and is particularly important for growth and blood formation. The balance of the iron metabolism is regulated primarily at the level of iron recovery from haemoglobin of aging erythrocytes and the duodenal absorption of iron in food. The released iron is absorbed via the intestine, in particular through specific transport systems (DMT-1, ferroportin, transferrin, transferrin receptors), transported in the bloodstream and relayed into the corresponding tissue and organs.

The element iron is very important to the human body, inter alia, for oxygen transport, oxygen uptake, cell functions such as mitochondrial electron transport, and ultimately for energy metabolism.

The human body contains on average 4 to 5 g of iron, which is present in enzymes, in haemoglobin and myoglobin, and as stored or reserve iron in the form of ferritin and haemosiderin.

About half of this iron (about 2 g) is in the form of haem iron bound in the haemoglobin of the red blood corpuscles. As these erythrocytes have only a limited life (75 to 150 days), new ones have to be formed continuously and old ones eliminated (new erythrocytes are formed at a rate of more than 2 million per second). This high regeneration capacity is achieved by means of macrophages in that the macrophages phagocytotically absorb and lyse the aging erythrocytes and can thus recycle the iron contained therein for the iron metabolism. The majority of the iron required for erythropoiesis, about 25 mg per day, is provided in this way.

The daily iron requirement of a human adult is between 0.5 and 1.5 mg per day, and small children and pregnant women require 2 to 5 mg of iron per day. The daily iron loss, for example due to the shedding of skin and epithelial cells, is comparatively slight, increased iron loss occurring in women for example during menstrual bleeding. In general, blood loss can considerably reduce iron metabolism, as about 1 mg of iron is lost per 2 ml of blood. The normal daily iron loss of about 1 mg is usually replaced in a healthy human adult through daily food intake. The iron metabolism is regulated by resorption, the resorption rate of the iron present in food being between 6 and 12%, and up to 25% in the case of iron deficiency. The resorption rate is regulated by the organism as a function of the iron requirement and the size of the iron store. The human organism uses both divalent and trivalent iron ions. Iron(III) compounds are conventionally dissolved in the stomach if the pH is sufficiently acidic and therefore made available for resorption. Resorption of the iron takes place through mucosal cells in the upper small intestine. In the process, trivalent non-haem iron is initially reduced to Fe²⁺ in the intestinal cell membrane, for example by ferrireductase (duodenal cytochrome b associated with the membrane) so that it can then be transported by the transport protein DMT1 (divalent metal transporter 1) into the intestinal cells. On the other hand, haem iron passes unchanged via the cell membrane into the enterocytes. In the enterocytes, iron is either stored in ferritin as deposited iron or released into the blood through the transport protein ferroportin, bound to transferrin. Hepcidin plays a crucial role in this process as it is the essential regulator of iron absorption. The divalent iron transported into the blood by the ferroportin is converted by oxidases (ceruloplasmin, hephaestin) into trivalent iron which is then transported to the relevant points in the organism by means of transferrin (see for example: “Balancing acts: molecular control of mammalian iron metabolism”. M. W. Hentze, Cell 117, 2004, 285-297.)

Regulation of iron levels is controlled or regulated by hepcidin.

Hepcidin is a peptide hormone produced in the liver. The predominant active form has 25 amino acids (see for example: “Hepcidin, a key regulator of iron metabolism and mediator of anaemia of inflammation”. T. Ganz Blood 102, 2003, 783-8), although two forms which are shortened at the amino end, hepcidin-22 and hepcidin-20, have been found. Hepcidin acts on the absorption of iron via the intestine and via the placenta and on the release of iron from the reticuloendothelial system. In the body, hepcidin is synthesised from what is known as pro-hepcidin in the liver, pro-hepcidin being coded by the gene known as the HAMP gene. If the organism is supplied with sufficient iron and oxygen, more hepcidin is formed. Hepcidin binds, in the small intestinal mucosal cells and in the macrophages, with ferroportin by means of which iron is conventionally transported from the interior of the cell into the blood.

The transport protein ferroportin is a transmembrane protein consisting of 571 amino acids which is formed in the liver, spleen, kidneys, heart, intestine and placenta and is localised. In particular, ferroportin is localised in the basolateral membrane of intestinal epithelial cells. Ferroportin bound in this way thus brings about the export of iron into the blood. In this case, it is most probable that ferroportin transports iron as Fe²⁺. If hepcidin binds to ferroportin, ferroportin is transported into the interior of the cell and broken down so that the release of iron from the cells is then almost completely blocked. If the ferroportin is inactivated by hepcidin so that it is unable to carry off the iron stored in the mucosal cells, the iron is lost with the natural shedding of cells via the stools. The absorption of iron in the intestine is therefore reduced by hepcidin. If the iron content in the serum is reduced, on the other hand, hepcidin production in the hepatocytes of the liver is reduced so that less hepcidin is released and less ferroportin is therefore inactivated, allowing a larger amount of iron to be transported into the serum.

In addition, ferroportin is markedly localised in the reticuloendothelial system (RES), to which the macrophages also belong.

Hepcidin plays an important part here when iron metabolism is impaired by chronic inflammation since, in particular, interleukin-6 is increased in the case of such inflammation, leading to an increase in hepcidin levels. As a result, more hepcidin is bound to the ferroportin of the macrophages, causing the release of iron to be blocked, which ultimately leads to anaemia of inflammation (ACD or AI).

As the mammalian organism cannot actively excrete iron, the iron metabolism is basically controlled via the cellular release of iron from macrophages, hepatocytes and enterocytes by means of hepcidin.

Hepcidin therefore has an important role in functional anaemia. In this case, the iron requirement of the bone marrow is not sufficiently satisfied for erythropoiesis even if the iron store is full. The reason for this is assumed to be an elevated hepcidin concentration which restricts iron transport from the macrophages, in particular by blocking ferroportin, and therefore greatly reduces the release of phagocytotically recycled iron.

A disorder of the hepcidin regulation mechanism therefore has a direct effect on iron metabolism in the organism. For example, if hepcidin expression is prevented, for example due to a genetic defect, this leads directly to an iron overload known as the iron storage disease haemochromatosis.

On the other hand, overexpression of hepcidin, for example due to inflammation processes, for example in chronic inflammation, leads directly to reduced serum iron levels. In pathological cases, this can lead to a reduced haemoglobin content, reduced erythrocyte production and therefore to anaemia.

The period of application of chemotherapy agents in cancer treatment may be considerably reduced by existing anaemia as the state of reduced red blood corpuscle formation, brought about by the chemotherapy agents used, will be further aggravated by existing anaemia.

Further symptoms of anaemia include fatigue, pallor and loss of concentration. The clinical symptoms of anaemia include low serum iron contents (hypoferraemia), low haemoglobin contents, low haematocryte level as well as a reduced number of red blood corpuscles, reduced reticulocytes, elevated soluble transferrin receptor values.

Iron deficiency disorders or iron anaemia are conventionally treated by the supply of iron. Iron substitution is effected by administering iron either orally or intravenously. Erythropoietin and other erythropoiesis-stimulating substances can also be used to boost the formation of red blood corpuscles in the treatment of anaemia.

Anaemia which is caused by chronic disease, for example chronic inflammatory disease, can only be treated inadequately by these conventional methods of treatment. In particular cytokines, in particular, inflammatory cytokines, play a significant part in anaemia based on chronic inflammation processes. Hepcidin overexpression occurs, in particular in these chronic inflammatory diseases, and is known to reduce the availability of iron for the formation of the red blood corpuscles.

There is therefore a need for an effective method of treating hepcidin-mediated anaemia, in particular anaemia which cannot be treated by conventional iron substitution, such as anaemia caused by chronic inflammatory disease (ACD and AI).

Anaemia is due, inter alia, to the aforementioned chronic inflammatory diseases and to malnutrition and low-iron diets or unbalanced, low-iron eating habits. Anaemia also occurs as a result of reduced or poor iron absorption, for example owing to gastrectomy or disorders such as Crohn's disease. Iron deficiency can also occur as a result of a substantial loss of blood, for example due to an injury, heavy menstrual bleeding or blood donation. An increased iron requirement is also known to occur in the growth phase of adolescents and children and in pregnant women. As an iron deficiency leads not only to reduced red blood corpuscle formation but also to a poor oxygen supply to the organism, which can lead to the above-mentioned symptoms such as fatigue, pallor and poor concentration and, among adolescents, even to long-term impairment of cognitive development, a particularly effective therapy apart from the known conventional substitution therapies is also of particular interest in this area.

Compounds which bind to hepcidin or ferroportin and therefore inhibit the binding of hepcidin to ferroportin and therefore in turn prevent the inactivation of ferroportin by hepcidin, or compounds which prevent the internalisation of the hepcidin-ferroportin complex, even if hepcidin is bound to ferroportin, and thus prevent the inactivation of ferroportin by hepcidin, can generally be described as hepcidin antagonists.

The use of these hepcidin antagonists also generally makes it possible to act directly on the hepcidin regulation mechanism, for example by inhibiting hepcidin expression or by blocking hepcidin-ferroportin interaction, and, via this method, thus to prevent blockage of the iron transport pathway from cell macrophages, liver cells and mucosal cells into the serum via the transport protein ferroportin. Hepcidin antagonists or hepcidin expression inhibitors of this type therefore represent substances which are suitable for the production of pharmaceutical compositions or medications for the treatment of anaemia, in particular anaemia in chronic inflammatory disease. These substances can be used for the treatment of such disorders and the resultant diseases as they directly influence the increase in the release of recycled haem iron through macrophages, and increase the absorption of iron released from food in the intestinal tract. Substances of this type, hepcidin expression inhibitors and hepcidin antagonists, can therefore be used for the treatment of iron metabolism disorders such as iron deficiency diseases, anaemia and anaemia-related diseases. In particular, this also includes anaemia caused by acute or chronic inflammatory diseases such as, for example, osteoarticular diseases such as rheumatoid polyarthritis or diseases associated with inflammatory syndromes. Substances of this type may therefore be of special benefit, in particular for cancers, particularly colorectal cancer, multiple myeloma, ovarian and endometrial cancer and prostate cancer, CKD 3-5 (chronic kidney disease stage 3-5), CHF (chronic heart failure), RA (rheumatoid arthritis), SLE (systemic lupus erythematosus) and IBD (inflammatory bowel disease).

### PRIOR ART

Hepcidin antagonists or compounds which have an inhibiting or supporting effect on the biochemical regulatory pathways in the iron metabolism are basically known from the prior art.

For example, WO2008/036933 describes double-stranded dsRNA which has an inhibitory effect on the expression of human HAMP genes in cells and therefore suppresses the formation of hepcidin, which is coded by the HAMP gene, at a very early stage in the iron metabolism pathway. Less hepcidin is therefore formed, so hepcidin is not available to inhibit ferroportin and iron can be transported unimpeded from the cell into the blood by ferroportin.

Further compounds which are directly intended to reduce hepcidin expression are known from US2005/020487, which discloses compounds that stabilise HIF-α and therefore lead to a reduction in hepcidin expression.

US2007/004618 relates to siRNA, which has a direct inhibiting effect on hepcidin-mRNA expression.

All these compounds and processes therefore start in the iron metabolism pathway before hepcidin is formed and reduce the general formation thereof at an early stage. In addition, however, substances and compounds are known and disclosed in the prior art which bind to hepcidin that has already formed in the body and therefore inhibit the binding thereof to the transmembrane protein ferroportin so that inactivation of the ferroportin by the hepcidin is no longer possible. These compounds are therefore known as hepcidin antagonists, members of this group based on hepcidin antibodies being known in particular. Prior art documents are also known which disclose various mechanisms for acting on hepcidin expression, for example using antisense-RNA or DNA molecules, ribozymes and anti-hepcidin antibodies. These are disclosed, for example, in EP 1 392 345.

WO09/058,797 further discloses anti-hepcidin antibodies and the use thereof for specific binding to human hepcidin-25 and therefore the use thereof for the therapeutic treatment of low iron levels, in particular of anaemia.

Further compounds which act as hepcidin antagonists and are formed from the group of hepcidin antibodies are known from EP 1 578 254, WO08/097,461, US2006/019339, WO09/044,284 or WO09/027,752.

In addition, antibodies are also known which bind to ferroportin-1 and therefore activate ferroportin so that it can promote the transport of iron from the cell into the serum. Ferroportin-1 antibodies of this type are known, for example, from US2007/218055.

All the described compounds which act as hepcidin antagonists or inhibit hepcidin expression are relatively high molecular weight compounds, in particular those which are obtainable predominantly by genetic engineering.

Low molecular weight compounds which play a part in iron metabolism and can have an inhibiting or promoting effect are also known.

WO08/109,840 accordingly discloses specific tricyclic compounds which may be used, in particular, for the treatment of iron metabolism disorders such as, for example, ferroportin disorders, these compounds being able to act by inhibition or activation by regulating DMT-1. The compounds in WO08/109,840 are described, in particular, as DMT-1 inhibitors, which means that they may be used preferably in the case of diseases involving elevated iron accumulation or iron storage diseases such as haemochromatosis.

Low molecular weight compounds which regulate the DMT-1 mechanism are also known from WO08/121,861. This document deals, in particular, with specific pyrazole and pyrrole compounds, the treatment of iron overload disorders based, for example, on ferroportin disorders, also being disclosed in particular herein.

In addition, US2008/234384 relates to specific diaryl and diheteroaryl compounds for the treatment of iron metabolism disorders such as, for example, ferroportin disorders which, by acting as DMT-1 inhibitors can also be used, in particular, for the treatment of disorders due to elevated iron accumulation. However, possible DMT-1 regulating mechanisms which can be used in the case of iron deficiency symptoms are also mentioned quite generally in this document.

The same applies to WO08/151,288 which discloses specific aromatic and heteroaromatic compounds that act on DMT-1 regulation and can therefore be used for the treatment of iron metabolism disorders.

Therefore, the low molecular weight compounds disclosed in the prior art, which act on the iron metabolism, are applied to DMT-1 regulating mechanisms and disclosed, in particular, for use as an agent for the treatment of iron accumulation disorders or iron overload syndromes such as haemochromatosis.

Chemical compounds based on the structure of quinoxalinones have hitherto not been disclosed in connection with the treatment of iron metabolism disorders. In addition, low molecular weight chemical structures which act as hepcidin antagonists and are thus suitable for the treatment of iron metabolism disorders have not yet been disclosed.

### OBJECT

The object of the present invention was to provide, in particular, compounds which can be used for the treatment of iron deficiency disorders or anaemia, in particular ACD and AI, and which act on the iron metabolism, in particular as hepcidin antagonists, and therefore antagonise and hence regulate the hepcidin-ferroportin interaction in the iron metabolism. A further object of the present invention, in particular, was to provide compounds which are selected from the group of low molecular weight compounds and can generally be produced by simpler methods of synthesis than the antagonistic hepcidin-inhibiting compounds such as RNA, DNA or antibodies obtainable by genetic engineering.

### DESCRIPTION OF THE INVENTION

The inventors have found that specific compounds from the group of quinoxalinones act as hepcidin antagonists.

The invention relates to compounds of general formula (I)

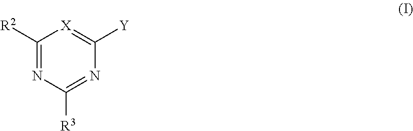

wherein
X is selected from the group consisting of N or C—R¹, wherein
R¹ is selected from the group consisting of:
hydrogen,
hydroxyl,
halogen,
carboxyl,
sulfonic acid residue (—SO₃H),
optionally substituted aminocarbonyl,
optionally substituted aminosulfonyl,
optionally substituted amino,
optionally substituted alkyl,
optionally substituted acyl,
optionally substituted alkoxycarbonyl,
optionally substituted acyloxy,
optionally substituted alkoxy,
optionally substituted alkenyl,
optionally substituted alkynyl,
optionally substituted aryl,
optionally substituted heterocyclyl;
   R² and R³ are the same or different and are each selected from the group consisting of:
hydrogen,
hydroxyl,
halogen,
carboxyl,
sulfonic acid residue (—SO₃H),
optionally substituted aminocarbonyl,
optionally substituted aminosulfonyl,
optionally substituted amino,
optionally substituted alkyl,
optionally substituted acyl,
optionally substituted alkoxycarbonyl,
optionally substituted acyloxy,
optionally substituted alkoxy,
optionally substituted alkenyl,
optionally substituted alkynyl,
optionally substituted aryl,
optionally substituted heterocyclyl;
Y is selected from the group consisting of:
hydrogen
hydroxyl,
halogen, preferably chlorine,
optionally substituted aryloxy, preferably phenoxy, and

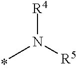

(* means here and in the subsequent description the point of binding of a given residue)
wherein
R⁴ and R⁵ are the same or different and are each selected from the group consisting of:
   hydrogen,
   optionally substituted amino,
   optionally substituted aminocarbonyl,
   optionally substituted alkyl-, aryl- or heterocyclylsulfonyl,
   optionally substituted alkyl,
   optionally substituted alkenyl,
   optionally substituted alkynyl,
   optionally substituted acyl,
   optionally substituted aryl,
   optionally substituted heterocyclyl or
   wherein R⁴ and R⁵, together with the nitrogen atom to which they are bound, form a saturated or unsaturated, optionally substituted 3- to 8-membered ring, which can optionally contain further heteroatoms;
      or pharmaceutically acceptable salts thereof.

The invention further relates, in particular, to compounds of general structural formula (I′)

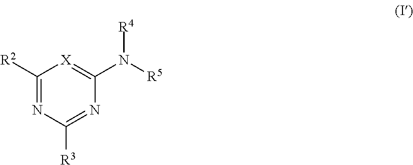

wherein
X is selected from the group consisting of N or C—R¹, wherein
R¹ is selected from the group consisting of:
hydrogen,
hydroxyl,
halogen,
carboxyl,
sulfonic acid residue (—SO₃H),
optionally substituted aminocarbonyl,
optionally substituted aminosulfonyl,
optionally substituted amino,
optionally substituted alkyl,
optionally substituted acyl,
optionally substituted alkoxycarbonyl,
optionally substituted acyloxy,
optionally substituted alkoxy
optionally substituted alkenyl,
optionally substituted alkynyl,
optionally substituted aryl,
optionally substituted heterocyclyl;
   R² and R³ are the same or different and are each selected from the group consisting of:
hydrogen,
hydroxyl,
halogen,
carboxyl,
sulfonic acid residue (—SO₃H),
optionally substituted aminocarbonyl,
optionally substituted aminosulfonyl,
optionally substituted amino,
optionally substituted alkyl,
optionally substituted acyl,
optionally substituted alkoxycarbonyl,
optionally substituted acyloxy,
optionally substituted alkoxy,
optionally substituted alkenyl,
optionally substituted alkynyl,
optionally substituted aryl,
optionally substituted heterocyclyl;
   R⁴ and R⁵ are the same or different and are each selected from the group consisting of:
hydrogen,
optionally substituted amino,
optionally substituted alkyl-, aryl- or heterocyclylsulfonyl,
optionally substituted alkyl,
optionally substituted alkenyl,
optionally substituted alkynyl,
optionally substituted acyl,
optionally substituted aryl,
optionally substituted heterocyclyl or
wherein R⁴ and R⁵, together with the nitrogen atom to which they are bound, form a saturated or unsaturated, optionally substituted 3- to 8-membered ring, which can optionally contain further heteroatoms;
   or pharmaceutically acceptable salts thereof.

Throughout the invention, the above-mentioned substituent groups are defined as follows:

Optionally substituted alkyl preferably includes:

straight-chain or branched alkyl preferably containing 1 to 8, more preferably 1 to 6, particularly preferably 1 to 4 carbon atoms. In an embodiment of the invention, optionally substituted straight-chain or branched alkyl can also include alkyl groups in which preferably 1 to 3 carbon atoms are replaced by corresponding nitrogen, oxygen or sulphur-containing heteroanalogous groups. This means, in particular, that, for example, one or more methylene groups in the aforementioned alkyl residues can be replaced by NH, O or S.

Optionally substituted alkyl further includes cycloalkyl containing preferably 3 to 8, more preferably 5 or 6, particularly preferably 6 carbon atoms.

Substituents of the above-defined optionally substituted alkyl preferably include 1 to 3 of the same or different substituents selected, for example, from the group consisting of: optionally substituted cycloalkyl, as defined below, hydroxy, halogen, cyano, alkoxy, as defined below, optionally substituted aryloxy, as defined below, optionally substituted heterocyclyloxy, as defined below, carboxy, optionally substituted acyl, as defined below, optionally substituted aryl, as defined below, optionally substituted heterocyclyl, as defined below, optionally substituted amino, as defined below, mercapto, optionally substituted alkyl, aryl or heterocyclylsulfonyl (R—SO₂—), as defined below.

Examples of alkyl residues containing 1 to 8 carbon atoms include: a methyl group, an ethyl group, an n-propyl group, an i-propyl group, an n-butyl group, an i-butyl group, a sec-butyl group, a t-butyl group, an n-pentyl group, an i-pentyl group, a sec-pentyl group, a t-pentyl group, a 2-methylbutyl group, an n-hexyl group, a 1-methylpentyl group, a 2-methylpentyl group, a 3-methylpentyl group, a 4-methylpentyl group, a 1-ethylbutyl group, a 2-ethylbutyl group, a 3-ethylbutyl group, a 1,1-dimethylbutyl group, a 2,2-dimethylbutyl group, a 3,3-dimethylbutyl group, a 1-ethyl-1-methylpropyl group, an n-heptyl group, a 1-methylhexyl group, a 2-methylhexyl group, a 3-methylhexyl group, a 4-methylhexyl group, a 5-methylhexyl group, a 1-ethylpentyl group, a 2-ethylpentyl group, a 3-ethylpentyl group, a 4-ethylpentyl group, a 1,1-dimethylpentyl group, a 2,2-dimethylpentyl group, a 3,3-dimethylpentyl group, a 4,4-dimethylpentyl group, a 1-propylbutyl group, an n-octyl group, a 1-methylheptyl group, a 2-methylheptyl group, a 3-methylheptyl group, a 4-methylheptyl group, a 5-methylheptyl group, a 6-methylheptyl group, a 1-ethylhexyl group, a 2-ethylhexyl group, a 3-ethylhexyl group, a 4-ethylhexyl group, a 5-ethylhexyl group, a 1,1-dimethylhexyl group, a 2,2-dimethylhexyl group, a 3,3-dimethylhexyl group, a 4,4-dimethylhexyl group, a 5,5-dimethylhexyl group, a 1-propylpentyl group, a 2-propylpentyl group, etc. Those containing 1 to 6 carbon atoms, in particular methyl, ethyl, n-propyl and i-propyl are preferred. C₁-C₄ alkyl, in particular, methyl, ethyl and i-propyl are most preferred.

Examples of alkyl groups obtained by replacement with one or more heteroanalogous groups such as —O—, —S— or —NH—, are preferably those in which one or more methylene groups are replaced by —O— with formation of one or more ether groups, such as methoxymethyl, ethoxymethyl, 2-methoxyethyl, etc. According to the invention, in particular polyether groups such as poly(ethyleneoxy) groups are also included in the definition of alkyl.

Cycloalkyl residues containing 3 to 8 carbon atoms preferably include: a cyclopropyl group, a cyclobutyl group, a cyclopentyl group, a cyclohexyl group, a cycloheptyl group and a cyclooctyl group. A cyclopropyl group, a cyclobutyl group, a cyclopentyl group and a cyclohexyl group are preferred. A cyclopentyl group and a cyclohexyl group are particularly preferred.

Within the meaning of the present invention, halogen includes fluorine, chlorine, bromine and iodine, preferably fluorine or chlorine.

Examples of a linear or branched alkyl residue substituted by halogen and containing 1 to 8 carbon atoms include:

a fluoromethyl group, a difluoromethyl group, a trifluoromethyl group, a chloromethyl group, a dichloromethyl group, a trichloromethyl group, a bromomethyl group, a dibromomethyl group, a tribromomethyl group, a 1-fluoroethyl group, a 1-chloroethyl group, a 1-bromoethyl group, a 2-fluoroethyl group, a 2-chloroethyl group, a 2-bromoethyl group, a 1,2-difluoroethyl group, a 1,2-dichloroethyl group, a 1,2-dibromoethyl group, a 2,2,2-trifluoroethyl group, a heptafluoroethyl group, a 1-fluoropropyl group, a 1-chloropropyl group, a 1-bromopropyl group, a 2-fluoropropyl group, a 2-chloropropyl group, a 2-bromopropyl group, a 3-fluoropropyl group, a 3-chloropropyl group, a 3-bromopropyl group, a 1,2-difluoropropyl group, a 1,2-dichloropropyl group, a 1,2-dibromopropyl group, a 2,3-difluoropropyl group, a 2,3-dichloropropyl group, a 2,3-dibromopropyl group, a 3,3,3-trifluoropropyl group, a 2,2,3,3,3-pentafluoropropyl group, a 2-fluorobutyl group, a 2-chlorobutyl group, a 2-bromobutyl group, a 4-fluorobutyl group, a 4-chlorobutyl group, a 4-bromobutyl group, a 4,4,4-trifluorobutyl group, a 2,2,3,3,4,4,4-heptafluorobutyl group, a perfluorobutyl group, a 2-fluoropentyl group, a 2-chloropentyl group, a 2-bromopentyl group, a 5-fluoropentyl group, a 5-chloropentyl group, a 5-bromopentyl group, a perfluoropentyl group, a 2-fluorohexyl group, a 2-chlorohexyl group, a 2-bromohexyl group, a 6-fluorohexyl group, a 6-chlorohexyl group, a 6-bromohexyl group, a perfluorohexyl group, a 2-fluoroheptyl group, a 2-chloroheptyl group, a 2-bromoheptoyl group, a 7-fluoroheptyl group, a 7-chloroheptyl group, a 7-bromoheptyl group, a perfluoroheptyl group, etc. Fluoroalkyl, difluoroalkyl and trifluoroalkyl are mentioned in particular, and trifluoromethyl is preferred.

Examples of a cycloalkyl residue substituted by halogen and containing 3 to 8 carbon atoms include: a 2-fluorocyclopentyl group, a 2-chlorocyclopentyl group, a 2-bromocyclopentyl group, a 3-fluorocyclopentyl group, a 3-chlorocyclopentyl group, a 3-bromocyclopentyl group, a 2-fluorocyclohexyl group, a 2-chlorocyclohexyl group, a 2-bromocyclohexyl group, a 3-fluorocyclohexyl group, a 3-chlorocyclohexyl group, a 3-bromocyclohexyl group, a 4-fluorocyclohexyl group, a 4-chlorocyclohexyl group, a 4-bromocyclohexyl group, a di-fluorocyclopentyl group, a di-chlorocyclopentyl group, a di-bromocyclopentyl group, a di-fluorocyclohexyl group, a di-chlorocyclohexyl group, a di-bromocyclohexyl group, a tri-fluorocyclohexyl group, a tri-chlorocyclohexyl group, a tri-bromocyclohexyl group, etc. Chlorocycloalkyl, dichlorocycloalkyl and trichlorocycloalkyl as well as fluorocycloalkyl, difluorocycloalkyl and trifluorocycloalkyl are mentioned in particular.

Examples of a hydroxy-substituted alkyl residue include the above-mentioned alkyl residues which contain 1 to 3 hydroxyl residues such as, for example, hydroxymethyl, 2-hydroxyethyl, 3-hydroxypropyl, etc.

Examples of an alkoxy-substituted alkyl residue include the above-mentioned alkyl residues which contain 1 to 3 alkoxy residues as defined below such as, for example, methoxymethyl, ethoxymethyl, 2-methoxyethylene, etc.

Examples of an aryloxy-substituted alkyl residue include the above-mentioned alkyl residues containing 1 to 3 aryloxy residues as defined below such as, for example, phenoxymethyl, 2-phenoxyethyl and 2- or 3-phenoxypropyl, etc. 2-phenoxyethyl is particularly preferred.

Examples of a heterocyclyloxy-substituted alkyl residue include the above-mentioned alkyl residues which contain 1 to 3 heterocyclyloxy residues as defined below such as, for example, pyridin-2-yloxymethyl, ethyl or propyl, pyridin-3-yloxymethyl, ethyl or propyl, thiophen-2-yloxymethyl, ethyl or propyl, thiophen-3-yloxymethyl, ethyl or propyl, furan-2-yloxymethyl, ethyl or propyl, furan-3-yloxymethyl, ethyl or propyl.

Examples of an acyl-substituted alkyl residue include the above-mentioned alkyl residues which contain 1 to 3 acyl residues as defined below.

Examples of a cycloalkyl-substituted alkyl group include the above-mentioned alkyl residues containing 1 to 3, preferably 1 (optionally substituted) cycloalkyl group such as, for example: cyclohexylmethyl, 2-cyclohexylethyl, 2- or 3-cyclohexylpropyl, etc.

Examples of an aryl-substituted alkyl group include the above-mentioned alkyl residues containing 1 to 3, preferably 1 (optionally substituted) aryl group, as defined below, such as, for example, phenylmethyl, 2-phenylethyl, 2- or 3-phenylpropyl, etc., phenylmethyl being preferred. Also particularly preferred are alkyl groups, as defined above, which are substituted by substituted aryl, as defined below, in particular by halogen-substituted aryl, such as particularly preferably 2-fluorophenylmethyl.

Examples of a heterocyclyl-substituted alkyl group include the above-mentioned alkyl residues containing 1 to 3, preferably 1 (optionally substituted) heterocyclyl group, as defined below, such as, for example, 2-pyridin-2-yl-ethyl, 2-pyridin-3-yl-ethyl, pyridin-2-yl-methyl, pyridin-3-yl-methyl, 2-furan-2-yl-ethyl, 2-furan-3-yl-ethyl, furan-2-yl-methyl, furan-3-yl-methyl, 2-thiophen-2-yl-ethyl, 2-thiophen-3-yl-ethyl, thiophen-2-yl-methyl, thiophen-3-yl-methyl, 2-morpholinylethyl, morpholinylmethyl.

Examples of an amino-substituted alkyl residue include the above-mentioned alkyl residues containing 1 to 3, preferably 1 (optionally substituted) amino group, as defined below, such as, for example, methylaminomethyl, methylaminoethyl, methylaminopropyl, 2-ethylaminomethyl, 3-ethylaminomethyl, 2-ethylaminoethyl, 3-ethylaminoethyl, etc.

Particularly preferred are alkyl groups, as defined above, which are substituted by substituted amino, as defined below, in particular by amino groups, which are substituted by optionally substituted aryl- or heterocyclyl, such as particularly preferably 6-trifluoromethyl-pyridin-2-yl-aminomethyl, 5-trifluoromethyl-pyridin-2-yl-aminomethyl, 4-trifluoromethyl-pyridin-2-yl-aminomethyl, 3-trifluoromethyl-pyridin-2-yl-aminomethyl, 6-trifluoromethyl-pyridin-3-yl-aminomethyl, 5-trifluoromethyl-pyridin-3-yl-aminomethyl, 4-trifluoromethyl-pyridin-3-yl-aminomethyl, 2-trifluoromethyl-pyridin-3-yl-aminomethyl, 2-[6-trifluoromethyl-pyridin-2-yl-amino]ethyl, 2-[5-trifluoromethyl-pyridin-2-yl-amino]ethyl, 2-[4-trifluoromethyl-pyridin-2-yl-amino]ethyl, 2-[3-trifluoromethyl-pyridin-2-yl-amino]ethyl, 2-[6-trifluoromethyl-pyridin-3-yl-amino]ethyl, 2-[5-trifluoromethyl-pyridin-3-yl-amino]ethyl, 2-[4-trifluoromethyl-pyridin-3-yl-amino]ethyl, 2-[2-trifluoromethyl-pyridin-3-yl-amino]ethyl.

Particularly preferred are 2-[5-trifluoromethyl-pyridin-2-yl-amino]ethyl:

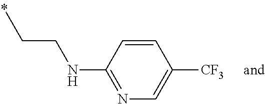

2-[4-trifluoromethyl-pyridin-2-yl-amino]ethyl:

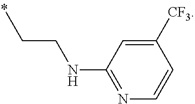

Optionally substituted alkoxy includes an optionally substituted alkyl-O-group, wherein reference may be made to the foregoing definition of the alkyl group. Preferred alkoxy groups are linear or branched alkoxy groups containing up to 6 carbon atoms such as a methoxy group, an ethoxy group, an n-propyloxy group, an i-propyloxy group, an n-butyloxy group, an i-butyloxy group, a sec-butyloxy group, a t-butyloxy group, an n-pentyloxy group, an i-pentyloxy group, a sec-pentyloxy group, a t-pentyloxy group, a 2-methylbutoxy group, an n-hexyloxy group, an i-hexyloxy group, a t-hexyloxy group, a sec-hexyloxy group, a 2-methylpentyloxy group, a 3-methylpentyloxy group, a 1-ethylbutyloxy group, a 2-ethylbutyloxy group, a 1,1-dimethylbutyloxy group, a 2,2-dimethylbutyloxy group, a 3,3-dimethylbutyloxy group, a 1-ethyl-1-methylpropyloxy group, as well as cycloalkyloxy groups such as a cyclopentyloxy group or a cyclohexyloxy group. A methoxy group, an ethoxy group, an n-propyloxy group, an i-propyloxy group, an n-butyloxy group, an i-butyloxy group, a sec-butyloxy group and a t-butyloxy group are preferred. The methoxy group is particularly preferred.

Optionally substituted aryloxy includes an optionally substituted aryl-O-group, wherein reference may be made to the following definition of optionally substituted aryl with respect to the definition of the aryl group. Preferred aryloxy groups include 5-membered and 6-membered aryl groups, of which phenoxy, which may optionally be substituted, is preferred.

Optionally substituted heterocyclyloxy includes an optionally substituted heterocyclyl-O-group, wherein reference may be made to the following definition of heterocyclyl with respect to the definition of the heterocyclyl group. Preferred heterocyclyloxy groups include saturated or unsaturated, such as aromatic 5-membered and 6-membered heterocyclyloxy groups, of which pyridin-2-yloxy, pyridin-3-yloxy, thiophen-2-yloxy, thiophen-3-yloxy, furan-2-yloxy and furan-3-yloxy are preferred.

Optionally substituted alkenyl throughout the invention preferably includes: straight-chain or branched alkenyl containing 2 to 8 carbon atoms and cycloalkenyl containing 3 to 8 carbon atoms which may optionally be substituted preferably by 1 to 3 of the same or different substituents, such as hydroxy, halogen or alkoxy. Examples include: vinyl, 1-methylvinyl, allyl, 1-butenyl, isopropenyl, cyclopropenyl, cyclobutenyl, cyclopentenyl, cyclohexenyl. Vinyl or allyl is preferred.

Throughout the invention, optionally substituted alkynyl preferably includes: straight-chain or branched alkynyl containing 2 to 8 carbon atoms and cycloalkynyl containing 5 to 8 carbon atoms which may optionally be substituted preferably by 1 to 3 of the same or different substituents. Reference is made to the foregoing definition of the optionally substituted alkyl containing more than one carbon atom with respect to the definition of the optionally substituted alkynyl, the optionally substituted alkynes comprising at least one C≡C triple bond. Examples include: ethynyl, propynyl, butynyl, pentynyl and optionally substituted variants thereof, as defined above. Ethynyl and optionally substituted ethynyl are preferred.

Throughout the invention, optionally substituted aryl preferably includes: aromatic hydrocarbon residues containing 6 to 14 carbon atoms (excluding the carbon atoms of the possible substituents), which may be monocyclic or bicyclic and may be substituted preferably by 1 to 3 of the same or different substituents selected from hydroxy, halogen, as defined above, cyano, optionally substituted amino, as defined below, mercapto, optionally substituted alkyl, as defined above, optionally substituted acyl, as defined below, and optionally substituted alkoxy, as defined above, optionally substituted aryloxy, as defined above, optionally substituted heterocyclyloxy, as defined above, optionally substituted aryl, as defined herein, optionally substituted heterocyclylyl, as defined below. Aromatic hydrocarbon residues containing 6 to 14 carbon atoms, include, for example: phenyl, naphthyl, phenanthrenyl and anthracenyl, which may optionally be singly or multiply substituted by the same or different residues. Optionally substituted phenyl is preferred, such as halogen-substituted phenyl.

Examples of an alkyl-substituted aryl group preferably include: aryl, as described above which is substituted by straight-chain or branched alkyl containing 1 to 8, preferably 1 to 4 carbon atoms, as described above. Toluyl is the preferred alkylaryl.

Examples of a hydroxy-substituted aryl group preferably include: aryl, as described above, which is substituted by 1 to 3 hydroxyl residues such as, for example 2-hydroxyphenyl, 3-hydroxyphenyl, 4-hydroxyphenyl, 2,4-di-hydroxyphenyl, 2,5-di-hydroxyphenyl, 2,6-di-hydroxyphenyl, 3,5-di-hydroxyphenyl, 3,6-di-hydroxyphenyl, 2,4,6-tri-hydroxyphenyl, etc. 2-hydroxyphenyl, 3-hydroxyphenyl and 2,4-di-hydroxyphenyl are preferred.

Examples of a halogen-substituted aryl group preferably include: aryl, as described above, which is substituted by 1 to 3 halogen atoms such as, for example 2-chloro- or fluorophenyl, 3-chloro- or fluorophenyl, 4-chloro- or fluorophenyl, 2,4-di-(chloro- and/or fluoro)phenyl, 2,5-di-(chloro- and/or fluoro)phenyl, 2,6-di-(chloro- and/or fluoro)phenyl, 3,5-di-(chloro- and/or fluoro)phenyl, 3,6-di-(chloro- and/or fluoro)phenyl, 2,4,6-tri-(chloro- and/or fluoro)phenyl, etc. 2-fluorophenyl, 3-fluorophenyl and 2,4-di-fluorophenyl are preferred.

Examples of an alkoxy-substituted aryl group preferably include: aryl, as described above, which is substituted by 1 to 3 alkoxy residues, as described above, such as preferably 2-methoxyphenyl, 3-methoxyphenyl, 4-methoxyphenyl, 2-ethoxyphenyl, 3-ethoxyphenyl, 4-ethoxyphenyl, 2,4-di-methoxyphenyl, etc.

Examples of a hydroxy- and alkoxy-substituted aryl group preferably include: aryl, as described above which is substituted by 1 to 2 alkoxy residues, as described above, and by 1 to 2 methoxy residues, as described above. 2-hydroxy-5-methoxyphenyl is preferred.

Throughout the invention, optionally substituted heterocyclyl preferably includes: Aliphatic, saturated or unsaturated heterocyclic 5- to 8-membered cyclic residues containing 1 to 3, preferably 1 to 2 hetero atoms, selected from N, O or S and which may optionally be substituted preferably by 1 to 3 substituents, wherein reference may be made to the definition of possible alkyl substituents with respect to possible substituents, 5- or 6-membered saturated or unsaturated, optionally substituted heterocyclic residues are preferred, such as tetrahydrofuran-2-yl, tetrahydrofuran-3-yl, tetrahydro-thiophen-2-yl, tetrahydro-thiophen-3-yl, pyrrolidin-1-yl, pyrrolidin-2-yl, pyrrolidin-3-yl, morpholin-1-yl, morpholin-2-yl, morpholin-3-yl, piperidin-1-yl, piperidin-2-yl, piperidin-3-yl, piperidin-4-yl, piperazin-1-yl, piperazin-2-yl, tetrahydropyran-2-yl, tetrahydropyran-3-yl, tetrahydropyran-4-yl, etc., which may optionally be condensed with aromatic rings.

Throughout the invention, optionally substituted heterocyclyl also includes heteroaromatic hydrocarbon residues containing 4 to 9 ring carbon atoms, which additionally preferably contain 1 to 3 of the same or different heteroatoms from the series S, O, N in the ring and therefore preferably form 5- to 12-membered heteroaromatic residues which may preferably be monocyclic but also bicyclic. Preferred aromatic heterocyclic residues include: pyridinyl, such as pyridin-2-yl, pyridin-3-yl and pyridin-4-yl, pyridyl-N-oxide, pyrimidyl, pyridazinyl, pyrazinyl, thienyl, furyl, pyrrolyl, pyrazolyl, imidazolyl, thiazolyl, oxazolyl or isoxazolyl, indolizinyl, indolyl, benzo[b]thienyl, benzo[b]furyl, indazolyl, quinolyl, isoquinolyl, naphthyridinyl, quinazolinyl, 5-membered or 6-membered aromatic heterocycles such as, for example, pyridinyl, in particular pyridin-2-yl, pyridyl-N-oxide, pyrimidyl, pyridazinyl, furyl and thienyl are preferred.

The heterocyclyl residues according to the invention may be substituted, preferably by 1 to 3 of the same or different substituents selected, for example, from hydroxy, halogen, as defined above, cyano, amino, as defined below, mercapto, alkyl, as defined above, acyl, as defined below, and alkoxy, as defined above, aryloxy, as defined above, heterocyclyloxy, as defined above, aryl, as defined above, heterocyclyl, as defined herein.

Heterocyclyl preferably includes: tetrahydrofuranyl, pyrrolidinyl, morpholinyl, piperidinyl or tetrahydropyranyl, pyridinyl, pyridyl-N-oxide, pyrimidyl, pyridazinyl, pyrazinyl, thienyl, furanyl, pyrrolyl, pyrazolyl, imidazolyl, thiazolyl, oxazolyl or isoxazolyl, indolizinyl, indolyl, benzo[b]thienyl, benzo[b]furyl, indazolyl, quinolyl, isoquinolyl, naphthyridinyl, quinazolinyl, quinoxazolinyl. 5-membered or 6-membered heterocycles such as, for example, morpholinyl and aromatic heterocycles such as, for example, pyridyl, pyridyl-N-oxide, pyrimidyl, pyridazinyl, furanyl and thienyl, as well as quinolyl and isoquinolyl are preferred. Morpholinyl, pyridyl, pyrimidyl and furanyl are preferred. The particularly preferred heterocyclyl includes: morpholinyl, pyridyl, such as pyridin-2-yl, pyridin-3-yl, pyridin-4-yl, pyrimidinyl, such as pyrimidin-2-yl and pyrimidin-5-yl, pyrazin-2-yl, thienyl, such as thien-2-yl and thien-3-yl as well as furanyl, such as furan-2-yl and furan-3-yl.

Examples of an alkyl-substituted heterocyclyl group preferably include: heterocyclyl, as described above, which is substituted by straight-chain or branched, optionally substituted alkyl containing 1 to 8, preferably 1 to 4 carbon atoms, as described above. Methylpyridinyl, trifluoromethylpyridinyl, in particular 3- or 4-trifluoromethylpyridin-2-yl, methylfuryl, methylpyrimidyl, methylpyrrolyl and methylquinolinyl, in particular 2-methylquinolin-6-yl are preferred:

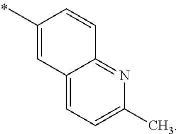

Examples of a hydroxy-substituted heterocyclyl group preferably include: heterocyclyl, as described above, which is substituted by 1 to 3 hydroxyl residues such as, for example 3-hydroxypyridyl, 4-hydroxypyridyl 3-hydroxyfuryl, 2-hydroxypyrimidyl 5-hydroxypyrimidyl, 3-hydroxypyrrolyl, 3,5-di-hydroxypyridyl, 2,5-di-hydroxypyrimidyl, etc.

Examples of an alkoxy-substituted heterocyclyl group preferably include: heterocyclyl, as described above, which is substituted by 1 to 3 alkoxy residues, as described above, such as, preferably 3-alkoxypyridyl, 4-alkoxypyridyl 3-alkoxyfuryl, 2-alkoxypyrimidyl 5-alkoxypyrimidyl, 3-alkoxypyrrolyl, 3,5-di-alkoxypyridin-2-yl, 2,5-di-alkoxypyrimidyl, etc.

Optionally substituted acyl here and hereinafter includes: formyl (—CH(═O)), optionally substituted aliphatic acyl (alkanoyl=alkyl-CO, wherein reference may be made to the foregoing definition of optionally substituted alkyl with respect to the alkyl group), optionally substituted aromatic acyl (aroyl=aryl-CO—, wherein reference may be made to the foregoing definition of optionally substituted aryl with respect to the aryl group) or heterocyclic acyl (heterocycloyl=heterocyclyl-CO—, wherein reference may be made to the foregoing definition of optionally substituted heterocyclyl with respect to the heterocyclyl group). Heteroaromatic acyl=heteroaryl-CO— is preferred.

Optionally substituted aliphatic acyl (alkanoyl) preferably includes: C₁ to C₆ alkanoyl, such as formyl, acetyl, propionyl, butyryl, isobutyryl, valeryl, isovaleryl, pivaloyl, hexanoyl, etc.

Examples of substituted aliphatic acyl include, for example: optionally aryl-substituted or heterocyclyl-substituted C₂ to C₆ alkanoyl, wherein reference may be made to the foregoing definitions of aryl, with respect to aryl, heterocyclyl and C₂ to C₆ alkanoyl, such as phenylacetyl, thiophen-2-yl-acetyl, thiophen-3-yl-acetyl, furan-2-yl-acetyl, furan-3-yl-acetyl, 2- or 3-phenylpropionyl, 2- or 3-thiophen-2-yl-propionyl, 2- or 3-thiophen-3-yl-propionyl, 2- or 3-furan-2-yl-propionyl, 2- or 3-furan-3-yl-propionyl, preferably thiophen-2-yl-acetyl.

Optionally substituted aromatic acyl (aroyl) includes: C₆ to C₁₀ aroyl, such as benzoyl, toluoyl, xyloyl, etc.

Optionally substituted heteroaromatic acyl (heteroaroyl) includes, in particular: C₆ to C₁₀ hetaroyl, such as furanoyl, pyridinoyl, etc.

Throughout the invention, optionally substituted amino preferably includes: amino, mono- or dialkylamino, mono- or diarylamino, (n-alkyl)(n-aryl)amino, mono- or diheterocyclylamino, (n-alkyl)(n-heterocyclyl)amino, (n-aryl)(n-heterocyclyl)amino, mono- or diacylamino, etc., wherein reference may be made to the corresponding foregoing definition of optionally substituted alkyl, optionally substituted aryl, optionally substituted heterocyclyl and optionally substituted acyl, with respect to alkyl, aryl, heterocyclyl and acyl, and substituted alkyl preferably includes aryl- or heterocyclyl-substituted alkyl in this case.

Mono- or dialkylamino includes, in particular: straight-chain or branched mono- or dialkylamino containing 1 to 8, preferably 1 to 4 saturated or unsaturated carbon atoms, optionally substituted as described above, in each alkyl group, in particular methylamino, dimethylamino, ethylamino, wherein the alkyl groups may be substituted preferably by one substituent.

Mono- or diarylamino includes, in particular: mono- or diarylamino with 3- to 8-, preferably 5- to 6-membered aryl residues, optionally substituted as described above, in particular phenylamino or diphenylamino, wherein the aryl groups may optionally be substituted by one or two substituents.

(N-alkyl)(N-aryl)amino describes in particular a substituted amino which is substituted in each case at the nitrogen atom by an alkyl residue and by an aryl residue, in particular, (N-methyl)(N-phenyl)amino.

Mono- or diheterocyclylamino includes, in particular: mono- or diheterocyclylamino with 3- to 8-, preferably 5- to 6-membered heterocyclyl residues, optionally substituted as described above, in particular pyridylamino or dipyridylamino.

(N-alkyl)(N-heterocyclyl)amino describes, in particular, a substituted amino which is substituted in each case at the nitrogen atom by an alkyl residue and by a heterocyclyl residue.

(N-alkyl)(N-heterocyclyl)amino describes, in particular, a substituted amino which is substituted in each case at the nitrogen atom by an aryl residue and by a heterocyclyl residue.

Mono- or diacylamino includes, in particular, a substituted amino which is substituted by one or two acyl residues.

Reference may be made to the corresponding foregoing definitions of optionally substituted alkyl, optionally substituted aryl and optionally substituted heterocyclyl and optionally substituted acyl, with respect to alkyl, aryl, heterocyclyl and acyl.

Optionally substituted amino further includes a preferably substituted methylene amino group:

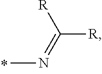

wherein R in this case is an organic group and/or hydrogen respectively, in particular R⁶ and R⁷, as defined below. In this case, R is preferably hydrogen and/or an optionally substituted alkyl-, aryl- or heterocyclyl group, which is as defined above in each case. In this case, it is particularly preferred if R is hydrogen and an optionally substituted aryl group or R is an optionally substituted alkyl group and an optionally substituted aryl group such as, for example:

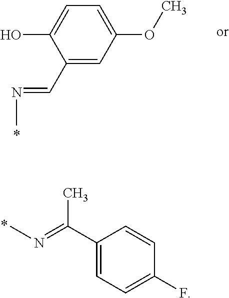

In the meaning of R⁵, the optionally substituted amino group, as described above, together with the nitrogen atom to which is it bound, preferably forms an optionally substituted hydrazine group (—NH—NH₂), such as hydrazinyl, an optionally substituted mono- or dialkylhydrazinyl group (—NH—NHR or —NH—NR₂), such as optionally substituted methylhydrazine, methylenehydrazine (—NH—N═CR₂), ethylhydrazine, propylhydrazine, etc. or (optionally substituted) aryl- and/or heterocyclylhydrazinyl such as, for example (optionally substituted) phenylhydrazine (—NH—NH-phenyl).

Optionally substituted amino groups are particularly preferred: amino, diphenylamino, (N-methyl)(N-phenyl)amino as well as amino groups of the formula

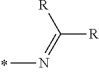

as defined above, preferably those in which R represents hydrogen, an optionally substituted alkyl group or an optionally substituted aryl group in this case, in particular:
2-hydroxy-phenyl-meth-(E or Z)-ylidene]-amino:

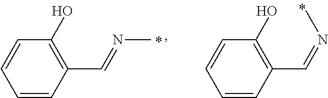

(3-hydroxy-phenyl)-meth-(E or Z)-ylidene]-amino:

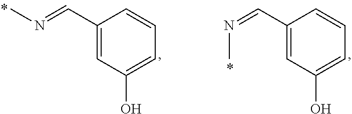

1-(2,4-dihydroxy-phenyl)-meth-(E or Z)-ylidene]-amino

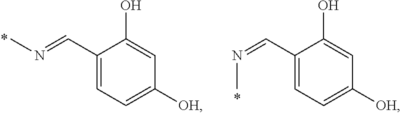

1-(2-hydroxy-5-methoxy-phenyl)-meth-(E or Z)-ylidene]-amino:

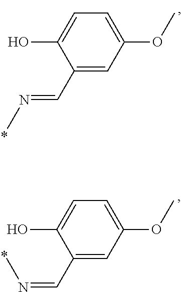

1-(4-fluorophenyl)-eth-(E or Z)-ylideneamino:

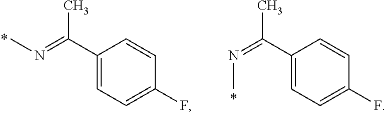

Throughout the invention, optionally substituted aminocarbonyl represents optionally substituted amino-CO—, wherein reference may be made to the foregoing definition with respect to the definition of optionally substituted amino. Optionally substituted aminocarbonyl preferably represents optionally substituted carbamoyl (H₂NCO—), such as H₂NCO—, mono- or dialkylaminocarbonyl (H(alkyl)N—CO— or (alkyl)₂N—CO—), mono- or diarylaminocarbonyl (H(aryl)N—CO— or (aryl)₂N—CO—) or mono- or diheterocyclylaminocarbonyl (H(heterocyclyl)N—CO— or (heterocyclyl)₂N—CO—), wherein reference may be made to the foregoing explanations of optionally substituted alkyl, aryl or heterocyclyl with respect to the definition of alkyl, aryl or heterocyclyl.

Throughout the invention, optionally substituted aminosulfonyl represents optionally substituted amino-SO₂—, wherein reference may be made to the foregoing definition with respect to the definition of optionally substituted amino. Optionally substituted sulfamoyl (H₂N—SO₂—), such as sulfamoyl (H₂N—SO₂—) or mono- or dialkylaminosulfonyl (alkyl)₂N—SO₂— are preferred, wherein reference may be made to the foregoing explanations of optionally substituted alkyl, with respect to the definition of alkyl.

Optionally substituted alkyl-, aryl- or heterocyclylsulfonyl (R—SO₂—, wherein R is optionally substituted alkyl, optionally substituted aryl or optionally substituted heterocyclyl, each as defined above) further preferably represents methylsulfonyl, ethylsulfonyl, phenylsulfonyl, tolylsulfonyl or benzylsulfonyl.

Optionally substituted alkoxycarbonyl (RO(O═)C—) includes the above-mentioned optionally substituted alkoxy, with respect to the definition of alkoxy.

Optionally substituted acyloxyl (R—C(═O)—O—) includes the above-mentioned optionally substituted acyl, with respect to the definition of acyl.

### Preferred Embodiments

In a preferred embodiment, the compound of formula (I) has the following definitions of substituents:

X has the meaning N or C—R¹, wherein
R¹ is selected from the group consisting of:
hydrogen,
halogen,
optionally substituted amino,
optionally substituted alkyl,
optionally substituted alkoxy
optionally substituted aryl,
optionally substituted heterocyclyl;
   R² and R³ are the same or different and are each selected from the group consisting of:
hydrogen,
halogen,
optionally substituted amino,
optionally substituted alkyl,
optionally substituted alkoxy,
optionally substituted aryl,
optionally substituted heterocyclyl;
   R⁴ and R⁵ are the same or different and are each selected from the group consisting of:
hydrogen,
optionally substituted amino,
optionally substituted alkyl,
optionally substituted aryl,
optionally substituted heterocyclyl or
R⁴ and R⁵ together with the nitrogen atom, to which they are bound, form a saturated or unsaturated, optionally substituted 5- to 6-membered ring, which can optionally contain further heteroatoms.

In a further more preferred embodiment, the compound of formula (I) has the following definitions of substituents:

X has the meaning N or C—R¹, wherein
R¹ is selected from the group consisting of:
hydrogen,
halogen,
optionally substituted alkyl,
optionally substituted alkoxy
optionally substituted aryl,
optionally substituted heterocyclyl;
   R² and R³ are the same or different and are each selected from the group consisting of:
hydrogen,
halogen,
optionally substituted amino,
optionally substituted alkyl,
optionally substituted aryl,
optionally substituted heterocyclyl;
   R⁴ and R⁵ are the same or different and are each selected from the group consisting of:
hydrogen,
optionally substituted amino,
optionally substituted alkyl,
optionally substituted aryl,
optionally substituted heterocyclyl or
R⁴ and R⁵ together with the nitrogen atom, to which they are bound, form a saturated or unsaturated, optionally substituted 5- to 6-membered ring, which can optionally contain one to two further heteroatoms.

In a further more preferred embodiment, the compound of formula (I) has the following definitions of substituents:

X has the meaning N or C—R¹, wherein
R¹ is selected from the group consisting of:
hydrogen,
halogen,
optionally substituted alkyl,
optionally substituted alkoxy,
   R² and R³ are the same or different and are selected from the group consisting of:
hydrogen,
optionally substituted amino,
optionally substituted alkyl,
optionally substituted heterocyclyl,
   R⁴ and R⁵ are the same or different and are each selected from the group consisting of:
hydrogen,
optionally substituted amino,
optionally substituted alkyl;
optionally substituted heterocyclyl; or
R⁴ and R⁵ together with the nitrogen atom, to which they are bound, form a saturated or unsaturated, optionally substituted 5- to 6-membered ring, which can optionally contain one to two further heteroatoms.

In further preferred embodiments of general formulae (I) and (I′), the individual substituents have the following definitions in each case:
1. Y has the meaning of —NR⁴R⁵.
2. X has the meaning of N and R², R³, R⁴ and R⁵ have the meaning of one of the above-described embodiments.
3. X has the meaning C—R¹ and R¹ is selected from the group consisting of:
   hydrogen,
   halogen,
   optionally substituted alkyl,
   optionally substituted alkoxy,
   and R², R³, R⁴ and R⁵ have the meaning of one of the above-described embodiments.
4. R² and R³ are the same or different and are selected from the group consisting of:
   hydrogen,
   optionally substituted amino,
   optionally substituted alkyl,
   optionally substituted heterocyclyl,
   and X, R¹, R⁴ and R⁵ have the meaning of one of the above-described embodiments.
5. R⁴ and R⁵ are the same or different and are each selected from the group consisting of:
   hydrogen,
   optionally substituted amino;
   optionally substituted alkyl;
   optionally substituted heterocyclyl; or
   R⁴ and R⁵ together with the nitrogen atom, to which they are bound, form a saturated or unsaturated, optionally substituted 5- to 6-membered ring, which can optionally contain one to two further heteroatoms
   and X, R¹, R² and R³ have the meaning of one of the above-described embodiments.

In preferred embodiments of general formula (I), the individual substituents have the following definitions in each case:

X represents N or C—R¹, wherein R¹ is selected from the group consisting of:
hydrogen,
halogen, in particular chlorine,
optionally substituted alkyl, in particular straight-chain or branched alkyl, as defined above, in particular preferably methyl, and which may optionally be substituted by (optionally substituted, for example alkyl-, halogen- and/or alkoxy-substituted) aryl, as defined above, in particular alkyl substituted by optionally alkyl-, halogen- and/or alkoxy-substituted aryl, such as benzyl, halogen-, alkyl- and/or alkoxy-substituted benzyl, such as, for example,

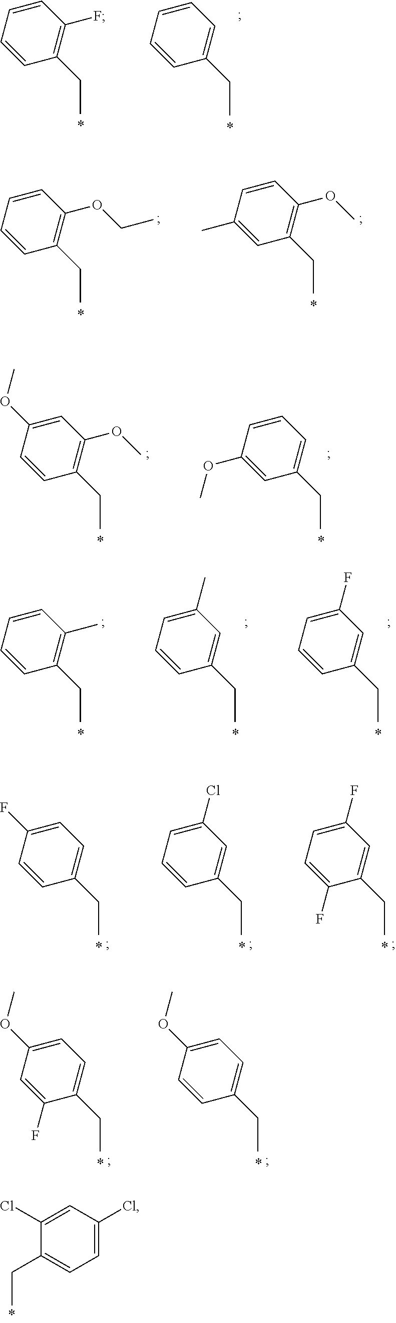

preferably 2-fluorophenylmethyl:

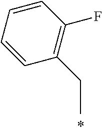

(* here and hereinafter denotes the respective binding position of the residue in this case of R¹);
or
optionally substituted alkoxy, such as isopropoxy, methoxy, in particular methoxy,
   R² is selected from the group consisting of:
hydrogen,
hydroxy,
halogen, such as chlorine,
optionally substituted alkyl, in particular, straight-chain or branched alkyl, as defined above, which may optionally be substituted, as described above, methyl in particular being preferred;
optionally substituted alkoxy, in particular, alkoxy substituted by optionally substituted aryl, such as

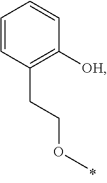

optionally substituted amino, such amino, mono- or dialkylamino, such as isopropylamino, in particular amino (—NH₂);
optionally substituted heterocyclyl, in particular aliphatic heterocyclyl, as described above, in which morpholinyl, in particular morpholinyl-4-yl:

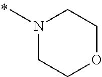

is preferred
   R³ is selected from the group consisting of:
hydrogen,
optionally substituted alkyl, in particular straight-chain or branched alkyl, as defined above, which may optionally be substituted, as described above, such as aminomethyl and methyl, methyl in particular being preferred;
optionally substituted amino, in particular diarylamino, wherein aryl may optionally be substituted, as described above, diphenylamino being preferred, or (N-alkyl)(N-aryl)amino, wherein alkyl and aryl may optionally be substituted, as described above, (N-methyl)(N-phenyl)amino being preferred;
or
optionally substituted aryl, such as phenyl
optionally substituted heterocyclyl, in particular aliphatic heterocyclyl, as described above, in which morpholinyl, in particular morpholinyl-4-yl:

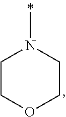

is preferred, or optionally substituted unsaturated and/or aromatic heterocyclyl, as described above, such as optionally substituted in particular nitrogen-containing heterocyclyl, such as

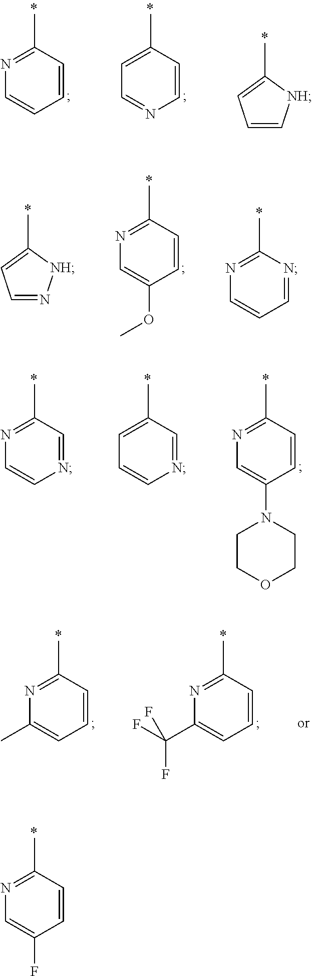

in which pyridinyl, in particular 2-pyridinyl

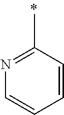

is particularly preferred;
   R⁴ and R⁵ are the same or different and represent:
hydrogen (preferably either R⁴ or R⁵ is hydrogen, or both are hydrogen),
optionally substituted alkyl, in particular straight-chain, branched and/or cyclic alkyl, as defined above, particularly preferably methyl, ethyl, n-propyl, isopropyl being particularly preferred

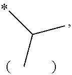

n-butyl, isobutyl

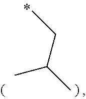

cyclopropylmethyl

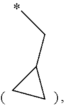

cyclohexylmethyl

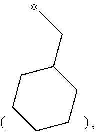

and which may optionally be substituted by (optionally substituted) amino, as defined above, in which in particular alkyl substituted by (optionally substituted) aryl- or heterocyclyl-substituted amino is preferred, in particular benzyl, phenethyl, phenylpropyl

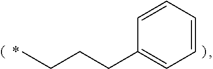

hydroxyphenethyl (such as

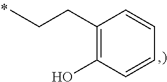

2-(5-trifluoromethyl-pyridin-2-ylamino)-ethyl:

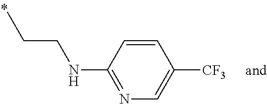

2-(4-trifluoromethyl-pyridin-2-ylamino)-ethyl:

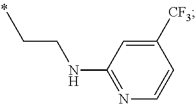

optionally substituted amino, such as an optionally substituted acylamino group, such as:

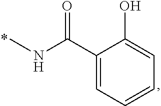

preferably a singly or doubly substituted methylene amino group:

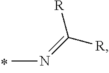

wherein R in this case is an organic group and/or hydrogen respectively, in particular R⁶ and R⁷, as defined below. R is preferably hydrogen and/or an optionally substituted alkyl-, aryl- or heterocyclyl group, which is as defined above in each case. In this case, it is particularly preferred if R is hydrogen and an optionally substituted aryl group or R is an optionally substituted alkyl group and an optionally substituted aryl group such as, for example:

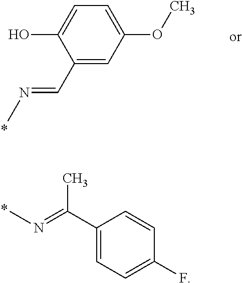

Particularly preferred optionally substituted amino groups for R⁵ are:
2-hydroxy-phenyl-meth-(E or Z)-ylidene]-amino:

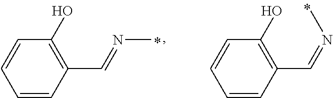

(3-hydroxy-phenyl-meth-(E or Z)-ylidene]-amino:

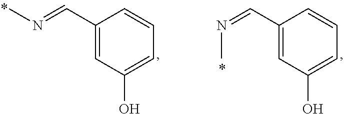

1-(2,4-dihydroxy-phenyl)-meth-(E or Z)-ylidene]-amino:

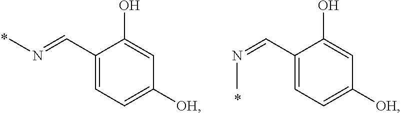

1-(2-hydroxy-5-methoxy-phenyl)-meth-(E or Z)-ylidene]-amino:

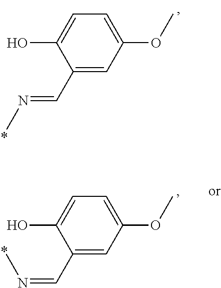

1-(4-fluorophenyl)-eth-(E or Z)-ylidene amino:

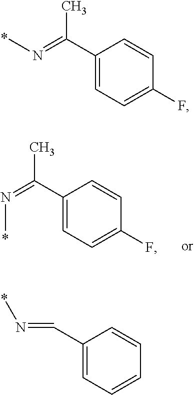

optionally substituted heterocyclyl, in particular aromatic heterocyclyl, as described above, in which in particular quinolyl or alkyl-substituted quinolyl such as 5-methylquinolyl is preferred;
optionally substituted acyl, in particular aliphatic or aromatic acyl, such as acetyl, benzoyl,
optionally substituted alkyl- or arylsulfonyl, methylsulfonyl, phenylsulfonyl,
optionally substituted aminocarbonyl, such as mono- or dialkyl and/or

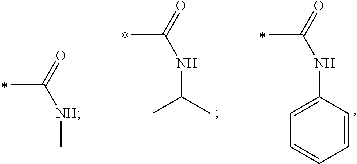

or
R⁴ and R⁵ together with the nitrogen atom, to which they are bound, form a saturated or unsaturated, optionally substituted 5- to 6-membered ring, which can optionally contain one to two further heteroatoms, in particular R⁴ and R⁵ preferably together with the nitrogen atom to which they are bound, form a saturated or unsaturated, such as an aromatic 5- to 6-membered heterocyclyl ring, in particular optionally substituted pyrazolyl, imidazolyl, triazolyl; piperidinyl, morpholinyl, piperazinyl, such as 4-methylpiperazinyl, pyrrolidinyl. It is particularly preferred that R⁴ and R⁵ together form residues of the formulae:

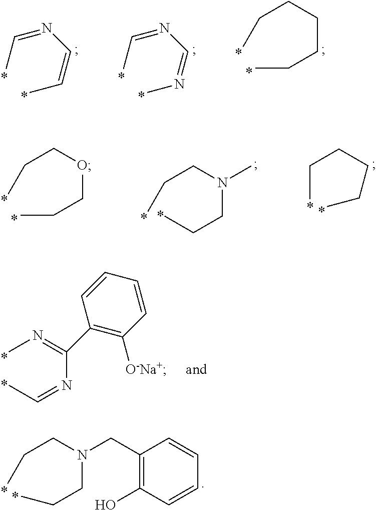

In a particularly preferred variant, R⁴ is hydrogen and R⁵ is isopropyl.

Particularly preferred compounds of general formula (I) are shown in the following table:

(I′)

| | | 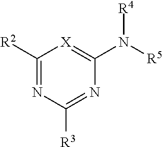 | | |
|---|---|---|---|---|
| Example | Compound | X | R¹ | R² |
| 1 | 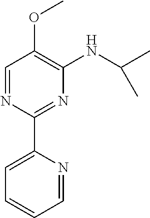 | C—R¹ | —OCH₃ | H |
| 2 | 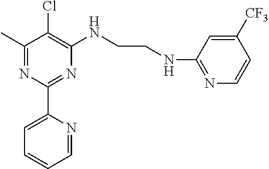 | C—R¹ | —Cl | —CH₃ |
| 3 | 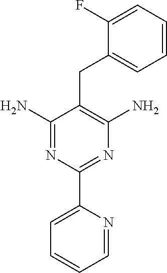 | C—R¹ | 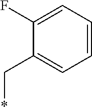 | —NH₂ |
| 4 | 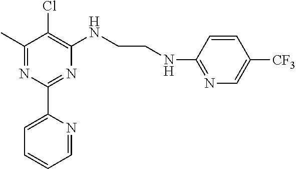 | C—R¹ | Cl | —CH₃ |
| 5 | 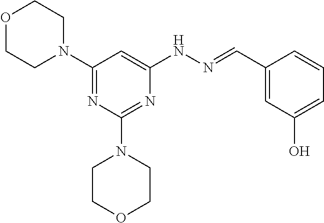 | C—R¹ | H | 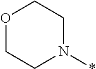 |
| 6 | 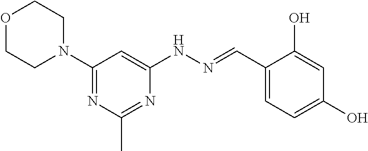 | C—R¹ | H | 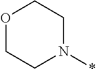 |
| 7 | 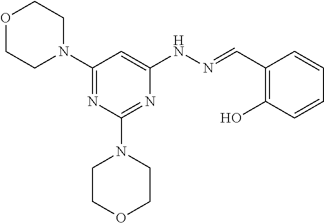 | C—R¹ | H | 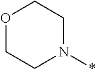 |
| 8 | 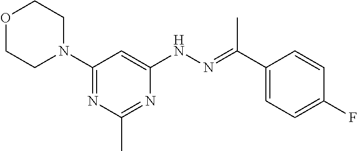 | C—R¹ | H | 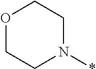 |
| 9 | 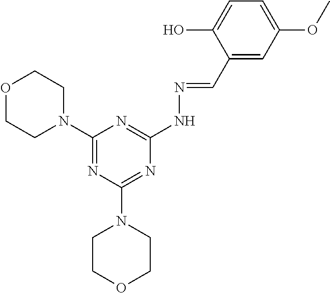 | N | — | 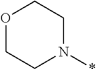 |
| 10 | 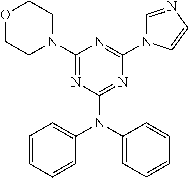 | N | — | 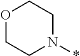 |
| 11 | 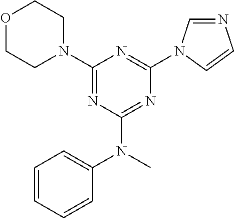 | N | — | 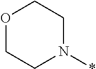 |
| 12 | 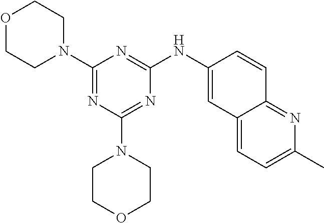 | N | — | 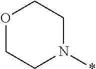 |
| 13 | 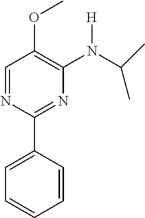 | CR¹ | —OCH₃ | H |
| 14 | 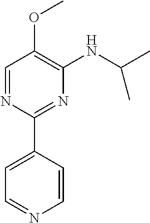 | CR¹ | —OCH₃ | H |
| 15 | 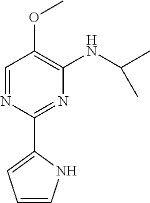 | CR¹ | —OCH₃ | H |
| 16 | 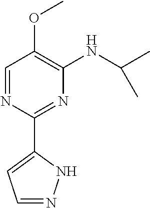 | CR¹ | —OCH₃ | H |
| 17 | 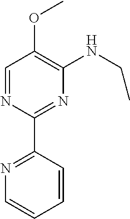 | CR¹ | —OCH₃ | H |
| 18 | 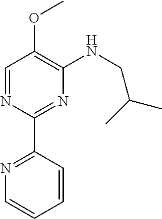 | CR¹ | —OCH₃ | H |
| 19 | 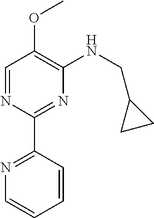 | CR¹ | —OCH₃ | H |
| 20 | 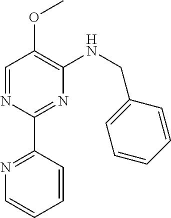 | CR¹ | —OCH₃ | H |
| 21 | 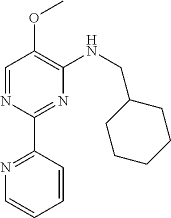 | CR¹ | —OCH₃ | H |
| 22 | 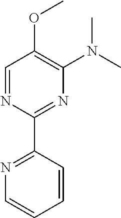 | CR¹ | —OCH₃ | H |
| 23 | 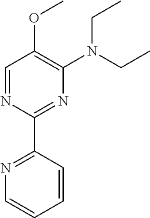 | CR¹ | —OCH₃ | H |
| 24 | 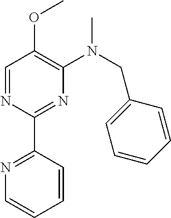 | CR¹ | —OCH₃ | H |
| 25 | 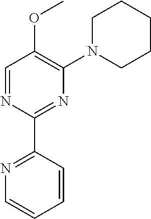 | CR¹ | —OCH₃ | H |
| 26 | 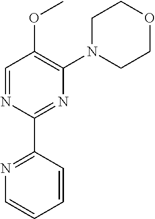 | CR¹ | —OCH₃ | H |
| 27 | 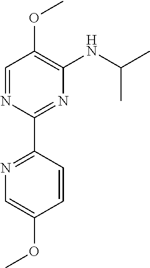 | CR¹ | —OCH₃ | H |
| 30 | 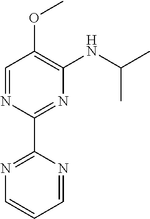 | CR¹ | —OCH₃ | H |
| 31 | 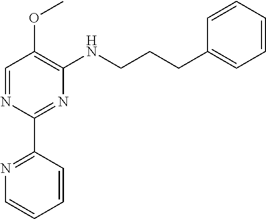 | CR¹ | —OCH₃ | H |
| 32 | 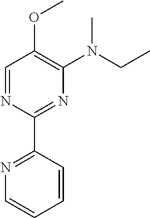 | CR¹ | —OCH₃ | H |
| 33 | 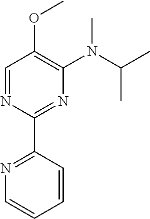 | CR¹ | —OCH₃ | H |
| 34 | 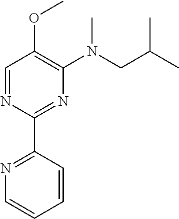 | CR¹ | —OCH₃ | H |
| 35 | 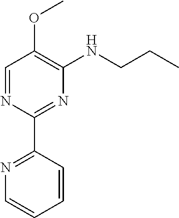 | CR¹ | —OCH₃ | H |
| 36 | 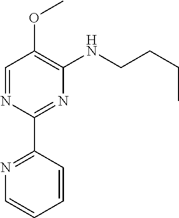 | CR¹ | —OCH₃ | H |
| 37 | 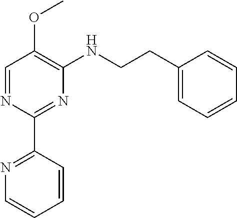 | CR¹ | —OCH₃ | H |
| 38 | 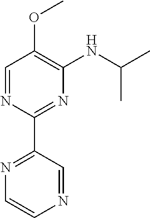 | CR¹ | —OCH₃ | H |
| 39 | 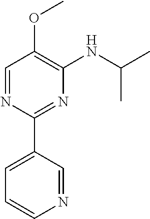 | CR¹ | —OCH₃ | H |
| 41 | 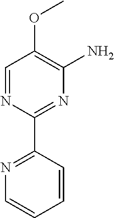 | CR¹ | —OCH₃ | H |
| 42 | 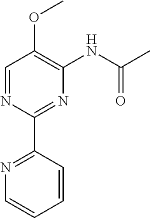 | CR¹ | —OCH₃ | H |
| 43 | 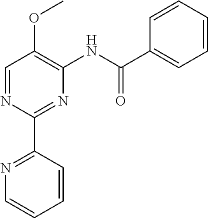 | CR¹ | —OCH₃ | H |
| 44 | 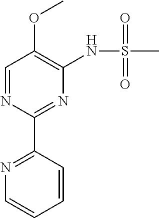 | CR¹ | —OCH₃ | H |
| 45 | 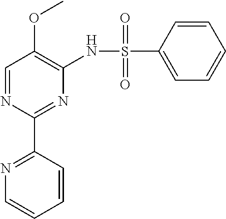 | CR¹ | —OCH₃ | H |
| 46 | 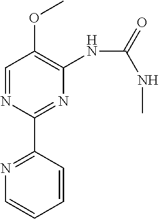 | CR¹ | —OCH₃ | H |
| 47 | 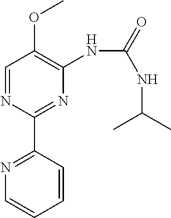 | CR¹ | —OCH₃ | H |
| 48 | 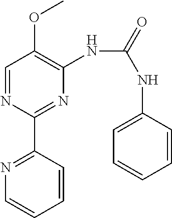 | CR¹ | —OCH₃ | H |
| 49 | 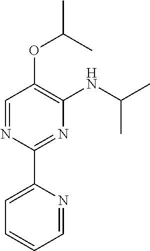 | CR¹ | 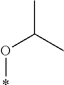 | H |
| 50 | 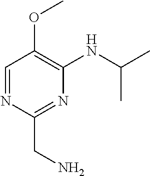 | CR¹ | —OCH₃ | H |
| 55 | 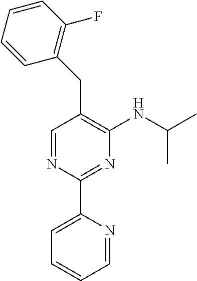 | CR¹ | 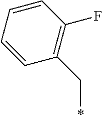 | H |
| 56 | 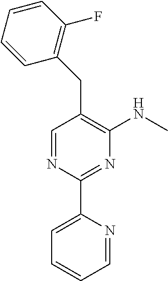 | CR¹ | 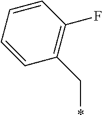 | H |
| 57 | 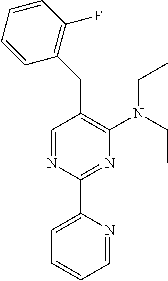 | CR¹ | 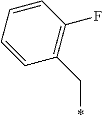 | H |
| 58 | 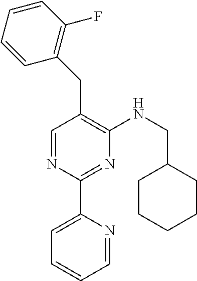 | CR¹ | 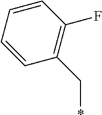 | H |
| 59 | 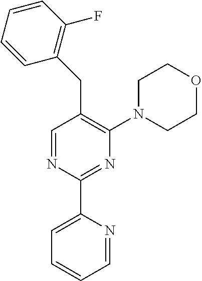 | CR¹ | 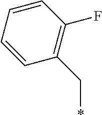 | H |
| 60 | 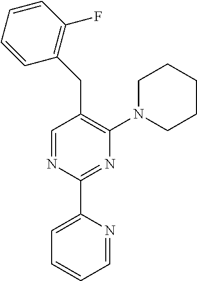 | CR¹ | 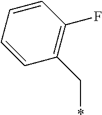 | H |
| 61 | 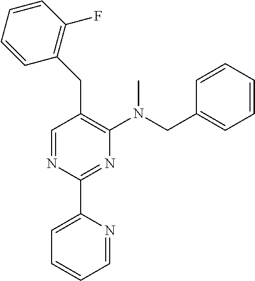 | CR¹ | 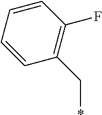 | H |
| 62 | 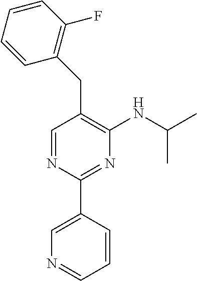 | CR¹ | 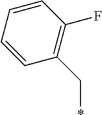 | H |
| 63 | 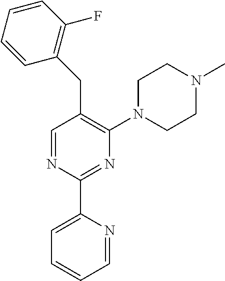 | CR¹ | 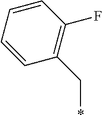 | H |
| 69 | 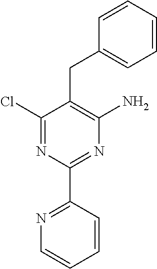 | CR¹ | 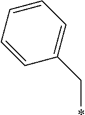 | Cl |
| 70 | 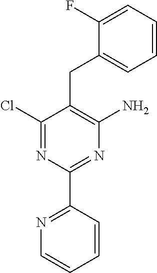 | CR¹ | 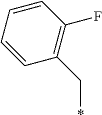 | Cl |
| 71 | 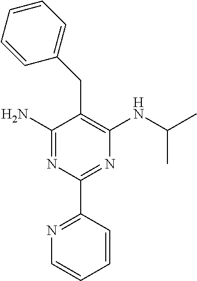 | CR¹ | 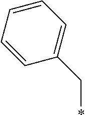 | —NH₂ |
| 72 | 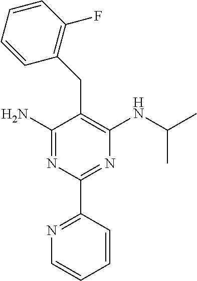 | CR¹ | 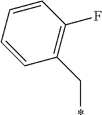 | —NH₂ |
| 73 | 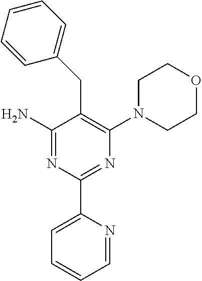 | CR¹ | 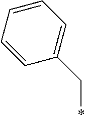 | —NH₂ |
| 74 | 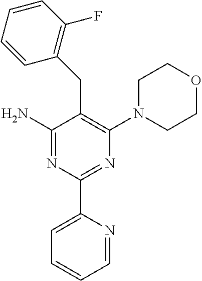 | CR¹ | 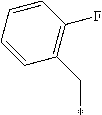 | —NH₂ |
| 75 | 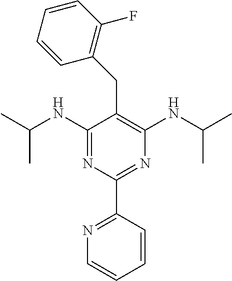 | CR¹ | 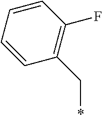 | 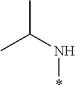 |
| 76 | 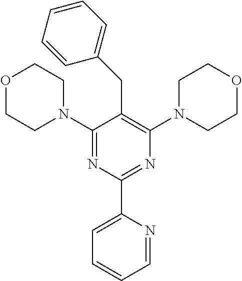 | CR¹ | 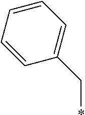 | 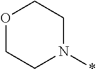 |
| 77 | 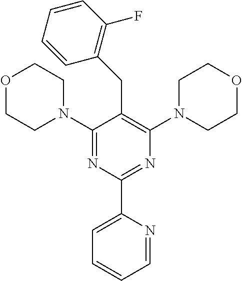 | CR¹ | 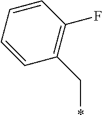 | 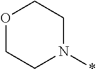 |
| 78 | 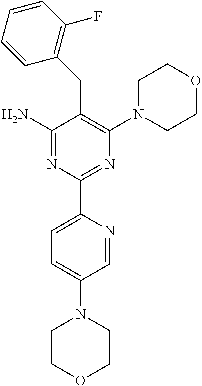 | CR¹ | 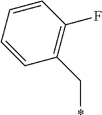 | —NH₂ |
| 79 | 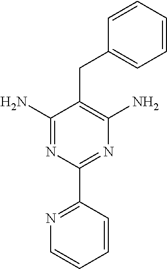 | CR¹ | 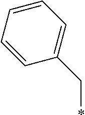 | —NH₂ |
| 80 | 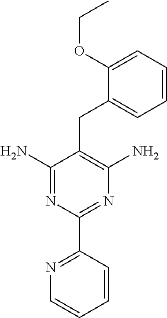 | CR¹ | 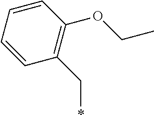 | —NH₂ |
| 81 | 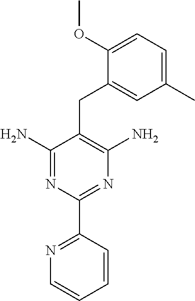 | CR¹ | 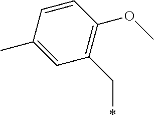 | —NH₂ |
| 82 | 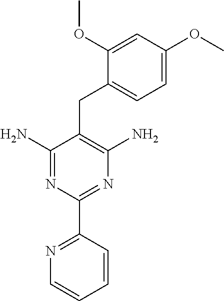 | CR¹ | 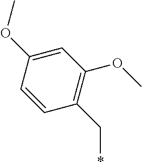 | —NH₂ |
| 83 | 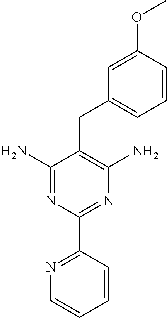 | CR¹ | 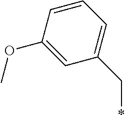 | —NH₂ |
| 84 | 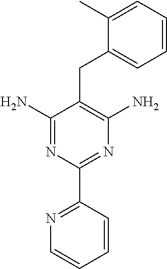 | CR¹ | 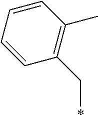 | —NH₂ |
| 85 | 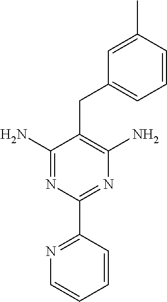 | CR¹ | 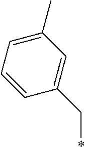 | —NH₂ |
| 86 | 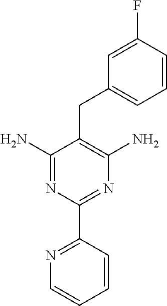 | CR¹ | 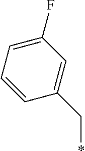 | —NH₂ |
| 87 | 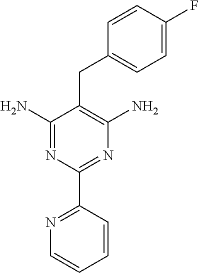 | CR¹ | 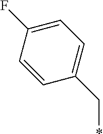 | —NH₂ |
| 88 | 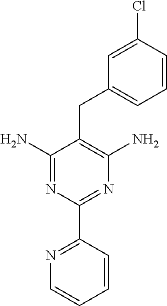 | CR¹ | 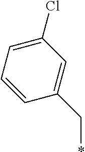 | —NH₂ |
| 89 | 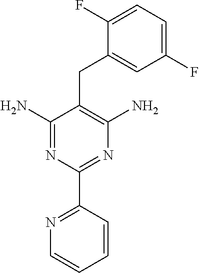 | CR¹ | 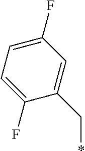 | —NH₂ |
| 90 | 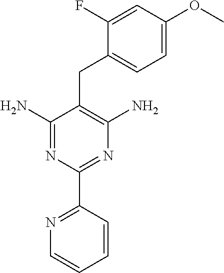 | CR¹ | 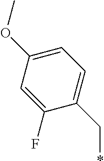 | —NH₂ |
| 91 | 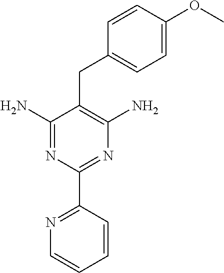 | CR¹ | 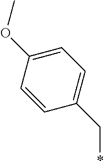 | —NH₂ |
| 92 | 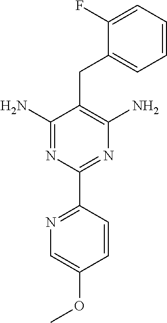 | CR¹ | 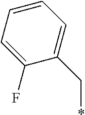 | —NH₂ |
| 93 | 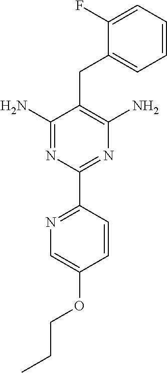 | CR¹ | 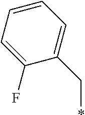 | —NH₂ |
| 94 | 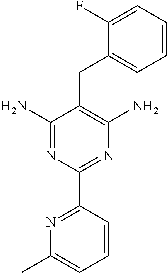 | CR¹ | 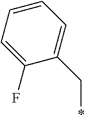 | —NH₂ |
| 95 | 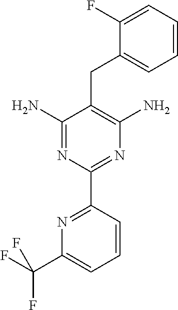 | CR¹ | 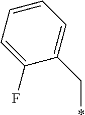 | —NH₂ |
| 97 | 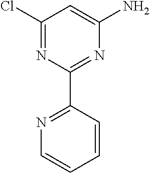 | CR¹ | H | —Cl |
| 98 | 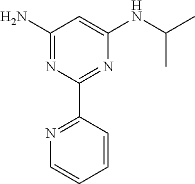 | CR¹ | H | —NH₂ |
| 99 | 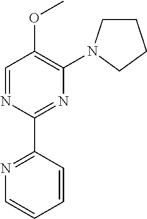 | CR¹ | —OCH₃ | H |
| 100 | 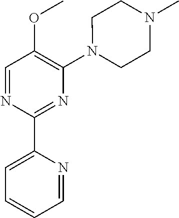 | CR¹ | —OCH₃ | H |
| 101 | 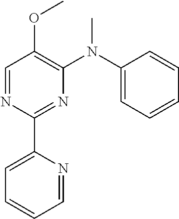 | CR¹ | —OCH₃ | H |
| 103 | 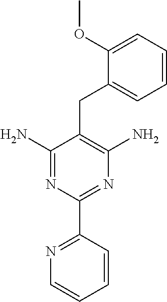 | C—R¹ | 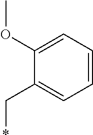 | —NH₂ |
| 104 | 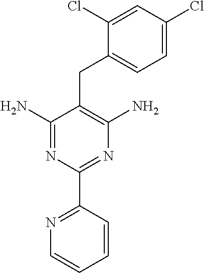 | C—R¹ | 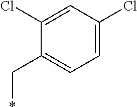 | —NH₂ |
| 105 | 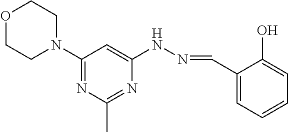 | C—R¹ | H | 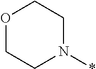 |
| 106 | 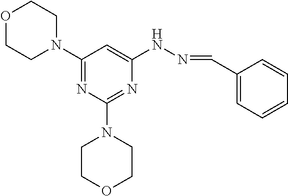 | C—R¹ | H | 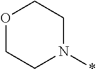 |
| 107 | 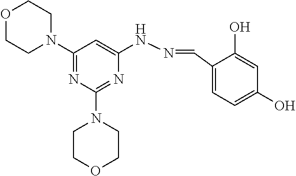 | C—R¹ | H | 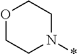 |
| 108 | 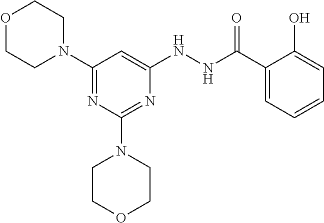 | C—R¹ | H | 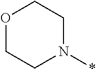 |
| 109 | 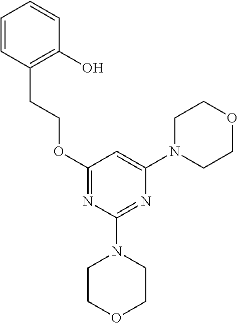 | C—R¹ | H | 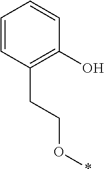 |
| 110 | 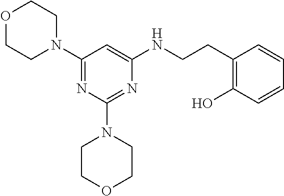 | C—R¹ | H | 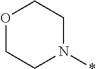 |
| 111 | 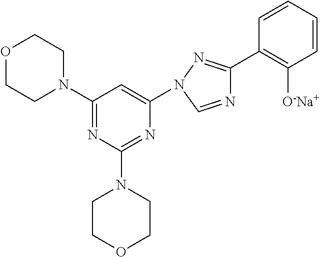 | C—R¹ | H | 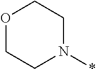 |
| 112 | 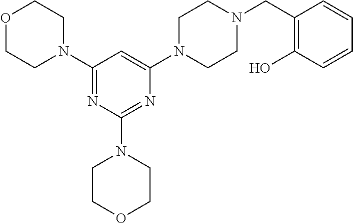 | C—R¹ | H | 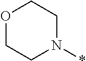 |
| 113 | 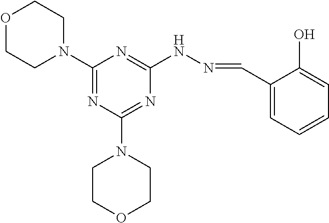 | N | — | 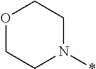 |
| 114 | 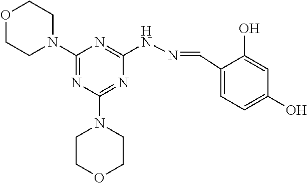 | N | — | 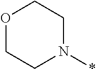 |
| 115 | 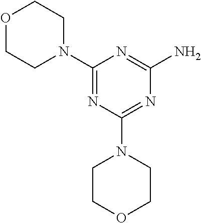 | N | — | 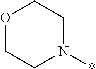 |
| 116 | 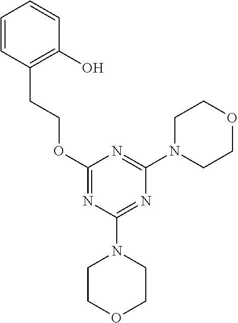 | N | — | 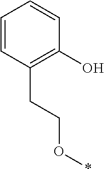 |
| 117 | 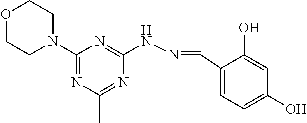 | N | — | 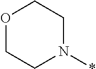 |
| | | (I) | | |
| | | 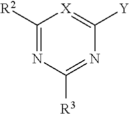 | | |
| Example | Compound | X | R¹ | R² |
| 28 | 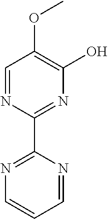 | C—R¹ | —OCH₃ | H |
| 29 | 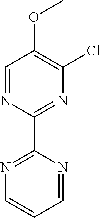 | C—R¹ | —OCH₃ | H |
| 40 | 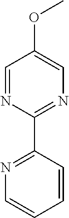 | C—R¹ | —OCH₃ | H |
| 51 | 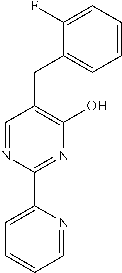 | C—R¹ | 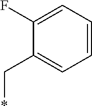 | H |
| 52 | 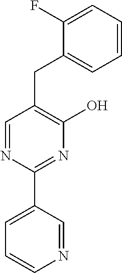 | C—R¹ | 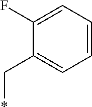 | H |
| 53 | 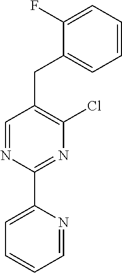 | C—R¹ | 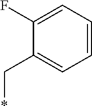 | H |
| 54 | 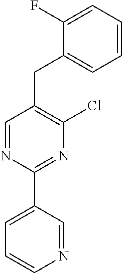 | C—R¹ | 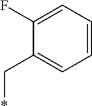 | H |
| 64 | 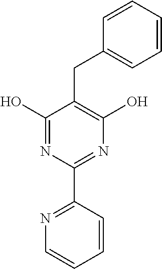 | C—R¹ | 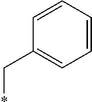 | —OH |
| 65 | 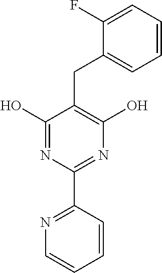 | C—R¹ | 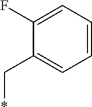 | —OH |
| 66 | 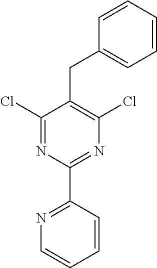 | C—R¹ | 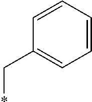 | —Cl |
| 67 | 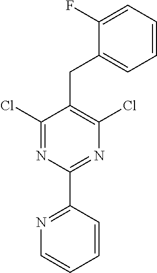 | C—R¹ | 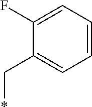 | —Cl |
| 68 | 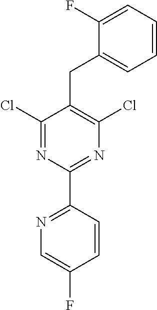 | C—R¹ | 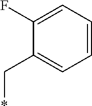 | —Cl |
| 96 | 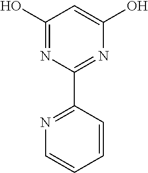 | C—R¹ | —H | —OH |
| 102 | 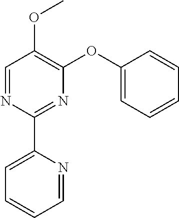 | C—R¹ | —OCH₃ | H |

(I′)

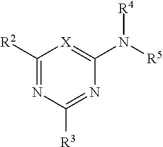

| Exam- | | | | |
|---|---|---|---|---|
| ple | Compound | R³ | R⁴ | R⁵ |
| 1 | 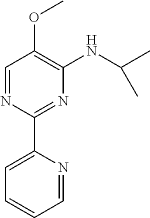 | 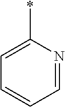 | H | 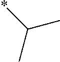 |
| 2 | 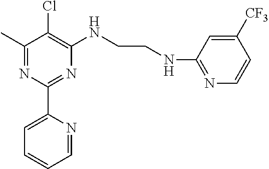 | 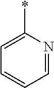 | H | 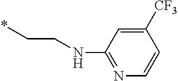 |
| 3 | 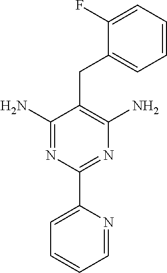 | 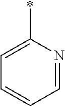 | H | H |
| 4 | 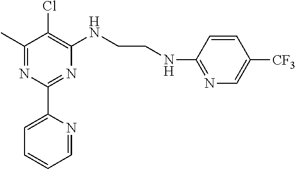 | 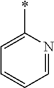 | H | 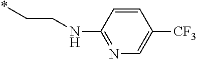 |
| 5 | 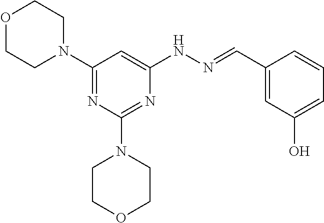 | 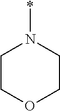 | H | 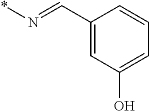 |
| 6 | 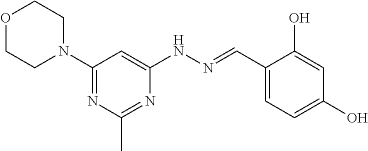 | —CH₃ | H | 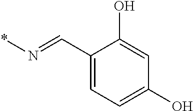 |
| 7 | 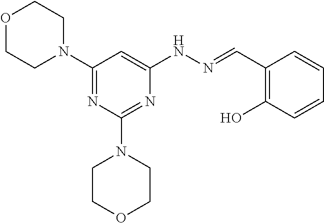 | 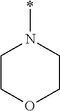 | H | 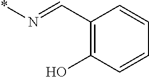 |
| 8 | 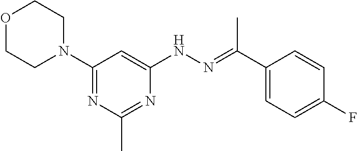 | —CH₃ | H | 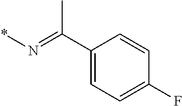 |
| 9 | 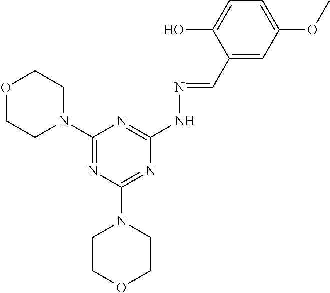 | 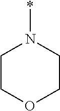 | H | 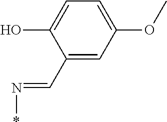 |
| 10 | 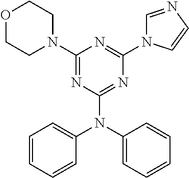 | 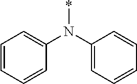 | 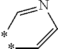 | |
| 11 | 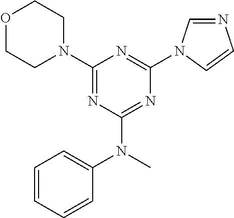 | 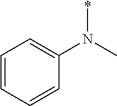 | 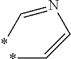 | |
| 12 | 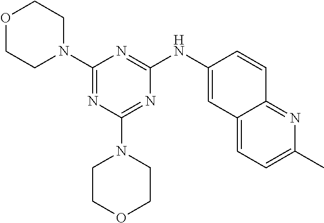 | 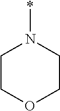 | H | 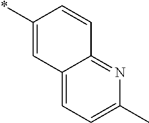 |
| 13 | 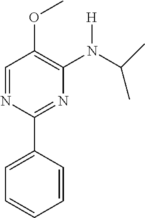 | Phenyl | H | 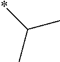 |
| 14 | 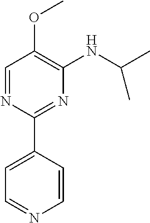 | 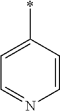 | H | 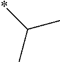 |
| 15 | 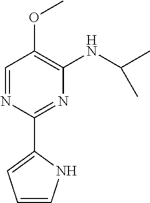 | 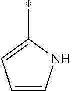 | H | 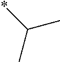 |
| 16 | 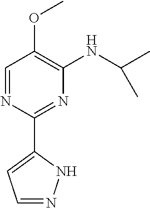 | 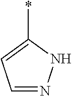 | H | 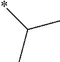 |
| 17 | 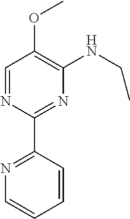 | 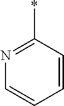 | H | 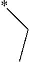 |
| 18 | 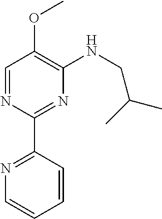 | 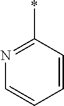 | H | 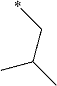 |
| 19 | 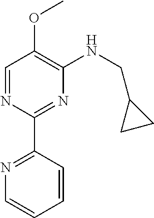 | 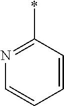 | H | 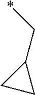 |
| 20 | 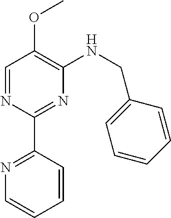 | 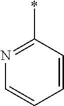 | H | 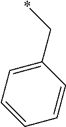 |
| 21 | 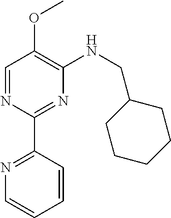 | 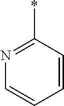 | H | 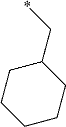 |
| 22 | 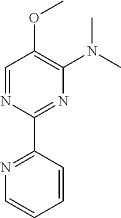 | 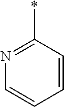 | —CH₃ | —CH₃ |
| 23 | 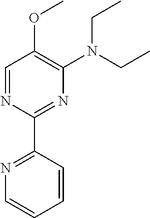 | 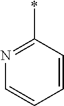 | Ethyl | Ethyl |
| 24 | 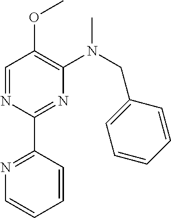 | 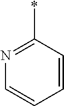 | Ethyl | Benzyl |
| 25 | 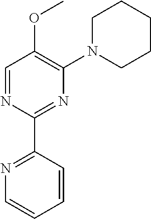 | 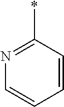 | 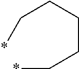 | |
| 26 | 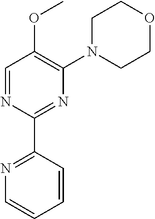 | 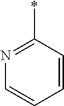 | 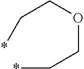 | |
| 27 | 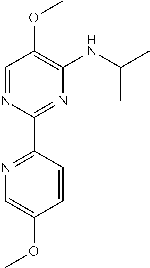 | 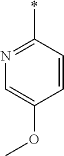 | H | 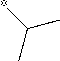 |
| 30 | 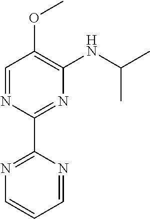 | 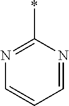 | H | 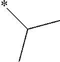 |
| 31 | 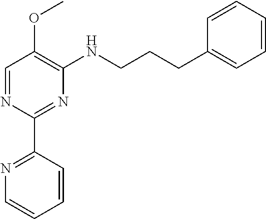 | 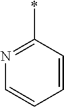 | H | 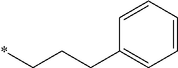 |
| 32 | 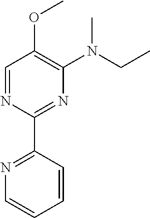 | 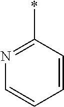 | —CH₃ | Ethyl |
| 33 | 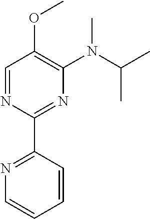 | 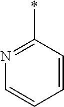 | —CH₃ | 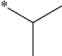 |
| 34 | 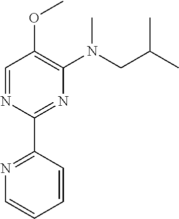 | 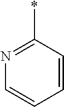 | —CH₃ | 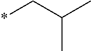 |
| 35 | 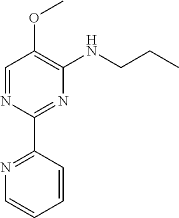 | 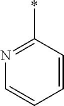 | H | 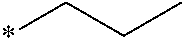 |
| 36 | 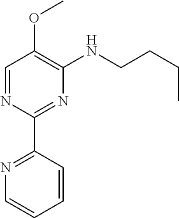 | 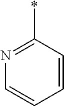 | H | 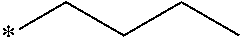 |
| 37 | 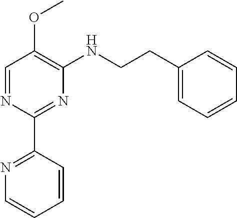 | 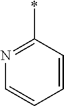 | H | 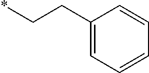 |
| 38 | 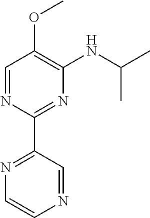 | 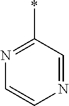 | H | 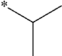 |
| 39 | 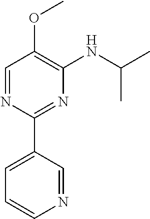 | 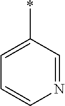 | H | 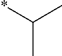 |
| 41 | 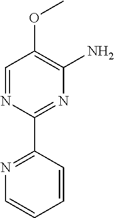 | 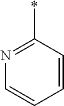 | H | H |
| 42 | 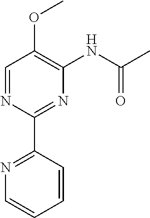 | 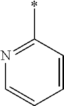 | H | 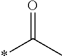 |
| 43 | 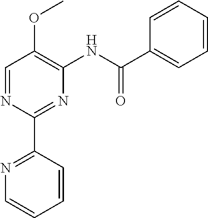 | 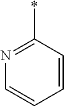 | H | 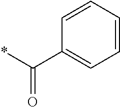 |
| 44 | 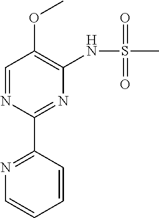 | 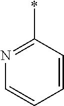 | H | 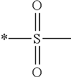 |
| 45 | 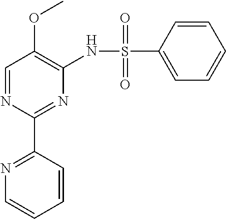 | 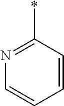 | H | 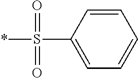 |
| 46 | 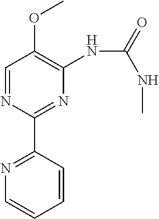 | 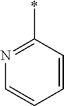 | H | 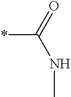 |
| 47 | 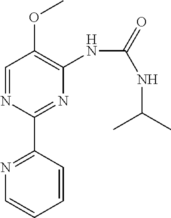 | 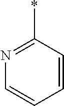 | H | 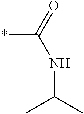 |
| 48 | 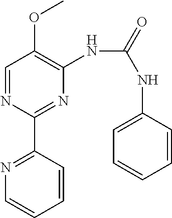 | 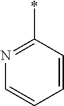 | H | 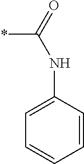 |
| 49 | 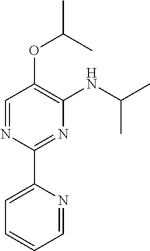 | 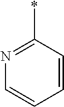 | H | 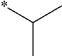 |
| 50 | 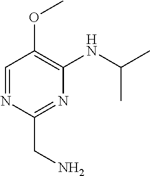 | 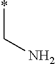 | H | 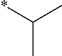 |
| 55 | 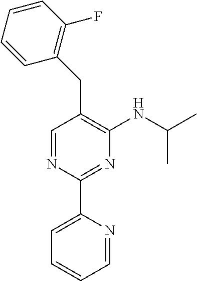 | 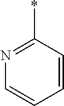 | H | 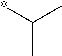 |
| 56 | 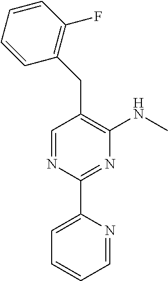 | 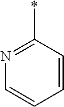 | H | —CH₃ |
| 57 | 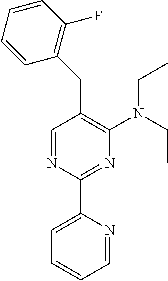 | 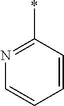 | Ethyl | Ethyl |
| 58 | 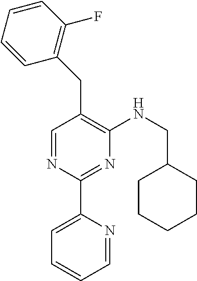 | 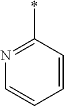 | H | 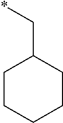 |
| 59 | 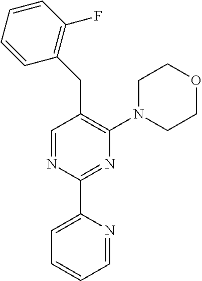 | 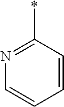 | 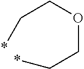 | |
| 60 | 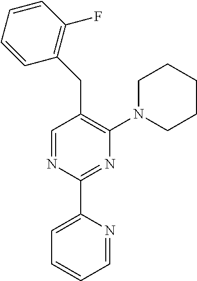 | 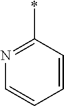 | 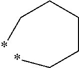 | |
| 61 | 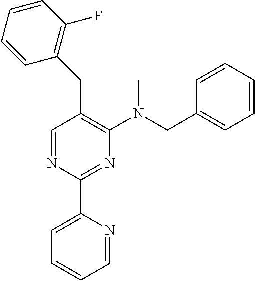 | 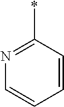 | —CH₃ | Benzyl |
| 62 | 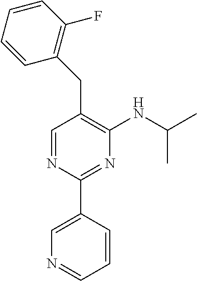 | 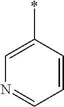 | H | 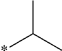 |
| 63 | 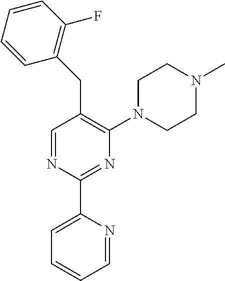 | 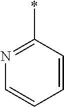 | 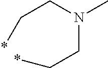 | |
| 69 | 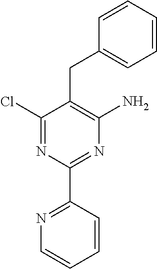 | 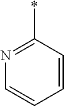 | H | H |
| 70 | 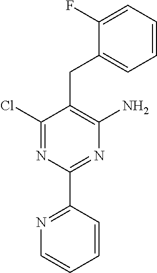 | 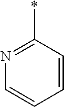 | H | H |
| 71 | 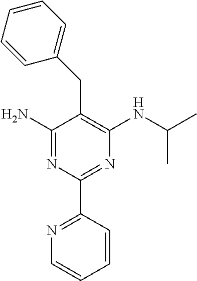 | 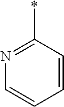 | H | 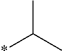 |
| 72 | 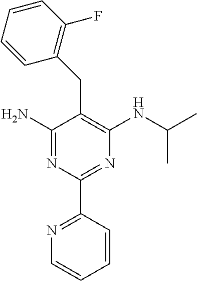 | 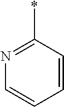 | H | 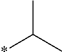 |
| 73 | 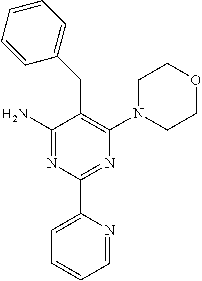 | 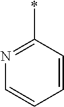 | 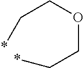 | |
| 74 | 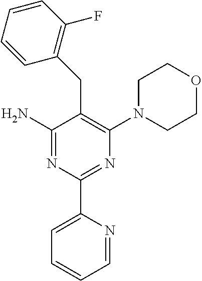 | 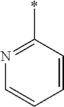 | 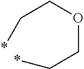 | |
| 75 | 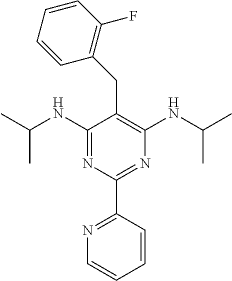 | 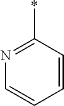 | H | 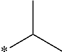 |
| 76 | 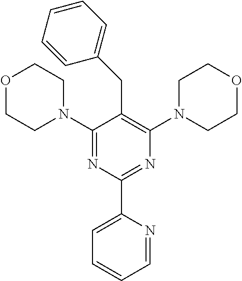 | 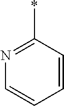 | 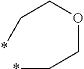 | |
| 77 | 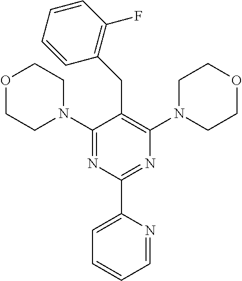 | 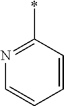 | 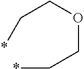 | |
| 78 | 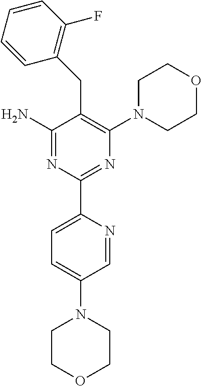 | 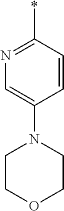 | 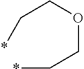 | |
| 79 | 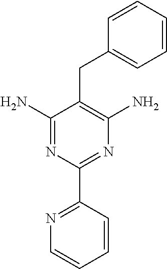 | 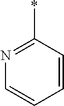 | H | H |
| 80 | 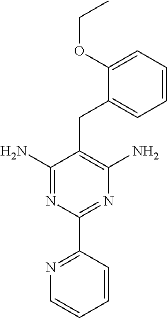 | 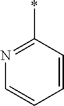 | H | H |
| 81 | 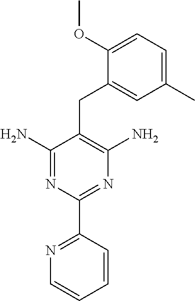 | 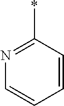 | H | H |
| 82 | 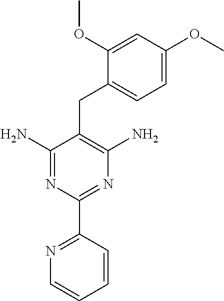 | 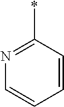 | H | H |
| 83 | 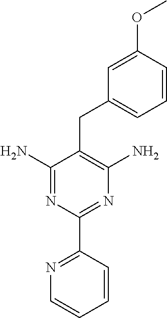 | 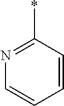 | H | H |
| 84 | 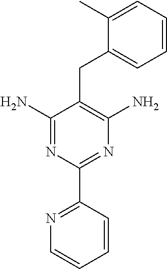 | 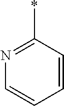 | H | H |
| 85 | 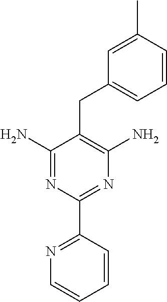 | 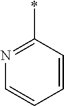 | H | H |
| 86 | 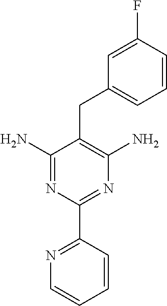 | 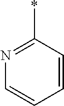 | H | H |
| 87 | 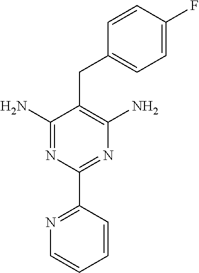 | 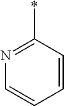 | H | H |
| 88 | 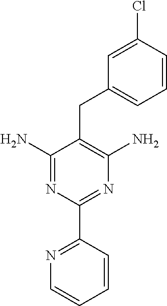 | 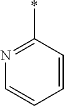 | H | H |
| 89 | 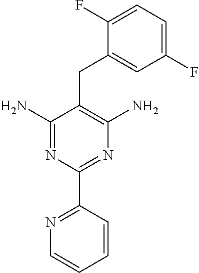 | 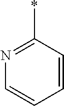 | H | H |
| 90 | 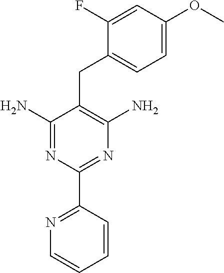 | 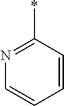 | H | H |
| 91 | 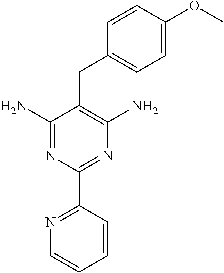 | 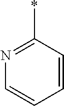 | H | H |
| 92 | 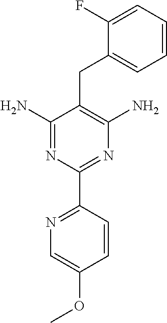 | 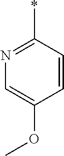 | H | H |
| 93 | 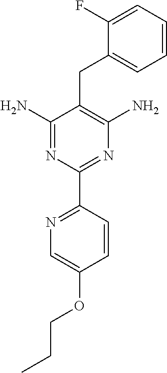 | 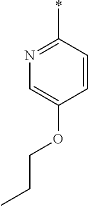 | H | H |
| 94 | 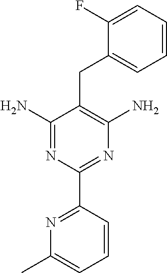 | 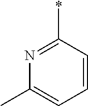 | H | H |
| 95 | 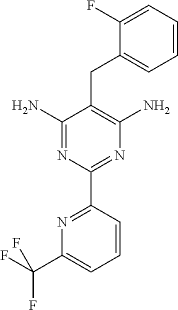 | 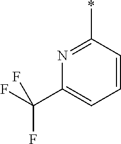 | H | H |
| 97 | 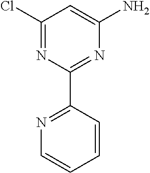 | 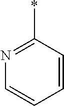 | H | H |
| 98 | 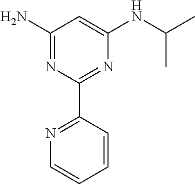 | 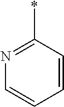 | H | 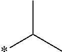 |
| 99 | 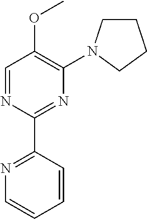 | 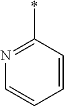 | 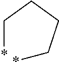 | |
| 100 | 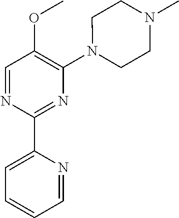 | 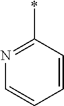 | 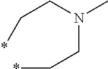 | |
| 101 | 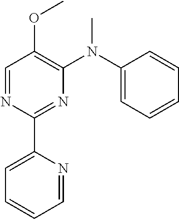 | 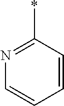 | —CH₃ | Phenyl |
| 103 | 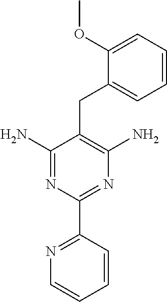 | 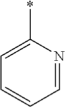 | H | H |
| 104 | 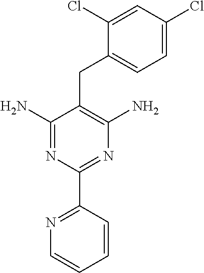 | 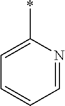 | H | H |
| 105 | 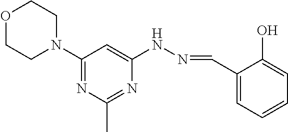 | —CH₃ | H | 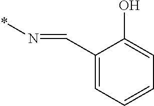 |
| 106 | 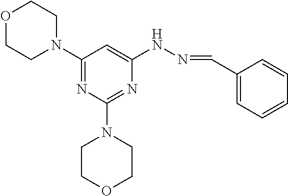 | 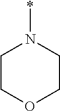 | H | 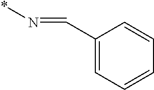 |
| 107 | 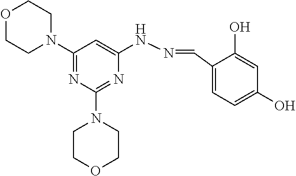 | 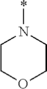 | H | 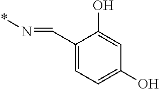 |
| 108 | 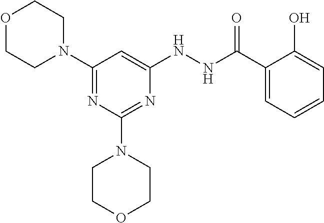 | 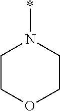 | H | 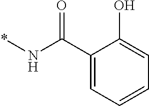 |
| 109 | 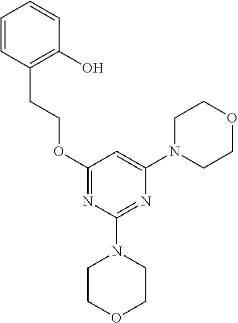 | 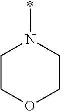 | 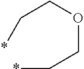 | |
| 110 | 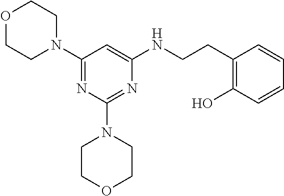 | 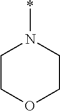 | H | 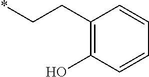 |
| 111 | 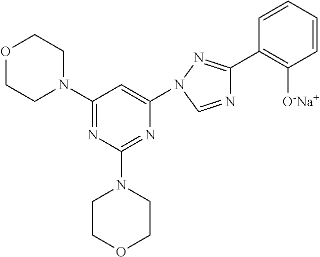 | 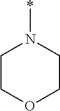 | 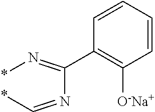 | |
| 112 | 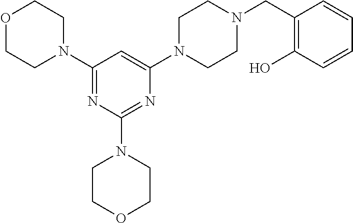 | 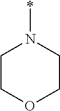 | 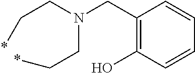 | |
| 113 | 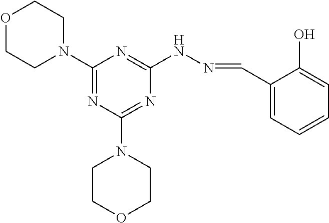 | 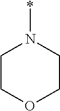 | H | 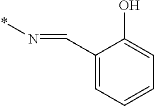 |
| 114 | 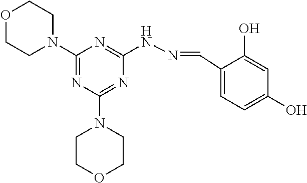 | 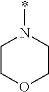 | H | 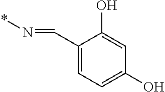 |
| 115 | 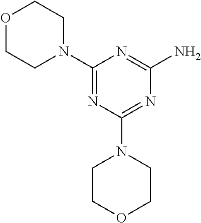 | 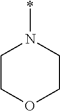 | H | H |
| 116 | 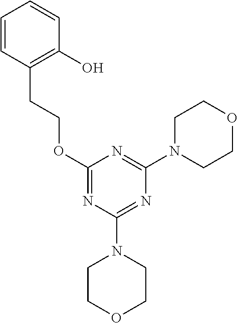 | 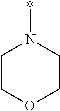 | 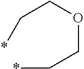 | |
| 117 | 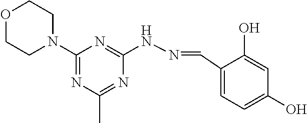 | —CH₃ | H | 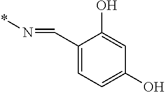 |

| | | (I) | |
|---|---|---|---|
| | | 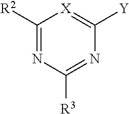 | |
| Example | Compound | R³ | Y |
| 28 | 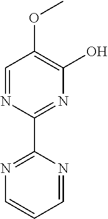 | 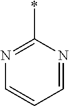 | —OH |
| 29 | 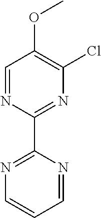 | 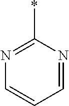 | —Cl |
| 40 | 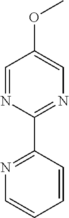 | 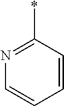 | —H |
| 51 | 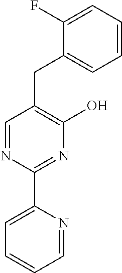 | 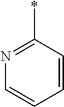 | —OH |
| 52 | 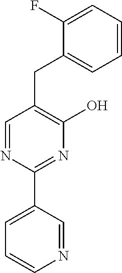 | 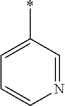 | —OH |
| 53 | 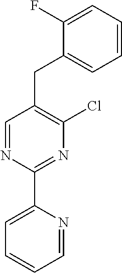 | 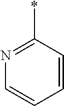 | —Cl |
| 54 | 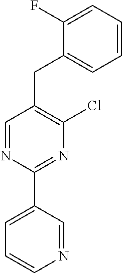 | 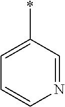 | —Cl |
| 64 | 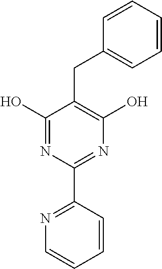 | 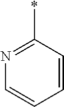 | —OH |
| 65 | 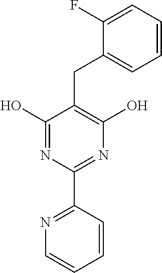 | 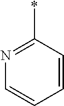 | —OH |
| 66 | 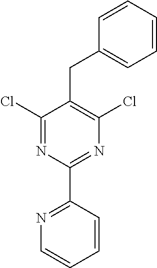 | 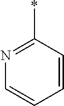 | —Cl |
| 67 | 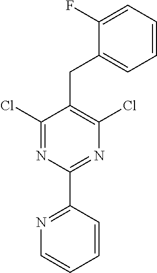 | 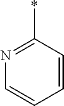 | —Cl |
| 68 | 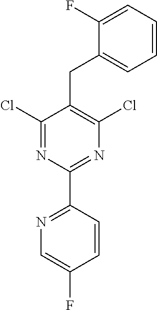 | 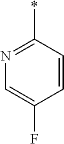 | —Cl |
| 96 | 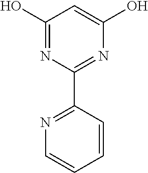 | 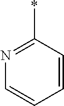 | —OH |
| 102 | 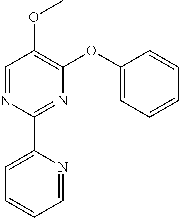 | 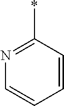 | —O-Phenyl |

(*= Binding position)

and pharmaceutically acceptable salts thereof.

Depending on their structure, the compounds according to the invention may exist in stereoisomeric forms (enantiomers, diastereomers) in the presence of asymmetric carbon atoms. The invention therefore includes the use of the enantiomers or diastereomers and the respective mixtures thereof. The pure-enantiomer forms may optionally be obtained by conventional processes of optical resolution, such as by fractional crystallisation of diastereomers thereof by reaction with optically active compounds. Since the compounds according to the invention may occur in tautomeric forms, the present invention covers the use of all tautomeric forms.

The compounds provided according to the invention may be present as mixtures of various possible isomeric forms, in particular of stereoisomers such as, for example, E- and Z-, syn and anti, as well as optical isomers. The E-isomers and also the Z-isomers as well as the optical isomers and any mixtures of these isomers are claimed.

The compounds according to the invention of general structural formula (I) may basically be obtained by the processes described below and the general procedures (see, for example corresponding stages of Routes 1 to 20 of Examples of Production 13 to 104, the corresponding stages of Routes 1 to 7 of Examples of Production 105 to 112, and also the corresponding stages of Routes 1 to 5 of Examples of Production 113 to 117):

processes, wherein
(a1) compounds of general formula

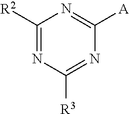

wherein R² and R³ are as defined above, A is a leaving group such as, in particular, halogen, preferably chlorine, are reacted with a compound of general formula

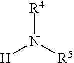

wherein R⁴ and R⁵ are as defined above,
to form compounds of general formula (Ia):

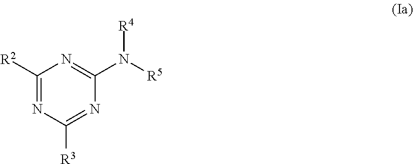

wherein R², R³, R⁴ and R⁵ are as defined above (see for example corresponding stages of Routes 1, 2, 3, 4, 6, 7, 10, 12, 13, 14, 15, 16, 19, 20 of Examples of Production 13 to 104 and also corresponding stages of Routes 1, 2, 3 of Examples of Production 105 to 112 and also the corresponding stages of Routes 1, 2, 3, 4, 5 of Examples of Production 113 to 117), or
   (a2) compounds of general formula

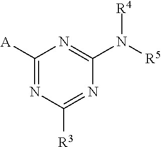

wherein R³, R⁴ and R⁵ are as defined above, A is a leaving group such as, in particular, halogen, preferably chlorine, are reacted with a compound of general formula

R²-E
wherein R² is as defined above, and E here and hereinafter throughout the invention is a suitable group or a suitable element which makes R² into a nucleophile such as, for example, H (particularly if R is an amino group), metals (particularly if R is a hydrocarbon radical), in particular alkali metals such as lithium, sodium and potassium, alkaline earth metals such as calcium or magnesium, —MgBr (Grignard compounds), which make the nucleophilic substitution of A by R² possible,
to form compounds of general formula (Ia), as defined above (see for example corresponding stages of Routes 1, 2, 3, 4, 6, 7, 10, 12, 13, 14, 15, 16, 19, 20 of Examples of Production 13 to 104 and also corresponding stages of Routes 1, 2, 3 of Examples of Production 105 to 112 and also the corresponding stages of Routes 1, 2, 3, 4, 5 of Examples of Production 113 to 117), or
   (a3) compounds of general formula

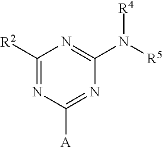

wherein R², R⁴ and R⁵ are as defined above, A is a leaving group such as, in particular, halogen, preferably chlorine, are reacted with a compound of general formula

R³-E
wherein R³ is as defined above, and E is a suitable leaving group, as defined above, which makes possible the substitution of A by R³,
to form compounds of general formula (Ia), as defined above (see for example corresponding stages of Routes 1, 2, 3, 4, 6, 7, 10, 12, 13, 14, 15, 16, 19, 20 of Examples of Production 13 to 104 and also corresponding stages of Routes 1, 2, 3 of Examples of Production 105 to 112 and also the corresponding stages of Routes 1, 2, 3, 4, 5 of Examples of Production 113 to 117), or
   (a4) compounds of general formula

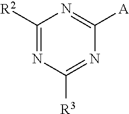

wherein R² and R³ are as defined above, A is a leaving group such as, in particular, halogen, preferably chlorine, are reacted with

H₂N—NH₂
to form a compound of general formula

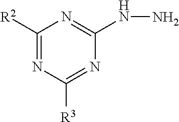

wherein R² and R³ are as defined above, which are subsequently reacted with a compound of formula

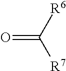

wherein R⁶ and R⁷ are the same or different and are selected from:
   hydrogen,
   optionally substituted alkyl,
   optionally substituted alkenyl,
   optionally substituted alkynyl,
   optionally substituted aryl, or
   optionally substituted heterocyclyl,
to form compounds of formula

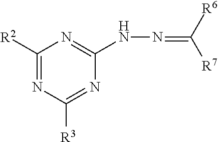

wherein R², R³, R⁶ and R⁷ are as defined above (see for example corresponding stages of Routes 1, 2, 3 of Examples of Production 105 to 112), or
   (a5) compounds of formula

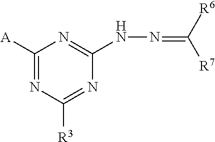

wherein A, R³, R⁶ and R⁷ are as defined above, are reacted with compounds of formula
R²-E, wherein R² is as defined above and E is a suitable leaving group, as defined above, which makes possible the substitution of A by R² to form compounds of formula

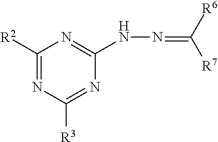

wherein R², R³, R⁶ and R⁷ are as defined above, or
   (a6) compounds of formula

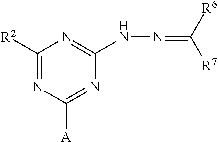

wherein A, R², R⁶ and R⁷ are as defined above, are reacted with compounds of formula
R³-E, wherein R³ is as defined above and E, as defined above, is a suitable leaving group which makes possible the substitution of A by R³ to form compounds of formula

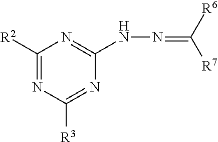

wherein R², R³, R⁶ and R⁷ are as defined above, or
   (b1) compounds of general formula

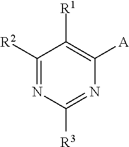

wherein R¹, R² and R³ are as defined above, A is a leaving group such as, in particular, halogen, preferably chlorine, are reacted with a compound of general formula

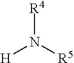

wherein R⁴ and R⁵ are as defined above,
to form compounds of general formula (Ib):

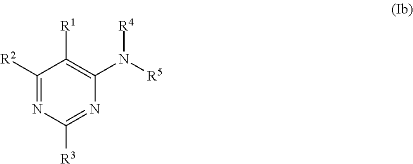

wherein R¹, R², R³, R⁴ and R⁵ are as defined above (see for example corresponding stages of Routes 1, 2, 3, 4, 6, 7, 10, 12, 13, 14, 15, 16, 19, 20 of Examples of Production 13 to 104 and also corresponding stages of Routes 1, 2, 3 of Examples of Production 105 to 112 and also the corresponding stages of Routes 1, 2, 3, 4, 5 of Examples of Production 113 to 117), or
   (b2) compounds of general formula

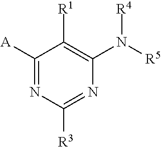

wherein R¹, R³, R⁴ and R⁵ are as defined above, A is a leaving group, in particular halogen, preferably chlorine, is reacted with a compound of general formula

R²-E
wherein R² is as defined above and E is a suitable leaving group, as defined above, which makes possible the substitution of A by R²,
to form compounds of general formula (Ib), as defined above (see for example corresponding stages of Routes 1, 2, 3, 4, 6, 7, 10, 12, 13, 14, 15, 16, 19, 20 of Examples of Production 13 to 104 and also corresponding stages of Routes 1, 2, 3 of Examples of Production 105 to 112 and also the corresponding stages of Routes 1, 2, 3, 4, 5 of Examples of Production 113 to 117), or
   (b3) compounds of general formula

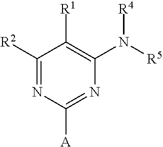

wherein R¹, R², R⁴ and R⁵ are as defined above, A is a leaving group, in particular halogen, preferably chlorine, is reacted with a compound of general formula

R³-E
wherein R³ is as defined above and E is a suitable leaving group, as defined above, which makes possible the substitution of A by R³,
to form compounds of general formula (Ib), as defined above (see for example corresponding stages of Routes 1, 2, 3, 4, 6, 7, 10, 12, 13, 14, 15, 16, 19, 20 of Examples of Production 13 to 104 and also corresponding stages of Routes 1, 2, 3 of Examples of Production 105 to 112 and also the corresponding stages of Routes 1, 2, 3, 4, 5 of Examples of Production 113 to 117), or
   (b4) compounds of general formula

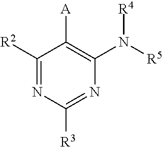

wherein R², R³, R⁴ and R⁵ are as defined above, A is a leaving group, in particular halogen, preferably chlorine, is reacted with a compound of general formula

R¹-E
wherein R¹ is as defined above and E is a suitable leaving group, as defined above, which makes possible the substitution of A by R¹,
to form compounds of general formula (Ib), as defined above (see for example corresponding stages of Routes 1, 2, 3, 4, 6, 7, 10, 12, 13, 14, 15, 16, 19, 20 of Examples of Production 13 to 104 and also corresponding stages of Routes 1, 2, 3 of Examples of Production 105 to 112 and also the corresponding stages of Routes 1, 2, 3, 4, 5 of Examples of Production 113 to 117), or
   (b5) compounds of general formula

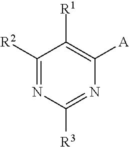

wherein R¹, R² and R³ are as defined above, A is a leaving group such as, in particular, halogen, preferably chlorine, are reacted with

H₂N—NH₂
to form compounds of general formula

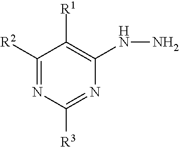

wherein R¹, R² and R³ are as defined above, which are subsequently reacted with a compound of formula

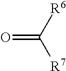

wherein R⁶ and R⁷ are the same or different and are as defined above, to form compounds of formula

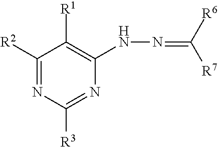

wherein R¹, R², R³, R⁶ and R⁷ are as defined above (see for example corresponding stages of Routes 1, 2, 3 of Examples of Production 105 to 112), or
   (b6) compounds of formula

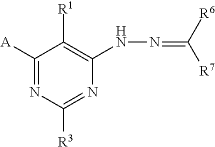

wherein A, R¹, R³, R⁶ and R⁷ are as defined above, are reacted with compounds of formula
R²-E, wherein R² is as defined above and E is a suitable leaving group, as defined above, which makes possible the substitution of A by R² to form compounds of formula

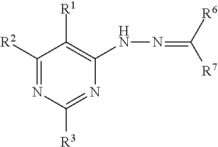

wherein R¹, R², R³, R⁶ and R⁷ are as defined above, or
   (b7) compounds of formula

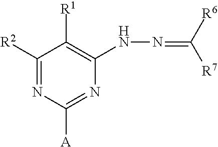

wherein A, R¹, R², R⁶ and R⁷ are as defined above, are reacted with compounds of formula
R³-E, wherein R³ is as defined above and E is a suitable leaving group, as defined above, which makes possible the substitution of A by R³ to form compounds of formula

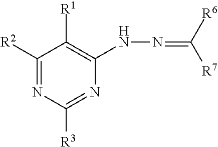

wherein R¹, R², R³, R⁶ and R⁷ are as defined above, or
   (b8) compounds of formula

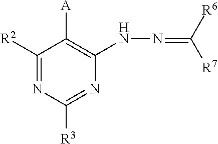

wherein A, R², R⁶ and R⁷ are as defined above, are reacted with compounds of formula
R¹-E, wherein R¹ is as defined above and E is a suitable leaving group, as defined above, which makes possible the substitution of A by R¹ to form compounds of formula

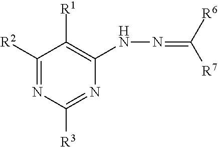

wherein R¹, R², R³, R⁶ and R⁷ are as defined above.

In particular, the compounds according to the invention of general structural formula (I) may be obtained by the processes described below.

A starting point for the synthesis of compounds of general formula (I), in which X represents C—R¹ and in which R¹ is selected from the group of alkoxy, halogen, optionally substituted alkyl, optionally substituted aryl or optionally substituted heterocyclyl, and wherein R², R³, R⁴ and R⁵ have one of the foregoing meanings, is commercial alkylimideamide of general formula (II), which may be cyclised under standard conditions [see for example: Henze et al, JOC, 17, 1952, 1320-1322; R. Ferris, JACS, 62, 1940, 606; S. Biggs, Journal of the Chemistry Society, 1959, 1849-1854] with 1,3-diketo compounds of general formula (III) to form pyrimidinone of general formula (IV).

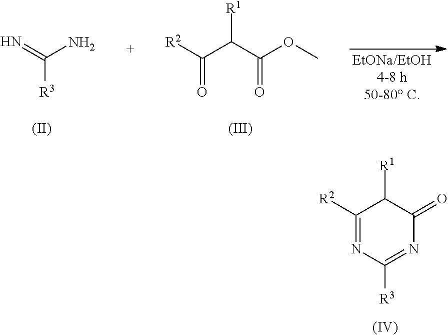

By subsequent treatment of the pyrimidinones of general formula (IV) with phosphoryl chloride by known methods [see for example: B. Singh, Heterocycles, 31, 1990, 2163-2172], it is possible to obtain the corresponding chlorine-substituted pyrimidines of general formula (V).

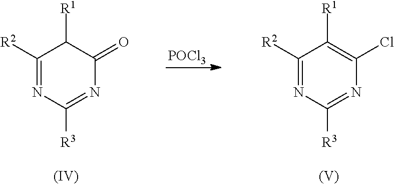

These may then be derivatised under standard conditions known to the person skilled in the art [see for example: K. A. Kolmakov, Journal of Heterocyclic Chemistry, 45, 2008, 533-539] under basic reaction conditions with amine of general formula (VI) to form the end compounds of general formula (I).

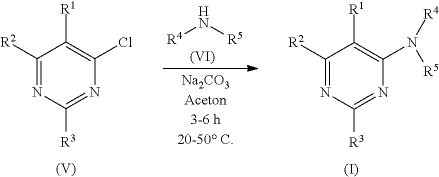

Further similar universally applicable processes for making up the pyrimidines are described, for example, in Routes 3, 4, 10, 13, 14, 17, 18, 19 and 20 of Examples of Production 13 to 104.

In the literature there is generally a large number of further methods of synthesising substituted pyrimidines. One of these methods of synthesis for making up highly substituted pyrimidines of general formulae (I) is as follows [see for example: A. G. Martinez, JOC, 57, 1992, 1627]:

Ketones of general formula (III') are condensed under trifluoroacetic acid anhydride catalysis with nitriles, in particular chlorocyan, to form the pyrimidines of general formula (V′).

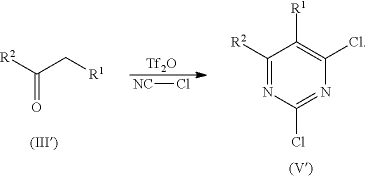

The compounds of general formula (V′) may then be reacted by suitable methods known to the person skilled in the art [see for example: B. Singh, Heterocycles, 31, 1990, 2163-2172] to form compounds of general formula (V) and also by known methods [see for example: K. A. Kolmakov, Journal of Heterocyclic Chemistry, 45, 2008, 533-539], as described above, to form compounds of general formula (I).

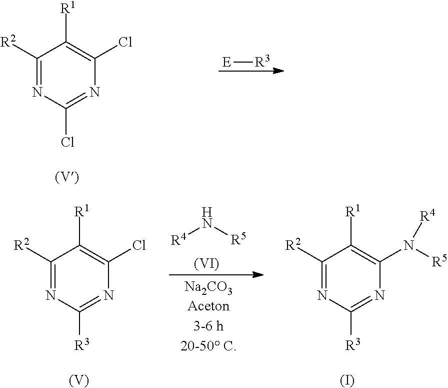

In this case, E, as stated above, represents a suitable leaving group which makes possible the substitution of Cl by R³.

The compounds according to the invention, in particular, are also obtainable in accordance with Examples 1, 2, 3 and 4 by the above-described synthesis pathways.

There is an additional procedure according to the invention which is suitable for the production of the compounds according to the invention of general formula (I), wherein X represents C—R¹ in which R¹ has the meaning of hydrogen, and wherein furthermore R² has the meaning of optionally substituted amino, as defined above, and wherein furthermore R³ has one of the foregoing meanings and wherein R⁴ and R⁵ also have one of the foregoing meanings, one of the substituents R⁴ or R⁵ having the meaning of optionally substituted amino and also being selected from the group thereof which, together with the nitrogen atom to which they are bound, to form an optionally substituted hydrazone group, originates.

[symbol]

The starting point for the synthesis of compounds of this type according to the invention is commercial 2,4,6-trichloropyrimidine (VII), which may be reacted by standard methods known to the person skilled in the art [see for example: B. Singh, Heterocycles, 31, 1990, 2163-2172] to form compounds of general formula (VIII'). These are then derivatised under conditions known to the person skilled in the art [see for example: T. J. Delia, Journal of Heterocyclic Chemistry, 36, 1999, 1259-1262] with compounds of formula R²—H, wherein R² represents an optionally substituted amino compound, to form compounds of general formula (VIII). These are then converted into the hydrazine of general formula (IX) in a further step with hydrazine hydrate under standard conditions [see for example: Chesterfield et al, Journal of the Chemical Society, 1955, 3478-3481], which is then reacted by reaction with aldehydes of general formula R⁶—(C═O)—R⁷, according to the procedure below, to form the corresponding hydrazones of general formula (X) [see for example: Claesen, Bulletin des Societés Chimiques Beiges, 68, 1959, 47-57; L. F. Kuyper, Bioorganic & Medicinal Chemistry, 4, 1996, 593-602]. It is basically also possible in the process to first react compounds of formula (VIII′) with hydrazine hydrate and aldehydes to form the corresponding hydrazones and then to carry out derivatisation with the compound R²-E. In the following procedure, E represents a suitable leaving group, as defined above, which makes possible the substitution of Cl by R² or R³, and R⁶ and R⁷ are the same or different and are selected from:
hydrogen,
optionally substituted alkyl,
optionally substituted alkenyl,
optionally substituted alkynyl,
optionally substituted aryl, or
optionally substituted heterocyclyl.

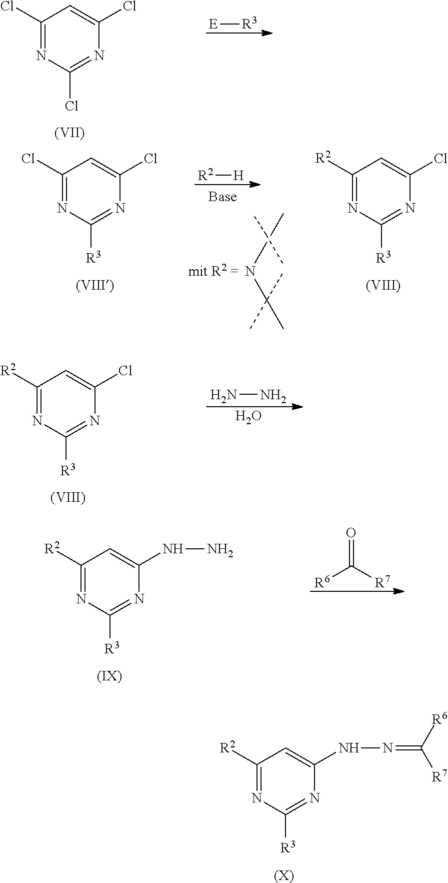

(The diction

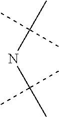

in this case and throughout the specification shall mean that the nitrogen atom has substituents, which are in accordance with the meanings as defined in the present invention.

Throughout the invention, if R²═R³, the reaction to the corresponding target compound with R² and R³ may basically also be carried out in one stage. (See for example corresponding stages of Routes 1, 2, 3 of Examples of Production 105 to 112)).

The compounds (X) obtainable in this way correspond to compounds according to the invention of formula (I), wherein X has the meaning of C where R¹=H, R² represents, in particular, an optionally substituted amino group, R³ has one of the foregoing meanings according to the invention and wherein one of the substituents R⁴ or R⁵ is hydrogen and the other respective substituent is an optionally substituted amino selected from the group thereof which, together with the nitrogen atom to which they are bound, form an optionally substituted hydrazone group:

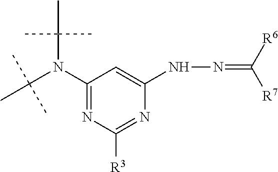

The compounds according to the invention in accordance with Examples 6 and 8, in particular, are also obtainable by the above-described synthesis pathway.

In order to obtain compounds according to the invention in which R³ also additionally represents an optionally substituted amino group, the reaction of the compound of formula (VII) is carried out in accordance with the foregoing synthesis procedure under conditions known to the person skilled in the art [see for example: T. J. Delia, Journal of Heterocyclic Chemistry, 36, 1999, 59-1262] using compounds of formula R³—H, wherein R³ represents an optionally substituted amino compound, to form compounds of general formula (VIII″) and subsequent derivatisation with R²-E, as defined above, and reaction to the corresponding hydrazone compounds as shown above.

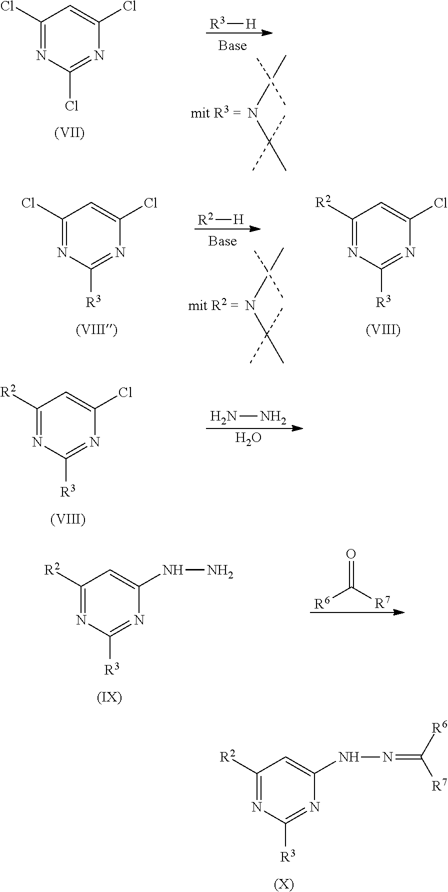

In compound (X) therein, both the substituent R² and the substituent R³ are bound to the pyrimidine ring via a respective nitrogen atom:

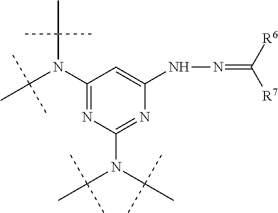

The compounds according to the invention in accordance with Examples 5 and 7, in particular, are also obtainable by this synthesis pathway.

The following synthesis pathway provides a process for producing compounds according to the invention of general formula (I), wherein X represents N and wherein the substituents R² and R³ represent optionally substituted amino compounds or optionally substituted heterocyclyl compounds, which are bound via a hetero nitrogen atom.

The starting point for the synthesis of compounds of this type of formula (I) is commercial 2,4,6-trichloro-1,3,5-triazine of formula (XI), which may be reacted via the described processes known to the person skilled in the art.

In the process, commercial triazine (×1) is initially reacted under basic reaction conditions with amine of general formula R⁴—NH—R⁵ by standard methods known to the person skilled in the art [see for example: K. A. Kolmakov, Journal of Heterocyclic Chemistry, 45, 2008, 533-539], to form compounds of general formula (XI′). The resulting amino triazine (XI′) may then be reacted analogously with further amines R³—H and R²—H under basic reaction conditions via diaminotriazine (XI″) to form the desired compound of general formula (I) [see for example: H. E. Birkett, Magnetic Resonance in Chemistry, 41, 2003, 324-336; J. P. Mathias, JACS, 116, 1994, 4326-4340].

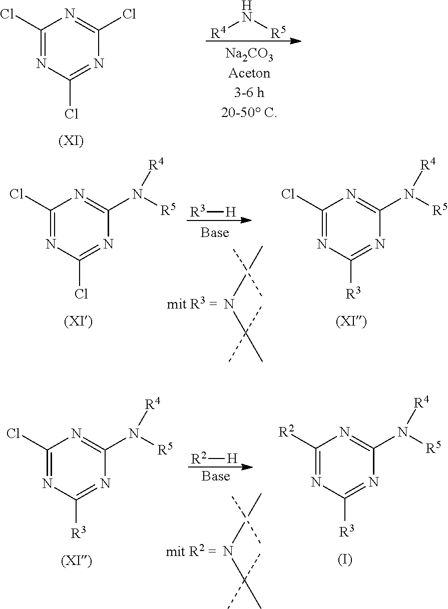

In compound (I) therein, both the substituent R² and the substituent R³ are bound to the triazine ring via a respective nitrogen atom within the meaning of general formula:

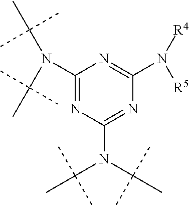

The compounds according to the invention in accordance with Examples 9, 10, 11 and 12, in particular, are also obtainable by this synthesis pathway. (See for example also corresponding stages of Routes 1 to 5 of Examples of Production 113 to 117).

In order to obtain corresponding triazine compounds in which either R² or R³ has another of the above-mentioned meanings for R² and R³ from that of an optionally substituted amino compound, the corresponding diaminotriazines (XI″) and (XI′″) may also be reacted with other nucleophiles to form compound (I) [see for example: P. A. Belyakoy, Russian Chemical Bulletin, 54, 2005, 2441-2451]:

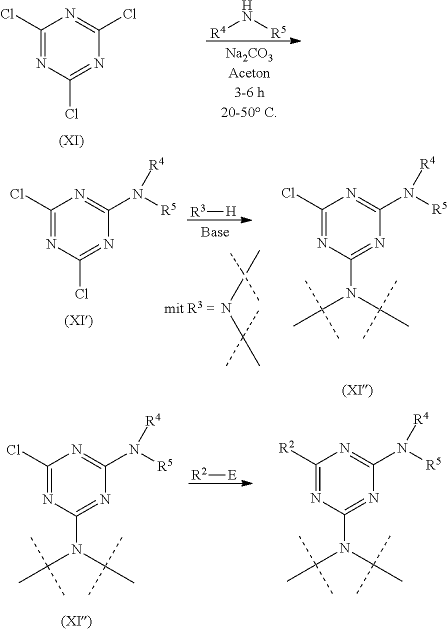

wherein R² has one of the foregoing meanings according to the invention and wherein E is a suitable leaving group, as defined above, or:

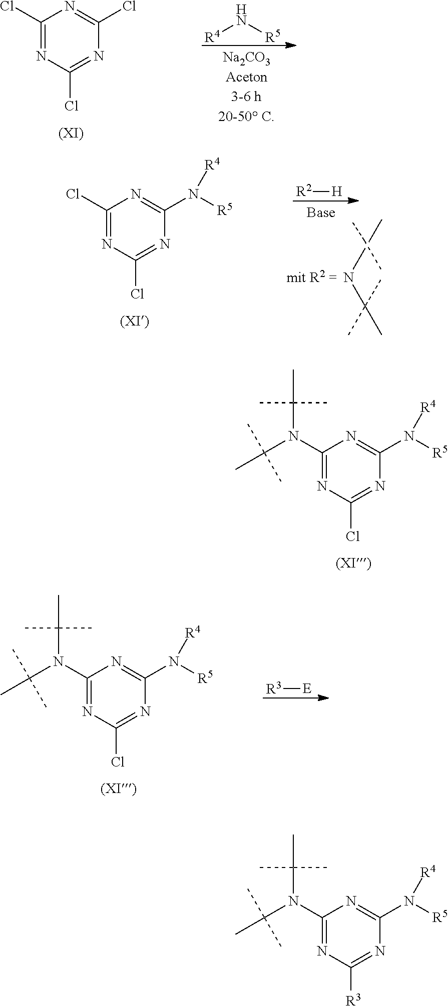

wherein R³ has one of the foregoing meanings according to the invention and wherein E is a suitable leaving group, as defined above.

In the context of the invention, compounds R-E, in particular R³-E and R²-E are those, in which R² and R³ have the meanings as defined above and in which E is a suitable leaving group which is capable, in particular, of substituting the chlorine atom in the corresponding triazinyl or pyrimidine parent substance by means of the group R, as defined above.

The reaction pathways shown here represent types of reaction which are known per se and may be carried out in a manner known per se. Corresponding salts are obtained by reaction with a pharmaceutically acceptable base or acid.

The reaction between the various reactants may be carried out in various solvents and is not subject to any restrictions in this respect. Examples of suitable solvents therefore include water, ethanol, acetone, dichloroethane, dichloromethane, dimethoxyethane, diglyme, acetonitrile, butyronitrile, THF, dioxane, ethylacetate, butylacetate, dimethylacetamide, toluene and chlorobenzene. It is also possible to carry out the reaction in a substantially homogeneous mixture of water and solvents, if the organic solvent is miscible with water.

The reaction according to the invention between the reactants is carried out, for example, at ambient temperature. However, temperatures above ambient temperature, for example up to 70° C., and temperatures below ambient temperature, for example down to −20° C. or less, may also be used.

The pH, at which the reaction according to the invention between the reactants, in particular R² and R³ substitution, is carried out, is suitably adjusted.

The pH is adjusted, in particular during R² and R³ substitution and also during amination with R⁴—NH—R⁵, preferably by addition of a base. Suitable bases include both organic and inorganic bases. Inorganic bases such as, for example, LiOH, NaOH, KOH, Ca(OH)₂, Ba(OH)₂, Li₂CO₃, K₂CO₃, Na₂CO₃, NaHCO₃, or organic bases such as amines (for example, preferably triethylamine, diethylisopropylamine), Bu₄NOH, piperidine, morpholine, alkylpyridines are preferably used. Inorganic bases are particularly preferably used, and Na₂CO₃, LiOH, NaOH and KOH are most preferably used.

The pH may optionally also be adjusted using acids, in particular during cyclisation to pyrimidinones. Suitable acids include both organic and inorganic acids. Inorganic acids such as, for example, HCl, HBr, HF, H₂SO₄, H₃PO₄ or organic acids such as CF₃COOH, CH₃COOH, p-toluenesulfonic acid and the salts thereof are preferably used. Inorganic acids such as HCl and H₂SO₄ and also organic acids such as trifluoroacetic acid (CF₃COOH), trifluoroacetic acid anhydride (Tf₂O) and acetic acid (CH₃COOH) or the sodium salt thereof (EtONa) are particularly preferably used.

A person skilled in the art is capable of selecting the most suitable solvent and the optimum reaction conditions, in particular with respect to temperature, pH, catalyst and solvent for the corresponding synthesis pathway.

The inventors have surprisingly found that the compounds forming the subject-matter of the present invention and corresponding to general structural formula (I) act as hepcidin antagonists and are therefore suitable for use as drugs for the treatment of hepcidin-mediated diseases and the accompanying or associated symptoms. In particular, the compounds according to the invention are suitable for the treatment of iron metabolism disorders, in particular for the treatment of iron deficiency diseases and/or anaemia, in particular in ACD and AI.

The drugs containing the compounds of general structural formula (I) are suitable for use in human and veterinary medicine.

The compounds according to the invention are therefore also suitable for the production of a medication for the treatment of patients suffering from symptoms of iron deficiency anaemia such as, for example: fatigue, listlessness, poor concentration, low cognitive efficiency, difficulty in finding the correct words, forgetfulness, unnatural pallor, irritability, accelerated heart rate (tachycardia), sore or swollen tongue, enlarged spleen, cravings in pregnancy (pica), headaches, loss of appetite, increased susceptibility to infection, depressive moods or an ACD or an AI.

The compounds according to the invention are therefore also suitable for the production of a medication for the treatment of patients suffering from symptoms of iron deficiency anaemia.

Administration can take place over a period of several months until there is an improvement in iron levels, as reflected, for example, by the patient's haemoglobin value, transferrin saturation and ferritin value, or there is a desired improvement in the health state impairment caused by iron deficiency anaemia or by ACD or AI.

The preparation according to the invention may be taken by children, adolescents and adults.

The compounds of the present invention may additionally also be used in combination with further active ingredients or drugs known for the treatment of iron metabolism disorders and/or with active ingredients or drugs which are administered as an accompaniment to agents for the treatment of diseases associated with iron metabolism disorders, in particular with iron deficiency and/or anaemia. Examples of such agents which may be used in combination for the treatment of iron metabolism disorders and other diseases associated with iron deficiency and/or anaemia may include, for example, iron-containing compounds such as, for example, iron salts, iron carbohydrate complexes such as iron-maltose or iron-dextrin complexes, vitamin D and/or derivatives thereof.

The compounds used in combination with the compounds according to the invention may be administered both orally and parenterally, or the compounds according to the invention and the compounds used in combination may be administered by a combination of said methods of administration.

The compounds according to the invention and the aforementioned combinations of compounds according to the invention with further active ingredients or drugs may be used in the treatment of iron metabolism disorders such as, in particular, iron deficiency diseases and/or anaemia, in particular anaemia in cancer, anaemia triggered by chemotherapy, anaemia triggered by inflammation (AI), anaemia in congestive heart failure (CHF), anaemia in chronic kidney disease stage 3-5 (CKD 3-5), anaemia triggered by chronic inflammation (ACD), anaemia in rheumatoid arthritis (RA), anaemia in systemic lupus erythematosus (SLE) and anaemia in inflammatory bowel disease (IBD), or for the production of medications for the treatment of these diseases.

The compounds according to the invention and the aforementioned combinations of compounds according to the invention with further active ingredients or drugs may be used, in particular, for the production of medications for the treatment of iron deficiency anaemia such as iron deficiency anaemia in pregnant women, latent iron deficiency anaemia in children and adolescents, iron deficiency anaemia due to gastrointestinal abnormalities, iron deficiency anaemia due to loss of blood, for example due to gastrointestinal bleeding (for example due to ulcers, carcinomas, haemorrhoids, inflammatory disorders, taking of acetylsalicylic acid), menstruation, injuries, iron deficiency anaemia due to psilosis (sprue), iron deficiency anaemia due to reduced iron absorption through food, in particular in the case of children and adolescents with selective eating, immunodeficiency due to iron deficiency anaemia, impairment of brain function due to iron deficiency anaemia, restless leg syndrome.

The use according to the invention leads to an improvement in iron, haemoglobin, ferritin and transferrin values which is accompanied by an improvement in short-term memory tests (STM), in long-term memory tests (LTM), in Raven's progressive matrices, in the Wechsler adult intelligence scale (WAIS) and/or in the emotional coefficient (Baron EQ-I, YV test; youth version), or by an improvement in neutrophile levels, antibody levels and/or lymphocyte function, in particular in adolescents and children, but also in adults.

The present invention further relates to pharmaceutical compositions containing one or more of the compounds according to the invention corresponding to formula (I), and optionally one or more further pharmaceutically active compounds and optionally one or more pharmacologically acceptable carriers and/or auxiliaries and/or solvents.

These are conventional pharmaceutical carriers, auxiliaries or solvents. Said pharmaceutical compositions are suitable, for example, for intravenous, intraperitoneal, intramuscular, intravaginal, intrabuccal, percutaneous, subcutaneous, mucocutaneous, oral, rectal, transdermal, topical, intradermal, intragastral or intracutaneous application and are present, for example, in the form of pills, tablets, enteric-coated tablets, film tablets, layer tablets, sustained-release formulations for oral administration, subcutaneous or cutaneous administration (in particular as plasters), extended-release formulations, dragees, pessaries, gels, ointments, syrup, granules, suppositories, emulsions, dispersions, microcapsules, microformulations, nanoformulations, liposomal formulations, capsules, enteric-coated capsules, powders, inhalation powders, microcrystalline formulations, inhalation sprays, powders, drops, nose drops, nasal sprays, aerosols, ampoules, solutions, juices, suspensions, infusion solutions or injection solutions, etc.

The compounds according to the invention and pharmaceutical compositions containing these compounds are preferably applied orally and/or parenterally, in particular intravenously.

For this purpose, the compounds according to the invention are preferably present in pharmaceutical compositions in the form of pills, tablets, enteric-coated tablets, film tablets, layer tablets, sustained-release formulations for oral administration, extended-release formulations, dragees, granules, emulsions, dispersions, microcapsules, microformulations, nanoformulations, liposomal formulations, capsules, enteric-coated capsules, powders, microcrystalline formulations, powders, drops, ampoules, solutions, suspensions, infusion solutions or injection solutions.

The compounds according to the invention may be administered in pharmaceutical compositions which may contain various organic or inorganic carriers and/or auxiliaries, of the type conventionally used for pharmaceutical purposes, in particular for solid drug formulations such as, for example, excipients (such as saccharose, starch, mannitol, sorbitol, lactose, glucose, cellulose, talc, calcium phosphate, calcium carbonate), binders (such as cellulose, methylcellulose, hydroxypropylcellulose, polypropylpyrrolidone, gelatin, gum arabic, polyethyleneglycol, saccharose, starch), disintegration agents (such as starch, hydrolysed starch, carboxymethylcellulose, calcium salt of carboxymethylcellulose, hydroxypropyl starch, sodium glycol starch, sodium bicarbonate, calcium phosphate, calcium citrate), lubricants or lubricating agents (such as magnesium stearate, talc, sodium laurylsulfate), a flavouring (such as citric acid, menthol, glycine, orange powder), preservatives (such as sodium benzoate, sodium bisulfite, methylparaben, propylparaben), stabilisers (such as citric acid, sodium citrate, acetic acid) and multicarboxylic acids from the titriplex series such as, for example, diethylenetriamine pentaacetic acid (DTPA), suspending agents (such as methylcellulose, polyvinylpyrrolidone, aluminium stearate), dispersants, diluents (such as water, organic solvents), beeswax, cocoa butter, polyethyleneglycol, white petrolatum, etc.

Liquid drug formulations such as solutions, suspensions and gels conventionally contain a liquid carrier such as water and/or pharmaceutically acceptable organic solvents. In addition, liquid formulations of this type may also contain pH-adjusting agents, emulsifiers or dispersing agents, buffering agents, preservatives, wetting agents, gelling agents (for example methylcellulose), colorants and/or flavourings. The compositions according to the invention may be isotonic, in other words they may have the same osmotic pressure as blood. The isotonicity of the composition may be adjusted by using sodium chloride or other pharmaceutically acceptable agents such as, for example, dextrose, maltose, boric acid, sodium tartrate, propyleneglycol or other inorganic or organic soluble substances. The viscosity of the liquid compositions may be adjusted using a pharmaceutically acceptable thickener such as methylcellulose. Other suitable thickeners include, for example, xanthan, carboxymethylcellulose, hydroxypropylcellulose, carbomer and the like. The preferred concentration of the thickener will depend on the selected agent. Pharmaceutically acceptable preservatives may be used to increase the stability of the liquid composition. Benzyl alcohol may be suitable, although a large number of preservatives including, for example, paraben, thimerosal, chlorobutanol or benzalkonium chloride may also be used.

The active ingredient may be administered, for example, in a unit dose of 0.001 mg/kg to 500 mg/kg body weight, for example up to 1 to 4 times per day. The dosage may be increased or reduced according to the age, weight, condition of the patient, severity of the disease or method of administration.

A preferred embodiment relates to the use of the compounds according to the invention, and of compositions containing the compounds according to the invention, and also of the combined preparations according to the invention containing the compounds and compositions according to the invention, for producing a drug for oral or parenteral administration.

The invention is illustrated in more detail by the following examples. The examples are merely explanatory, and the person skilled in the art can extend the specific examples to further claimed compounds.

### EXAMPLES

### Pharmacological Assays

The following materials were used:

| Reagents | Batch No. | Comments |
|---|---|---|
| MDCK-FPN-HaloTag Clone 7 | | |
| Hepcidin 100 μM Stock | Batch# 571007 | Peptides International |
| solution in water | | |
| HaloTag ®TMR Ligand | Batch# 257780 | Promega, Cat#G8251 |
| Opera confocal plate imager | | PerkinElmer |
| Perkin Elmer 384 Cell | | Cat#6007430 |
| carrier plates | | |
| Paraformaldehyde | Batch# 080416 | Electron Microscopy |
| | | Sciences |
| | | Cat#15710-S |
| Draq5 | | Biostatus, Cat No: |
| | | DR51000 |

The antagonistic effect against hepcidin of the pyrimidine and triazine compounds of the present invention was determined by means of the ferroportin internalisation assay described below.

### Principle of the Ferroportin Internalisation Assay

Low molecular weight organic compounds which counteract the biological effects of hepcidin on its receptor, the iron exporter ferroportin (Fpn) were identified on the basis of their ability to inhibit hepcidin-induced internalisation of Fpn in living cells. A stable cell line (Madin-Darby Canine Kidney, MDCK) was produced for this purpose to express constitutively human ferroportin which is fused recombinantly with a fluorescent reporter protein (HaloTag®, Promega Corp.) at its C terminus. The internalisation of Fpn was monitored by marking these cells with fluorescent ligands (HaloTag® TMR, tetramethylrhodamine) which attach themselves covalently to the HaloTag reporter gene fused with the Fpn. Images produced by confocal fluorescence microscopes showed cell surface localisation of Fpn in the absence of hepcidin and the absence of Fpn surface colouring in the presence of hepcidin. Optimised image analysis algorithms were used to detect the cell surface and to quantify the corresponding membrane fluorescence associated with the Fpn-HaloTag fusion protein. This assay allows quantitative image-based analysis for quickly evaluating compounds capable of blocking hepcidin-induced internalisation of Fpn. This assay is a direct in vitro equivalent of the in vivo action mechanism proposed for drug candidates, and is therefore suitable as an initial assay with a high throughput for identifying compounds which counteract the effect of hepcidin on its receptor ferroportin.

Details of assay procedure
7500 cells per well (MDCK-FPN-HaloTag) were transferred per well in 50 μl DMEM medium (Dulbeccos Modified Eagle Medium with 10% foetal bovine serum (FBS) containing 1% penicillin, 1% streptomycin and 450 μg/ml G-418) in microtitre plates with 384 wells (384 cell carrier plates, Perkin Elmer, Cat. No. 6007430), then incubated overnight at 37° C./5% CO₂.
The volume of the medium was reduced to 10 μl and 10 μl of 5 μM HaloTag-TMR ligand (Promega, Cat. No. G 8251) were added in DMEM medium in order to stain the Fpn-HaloTag fusion protein.
15 min incubation at 37° C./5% CO₂.
HaloTag-TMR ligand was removed, the cells were washed with fresh DMEM medium, and the volume was reduced to 20 μl DMEM medium.
3 μl of a solution of the test compound (dissolved DMSO) were added per well (10 μl final volume).
7 μl of 43 μm hepcidin (Peptides International, Cat. No. PLP-4392-s, 100 μM stock solution in water diluted in DMEM medium) were added per well to a final hepcidin concentration of 100 nM.
The cells were incubated overnight at 37° C./5% CO₂.
The cells were fixed by adding paraformaldehyde (PFA, Electron Microscopy Sciences, Cat. No. 15710-S) directly to the cells to give a final concentration of 4%, and then incubated for 15-20 minutes at room temperature.
The PFA solution was removed and the cells washed with PBS (phosphate-buffered saline solution), 30 μl remained in the plate in each case.
20 μl Draq5 (Biostatus, Cat. No. DR 51000) were added to give a final concentration of 2.5 μM in order to stain the nuclei, and the plates were sealed with foil plate seals.
The plates were analysed using the Opera plate imager (Opera Confocal Plate Imager, Perkin Elmer) with 7 images per well; 440 ms exposure time per image, 1 μM focal height.

### Data Analysis

Optimised algorithms were used for image analysis to detect and quantify the fluorescence associated with the cell surface as a measure of the cell surface localisation of Fpn-Halotag.
The final display corresponded to the percentage of cells which exhibited membrane fluorescence: wells treated with 100 nM hepcidin produced the lowest values (negative control display=0% inhibition of Fpn internalisation) and wells which had not been treated with hepcidin produced the maximum percentage of cells with membrane fluorescence (positive control display=100% inhibition of Fpn internalisation).
On each plate, the median of the 6 positive and 6 negative control values was used to calculate the percentage inhibition of tested compounds in accordance with the following formula:

$$I = 100 \times \frac{{R}_{neg} - {R}_{compound}}{{R}_{neg} - {R}_{pos}}$$

wherein,
   Rₚₒₛ positive control display value (median)
   R_{neg} negative control display value (median)
   R_{compound} display value of the tested compound
   I percentage inhibition of the respective compound
In dose activity assays dilution series (11 concentrations, 1:2 dilution steps) of the compounds were tested (concentration range from 0.04 to 40 μM), and standard signal values of replicated tests (on average 6 titrations on independent plates) were used for curve adaptation according to a robust standard dose action model with four parameters (lower asymptote, upper asymptote, IC50, gradient).

The following results were obtained for the Examples:

| | | | I [%] |
|---|---|---|---|
| | | | (Median |
| | | | Inhibition [%] |
| | | | at 10 μM |
| | | | substance |
| Example | Compound | IC50 [μM] | conc.) |
| 1 | 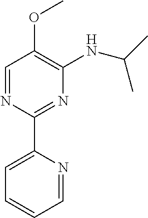 | <50 | >50 |
| 2 | 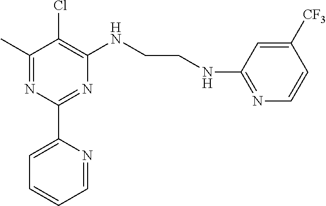 | >40 | <50 |
| 3 | 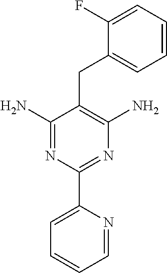 | >40 | <50 |
| 4 | 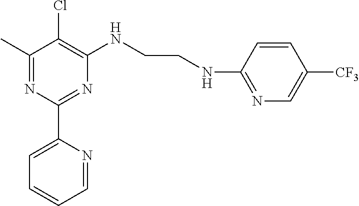 | >40 | <50 |
| 5 | 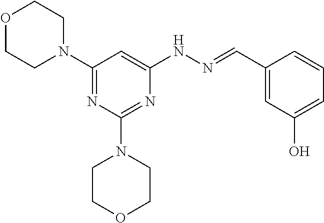 | >40 | >50 |
| 6 | 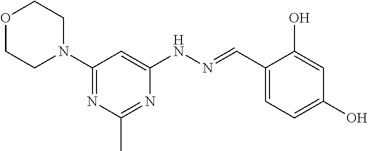 | >40 | >50 |
| 7 | 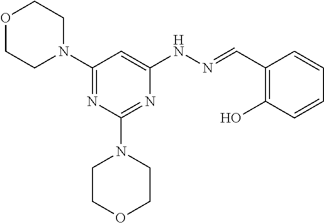 | <50 | >50 |
| 8 | 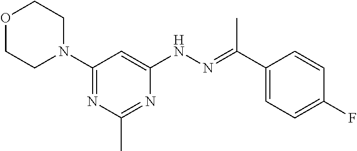 | >40 | <50 |
| 9 | 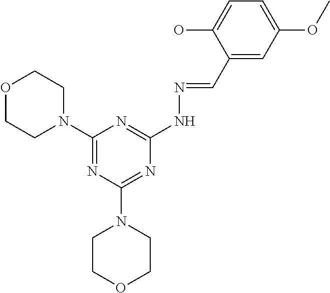 | <50 | >50 |
| 10 | 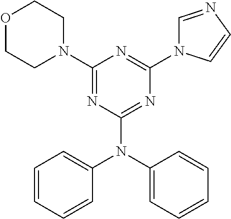 | <50 | >50 |
| 11 | 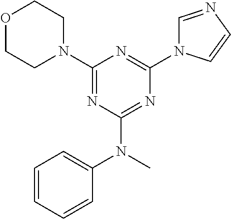 | <50 | <50 |
| 12 | 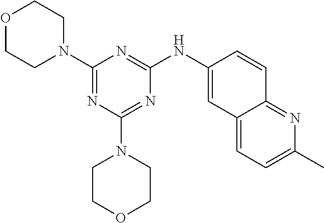 | <50 | <50 |
| 13 | 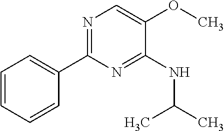 | >50 | |
| 14 | 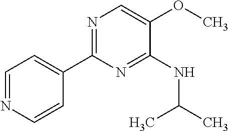 | >50 | |
| 15 | 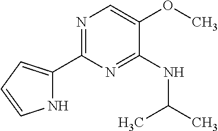 | >50 | |
| 16 | 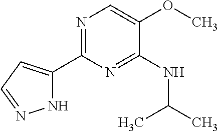 | >50 | |
| 17 | 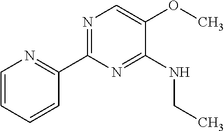 | <50 | |
| 18 | 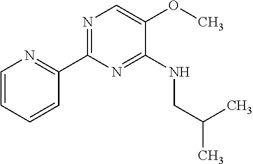 | <50 | |
| 19 | 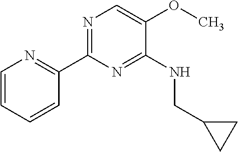 | <50 | |
| 20 | 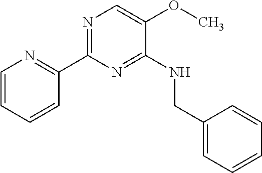 | <50 | |
| 21 | 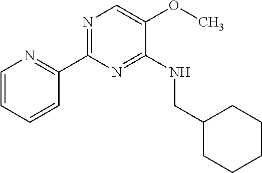 | <50 | |
| 22 | 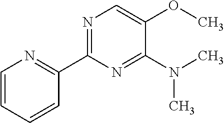 | <50 | |
| 23 | 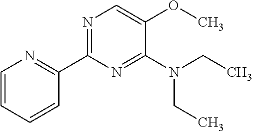 | >50 | |
| 24 | 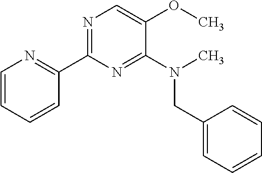 | | |
| 25 | 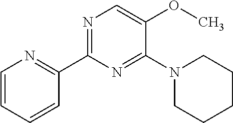 | >50 | |
| 26 | 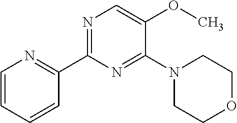 | <50 | |
| 27 | 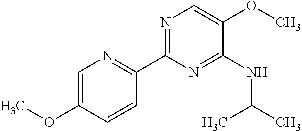 | <50 | |
| 28 | 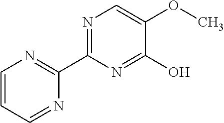 | >50 | |
| 29 | 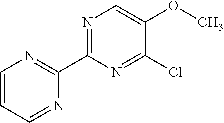 | >50 | |
| 30 | 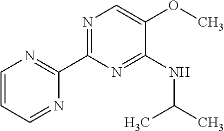 | >50 | |
| 31 | 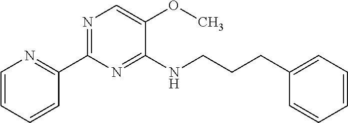 | >50 | |
| 32 | 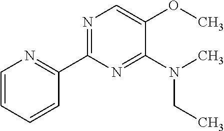 | <50 | |
| 33 | 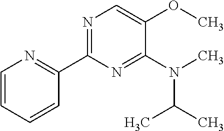 | >50 | |
| 34 | 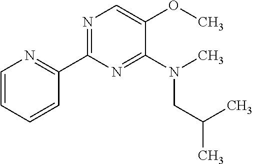 | >50 | |
| 35 | 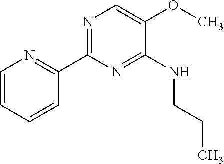 | <50 | |
| 36 | 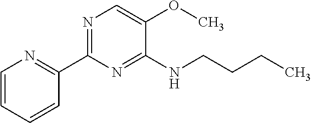 | <50 | |
| 37 | 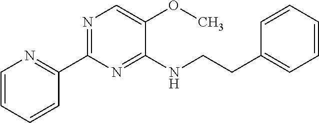 | <50 | |
| 38 | 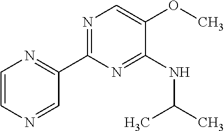 | >50 | |
| 39 | 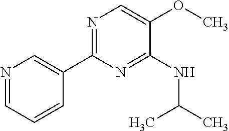 | >50 | |
| 40 | 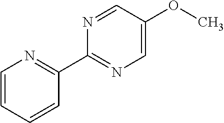 | >50 | |
| 41 | 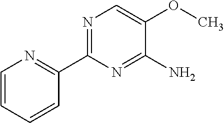 | >50 | |
| 42 | 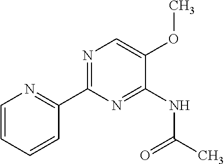 | >50 | |
| 43 | 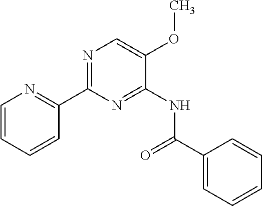 | <50 | |
| 44 | 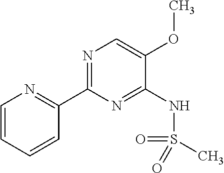 | <50 | |
| 45 | 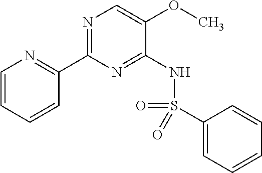 | <50 | |
| 46 | 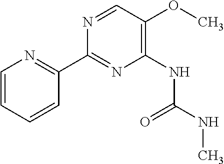 | >50 | |
| 47 | 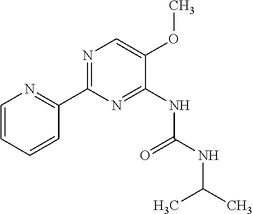 | >50 | |
| 48 | 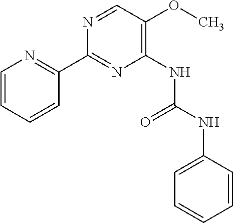 | >50 | |
| 49 | 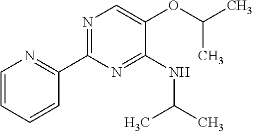 | >50 | |
| 50 | 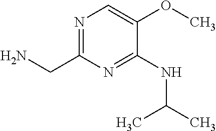 | <50 | |
| 51 | 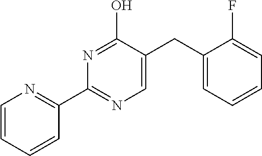 | <50 | |
| 52 | 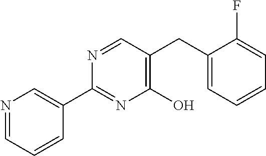 | >50 | |
| 53 | 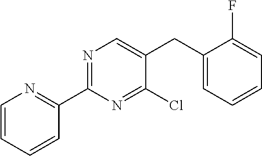 | <50 | |
| 54 | 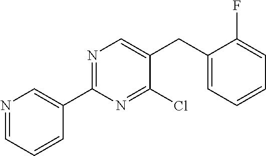 | >50 | |
| 55 | 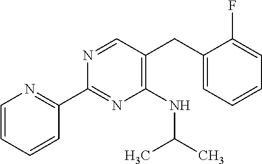 | <50 | |
| 56 | 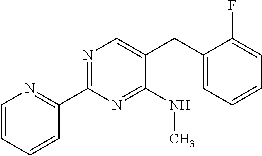 | >50 | |
| 57 | 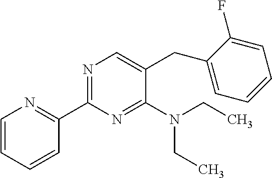 | <50 | |
| 58 | 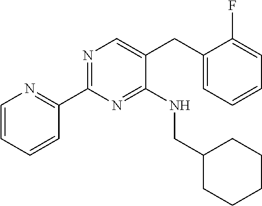 | >50 | |
| 59 | 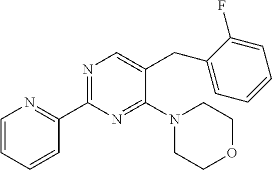 | <50 | |
| 60 | 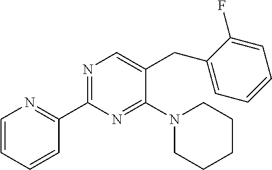 | <50 | |
| 61 | 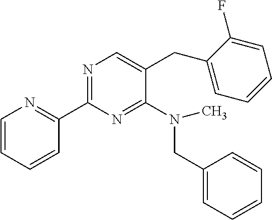 | >50 | |
| 62 | 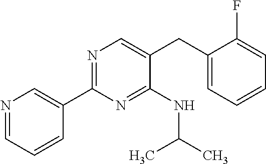 | >50 | |
| 63 | 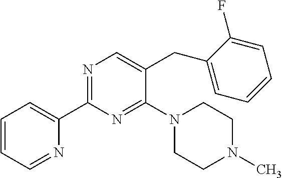 | <50 | |
| 64 | 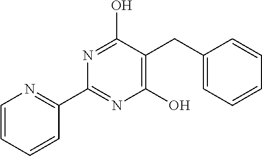 | >50 | |
| 65 | 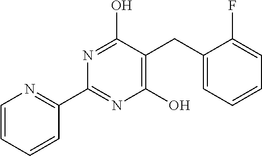 | >50 | |
| 66 | 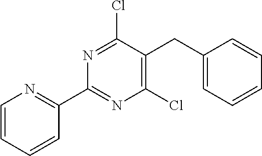 | >50 | |
| 67 | 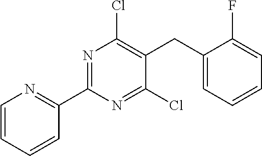 | >50 | |
| 68 | 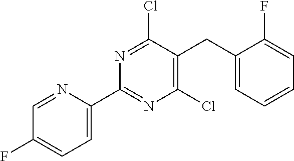 | >50 | |
| 69 | 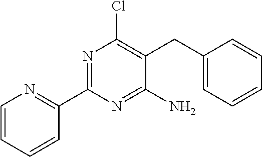 | >50 | |
| 70 | 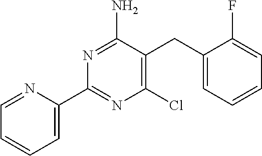 | >50 | |
| 71 | 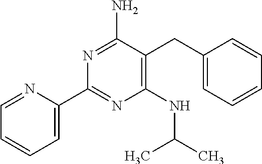 | <50 | |
| 72 | 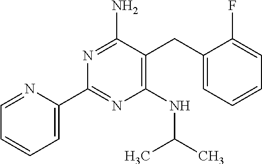 | <50 | |
| 73 | 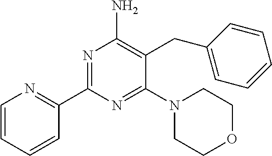 | <50 | |
| 74 | 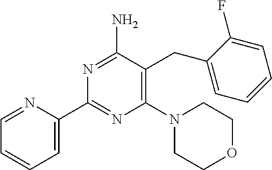 | <50 | |
| 75 | 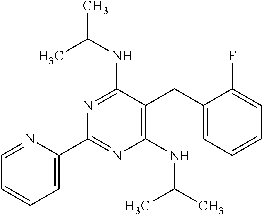 | >50 | |
| 76 | 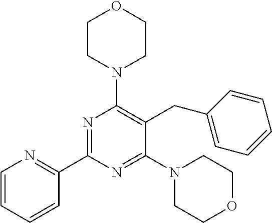 | >50 | |
| 77 | 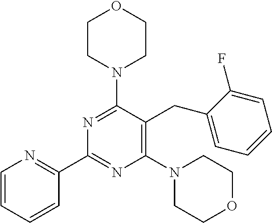 | >50 | |
| 78 | 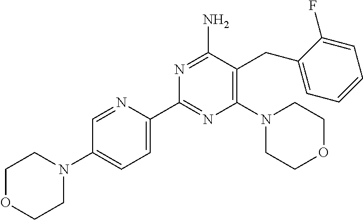 | >50 | |
| 79 | 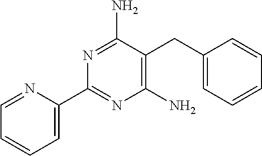 | <50 | |
| 80 | 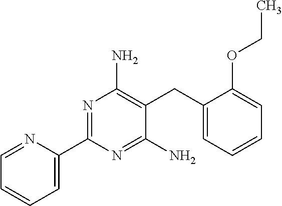 | >50 | |
| 81 | 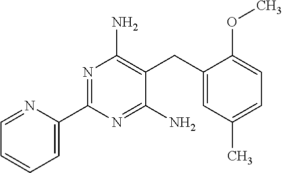 | >50 | |
| 82 | 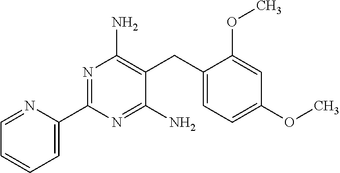 | <50 | |
| 83 | 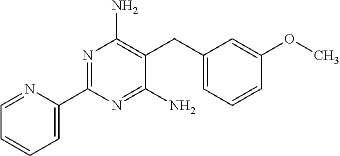 | <50 | |
| 84 | 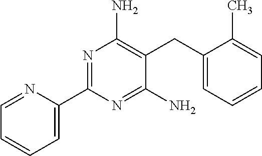 | <50 | |
| 85 | 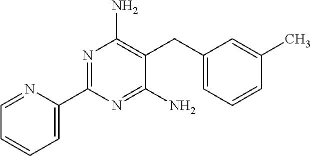 | <50 | |
| 86 | 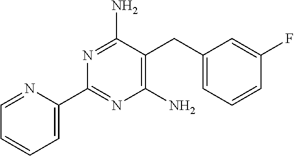 | <50 | |
| 87 | 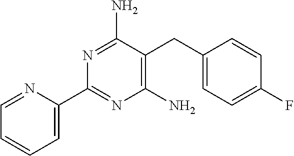 | <50 | |
| 88 | 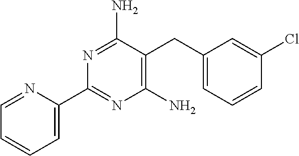 | <50 | |
| 89 | 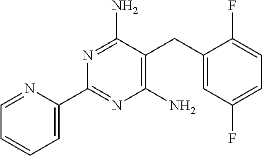 | <50 | |
| 90 | 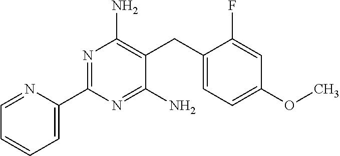 | <50 | |
| 91 | 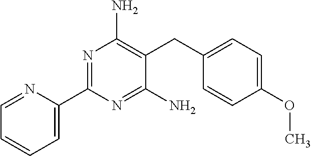 | >50 | |
| 92 | 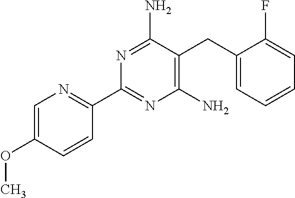 | <50 | |
| 93 | 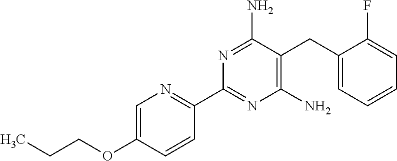 | <50 | |
| 94 | 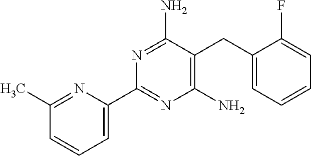 | >50 | |
| 95 | 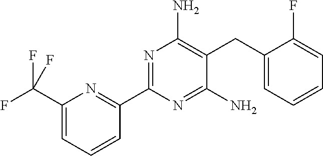 | >50 | |
| 96 | 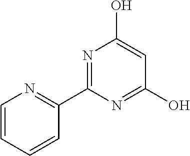 | >50 | |
| 97 | 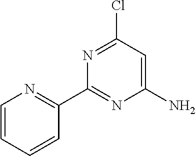 | >50 | |
| 98 | 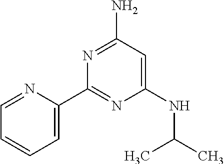 | <50 | |
| 99 | 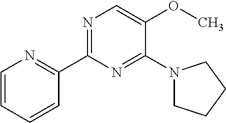 | <50 | |
| 100 | 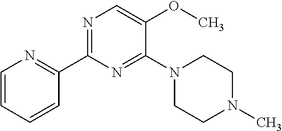 | >50 | |
| 101 | 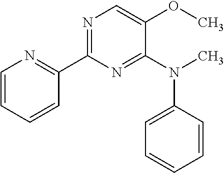 | <50 | |
| 102 | 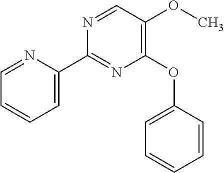 | >50 | |
| 103 | 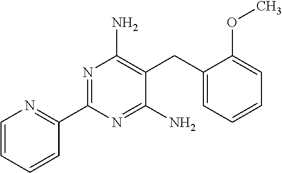 | <50 | |
| 104 | 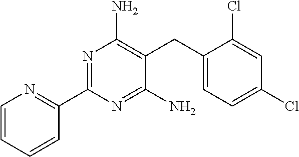 | <50 | |
| 105 | 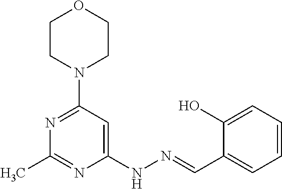 | >50 | |
| 106 | 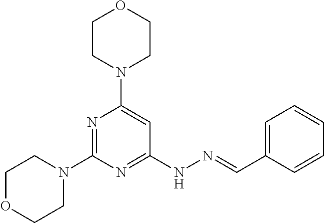 | >50 | |
| 107 | 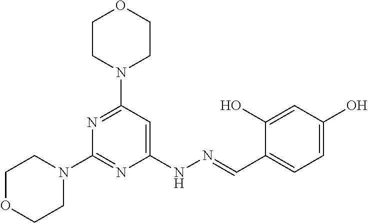 | >50 | |
| 108 | 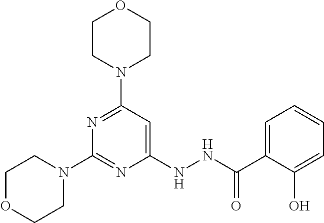 | >50 | |
| 109 | 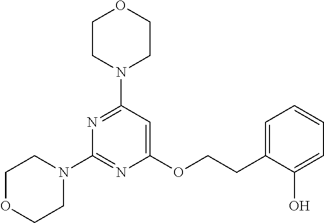 | >50 | |
| 110 | 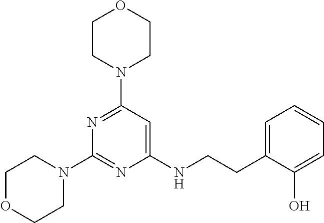 | >50 | |
| 111 | 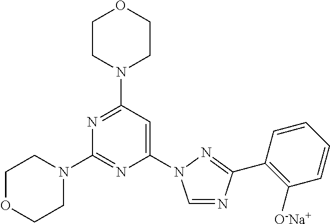 | >50 | |
| 112 | 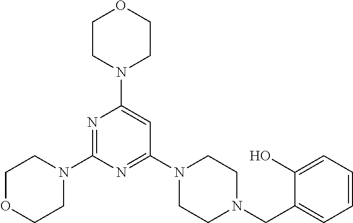 | >50 | |
| 113 | 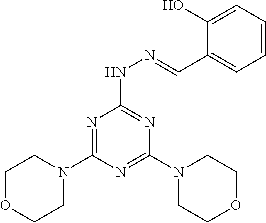 | >50 | |
| 114 | 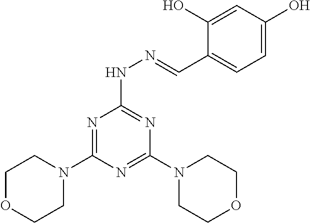 | >50 | |
| 115 | 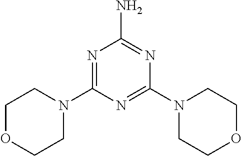 | >50 | |
| 116 | 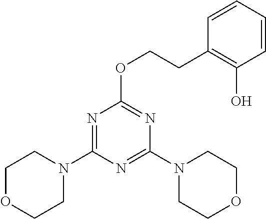 | >50 | |
| 117 | 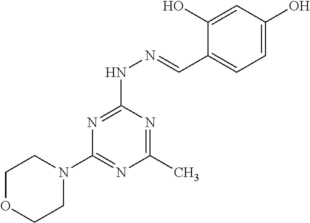 | >50 | |

### Examples of Production 1 to 12

The identification and the purity of compounds 1 to 12 were analysed by HPLC-MS (high performance liquid chromatography with mass spectrometry) or by HPLC with UV detection (PDA: photodiode array).

The following method was used here:

Method: MS19_{—}7MIN_HIRES—POS/High resolution method
Stationary phase/column: Waters Atlantis dC18 100×2.1 mm,
3 μm column, 40° C.
   Mobile phase: A—0.1% formic acid (water)
B—0.1% formic acid (acetonitrile)
   Flow rate: 0.6 ml/min
   Injection volume: 3 μl
   UV detector: 215 nm (nominal)
   or
   MS detection: TIC (total ion count)

| | | Organic |
|---|---|---|
| | | content |
| Gradient | Time (min) | (%) |
| | 0.00 | 5 |
| | 5.00 | 100 |
| | 5.40 | 100 |
| | 5.42 | 5 |

HPLC-MS System: Shimadzu LCMS 2010EV system
Mass range: 100-1000 m/z
Scan rate: 2000 amu/sec

### Compound According to Example 1

### Isopropyl-(5-methoxy-2-pyridin-2-yl-pyrimidin-4-yl)-amine

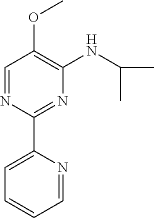

### HP-B002012-001

### MW: 244.29

### Manufacturer BIONET

UV spectrum: λ max [nm]: 214, 235, 321, 345.
HPLC-MS: [m/z]: 245

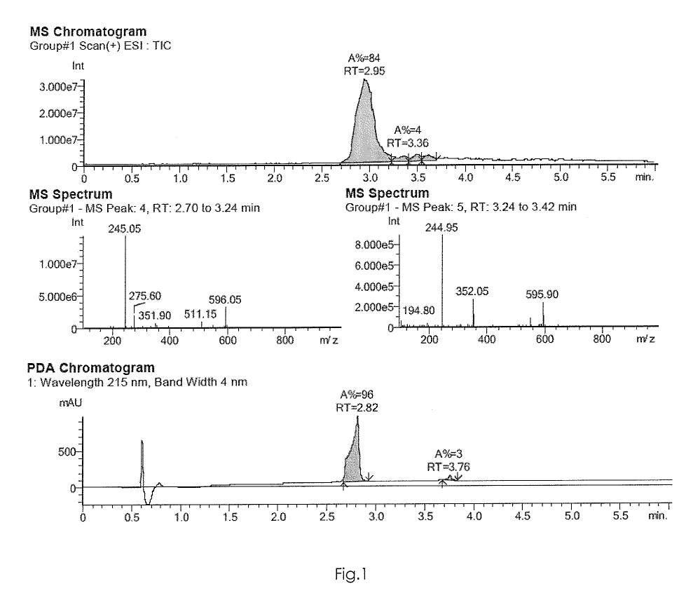

The result is shown in FIG. 1.

### Compound According to Example 2

### N-(5-Chloro-6-methyl-2-pyridin-2-yl-pyrimidin-4-yl)-N′-(4-trifluoromethyl-pyridin-2-yl)-ethane-1,2-diamine

### Route 21

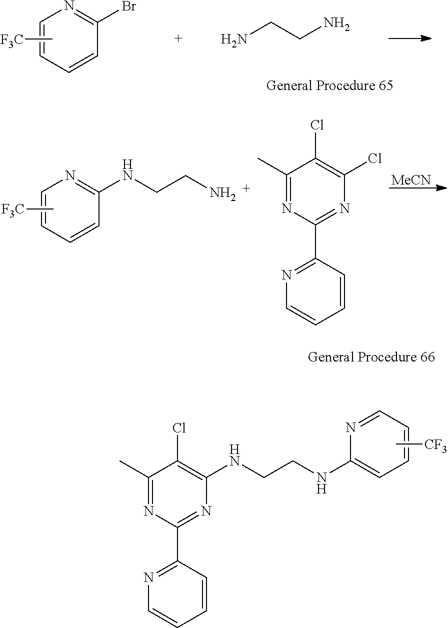

### General Procedure 65:

### N*1*-(4-Trifluoromethyl-pyridin-2-yl)-ethane-1,2-diamine

2-Bromo-4-(trifluoromethyl)pyridine (500 mg, 2.2 mmol) and ethane-1,2-diamine (12.5 ml, 187.5 mmol) were heated under reflux for 2 h. After cooling, the mixture was concentrated in vacuo and the residue was partitioned between DCM and water. The aqueous phase was extracted with DCM and the combined organic phases were washed with water, dried (MgSO₄) and concentrated in vacuo to give the title compound (330 mg, 72%) which was used without further purification. The compound could not be detected by HPLCMS therefore structure was confirmed by NMR.

### General Procedure 66:

### N-(5-Chloro-6-methyl-2-pyridin-2-yl-pyrimidin-4-yl)-N′-(4-trifluoromethyl-pyridin-2-yl)-ethane-1,2-diamine (Example 2)

4,5-Dichloro-6-methyl-2-pyridin-2-yl-pyrimidine (144 mg, 0.63 mmol) was added to a solution of N*1*-(4-trifluoromethyl-pyridin-2-yl)-ethane-1,2-diamine (120 mg, 0.63 mmol) in MeCN (5 ml) and the mixture was stirred at room temperature for 18 h followed by heating under reflux for 4 h. After cooling, the mixture was concentrated in vacuo. The crude residue was purified by column chromatography with EtOAc/heptane (0:100-100:0) as the eluent to give the title compound (35 mg, 13%).

### MW: 408.8

HPLCMS (Method A as described for the compounds of examples 13-104):
[m/z]: 408.9

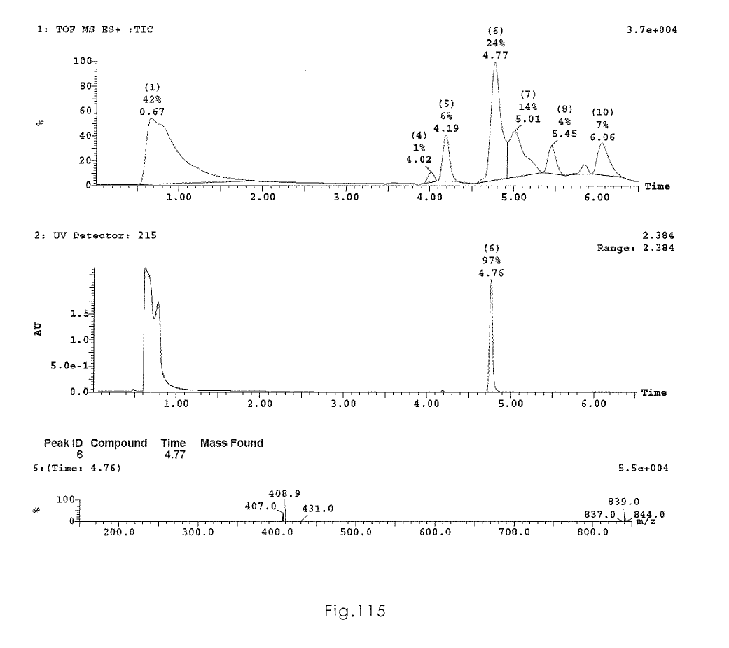

FIG. 115 shows the chromatograms/spectra of the compound of example 2.

### IC50 [μM]: >40

### Compound According to Example 3

### 5-(2-Fluoro-benzyl)-2-pyridin-2-yl-pyrimidine-4,6-diamine

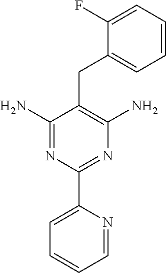

### HP-AB002020-B09

### MW: 295.31

### Manufacturer BIONET

UV spectrum: λ max [nm]: 195, 225, 293
HPLC-MS: [m/z]: 296

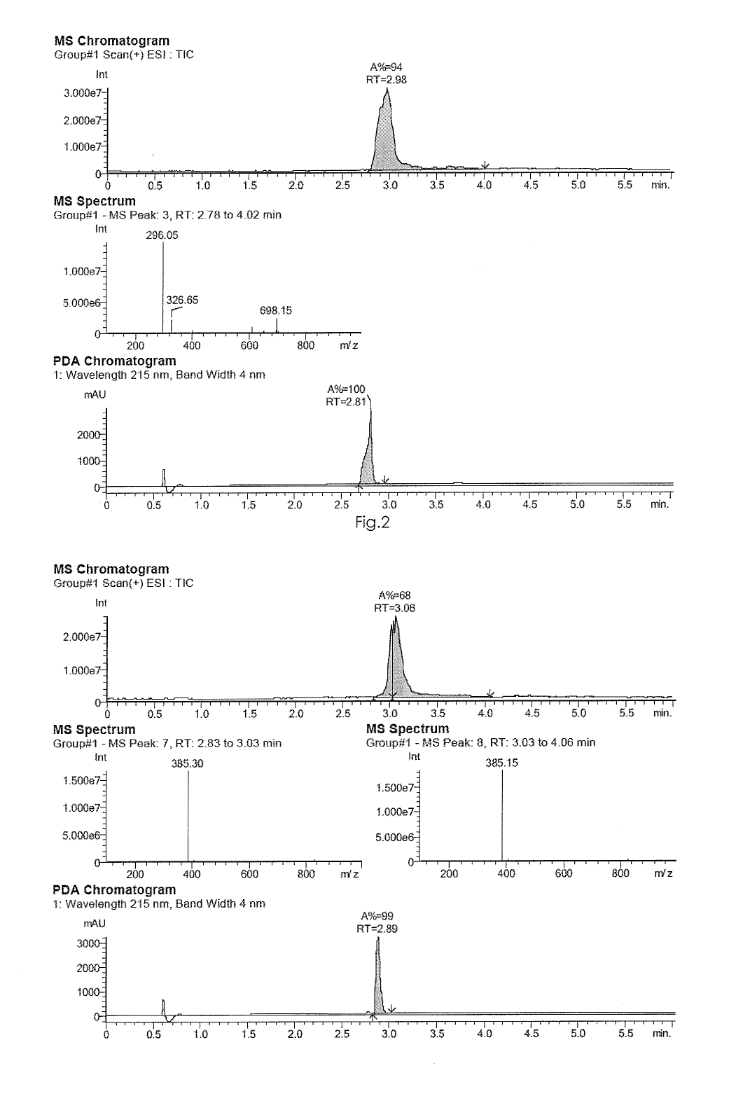

The result is shown in FIG. 2.

### Compound According to Example 4

### N*1*-(5-Trifluoromethyl-pyridin-2-yl)-ethane-1,2-diamine

In a similar fashion using route 21 general procedure 65 (see example 2), 2-bromo-5-(trifluoromethyl)pyridine (100 mg, 0.44 mmol) and ethane-1,2-diamine (2.5 ml, 37.5 mmol) gave the title compound (60 mg, 65%) which was used without further purification. The compound could not be detected by HPLCMS therefore structure was confirmed by NMR.

### N-(5-Chloro-6-methyl-2-pyridin-2-yl-pyrimidin-4-yl)-N′-(5-trifluoromethyl-pyridin-2-yl)-ethane-1,2-diamine (Example 4)

In a similar fashion using route 21 general procedure 66 (see example 2), N*1*-(5-trifluoromethyl-pyridin-2-yl)-ethane-1,2-diamine (60 mg, 0.32 mmol) and 4,5-dichloro-6-methyl-2-pyridin-2-yl-pyrimidine (77 mg, 0.32 mmol) in dioxane (5 ml) gave the title compound.

### MW: 408.8

HPLCMS (Method A as described for the compounds of examples 13-104):
[m/z]: 409

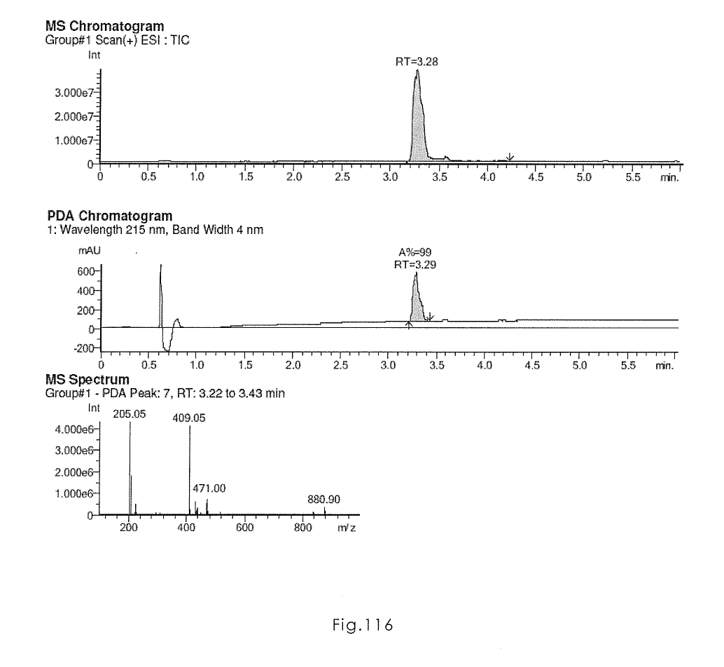

FIG. 116 shows the MS chromatogram, the MS spectrum and the PDA chromatogram of the compound of example 4.

### IC50 [μM]: >40

### Compound According to Example 5

### 3-[(2,6-Dimorpholin-4-yl-pyrimidin-4-yl)-hydrazonomethyl]-phenol

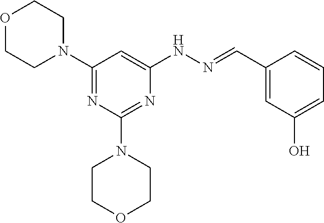

### HP-AN003030-E11

### MW: 384.43

### Manufacturer VITAS M LABS

UV spectrum: λ max [nm]: 214, 235, 321, 345.
HPLC-MS: [m/z]: 385

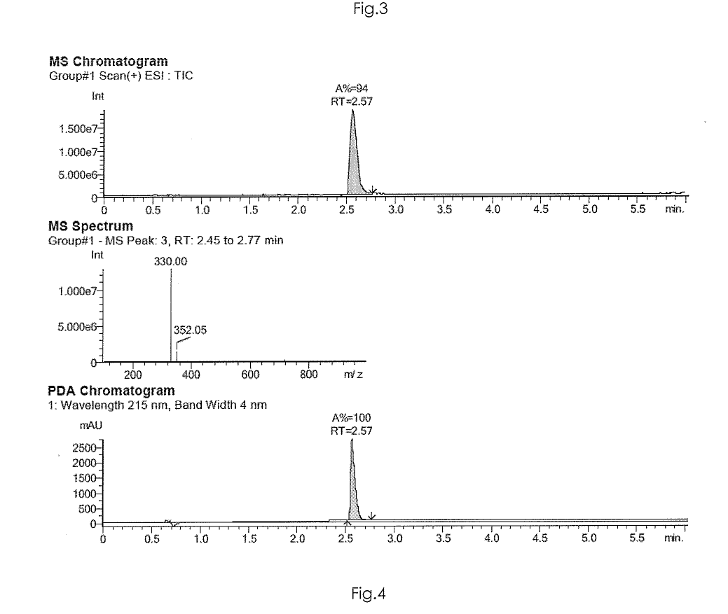

The result is shown in FIG. 3.

### Compound According to Example 6

### 4-[(2-Methyl-6-morpholin-4-yl-pyrimidin-4-yl)-hydrazonomethyl]-benzene-1,3-diol

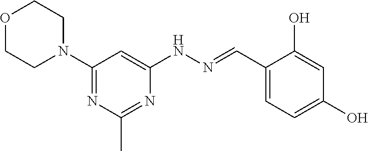

### HP-AA004168-B11

### MW: 329.35

### Manufacturer ASINEX

UV spectrum: λ max [nm]: 212, 241, 346
HPLC-MS: [m/z]: 330

The result is shown in FIG. 4.

### Compound According to Example 7

### 2-[(2,6-Dimorpholin-4-yl-pyrimidin-4-yl)-hydrazonomethyl]-phenol

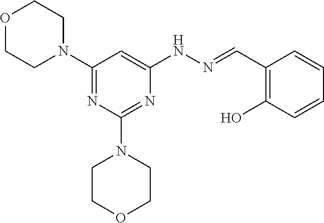

### HP-AN003030-F11

### MW: 384.43

### Manufacturer VITAS M LABS

UV spectrum: λ max [nm]: 222, 284,332
HPLC-MS: [m/z]: 385

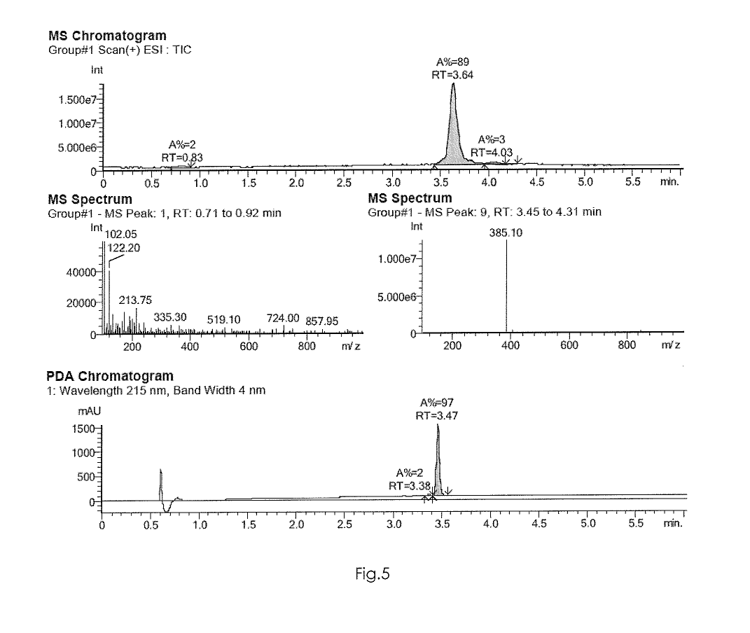

The result is shown in FIG. 5.

### Compound According to Example 8

### N-[1-(4-Fluoro-phenyl)-ethylidene]-N′-(2-methyl-6-morpholin-4-yl-pyrimidin-4-yl)-hydrazine

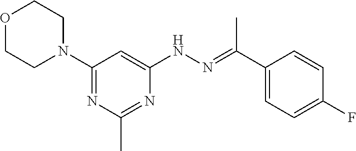

### HP-AA004168-D11

### MW: 329.37

### Manufacturer ASINEX

UV spectrum: λ max [nm]: 198, 230, 322
HPLC-MS: [m/z]: 330

The result is shown in FIG. 6.

### Compound According to Example 9

### 2-[(4,6-Dimorpholin-4-yl-[1,3,5]triazin-2-yl)-hydrazonomethyl]-4-methoxy-phenol

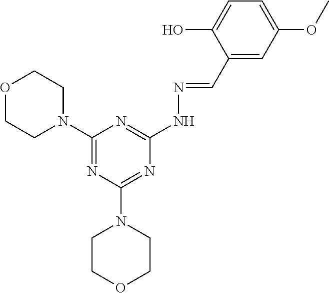

### HP-AA004154-A01

### MW: 415.45

### Manufacturer ASINEX

UV spectrum: λ max [nm]: 232, 290, 343
HPLC-MS: [m/z]: 416

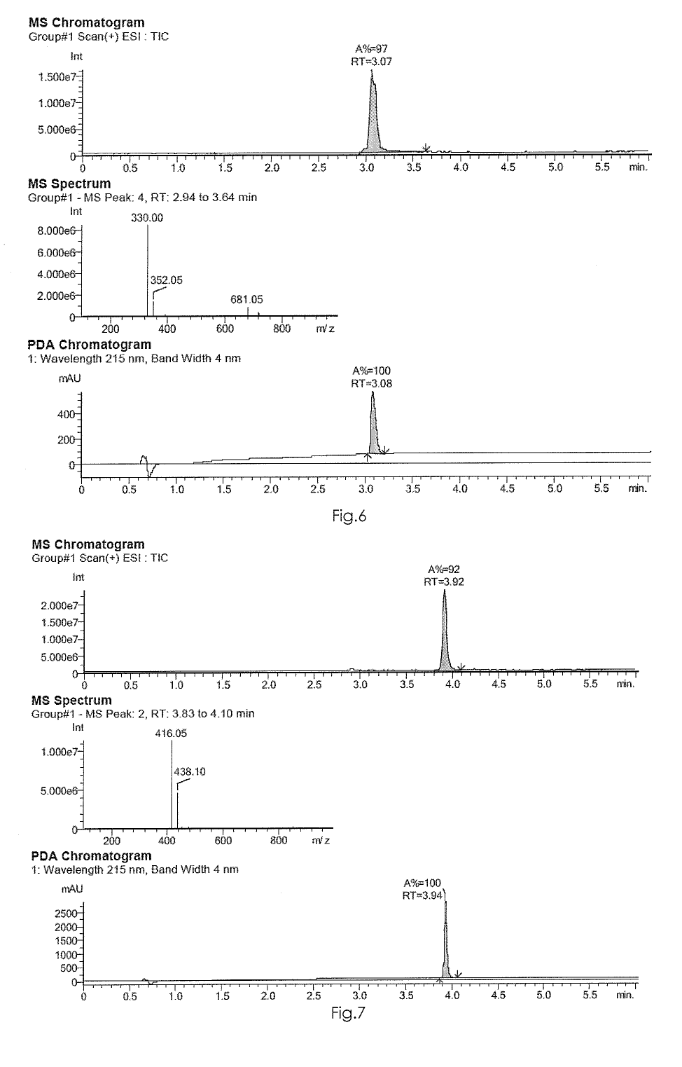

The result is shown in FIG. 7.

### Compound According to Example 10

### (4-Imidazol-1-yl-6-morpholin-4-yl-[1,3,5]triazin-2-yl)-diphenyl-amine

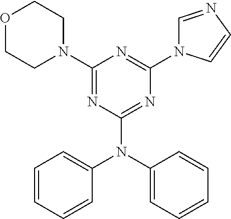

### HP-AN004039-H04

### MW: 399.48

### Manufacturer VITASMLAB

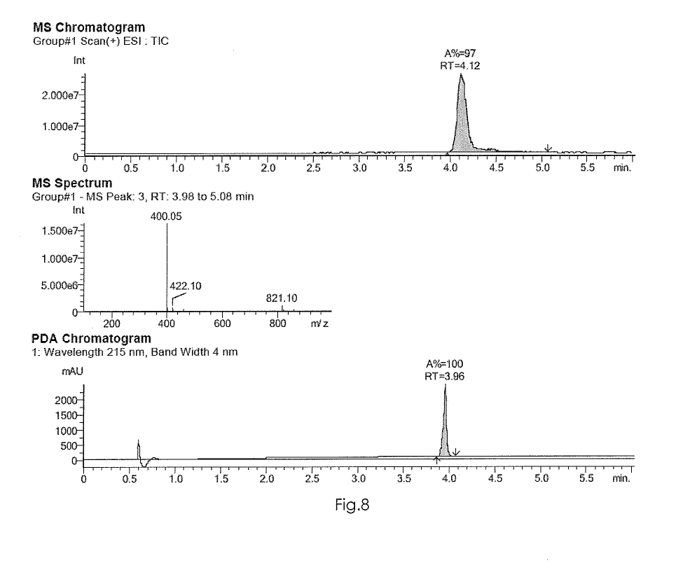

UV spectrum: λ max [nm]: 195, 239
HPLC-MS: [m/z]: 400
The result is shown in FIG. 8.

### Compound according to Example 11

### (4-Imidazol-1-yl-6-morpholin-4-yl-[1,3,5]triazin-2-yl)-methyl-phenyl-amine

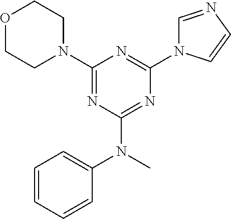

### HP-AN004039-F04

### MW: 337.38

### Manufacturer VITASMLAB

UV spectrum: λ max [nm]: 190, 202, 235
HPLC-MS: [m/z]: 338

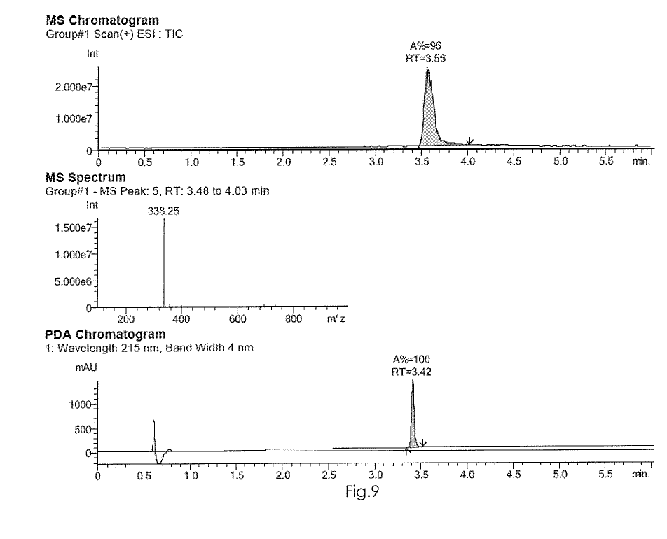

The result is shown in FIG. 9.

### Example 12

### (4,6-Dimorpholin-4-yl-[1,3,5]triazin-2-yl)-(2-methyl-quinolin-6-yl)-amine

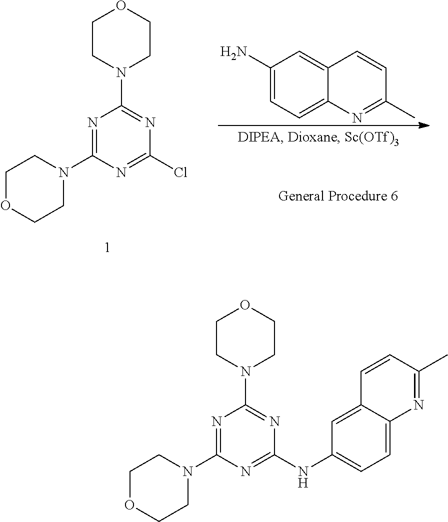

6-amino-2-methylquinoline (30 mg, 0.19 mmol) was added to a solution of 2-chloro-4,6-dimorpholin-4-yl-[1,3,5]triazine (50 mg, 0.18 mmol) in dioxane (0.5 ml) followed by DIPEA (92 μl, 0.53 mmol) and the mixture was heated at 50° C. for 1 h. The temperature was increased to 90° C. for 1 h and 100° C. for 18 h. Only 4% conversion to desired product had occurred therefore the mixture was transferred to a microwave tube together with an excess of 6-amino-2-methylquinoline and catalytic scandium triflate. The mixture was heated at 150° C. in the microwave for a total of 3.5 h. After cooling, the mixture was concentrated in vacuo. The crude residue was triturated from MeOH to give the title compound (17 mg, 24%).

### MW: 407.48

HPLCMS (Method A as described for the compounds of examples 113-117): [m/z]: 408

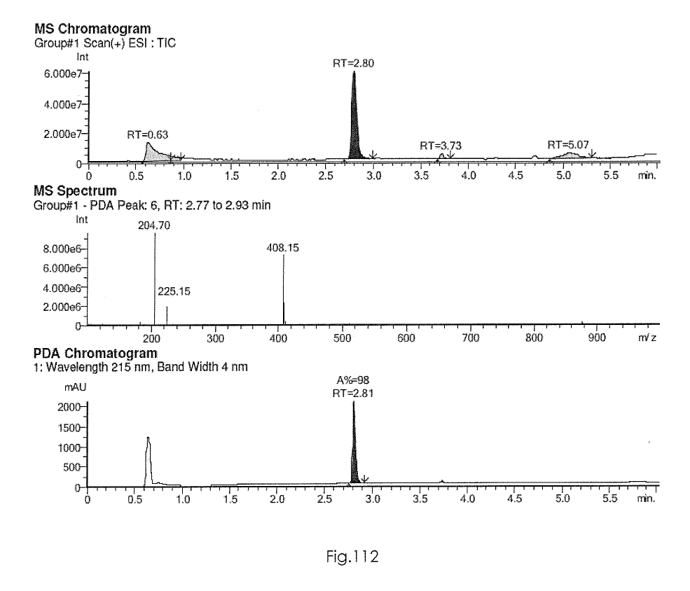

FIG. 112 shows the MS chromatogram, the MS spectrum and the PDA chromatogram of the compound of example 12.

### IC50 [μM]: <50

### Examples of Production 13 to 104

The following analytical methods were adopted in Examples 13 to 104 below:

### Analytical HPLC-MS

### Method A

Column: Waters Atlantis dC18 (2.1×100 mm, 3 μm column)
Flow rate 0.6 ml/min
Solvent A: 0.1% formic acid/water
Solvent B: 0.1% formic acid/acetonitrile
Injection volume: 3 μl
Column temperature 40° C.
UV detection wavelength: 215 nm
Eluent: 0 min (=minutes) to 5 min, constant gradient from 95% solvent A+5% solvent B to 100% solvent B; 5 min to 5.4 min, 100% solvent B; 5.4 min to 5.42 min, constant gradient from 100% solvent B to 95% solvent A+5% solvent B; 5.42 min to 7.00 min, 95% solvent A+5% solvent B

### Method B

Column: Waters Atlantis dC18 (2.1×50 mm, 3 μm)
Solvent A: 0.1% formic acid/water
Solvent B: 0.1% formic acid/acetonitrile
Flow rate 1 ml/min
Injection volume 3 μl
UV detection wavelength: 215 nm
Eluent: 0 to 2.5 min, constant gradient from 95% solvent A+5% solvent B to 100% solvent B; 2.5 min to 2.7 min, 100% solvent B; 2.71 to 3.0 min, 95% solvent A+5% solvent B.

### Method C

Column: Waters Atlantis dC18 (2.1×30 mm, 3 μm column)
Flow rate 1 ml/min
Solvent A: 0.1% formic acid/water
Solvent B: 0.1% formic acid/acetonitrile
Injection volume: 3 μl

UV detection wavelength: 215 nm

Eluent: 0 min to 1.5 min, constant gradient from 95% solvent A+5% solvent B to 100% solvent B; 1.5 min to 1.6 min, 100% solvent B; 1.60 min to 1.61 min, constant gradient from 100% solvent B to 95% solvent A+5% solvent B; 1.61 min to 2.00 min, 95% solvent A+5% solvent B.
MS detection using Waters LCT or LCT Premier, or ZQ or ZMD
UV detection using Waters 2996 photodiode array or Waters 2787 UV or Waters 2788 UV

### Method D

Column: Atlantis dC18 50 mm×3 mm; 3 μm
Mobile phase A: 0.1% formic acid/water
Mobile phase B: 0.1% formic acid/acetonitrile
Flow rate 0.8 ml/min.
Detection wavelength: Diode array spectrum λ max (with scan in range of 210-350 nm)
Sampling rate: 5
Column temperature: 35° C.
Injection volume: 5 μl
Eluent: 0 min 95% solvent A+5% solvent B, 0.2 min 95% solvent A+5% solvent B; 0.2 min to 3.2 min constant gradient from 95% solvent A+5% solvent B to 5% solvent A and 95% solvent B; 5 min 5% solvent A and 95% solvent B; 5 min to 5.2 min constant gradient from 5% solvent A and 95% solvent B to 95% solvent A+5% solvent B; 5.5 min 95% solvent A and 5% solvent B.
MS detection using Waters LCT or LCT Premier, or ZQ or ZMD
UV detection using Waters 2996 photodiode array or Waters 2787 UV or Waters 2788 UV

### Method E

### Column: Phenomenex Gemini C18 2.0×100 mm; 3 μm

Mobile phase A: 2 mM ammonium bicarbonate, buffered to pH=10
Mobile phase A: acetonitrile
Flow rate 0.5 ml/min.
UV detection wavelength: 215 nm
Column temperature: 60° C.
Injection volume: 3 μl
Eluent: 0 min 95% solvent A+5% solvent B, 0.2 min to 5.50 min, constant gradient from 95% solvent A+5% solvent B to 100% solvent B; 5.50-5.90 min 100% solvent B; 5.90-5.92 min gradient from 100% solvent B to 95% solvent A+5% solvent B.

### Preparative HPLC—Neutral Conditions

### Column: Waters SunFire Prep C18 OBD (5 μm 19×100 mm)

Flow rate 20 ml/min

### Solvent A: Water

Solvent B: acetonitrile
Injection volume: 1000 μl
Column temperature: ambient temperature

### Detection: UV-based

Eluent: 0 min to 2 min, 5% solvent B+95% solvent A; 2 min to 2.5 min constant gradient to 10% solvent B+90% solvent A, 2.5 min to 14.5 min constant gradient to 100% solvent B; 14.5 min to 16.5 min, 100% solvent B; 16.5 to 16.7 min constant gradient to 5% B+95% A; 16.7 min to 17.2 min, 5% solvent B+95% solvent A. Gilson semi-preparative HPLC module with 119 UV detector and 5.11 Unipoint control software

### Preparative HPLC—Acidic Conditions

### Column: Waters SunFire Prep C18 OBD (5 μm 19×100 mm)

Flow rate 26 ml/min

### Solvent A: 0.1% TFA/water

### Solvent B: 0.1% TFA/acetonitrile

Injection volume: 1000 μl
Column temperature: ambient temperature
Detection: based on mass
Eluent: 0 min to 1 min 90% solvent A+10% solvent B; 1 min to 7.5 min, constant gradient from 90% solvent A+10% solvent B to 100% solvent B; 7.5 min to 9 min, 100% solvent B; 9 min to 9.1 min, constant gradient from 1000% solvent B to 90% solvent A+10% solvent B; 9.1 min to 10 min, 90% solvent A+10% solvent B.
Waters Micromass platform LCZ single quadrupole mass spectrometer.
Waters 600 solvent delivery system
Waters 515 auxiliary pumps
Waters 2487 UV detector
Gilson 215 autosampler and fraction collector

### Preparative HPLC—Basic Conditions

### Column: XBridge Prep C18 OBD (5 μm 19×100 mm)

Flow rate 20 ml/min
Solvent A: Water+0.2% ammonium hydroxide
Solvent B: acetonitrile+0.2% ammonium hydroxide
Injection volume: 1000 μl
Column temperature: ambient temperature
Detection: directed UV
Eluent: 0 min to 2 min, 5% solvent B+95% solvent A; 2 min to 2.5 min constant gradient to 10% solvent B+90% solvent A, 2.5 min to 14.5 min constant gradient to 100% solvent B; 14.5 min to 16.5 min, 100% solvent B; 16.5 to 16.7 min constant gradient to 5% B+95% A; 16.7 min to 17.2 min 5% solvent B+95% solvent A.
Gilson semi-preparative HPLC module with 119 UV detector and 5.11 Unipoint control software
Flash silica gel chromatography was carried out on silica gel 230-400 mesh or on pre-packed silica cartridges.
Microwave reactions were carried out using a CEM Discover or Explorer focussed microwave device.

### Naming of Compounds

Some compounds were isolated as TFA or HCl salts, but this is not reflected in their chemical names. In the context of the present invention, the chemical name therefore denotes the compound in neutral form and as the TFA salt or some other salt, in particular a pharmaceutically acceptable salt, where applicable.

### ABBREVIATIONS

nBuLi n-butyllithium
nBuOH n-butanol
cat catalytic
mCPBA m-chloroperoxybenzoic acid
DCM dichloromethane
DIPEA N,N-diisopropylethylamine
DMF N,N-dimethylformamide
Et₂O diethylether
EtOAc ethyl acetate
EtOH ethanol
h hour(s)
HPLC high performance liquid chromatography
LiHMDS lithium hexamethyldisilazide
MeCN acetonitrile
MeOH methanol
min minute(s)
MW molecular weight
NaOMe sodium methoxide
Pd₂(dba)₃ tris(dibenzylidene acetone)dipalladium(0)
nPrOH n-propanol
Py pyridine
TEA triethylamine
THF tetrahydrofuran
TMSOTf trimethylsilyltrifluoromethanesulfonate
IC50 [μM] values were determined in the above-described manner.

Some starting compounds are commercially available, for example some dichloropyrimidines and trichloropyrimidines. These were reacted by a method similar to the generally described methods of synthesis (see patent text and following general procedures), as known to the person skilled in the art, to form the end products. 4,6-dichloropyrimidine [1193-21-1] and 2,4,6-trichloropyrimidine [3764-01-01] from Sigma Aldrich are mentioned as examples of commercial starting compounds.

### Example 13

The compound of Example 13 was produced in accordance with the following Route 1:

### Route 1

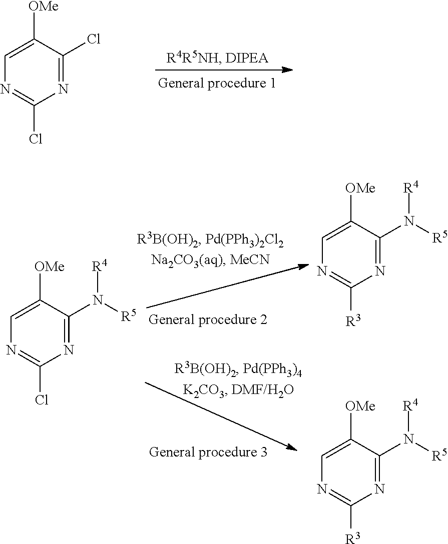

### General Procedure 1:

### 2-(Chloro-5-methoxy-pyrimidin-4-yl)-isopropyl-amine

Iso-propylamine (0.86 ml, 10.02 mmol) was added dropwise to a solution of 2,4-dichloro-5-methoxy-pyrimidine (1.63 g, 9.11 mmol) and DIPEA (1.91 ml, 10.93 mmol) in EtOH (33 ml). The reaction mixture was stirred at room temperature for 29 h and concentrated in vacuo. The residue was dissolved in EtOAc and washed with saturated aqueous NaHCO₃ solution and brine. The organic phase was dried (Na₂SO₄) and concentrated in vacuo. The crude product was purified by column chromatography, with EtOAc/heptane (45:55) as the eluent to give the title compound (1.1 g, 60%).

### MW: 201.66

HPLCMS (Method B): [m/z]: 202

### General Procedure 2:

### Isopropyl-(5-methoxy-2-phenyl-pyrimidin-4-yl)-amine (Example 13)

Bis(triphenylphosphine)palladium(II) dichloride (27 mg, 36 μmol) was added to a mixture of (2-chloro-5-methoxy-pyrimidin-4-yl)-isopropyl-amine (150 mg, 0.75 mmol), phenyl boronic acid (90 mg, 0.75 mmol), Na₂CO₃ (1M solution in water, 0.75 ml, 1.50 mmol) and MeCN (1.5 ml) in a microwave tube. The mixture was de-gassed with N₂ for 5 min. The reaction mixture was heated at 150° C. for 5 min in the microwave. The reaction mixture was filtered and the organic phase of the filtrate was separated. The aqueous phase was extracted with EtOAc (×3). The combined organic phases were dried (Na₂SO₄) and concentrated in vacuo. The crude product was purified by preparative HPLC (neutral conditions) to give the title compound (95 mg, 52%).

### MW: 243.31

HPLCMS (Method A): [m/z]: 244

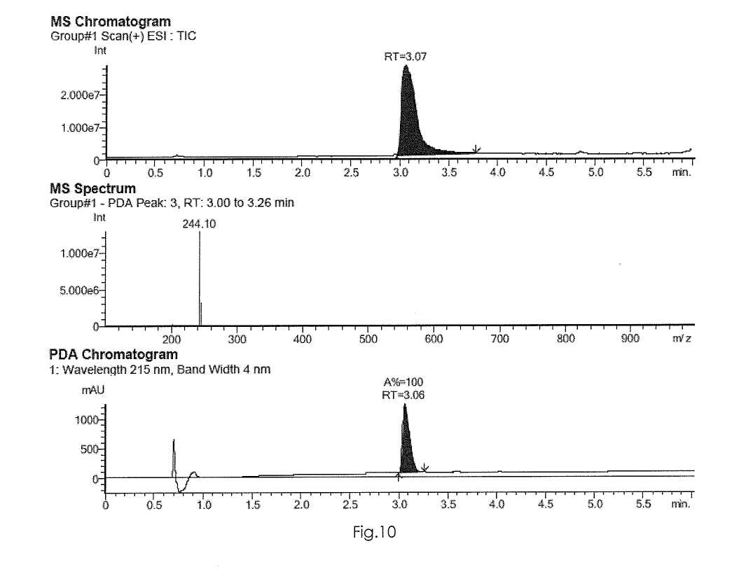

FIG. 10 shows the MS chromatogram, the MS spectrum and the PDA chromatogram of the compound of example 13.

### IC50 [μM]: >50

### Example 14

### Isopropyl-(5-methoxy-2-pyridin-4-yl-pyrimidin-4-yl)-amine

In a similar fashion using route 1 general procedure 2, bis(triphenylphosphine)palladium(II) dichloride (36 mg, 51 μmol), (2-chloro-5-methoxy-pyrimidin-4-yl)-isopropyl-amine (200 mg, 1.0 mmol), pyridin-4-yl boronic acid (120 mg, 1.0 mmol), Na₂CO₃ (1M solution in water, 0.5 ml, 2.0 mmol) gave the title compound (20 mg, 7%) after purification by preparative HPLC (neutral conditions).

### MW: 244.30

HPLCMS (Method A): [m/z]: 245

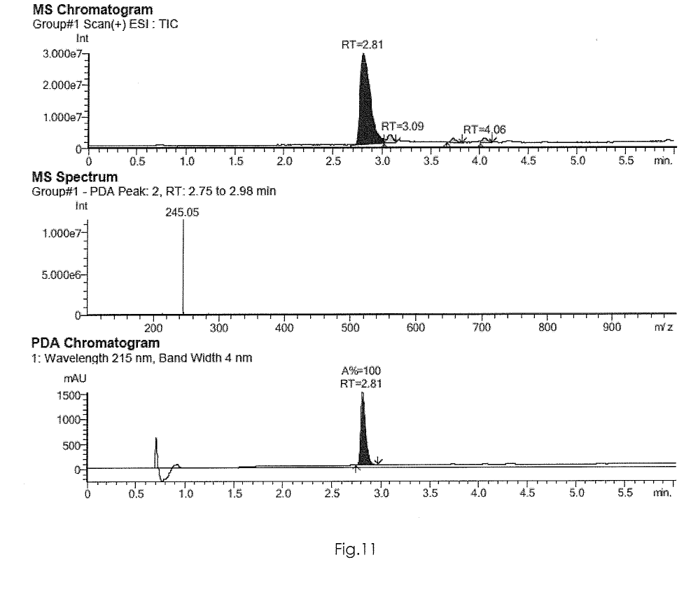

FIG. 11 shows the MS chromatogram, the MS spectrum and the PDA chromatogram of the compound of example 14.

IC50 [μM]: >50

### Example 15

### General Procedure 3:

### Isopropyl-[5-methoxy-2-(1H-pyrrol-2-yl)pyrimidin-4-yl]-amine

(2-Chloro-5-methoxy-pyrimidin-4-yl)-isopropyl-amine (0.2 g, 0.99 mmol), potassium carbonate (0.27 g, 1.9 mmol), N-Boc-2-pyrrole boronic acid (0.31 g, 1.4 mmol), in DMF (3 ml) and water (1.5 ml) were de-gassed and tetrakis(triphenylphosphine)palladium(0) (57 mg, 0.05 mmol) was added under argon. The reaction mixture was heated for 10 min at 150° C. in the microwave. Water (10 ml) was added and the aqueous phase was extracted with DCM (×3). The combined organic phases were dried (Na₂SO₄) and concentrated in vacuo. The crude residue was purified by column chromatography with EtOAc/hexane (1:9-3:7) as the eluent to give the title compound (0.048 g, 21%).

### MW: 232.28

HPLCMS (Method A): [m/z]: 233

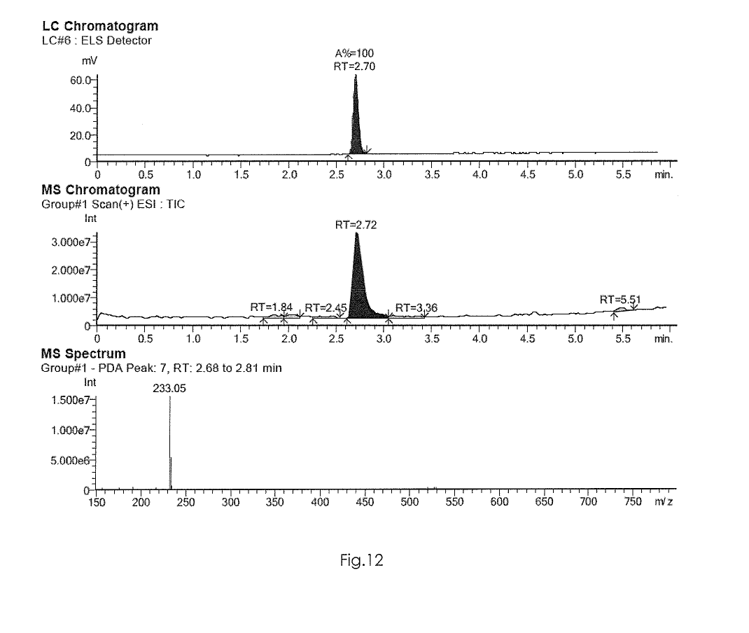

FIG. 12 shows the LC chromatogram, the MS spectrum and the MS chromatogram of the compound of example 15.

### IC50 [μM]: >50

### Example 16

### Isopropyl-[5-methoxy-2-(1H-pyrazol-5-yl)pyrimidin-4-yl]-amine

In a similar fashion using route 1, general procedure 3, (2-chloro-5-methoxy-pyrimidin-4-yl)-isopropyl-amine (0.1 g, 0.4 mmol), potassium carbonate (0.14 g, 0.98 mmol), 1H-pyrrazole-5-boronic acid (82 mg, 0.68 mmol) and tetrakis(triphenylphosphine)palladium(0) (0.06 g, 0.034 mmol) gave the title compound (27 mg, 25%) after purification by column chromatography with DCM/MeOH (98:2) as the eluent.

### MW: 233.27

HPLCMS (Method A): [m/z]: 234

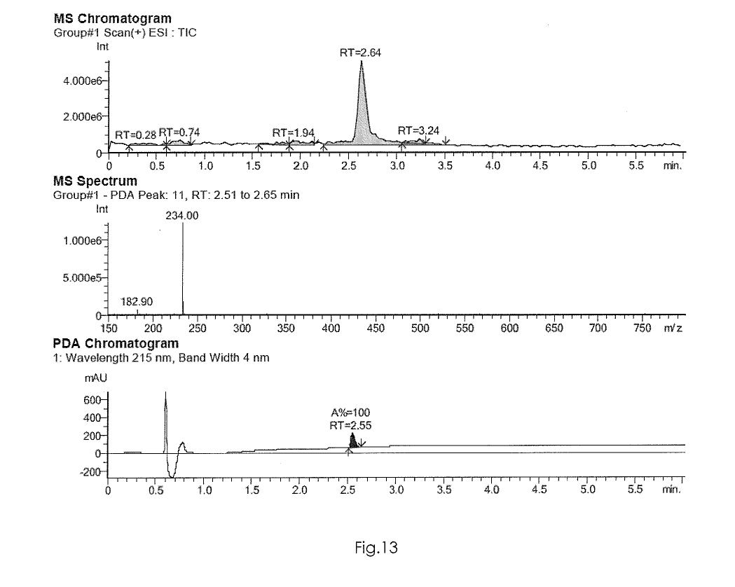

FIG. 13 shows the MS chromatogram, the MS spectrum and the PDA chromatogram of the compound of example 16.

### IC50 [μM]: >50

### Route 2

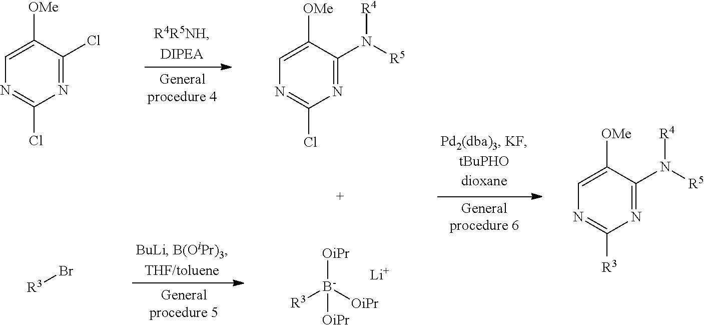

### General Procedure 4:

### (2-Chloro-5-methoxy-pyrimidin-4-yl)-ethyl-amine

2,4-Dichloro-5-methoxypyrimidine (0.1 g, 0.56 mmol), ethylamine (27 mg, 0.64 mmol) and DIPEA (0.12 ml, 0.67 mmol) were dissolved in ethanol (2 ml) and the mixture was stirred at room temperature for 15 h. The mixture was concentrated in vacuo. The residue was diluted with water (15 ml) and the reaction mixture was extracted with EtOAc (×3). The combined organic phases were dried (Na₂SO₄) and concentrated in vacuo to give the title compound (104 mg, 100%).

### MW: 187.63

HPLCMS (Method D): [m/z]: 188

### (2-Chloro-5-methoxy-pyrimidin-4-yl)-isobutyl-amine

In a similar fashion using route 2 general procedure 4, 2,4-dichloro-5-methoxypyrimidine (0.3 g, 1.6 mmol), iso-butylamine (0.13 g, 1.84 mmol) and DIPEA (0.58 ml, 3.3 mmol) gave the title compound (0.36 g, 99%).

### MW: 215.68

HPLCMS (Method D): [m/z]: 216

### (2-Chloro-5-methoxy-pyrimidin-4-yl)-cyclopropylmethyl-amine

In a similar fashion using route 2 general procedure 4, 2,4-dichloro-5-methoxypyrimidine (0.3 g, 1.6 mmol), cyclopropanemethylamine hydrochloride (0.20 g, 1.84 mmol) and DIPEA (0.58 ml, 3.3 mmol) gave the title compound (0.36 g, 99%).

### MW: 213.67

HPLCMS (Method D): [m/z]: 214

### Benzyl-(2-chloro-5-methoxy-pyrimidin-4-yl)-amine

In a similar fashion using route 2 general procedure 4, 2,4-dichloro-5-methoxypyrimidine (0.3 g, 1.6 mmol), benzylamine (0.20 g, 1.84 mmol) and DIPEA (0.58 ml, 3.3 mmol) gave the title compound (0.42 g, 97%).

### MW: 249.70

HPLCMS (Method D): [m/z]: 250

### (2-Chloro-5-methoxy-pyrimidin-4-yl)-cyclohexylmethyl-amine

In a similar fashion using route 2 general procedure 4, 2,4-dichloro-5-methoxypyrimidine (0.3 g, 1.6 mmol), cyclohexanemethylamine (0.21 g, 1.84 mmol) and DIPEA (0.58 ml, 3.3 mmol) gave the title compound (0.43 g, 100%).

### MW: 255.75

HPLCMS (Method D): [m/z]: 258

### (2-Chloro-5-methoxy-pyrimidin-4-yl)-dimethyl-amine

In a similar fashion using route 2 general procedure 4, 2,4-dichloro-5-methoxypyrimidine (0.3 g, 1.6 mmol), dimethylamine (83 mg, 1.84 mmol) and DIPEA (0.58 ml, 3.3 mmol) gave the title compound (0.31 g, 97%).

### MW: 187.63

HPLCMS (Method D): [m/z]: 188

### (2-Chloro-5-methoxy-pyrimidin-4-yl)-diethyl-amine

In a similar fashion using route 2 general procedure 4, 2,4-dichloro-5-methoxypyrimidine (0.3 g, 1.6 mmol), diethylamine (0.13 g, 1.84 mmol) and DIPEA (0.58 ml, 3.3 mmol) gave the title compound (0.34 g, 94%).

### MW: 215.68

HPLCMS (Method D): [m/z]: 216

### Benzyl-(2-chloro-5-methoxy-pyrimidin-4-yl)-methyl-amine

In a similar fashion using route 2 general procedure 4, 2,4-dichloro-5-methoxypyrimidine (0.3 g, 1.6 mmol), N-methylbenzylamine (0.22 g, 1.84 mmol) and DIPEA (0.58 ml, 3.3 mmol) gave the title compound (0.37 g, 83%).

### MW: 263.73

HPLCMS (Method D): [m/z]: 264

### 2-Chloro-5-methoxy-4-piperidin-1-yl-pyrimidine

In a similar fashion using route 2 general procedure 4, 2,4-dichloro-5-methoxypyrimidine (0.3 g, 1.6 mmol), piperidine (0.16 g, 1.84 mmol) and DIPEA (0.58 ml, 3.3 mmol) gave the title compound (0.37 g, 96%).

### MW: 227.7

HPLCMS (Method D): [m/z]: 228

### 4-(2-Chloro-5-methoxy-pyrimidin-4-yl)-morpholine

In a similar fashion using route 2 general procedure 4, 2,4-dichloro-5-methoxypyrimidine (0.3 g, 1.6 mmol), morpholine (0.16 g, 1.84 mmol) and DIPEA (0.58 ml, 3.3 mmol) gave the title compound (0.38 g, 98%).

### MW: 229.67

HPLCMS (Method D): [m/z]: 230

### General Procedure 5:

### Lithium tris(propan-2-yloxy)(pyridin-2-yl)borate n-BuLi (791 μl, 1.74 mmol) was added dropwise to a solution of triisopropoxy borate (400 μl, 1.74 mmol) and 2-bromopyridine (250 mg, 1.58 mmol) in THF/toluene (1:4, 7.5 ml) at −78° C. The reaction was stirred at −78° C. for 1.5 h and then allowed to warm to room temperature overnight. The reaction was concentrated in vacuo to give the title compound (421 mg, 88%) which was used without further purification. The compound could not be detected by HPLCMS therefore structure was confirmed by NMR.

### Lithium (5-methoxypyridin-2-yl)tris(propan-2-yloxy)borate

In a similar fashion using route 2 general procedure 5, n-BuLi (791 μl, 1.74 mmol), triisopropoxy borate (400 μl, 1.74 mmol) and 2-bromo-5-methoxy-pyridine (198 mg, 1.58 mmol) gave the title compound (404 mg, 94%) which was used without further purification. The compound could not be detected by HPLCMS therefore structure was confirmed by 1H-NMR.

### General Procedure 6:

### Example 17

### Ethyl-(5-methoxy-2-pyridin-2-yl-pyrimidin-4-yl)-amine

Pd₂(dba)₃ (10 mg, 0.01 mmol) was added to a mixture of lithium tris(propan-2-yloxy)(pyridin-2-yl)borate (367 mg, 1.50 mmol), KF (87 mg, 1.50 mmol), t-Bu₂PHO (10 mg, 0.06 mmol) and (2-chloro-5-methoxy-pyrimidin-4-yl)-ethyl-amine (94 mg, 0.50 mmol) in degassed dioxane (2 ml). The reaction was heated to 110° C. for 48 h. The reaction mixture was allowed to cool and was filtered. The filter cake was washed with EtOAc and the filtrate was washed with water. The aqueous washings were extracted with EtOAc (×2). The combined organic phases were dried (Na₂SO₄) and concentrated in vacuo. The crude residue was purified by preparative HPLC (neutral conditions) to give the title compound (9 mg, 8%).

### MW: 230.26

HPLCMS (Method A): [m/z]: 231

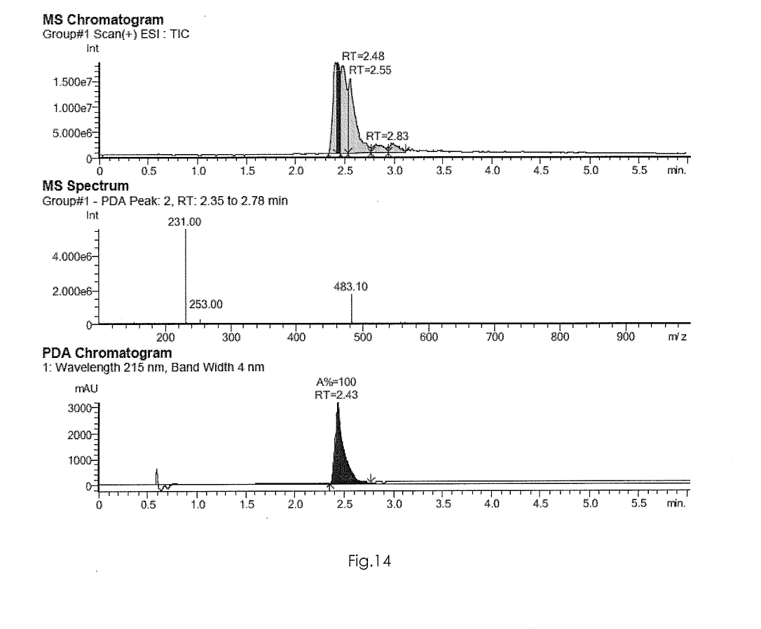

FIG. 14 shows the MS chromatogram, the MS spectrum and the PDA chromatogram of the compound of example 17.

### IC50 [μM]: <50.

### Example 18

### Isobutyl-(5-methoxy-2-pyridin-2-yl-pyrimidin-4-yl)-amine

In a similar fashion using route 2 general procedure 6, Pd₂(dba)₃ (10 mg, 0.01 mmol), lithium tris(propan-2-yloxy)(pyridin-2-yl)borate (367 mg, 1.50 mmol), KF (87 mg, 1.50 mmol), t-Bu₂PHO (10 mg, 0.06 mmol) and (2-chloro-5-methoxy-pyrimidin-4-yl)-isobutyl-amine (101 mg, 0.50 mmol) gave the title compound (5 mg, 4%) after purification by preparative HPLC (neutral conditions).

### MW: 258.32

HPLCMS (Method A): [m/z]: 259

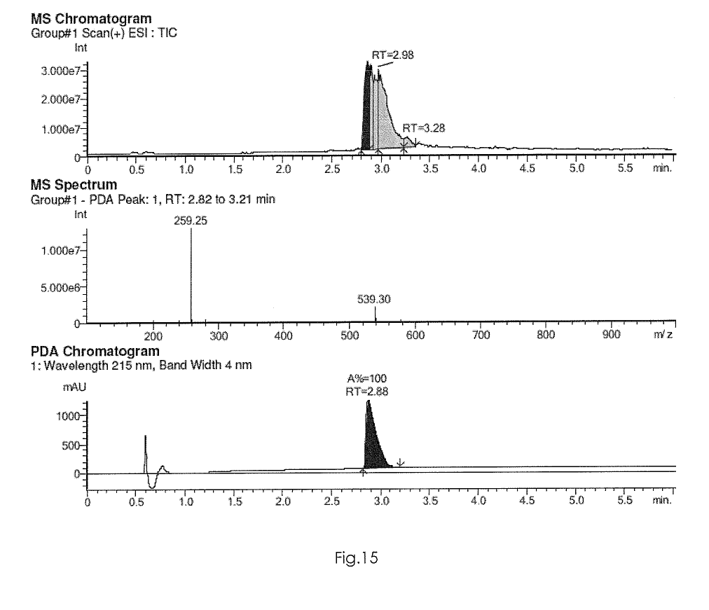

FIG. 15 shows the MS chromatogram, the MS spectrum and the PDA chromatogram of the compound of example 18.

### IC50 [μM]: <50.

### Example 19

### Cyclopropylmethyl-(5-methoxy-2-pyridin-2-yl-pyrimidin-4-yl)-amine

In a similar fashion using route 2 general procedure 6, Pd₂(dba)₃ (10 mg, 0.01 mmol), lithium tris(propan-2-yloxy)(pyridin-2-yl)borate (367 mg, 1.50 mmol), KF (87 mg, 1.50 mmol), t-Bu₂PHO (10 mg, 0.06 mmol) and (2-chloro-5-methoxy-pyrimidin-4-yl)-cyclopropylmethyl-amine (107 mg, 0.50 mmol) gave the title compound (4 mg, 3%) after purification by preparative HPLC (neutral conditions).

### MW: 256.30

HPLCMS (Method A): [m/z]: 257

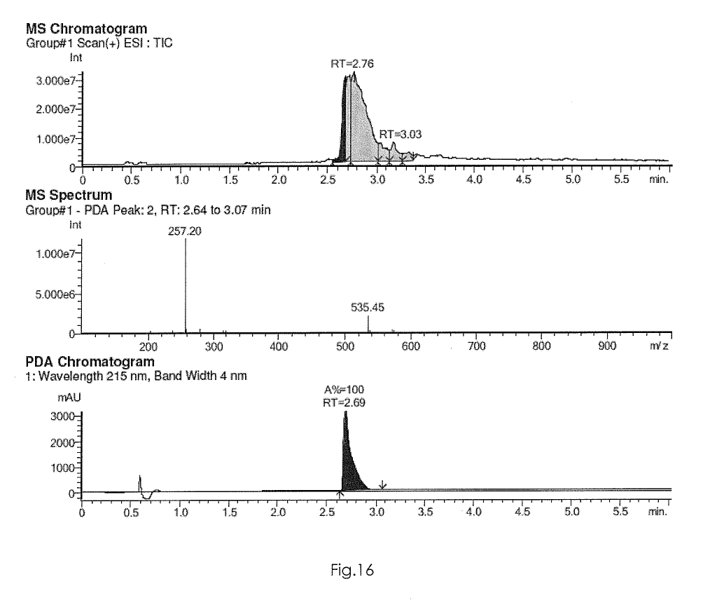

FIG. 16 shows the MS chromatogram, the MS spectrum and the PDA chromatogram of the compound of example 19.

### IC50 [μM]: <50.

### Example 20

### Benzyl-(5-methoxy-2-pyridin-2-yl-pyrimidin-4-yl)-amine

In a similar fashion using route 2 general procedure 6, Pd₂(dba)₃ (19 mg, 0.02 mmol), lithium tris(propan-2-yloxy)(pyridin-2-yl)borate (780 mg, 3.19 mmol), KF (185 mg, 3.19 mmol), t-Bu₂PHO (21 mg, 0.13 mmol) and benzyl-(2-chloro-5-methoxy-pyrimidin-4-yl)-amine (265 mg, 1.06 mmol) gave the title compound (4 mg, 3%) after purification by preparative HPLC (acidic conditions).

### MW: 292.34

HPLCMS (Method A): [m/z]: 293

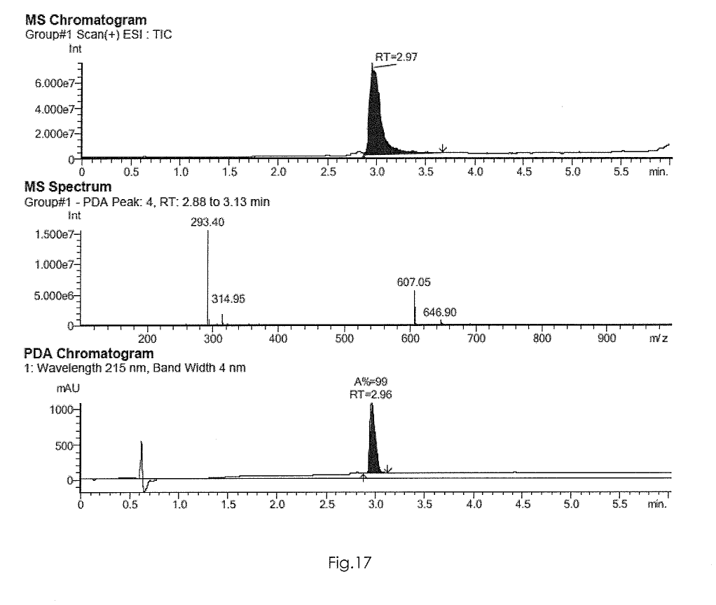

FIG. 17 shows the MS chromatogram, the MS spectrum and the PDA chromatogram of the compound of example 20.

### IC50 [μM]: <50.

### Example 21

### Cyclohexylmethyl-(5-methoxy-2-pyridin-2-yl-pyrimidin-4-yl)-amine

In a similar fashion using route 2 general procedure 6, Pd2(dba)₃ (10 mg, 0.01 mmol), lithium tris(propan-2-yloxy)(pyridin-2-yl)borate (367 mg, 1.50 mmol), KF (87 mg, 1.50 mmol), t-Bu₂PHO (10 mg, 0.06 mmol) and (2-chloro-5-methoxy-pyrimidin-4-yl)-cyclohexylmethyl-amine (128 mg, 0.50 mmol) gave the title compound (9 mg, 6%) after purification by preparative HPLC (acidic conditions).

### MW: 298.38

HPLCMS (Method A): [m/z]: 299

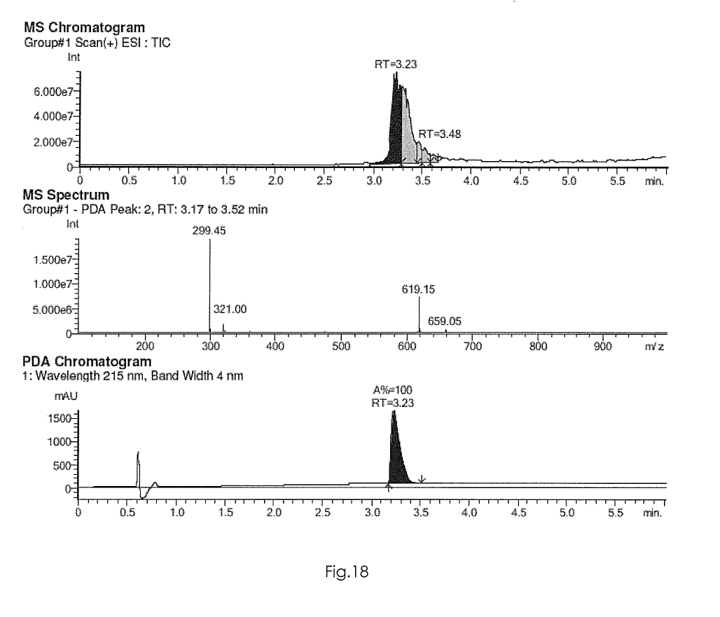

FIG. 18 shows the MS chromatogram, the MS spectrum and the PDA chromatogram of the compound of example 21.

### IC50 [μM]: <50.

### Example 22

### (5-Methoxy-2-pyridin-2-yl-pyrimidin-4-yl)-dimethyl-amine

In a similar fashion using route 2 general procedure 6, Pd2(dba)₃ (20 mg, 0.02 mmol), lithium tris(propane-2-yloxy)(pyridin-2-yl)borate (790 mg, 3.25 mmol), KF (189 mg, 3.25 mmol), t-Bu₂PHO (217 mg, 0.13 mmol) and (2-chloro-5-methoxy-pyrimidin-4-yl)-dimethyl-amine (203 mg, 1.08 mmol) gave the title compound (27 mg, 23%) after purification by preparative HPLC (acidic conditions).

### MW: 230.27

HPLCMS (Method A): [m/z]: 230.95

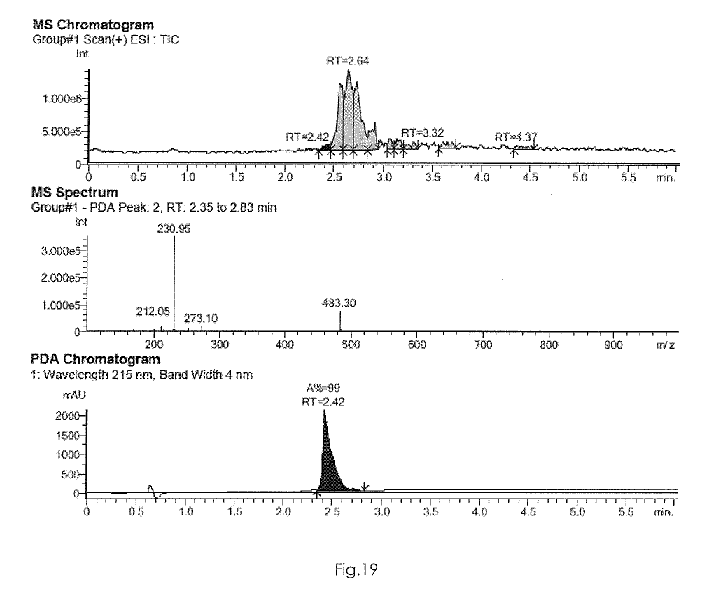

FIG. 19 shows the MS chromatogram, the MS spectrum and the PDA chromatogram of the compound of example 22.

### IC50 [μM]: <50.

### Example 23

### Diethyl-(5-methoxy-2-pyridin-2-yl-pyrimidin-4-yl)-amine

In a similar fashion using route 2 general procedure 6, Pd2(dba)₃ (10 mg, 0.01 mmol), lithium tris(propan-2-yloxy)(pyridin-2-yl)borate (367 mg, 1.50 mmol), KF (87 mg, 1.50 mmol), t-Bu₂PHO (10 mg, 0.06 mmol) and (2-chloro-5-methoxy-pyrimidin-4-yl)-diethyl-amine (108 mg, 0.50 mmol) gave the title compound (11 mg, 9%) after purification by preparative HPLC (acidic conditions).

### MW: 258.32

HPLCMS (Method A): [m/z]: 259

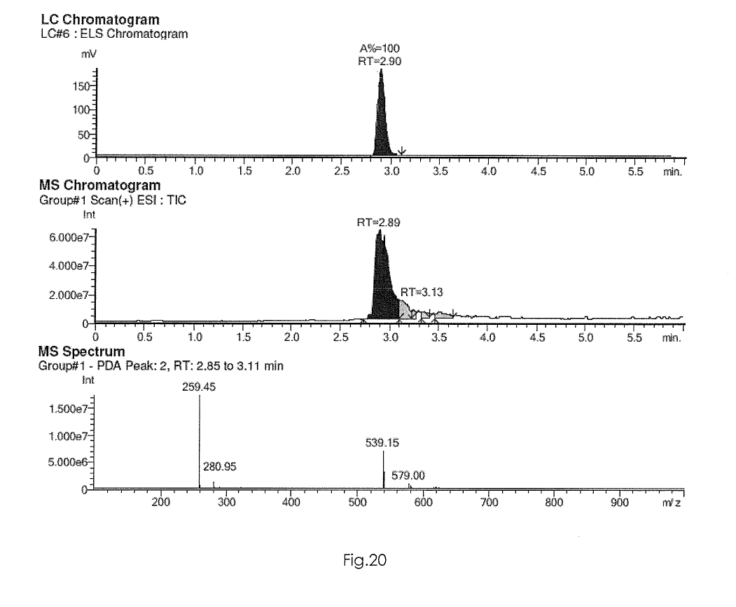

FIG. 20 shows the MS chromatogram, the MS spectrum and the PDA chromatogram of the compound of example 23.

### IC50 [μM]: >50.

### Example 24

### Benzyl-(5-methoxy-2-pyridin-2-yl-pyrimidin-4-yl)-methyl-amine

In a similar fashion using route 2 general procedure 6, Pd2(dba)₃ (10 mg, 0.01 mmol), lithium tris(propan-2-yloxy)(pyridin-2-yl)borate (367 mg, 1.50 mmol), KF (87 mg, 1.50 mmol), t-Bu₂PHO (10 mg, 0.06 mmol) and benzyl-(2-chloro-5-methoxy-pyrimidine-4-yl)-methyl-amine (132 mg, 0.50 mmol) gave the title compound (16 mg, 10%) after purification by preparative HPLC (acidic conditions).

### MW: 306.36

HPLCMS (Method A): [m/z]: 307

FIG. 21 shows the MS chromatogram, the MS spectrum and the PDA chromatogram of the compound of example 24.

### IC50 [μM]: >50.

### Example 25

### 5-Methoxy-4-piperidin-1-yl-2-pyridin-2-yl-pyrimidine

In a similar fashion using route 2 general procedure 6, Pd2(dba)₃ (10 mg, 0.01 mmol), lithium tris(propan-2-yloxy)(pyridin-2-yl)borate (367 mg, 1.50 mmol), KF (87 mg, 1.50 mmol), t-Bu₂PHO (10 mg, 0.06 mmol) and 2-chloro-5-methoxy-4-piperidin-1-yl-pyrimidine (114 mg, 0.50 mmol) gave the title compound (20 mg, 15%) after purification by preparative HPLC (acidic conditions).

### MW: 270.33

HPLCMS (Method A): [m/z]: 271

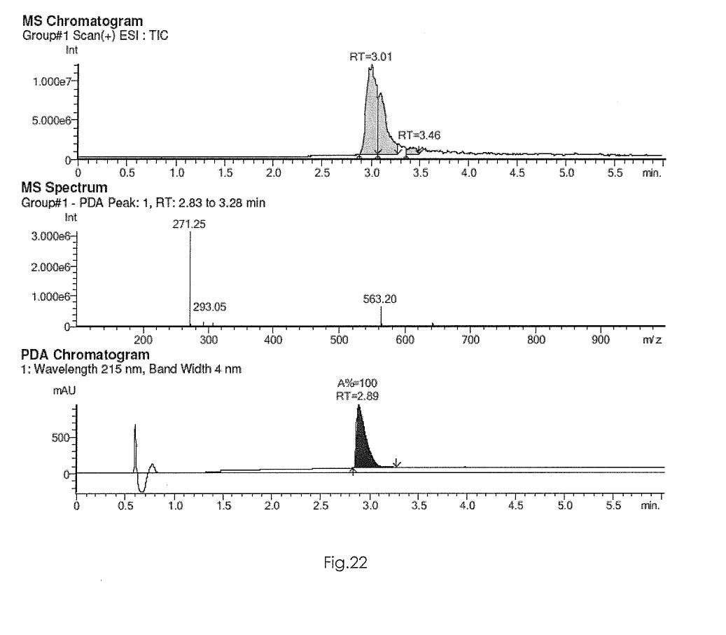

FIG. 22 shows the MS chromatogram, the MS spectrum and the PDA chromatogram of the compound of example 25.

### IC50 [μM]: >50.

### Example 26

### 4-(5-Methoxy-2-pyridin-2-yl-pyrimidin-4-yl)-morpholine

In a similar fashion using route 2 general procedure 6, Pd2(dba)₃ (20 mg, 0.02 mmol), lithium tris(propan-2-yloxy)(pyridin-2-yl)borate (820 mg, 3.35 mmol), KF (194 mg, 3.35 mmol), t-Bu₂PHO (22 mg, 0.13 mmol) and 4-(2-chloro-5-methoxy-pyrimidin-4-yl)-morpholine (256 mg, 1.12 mmol) gave the title compound (42 mg, 15%) after purification by preparative HPLC (acidic conditions).

### MW: 272.30

HPLCMS (Method A): [m/z]: 273

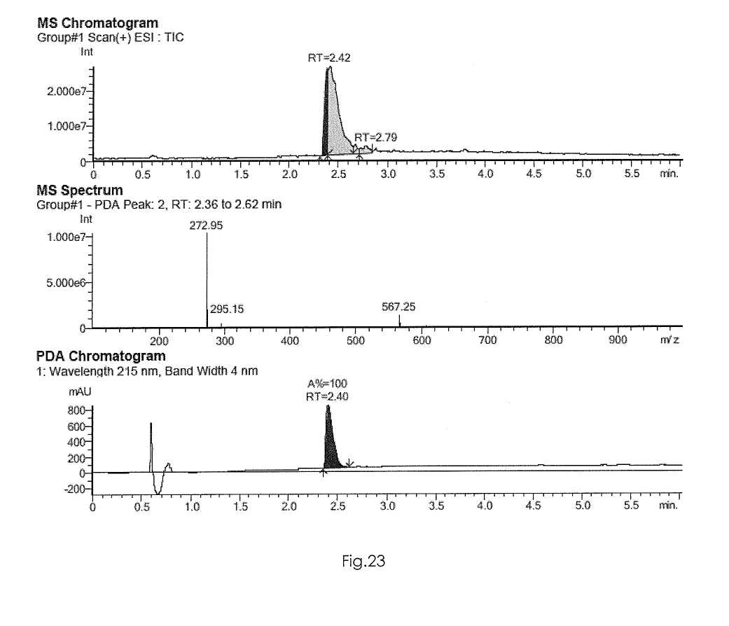

FIG. 23 shows the MS chromatogram, the MS spectrum and the PDA chromatogram of the compound of example 26.

### IC50 [μM]: <50.

### Example 27

### Isopropyl-[5-methoxy-2-(5-methoxy-pyridin-2-yl)-pyrimidin-4-yl]-amine

In a similar fashion using route 2 general procedure 6, Pd2(dba)₃ (18 mg, 0.02 mmol), lithium (5-methoxypyridin-2-yl)tris(propan-2-yloxy)borate (902 mg, 2.98 mmol), KF (173 mg, 2.98 mmol), t-Bu₂PHO (19 mg, 0.12 mmol) and (2-chloro-5-methoxy-pyrimidin-4-yl)-isopropyl-amine (200 mg, 0.9 mmol) gave the title compound (55 mg, 20%) after purification by preparative HPLC (acidic conditions).

### MW: 274.32

HPLCMS (Method A): [m/z]: 275

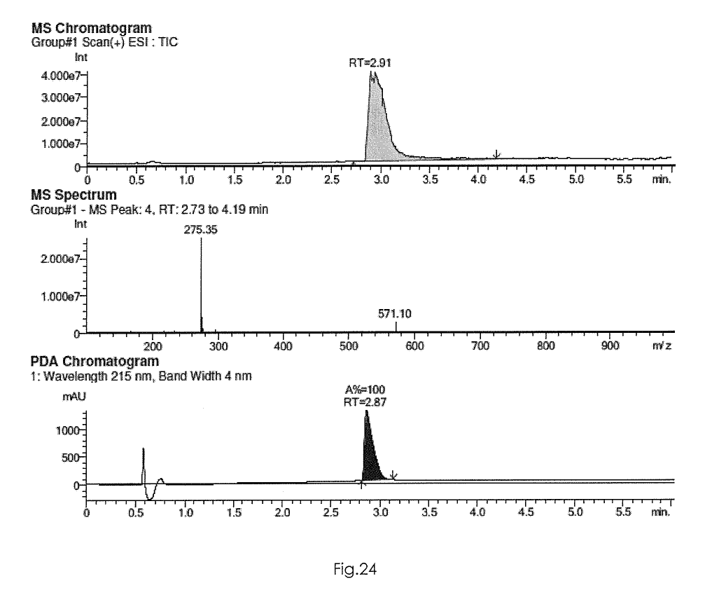

FIG. 24 shows the MS chromatogram, the MS spectrum and the PDA chromatogram of the compound of example 27.

### IC50 [μM]: <50.

### Route 3

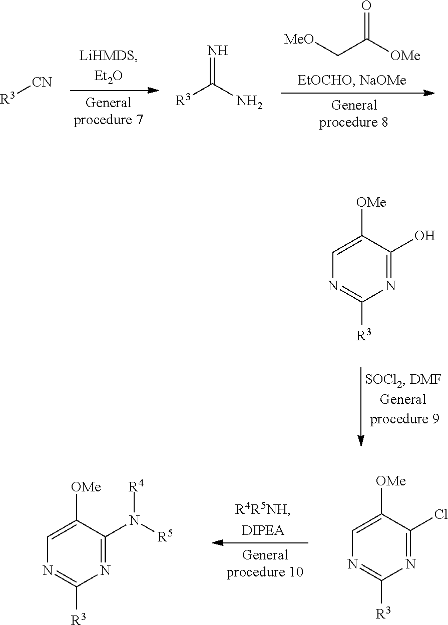

### General Procedure 7:

### Example 28

### Pyrimidine-2-carboxamidine (starting material)

Lithium hexamethyl disilazide (1M solution in THF, 20.0 ml, 20.0 mmol) was added to a solution of pyrimidine-2-carbonitrile (1.0 g, 9.5 mmol) in Et₂O (30 ml) at 0° C. The reaction was allowed to warm to room temperature overnight. The reaction was cooled to 0° C. and 3 M HCl (54 ml) was added and the reaction was stirred for 30 min. Water (135 ml) was added and the organic phase was separated and discarded. The aqueous phase was basified to pH 14 with saturated aqueous NaOH and extracted with DCM (×3). The combined organic extracts were dried (Na₂SO₄) and concentrated in vacuo to give the title compound (0.46 g, 40%).

### MW: 122.13

HPLCMS (Method B): [m/z]: 123

### General Procedure 8:

### 5-Methoxy-[2,2′]bipyrimidinyl-4-ol (Example 28)

NaOMe (0.49 g, 9.00 mmol) was added to a solution of methyl methoxy acetate (0.81 ml, 8.19 mmol) and ethyl formate (0.99 ml, 12.28 mmol) in MeOH (10 ml). The reaction mixture was stirred at room temperature for 5 h. A solution of pyrimidine-2-carboxamidine (1.0 g, 8.19 mmol) in MeOH (5 ml) was added followed by NaOMe (0.44 g, 8.19 mmol). The mixture was heated under reflux for 18 h and was concentrated in vacuo. The crude residue was purified by column chromatography with MeOH/DCM (5:95-50:50) as the eluent to give the title compound (0.55 g, 22%).

### MW: 204.19

HPLCMS (Method A): [m/z]: 205

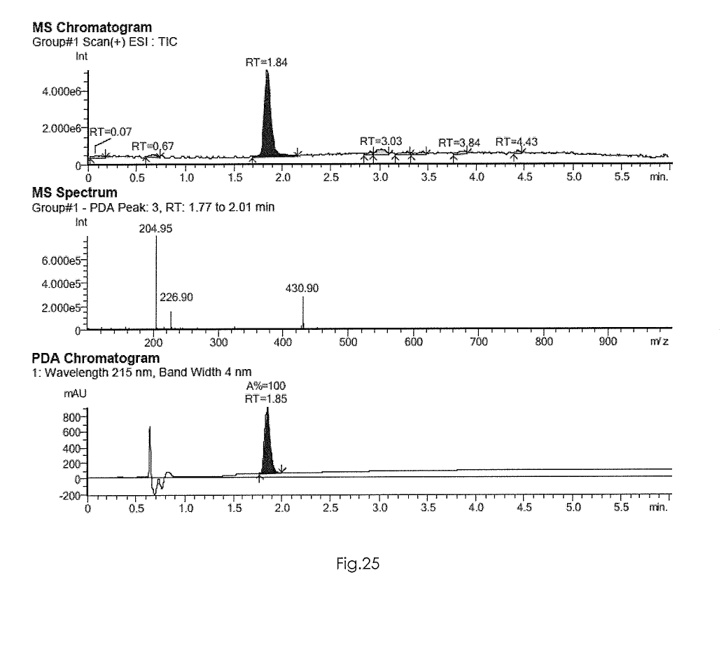

FIG. 25 shows the MS chromatogram, the MS spectrum and the PDA chromatogram of the compound of example 28.

### IC50 [μM]: >50.

### Example 29

### General Procedure 9:

### 4-Chloro-5-methoxy-[2,2′]bipyrimidinyl

DMF (cat) was added to a solution of 5-methoxy-[2,2]bipyrimidinyl-4-ol (520 mg, 2.55 mmol) in thionyl chloride (5 ml) and the mixture was heated at 80° C. for 15 min. The mixture was concentrated in vacuo. The residue was basified with saturated aqueous NaHCO₃ solution (50 ml) and extracted with DCM (×3). The combined organic phases were dried (Na₂SO₄) and concentrated in vacuo to give the title compound (570 mg, 100%).

### MW: 222.64

HPLCMS (Method A): [m/z]: 223

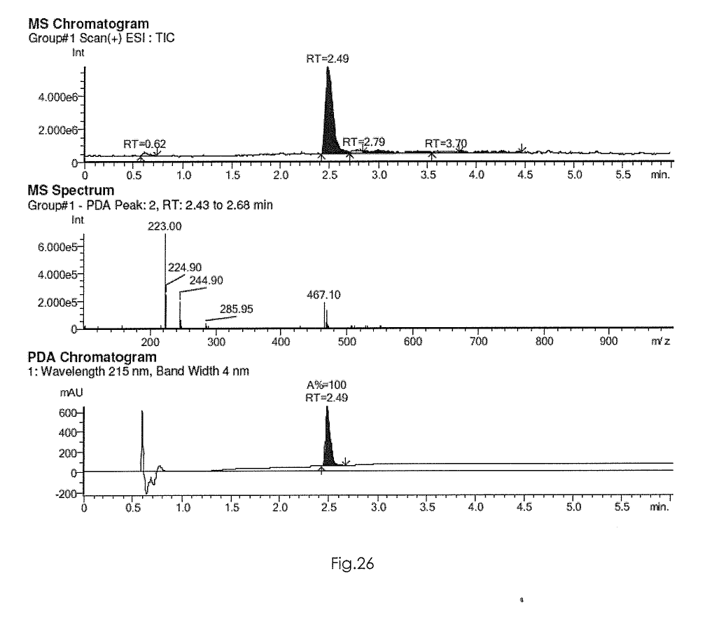

FIG. 26 shows the MS chromatogram, the MS spectrum and the PDA chromatogram of the compound of example 29.

### IC50 [μM]: >50.

### Example 30

### General Procedure 10:

### Isopropyl-(5-methoxy-[2,2′]bipyrimidinyl-4-yl)-amine

Diisopropylamine (173 μl, 2.02 mmol) was added to a solution of 4-chloro-5-methoxy-[2,2′]bipyrimidinyl (100 mg, 0.45 mmol) in EtOH (1.0 ml) and the mixture was heated under reflux for 18 h. The reaction mixture was concentrated in vacuo. The residue was basified with saturated aqueous NaHCO₃ solution (1 ml) and extracted with DCM (×3). The organic phase was washed with water (×2), dried (Na₂SO₄) and concentrated in vacuo to give the title compound (89 mg, 81%).

### MW: 245.29

HPLCMS (Method A): [m/z]: 246

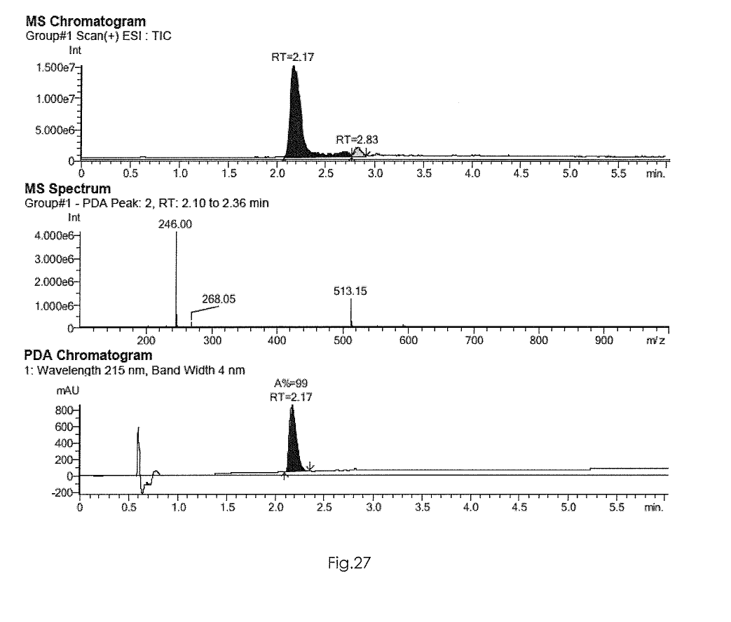

FIG. 27 shows the MS chromatogram, the MS spectrum and the PDA chromatogram of the compound of example 30.

### IC50 [μM]: >50.

### Route 4

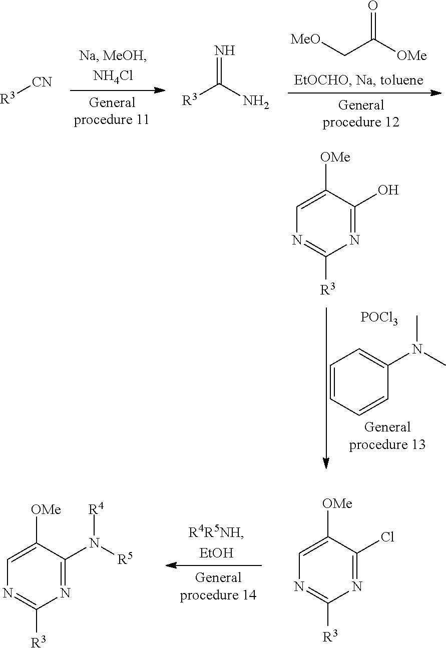

### General Procedure 11:

### Pyridine-2-carboxamidine

A solution of sodium metal (74 mg, 3.2 mmol) in MeOH (5 ml) was added to a solution of 2-cyanopyridine (3 g, 28 mmol) in MeOH (25 ml) and the mixture was stirred for 16 h at room temperature. Ammonium chloride (4.5 g, 84 mmol) was added and the mixture was stirred at 70° C. for 3 h. After cooling, the mixture was concentrated in vacuo. The residue was diluted with EtOH (40 ml) and the mixture was heated under reflux for 0.5 h. After cooling, the mixture was filtered and the filtrate was concentrated in vacuo. The crude residue was washed with Et₂O/iso-propanol (4:1) and dried under high vacuum to obtain the title compound as the HCl salt (4.5 g, 99%).

### MW: 121.4

HPLCMS (Method D): [m/z]: 122

### Pyrazine-2-carboxamidine

In a similar fashion using route 4 general procedure 11, pyrazine-2-carbonitrile (2 g, 19 mmol), sodium metal (49 mg, 2.15 mmol), MeOH (23 ml) and ammonium chloride (3.05 g, 57.1 mmol) gave the title compound (2.7 g, 93%) after trituration from EtOH.

### MW: 122.13

HPLCMS (Method D): [m/z]: 122

### General Procedure 12:

### 5-Methoxy-2-pyridine-2-yl-3H-pyrimidin-4-one

Methyl methoxyacetate (4.0 g, 38 mmol) and ethyl formate (2.81 g, 38 mmol) were added simultaneously to a stirring suspension of sodium (0.87 g, 38 mmol) in toluene (20 ml) and the mixture was stirred at room temperature for 12 h. The toluene was decanted, the residue was diluted with EtOH (20 ml) and pyridine-2-carboxamidine (4.7 g, 30 mmol) was added followed by a solution of sodium ethoxide (prepared from Na 1.39 g, 60 mmol and 5 ml of ethanol). The reaction mixture was heated under reflux for 15 h. After cooling, the mixture was filtered and the residue neutralized with 1N HCl (10 ml). The mixture was concentrated in vacuo. The crude residue was diluted with MeOH (20 ml), stirred for 0.25 h and filtered through celite. The filtrate was concentrated in vacuo to give the title compound (3.7 g, 61%).

### MW: 203.19

HPLCMS (Method D): [m/z]: 204

### 5-Methoxy-2-pyrazin-2-yl-3H-pyrimidin-4-one

In a similar fashion using route 4 general procedure 12, methyl methoxyacetate (1.0 g, 9.6 mmol), ethyl formate (0.71 g, 9.6 mmol) and sodium (0.22 g, 9.6 mmol) followed by pyrazine-2-carboxamidine (1.2 g, 7.6 mmol) and sodium ethoxide (prepared from Na 0.17 g, 7.6 mmol and 5 ml of ethanol) gave the title compound (0.75 g, 38%) after purification by trituration from MeOH.

### MW: 204.18

HPLCMS (Method A): [m/z]: 205

### 5-Methoxy-2-pyridin-3-yl-3H-pyrimidin-4-one

In a similar fashion using route 4 general procedure 12, methyl methoxyacetate (2.0 g, 19.2 mmol), ethyl formate (1.42 g, 19.2 mmol), sodium (0.44 g, 19.2 mmol) in toluene (20 ml) nicotinamidine hydrochloride (2.4 g, 15 mmol) gave the title compound (1.23 g, 39%).

### MW: 203.19

HPLCMS (Method D): [m/z]: 204

### General Procedure 13:

### 4-Chloro-5-methoxy-2-pyridin-2-yl-pyrimidine

5-Methoxy-2-pyridin-2-yl-3H-pyrimidin-4-one (4.2 g, 20.68 mmol) and POCl₃ (31.58 g, 206 mmol) in N,N-dimethyl aniline (6 ml) was heated under reflux for 1 h. After cooling, the mixture was poured into ice (200 ml) and the mixture was basified to pH 8-9 with saturated aqueous NaHCO₃. The aqueous phase was extracted with EtOAc (×3). The combined organic phases were dried (Na₂SO₄) and concentrated in vacuo. The crude residue was purified by column chromatography with DCM/MeOH (97:3) as the eluent to give the title compound (2.2 g, 48%).

### MW: 221.64

HPLCMS (Method D): [m/z]: 223

### 4-Chloro-5-methoxy-2-pyrazin-2-yl-pyrimidine

In a similar fashion using route 4 general procedure 13, 5-methoxy-2-pyrazin-2-yl-3H-pyrimidin-4-one (0.6 g, 2.94 mmol), POCl₃ (4.5 g, 29.4 mmol) and N,N-dimethyl aniline (0.8 ml) gave the title compound (44 mg, 6%) after purification by column chromatography with EtOAc/hexane (3:7) as the eluent.

### MW: 222.63

HPLCMS (Method D): [m/z]: 223

### 4-Chloro-5-methoxy-2-pyridin-3-yl-pyrimidine

In a similar fashion using route 4 general procedure 13, 5-methoxy-2-pyridin-3-yl-3H-pyrimidin-4-one (0.4 g, 19 mmol), POCl₃ (3 g, 19 mmol) and N,N-dimethyl aniline (0.3 ml) gave the title compound (0.16 g, 43%) after purification by column chromatography with DCM/MeOH (95:5).

### MW: 221.64

HPLCMS (Method D): [m/z]: 222

### Example 31

### General Procedure 14:

### (5-Methoxy-2-pyridin-2-yl-pyrimidin-4-yl)-(3-phenyl-propyl)-amine

4-Chloro-5-methoxy-2-pyridin-2-yl-pyrimidine (0.1 g, 0.45 mmol), 3-phenylpropan-1-amine (73 mg, 0.54 mmol) and DIPEA (0.12 g, 0.9 mmol) were dissolved in EtOH (2 ml) and the mixture was stirred at 80° C. for 15 h. After cooling, the mixture was concentrated in vacuo. The residue was diluted with water (15 ml) and the aqueous phase was extracted with EtOAc (×3). The combined organic phases were dried (Na₂SO₄) and concentrated in vacuo. The crude residue was purified by column chromatography with DCM/MeOH (95:5) as the eluent to give the title compound (65 mg, 45%).

### MW: 320.38

HPLCMS (Method A): [m/z]: 321

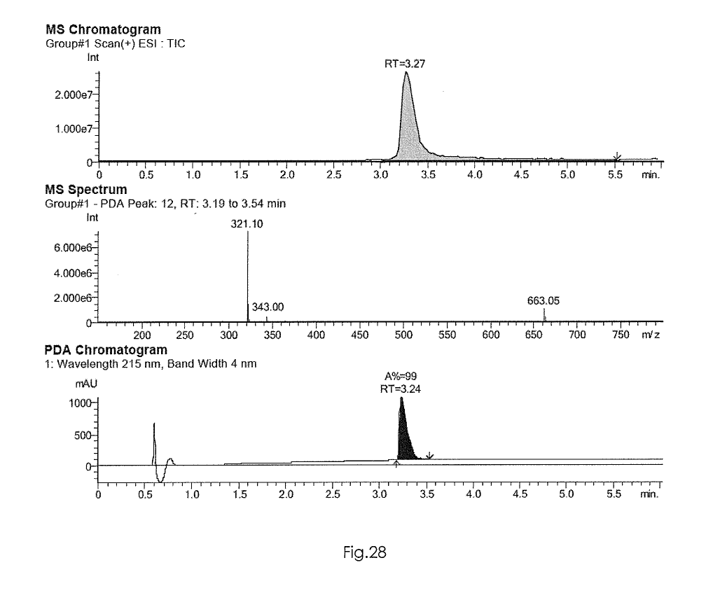

FIG. 28 shows the MS chromatogram, the MS spectrum and the PDA chromatogram of the compound of example 31.

### IC50 [μM]: >50.

### Example 32

### Ethyl-(5-methoxy-2-pyridin-2-yl-pyrimidin-4-yl)-methyl-amine

In a similar fashion using route 4 general procedure 14, 4-chloro-5-methoxy-2-pyridin-2-yl-pyrimidine (50 mg, 0.22 mmol), N-methyl ethylamine (15 μl, 0.27 mmol) and DIPEA (50 μl, 0.27 mmol) gave the title compound (29 mg, 53%) after purification by column chromatography with DCM/1% NH₃ in MeOH (95:5) as the eluent.

### MW: 244.29

HPLCMS (Method A): [m/z]: 245

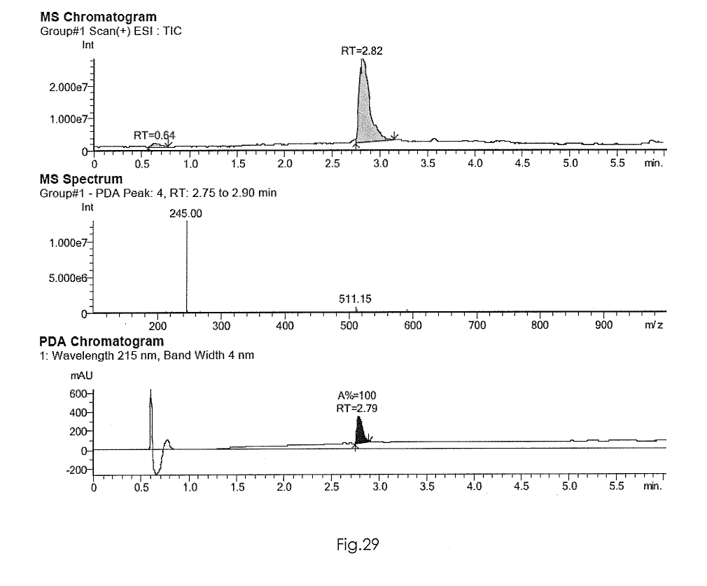

FIG. 29 shows the MS chromatogram, the MS spectrum and the PDA chromatogram of the compound of example 32.

### IC50 [μM]: <50.

### Example 33

### Isopropyl-(5-methoxy-2-pyridin-2-yl-pyrimidin-4-yl)-methyl-amine

In a similar fashion using route 4 general procedure 14, 4-chloro-5-methoxy-2-pyridin-2-yl-pyrimidine (50 mg, 0.22 mmol), N-methyl-iso-propylamine (19 mg, 0.27 mmol) and DIPEA (0.05 ml, 0.27 mmol) gave the title compound (23 mg, 39%) after purification by column chromatography with DCM/1% NH₃ in MeOH (95:5) as the eluent.

### MW: 258.31

HPLCMS (Method A): [m/z]: 259

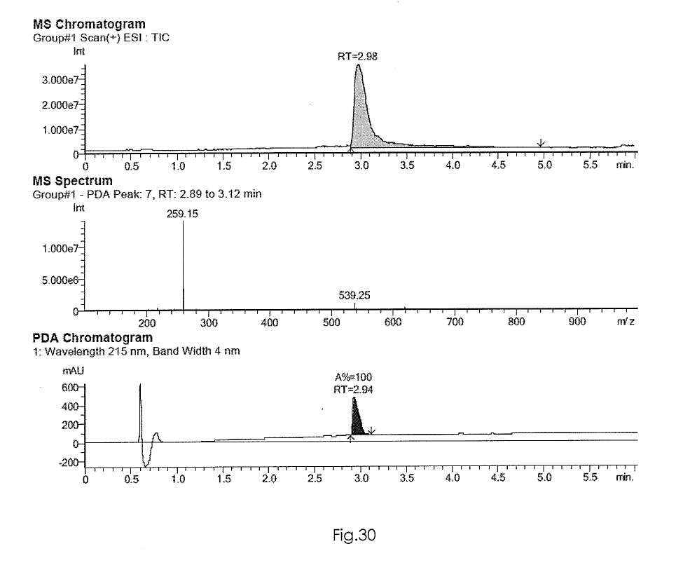

FIG. 30 shows the MS chromatogram, the MS spectrum and the PDA chromatogram of the compound of example 33.

### IC50 [μM]: >50.

### Example 34

### Isobutyl-(5-methoxy-2-pyridin-2-yl-pyrimidin-4-yl)-methyl-amine

In a similar fashion using route 4 general procedure 14, 4-chloro-5-methoxy-2-pyridin-2-yl-pyrimidine (50 mg, 0.22 mmol), N-methyl-iso-butylamine (20 μl, 0.27 mmol) and DIPEA (50 μl, 0.27 mmol) gave the title compound (30 mg, 49%) after purification by column chromatography with DCM/1% NH₃ in MeOH (95:5) as the eluent.

### MW: 272.35

HPLCMS (Method A): [m/z]: 273

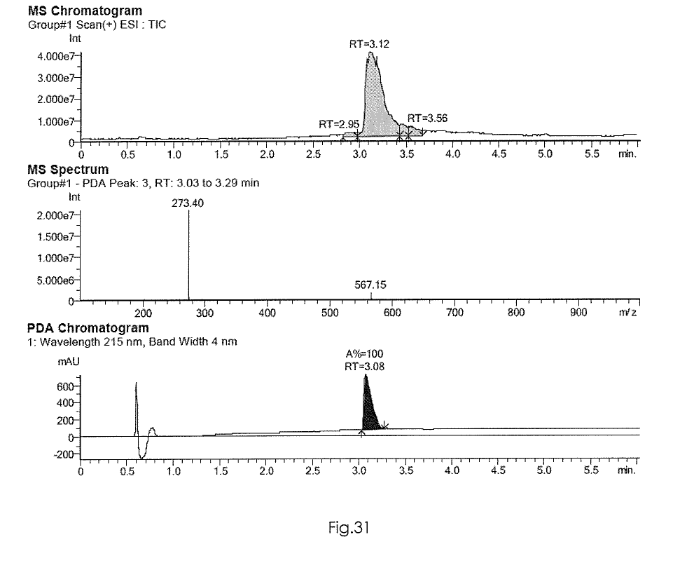

FIG. 31 shows the MS chromatogram, the MS spectrum and the PDA chromatogram of the compound of example 34.

### IC50 [μM]: >50.

### Example 35

### (5-Methoxy-2-pyridin-2-yl-pyrimidin-4-yl)-propyl-amine

In a similar fashion using route 4 general procedure 14, 4-chloro-5-methoxy-2-pyridin-2-yl-pyrimidine (50 mg, 0.22 mmol), propylamine (15 μl, 0.27 mmol) and DIPEA (50 μl, 0.27 mmol) gave the title compound (24 mg, 44%) after purification by column chromatography with DCM/1% NH₃ in MeOH (95:5) as the eluent.

### MW: 244.29

HPLCMS (Method A): [m/z]: 245

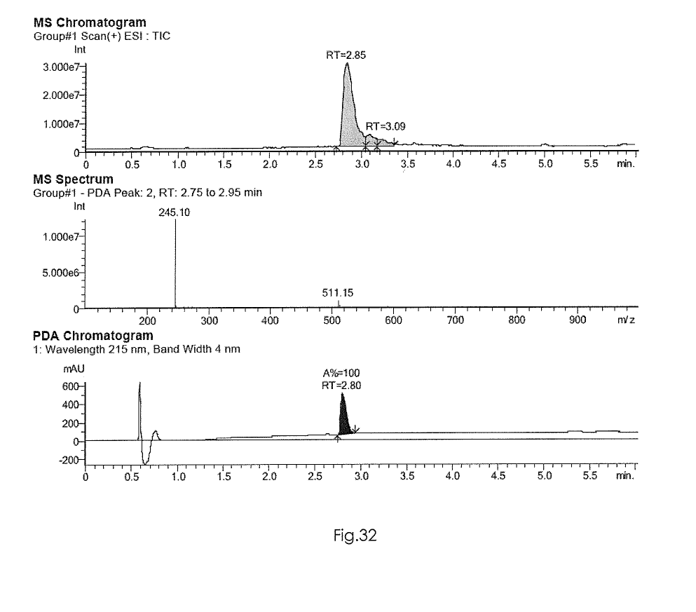

FIG. 32 shows the MS chromatogram, the MS spectrum and the PDA chromatogram of the compound of example 35.

### IC50 [μM]: <50.

### Example 36

### Butyl-(5-methoxy-2-pyridin-2-yl-pyrimidin-4-yl)-amine

In a similar fashion using route 4 general procedure 14, 4-chloro-5-methoxy-2-pyridin-2-yl-pyrimidine (50 mg, 0.22 mmol), butylamine (20 μl, 0.27 mmol) and DIPEA (50 μl, 0.27 mmol) gave the title compound (26 mg, 45%) after purification by column chromatography with DCM/1% NH₃ in MeOH (95:5) as the eluent.

### MW: 258.31

HPLCMS (Method A): [m/z]: 259

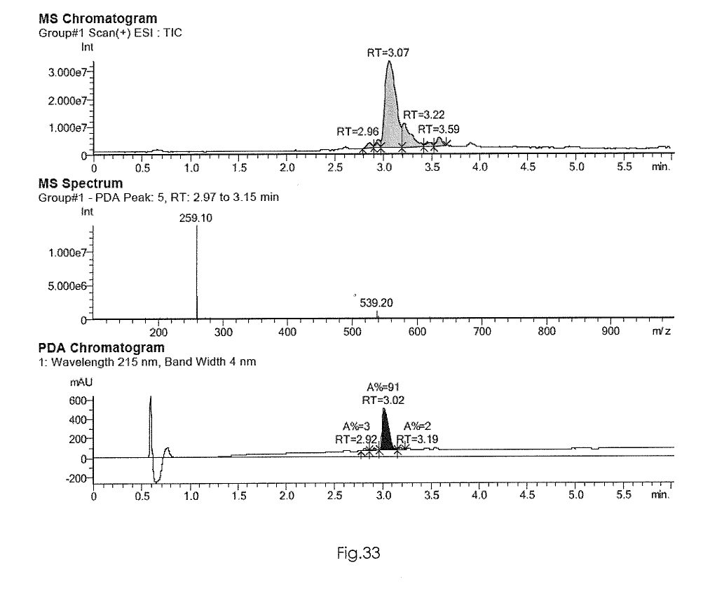

FIG. 33 shows the MS chromatogram, the MS spectrum and the PDA chromatogram of the compound of example 36.

### IC50 [μM]: <50.

### Example 37

### (5-Methoxy-2-pyridin-2-yl-pyrimidin-4-yl)-phenethyl-amine

In a similar fashion using route 4 general procedure 14, 4-chloro-5-methoxy-2-pyridin-2-yl-pyrimidine (50 mg, 0.22 mmol), phenylethylamine (30 μl, 0.27 mmol) and DIPEA (50 μl, 0.27 mmol) gave the title compound (28 mg, 48%) after purification by column chromatography with DCM/1% NH₃ in MeOH (95:5) as the eluent.

### MW: 306.32

HPLCMS (Method A): [m/z]: 307

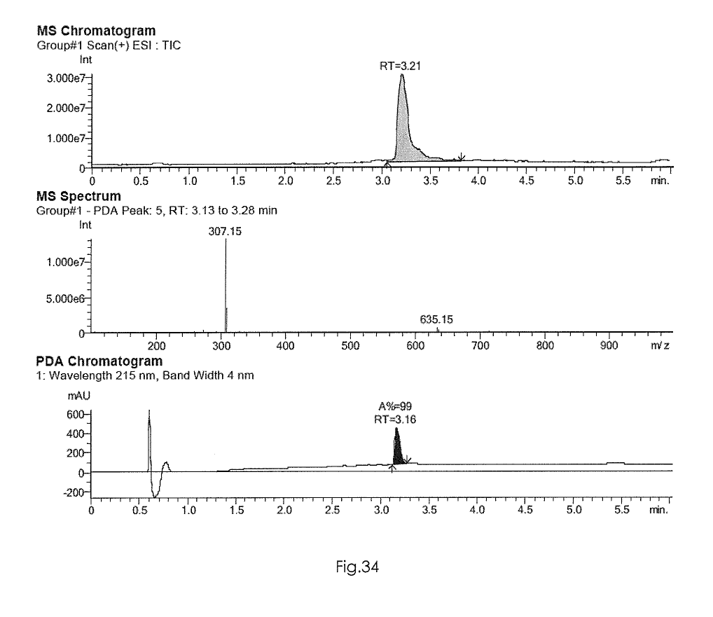

FIG. 34 shows the MS chromatogram, the MS spectrum and the PDA chromatogram of the compound of example 37.

### IC50 [μM]: <50.

### Example 38

### Isopropyl-(5-methoxy-2-pyrazin-2-yl-pyrimidin-4-yl)-amine

In a similar fashion using route 4 general procedure 14, 4-chloro-5-methoxy-2-pyrazin-2-yl-pyrimidine (44 mg, 0.19 mmol), isopropylamine (25 μl, 0.29 mmol) and DIPEA (67 μl, 0.39 mmol) gave the title compound (27 mg, 60%) after purification by column chromatography with DCM/MeOH (95:5) as eluent.

### MW: 245.28

HPLCMS (Method A) [m/z]: 246

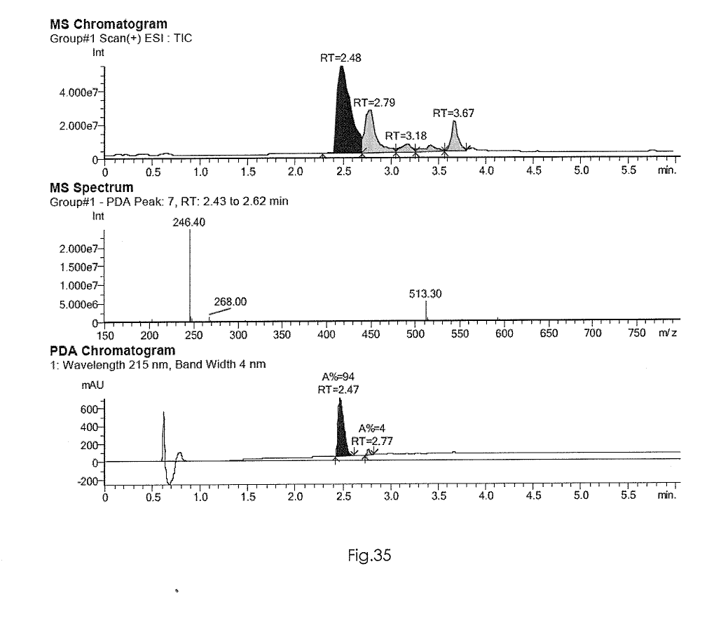

FIG. 35 shows the MS chromatogram, the MS spectrum and the PDA chromatogram of the compound of example 38.

### IC50 [μM]: >50.

### Example 39

### Isopropyl-(5-methoxy-2-pyridin-3-yl-pyrimidin-4-yl)-amine

In a similar fashion using general procedure 14, 4-chloro-5-methoxy-2-pyridin-3-yl-pyrimidine (0.15 g, 0.67 mmol), isopropylamine (43 μl, 0.74 mmol) and DIPEA (0.13 ml, 0.81 mmol) gave the title compound (39 mg, 24%) after purification by column chromatography with DCM/MeOH (98:2) as the eluent.

### MW: 244.29

HPLCMS (Method A): [m/z]: 245

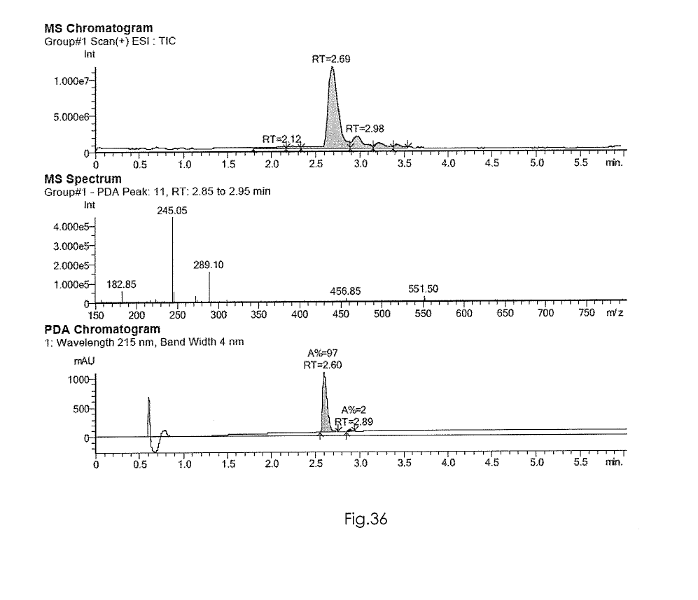

FIG. 36 shows the MS chromatogram, the MS spectrum and the PDA chromatogram of the compound of example 39.

### IC50 [μM]: >50.

### Route 5

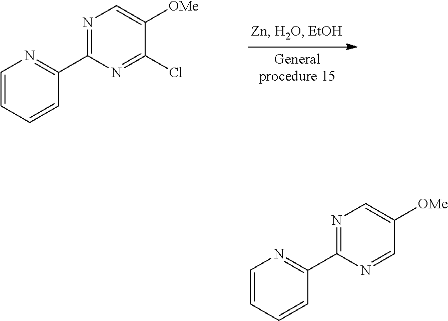

### Example 40

### General Procedure 15:

### 5-Methoxy-2-pyridin-2-yl-pyrimidine

Zinc power (1.0 g, 15.8 mmol) and water (2.4 ml) were added to a solution of 4-chloro-5-methoxy-2-pyridin-2-yl-pyrimidine (0.2 g, 0.9 mmol) in EtOH (5.4 ml) and the mixture was heated at 60° C. for 5 h. After cooling, the mixture was filtered and the filtrate was concentrated in vacuo. The crude residue was purified by column chromatography with DCM/1% NH₃ in MeOH (95:5) as the eluent to give the title compound (23 mg, 14%).

### MW: 187.20

HPLCMS (Method A): [m/z]: 188

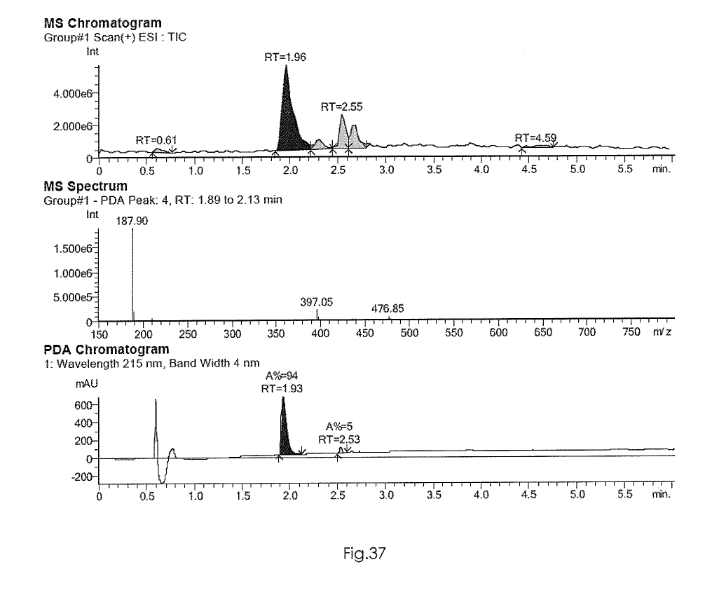

FIG. 37 shows the MS chromatogram, the MS spectrum and the PDA chromatogram of the compound of example 40.

### IC50 [μM]: >50.

### Route 6

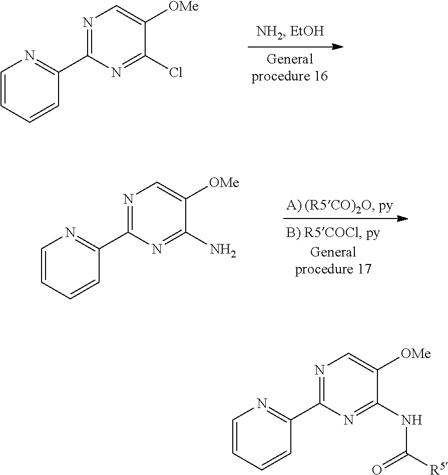

### General Procedure 16:

### Example 41

### 5-Methoxy-2-pyridin-2-yl-pyrimidin-4-ylamine

4-Chloro-5-methoxy-2-pyridin-2-yl-pyrimidine (1.0 g, 4.52 mmol) in EtOH (5 ml) was purged with ammonia gas at 0° C. for 0.3 h. The reaction mixture was heated at 140° C. for 12 h. After cooling, the mixture was concentrated in vacuo. The crude residue was purified by column chromatography with DCM/1% NH₃ in MeOH (97:3) as the eluent to give the title compound (0.7 g, 78%).

### MW: 202.21

HPLCMS (Method A): [m/z]: 203

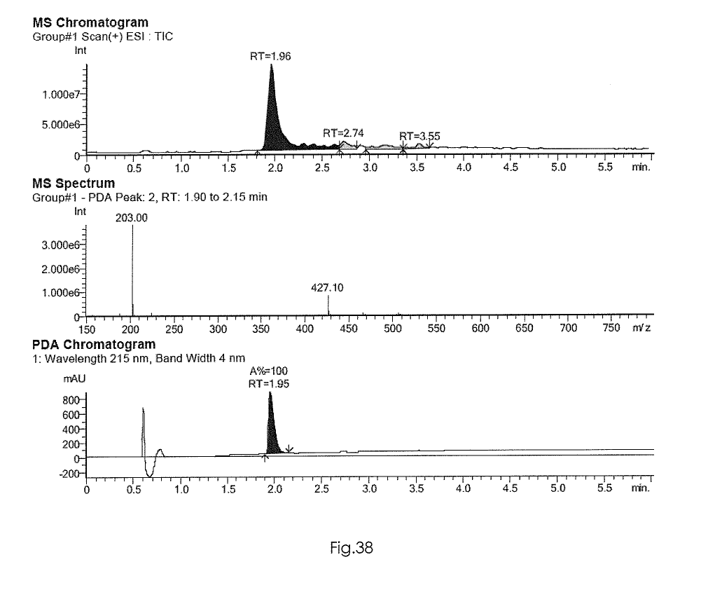

FIG. 38 shows the MS chromatogram, the MS spectrum and the PDA chromatogram of the compound of example 41.

### IC50 [μM]: >50.

### Example 42

### General Procedure 17:

### N-(5-Methoxy-2-pyridin-2-yl-pyrimidin-4-yl)-acetamide

Acetic anhydride (0.05 g, 0.49 mmol) was added to a solution of 5-methoxy-2-pyridin-2-yl-pyrimidin-4-ylamine (0.05 g, 0.25 mmol) in pyridine (0.5 ml) at 0° C. and the mixture was stirred at room temperature for 12 h. The mixture was diluted with water (7 ml) and the aqueous phase was extracted with DCM (×3). The combined organic phases were dried (Na₂SO₄) and concentrated in vacuo. The crude residue was purified by column chromatography with DCM/1% NH₃ in MeOH (95:5) and 1% ammonia as the eluent to give the title compound (25 mg, 41%).

### MW: 244.29

HPLCMS (Method A): [m/z]: 245

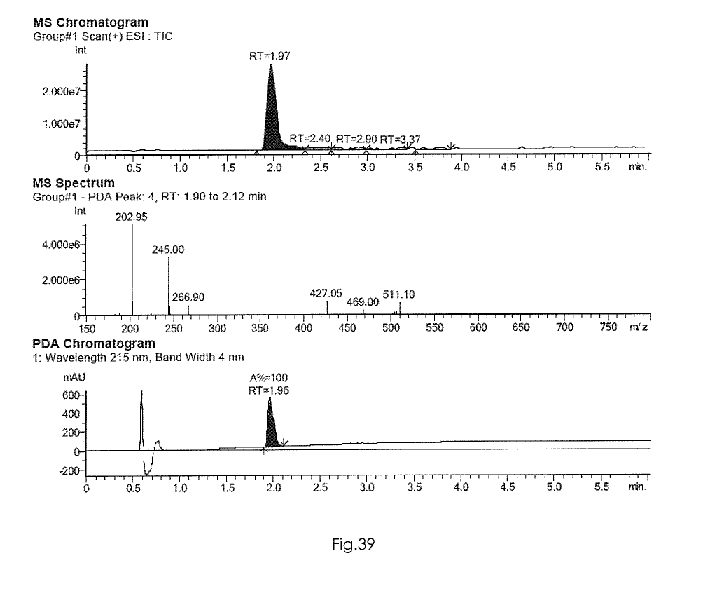

FIG. 39 shows the MS chromatogram, the MS spectrum and the PDA chromatogram of the compound of example 42.

### IC50 [μM]: >50.

### Example 43

### N-(5-Methoxy-2-pyridin-2-yl-pyrimidin-4-yl)-benzamide

In a similar fashion using route 6 general procedure 17, 5-methoxy-2-pyridin-2-yl-pyrimidin-4-ylamine (45 mg, 0.22 mmol), benzoyl chloride (59 mg, 0.42 mmol) and pyridine (0.5 ml) gave the title compound (20 mg, 29%) after purification by column chromatography with DCM/MeOH (95:5) as the eluent.

### MW: 306.31

HPLCMS (Method A): [m/z]: 307

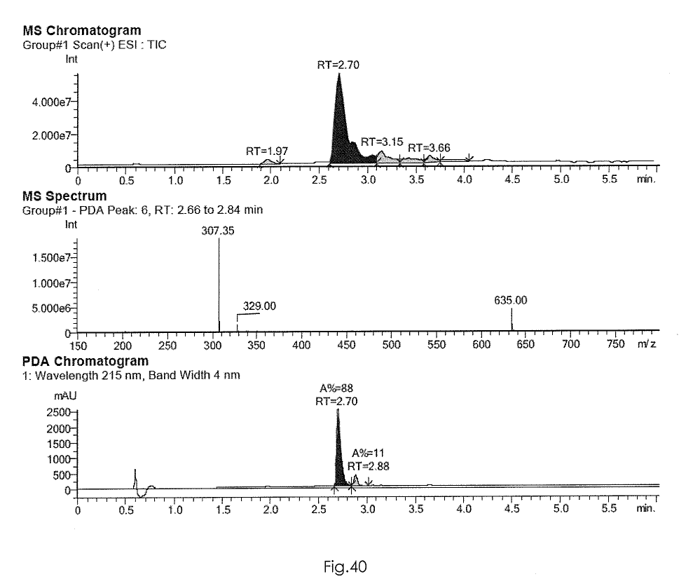

FIG. 40 shows the MS chromatogram, the MS spectrum and the PDA chromatogram of the compound of example 43.

### IC50 [μM]: <50.

### Route 7

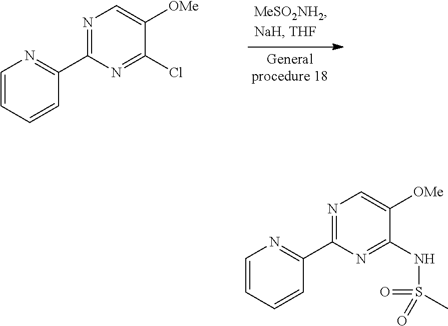

### General Procedure 18:

### Example 44

### Synthesis of N-(5-Methoxy-2-pyridin-2-yl-pyrimidin-4-yl)-methanesulfonamide

Methanesulfonamide (47 mg, 0.49 mmol) was added into a solution of sodium hydride (60% in mineral oil, 20 mg, 0.5 mmol) in THF (0.5 ml) and the mixture was stirred at room temperature for 0.5 h. 4-chloro-5-methoxy-2-pyridin-2-yl-pyrimidine (0.10 g, 0.45 mmol) in DMSO (0.5 ml) was added and the mixture was heated at 120° C. for 1 h. After cooling, the mixture was concentrated in vacuo. The crude residue was purified by column chromatography with DCM/1% NH₃ in MeOH (97:3) as the eluent to give the title compound (27 mg, 27%).

### MW: 280.30

HPLCMS (Method A): [m/z]: 281

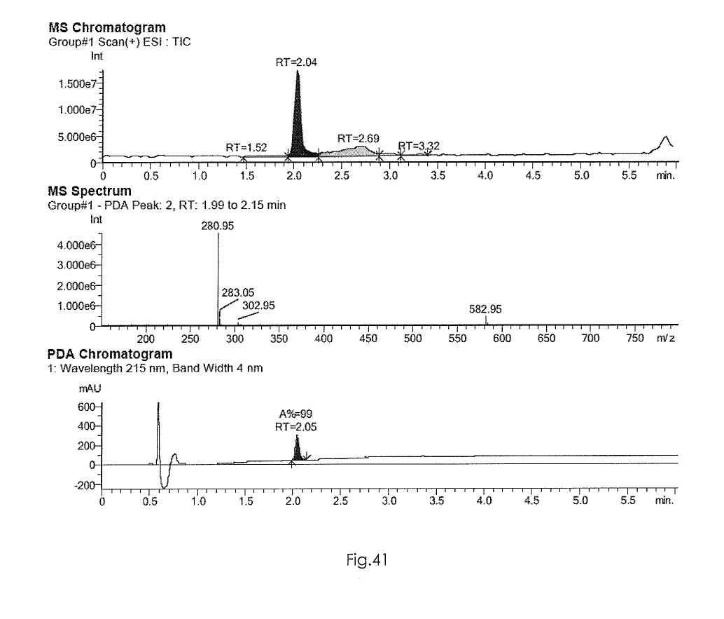

FIG. 41 shows the MS chromatogram, the MS spectrum and the PDA chromatogram of the compound of example 44.

### IC50 [μM]: <50.

### Route 8

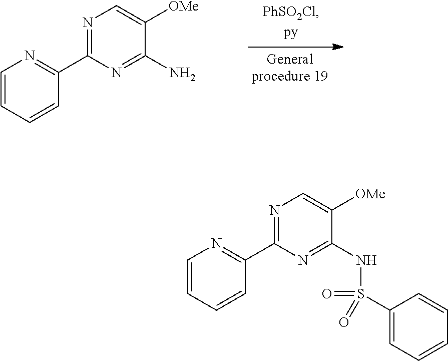

### General Procedure 19:

### Example 45

### N-(5-Methoxy-2-pyridin-2-yl-pyrimidin-4-yl)-benzenesulfonamide

Benzene sulfonyl chloride (43 mg, 0.24 mmol) was added to a solution of 5-methoxy-2-pyridin-2-yl-pyrimidin-4-ylamine (50 mg, 0.24 mmol) in pyridine (0.3 ml) and the mixture was heated at 80° C. for 16 h. After cooling, the reaction mixture was diluted with water (10 ml) and the aqueous phase was extracted with DCM (×3). The combined organic phases were dried (Na₂SO₄) and concentrated in vacuo. The crude residue was purified by column chromatography with DCM/1% NH₃ in MeOH (95:5) as the eluent to give the title compound (15 mg, 18%).

### MW: 342.37

HPLCMS (Method A): [m/z]: 343

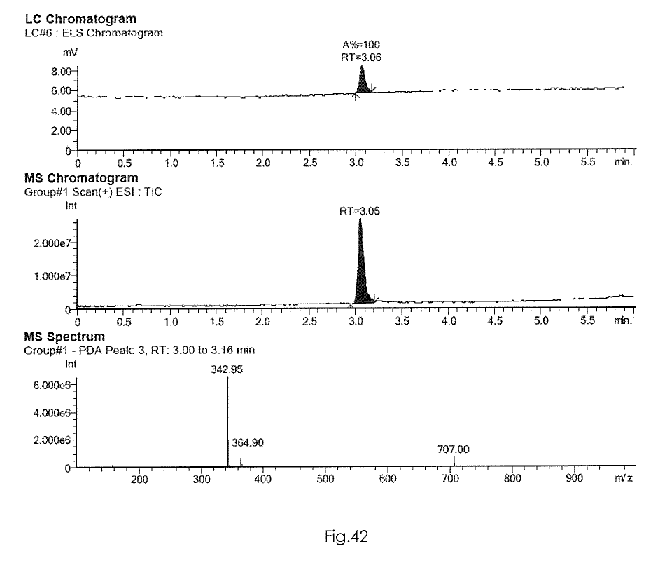

FIG. 42 shows the MS chromatogram, the MS spectrum and the PDA chromatogram of the compound of example 45.

### IC50 [μM]: <50.

### Route 9

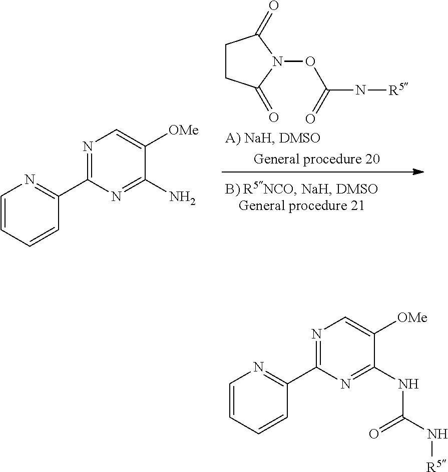

### General Procedure 20:

### Example 46

### 1-(5-Methoxy-2-pyridin-2-yl-pyrimidin-4-yl)-3-methyl-urea

Sodium hydride (60% in mineral oil, 12 mg, 0.29 mmol) was added at 0° C. to a solution of 5-methoxy-2-pyridin-2-yl-pyrimidin-4-ylamine (50 mg, 0.24 mmol) in DMSO (1 ml) and the mixture was stirred for 0.25 h. N-succinimidyl-N-methyl carbamate (51 mg, 0.29 mmol) was added dropwise and the mixture was stirred at room temperature for 4 h. The reaction mixture was diluted with ice-water (10 ml) and the aqueous phase was extracted with EtOAc (×2). The combined organic phases were washed with brine, dried (Na₂SO₄) and concentrated in vacuo. The crude residue was purified by column chromatography with DCM/0.1% NH₃ in MeOH (97:3) as the eluent to give the title compound (21 mg, 32%).

### MW: 259.26

HPLCMS (Method A): [m/z]: 260

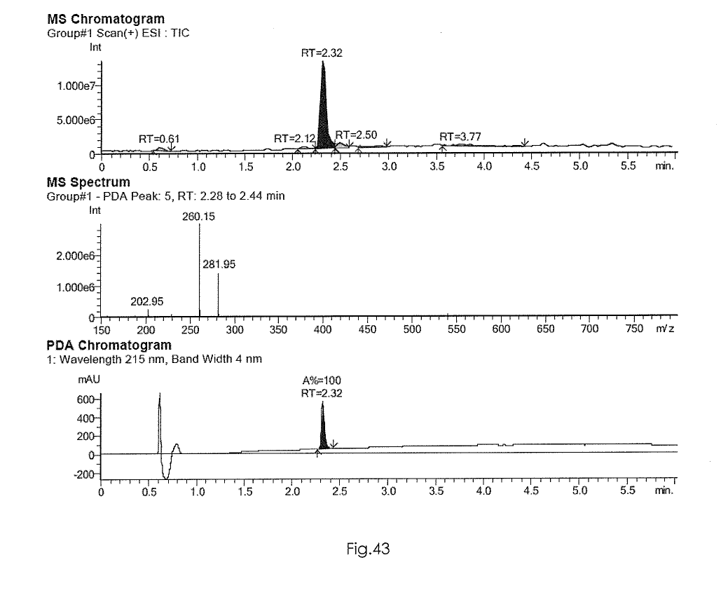

FIG. 43 shows the MS chromatogram, the MS spectrum and the PDA chromatogram of the compound of example 46.

### IC50 [μM]: >50.

### Example 47

### General Procedure 21:

### 1-Isopropyl-3-(5-methoxy-2-pyridin-2-yl-pyrimidin-4-yl)-urea

Sodium hydride (60% in mineral oil, 13 mg, 0.3 mmol) was added to a solution of 5-methoxy-2-pyridin-2-yl-pyrimidin-4-ylamine (50 mg, 0.24 mmol) in DMSO (1 ml) and the mixture was stirred at room temperature for 0.25 h. Iso-propyl isocyanate (42 mg, 0.49 mmol) was added at room temperature and the mixture was stirred at 80° C. for 14 h. The mixture was diluted with water (10 ml) and the aqueous phase was extracted with EtOAc (×2). The combined organic phases were washed with brine, dried (Na₂SO₄) and concentrated in vacuo. The crude residue was purified by column chromatography with DCM/3% NH₃ in MeOH (95:5) as the eluent to give the title compound (23 mg, 32%).

### MW: 287.31

HPLCMS (Method A): [m/z]: 288

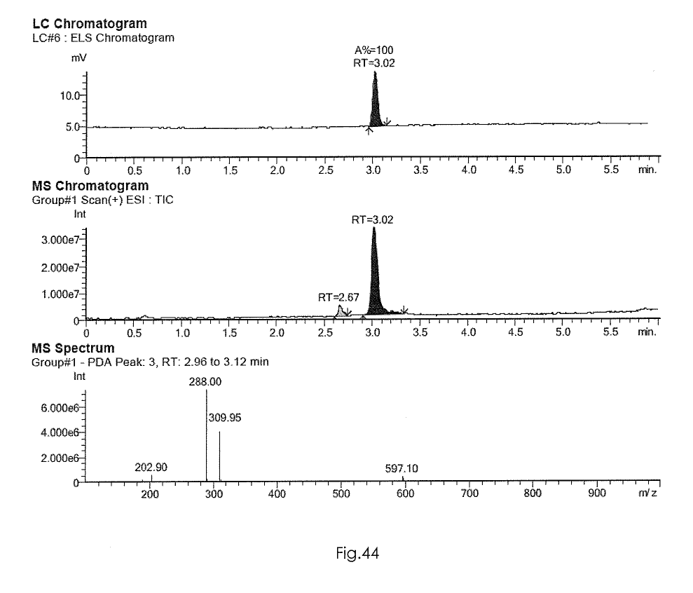

FIG. 44 shows the MS chromatogram, the MS spectrum and the PDA chromatogram of the compound of example 47.

### IC50 [μM]: >50.

### Example 48

### Synthesis of 1-(5-Methoxy-2-pyridin-2-yl-pyrimidin-4-yl)-3-phenyl-urea

In a similar fashion route 9 general procedure 21, 5-methoxy-2-pyridin-2-yl-pyrimidin-4-ylamine (50 mg, 0.24 mmol), sodium hydride (60% in mineral oil, 12 mg, 0.29 mmol) and phenyl isocyanate (35 mg, 0.29 mmol) gave the title compound (16 mg, 20%) after purification by column chromatography with DCM/0.1% NH₃ in MeOH (95:5) as the eluent.

### MW: 321.33

HPLCMS (Method A): [m/z]: 322

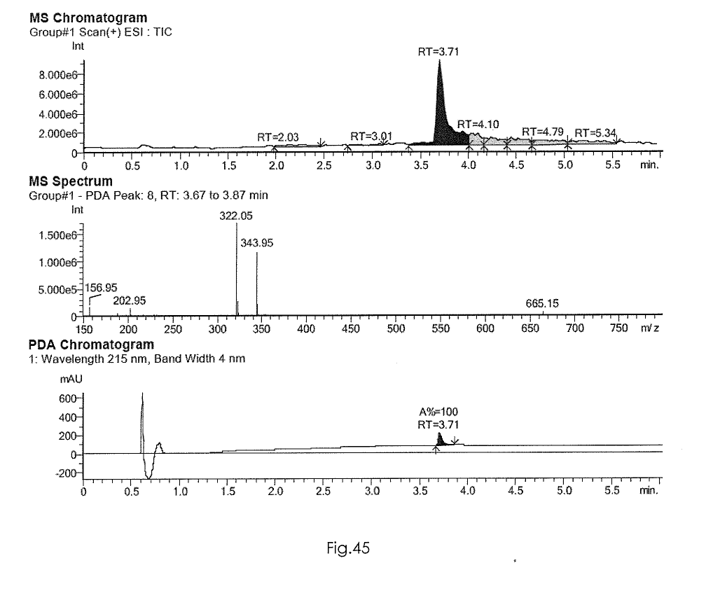

FIG. 45 shows the MS chromatogram, the MS spectrum and the PDA chromatogram of the compound of example 48.

### IC50 [μM]: >50.

### Route 10

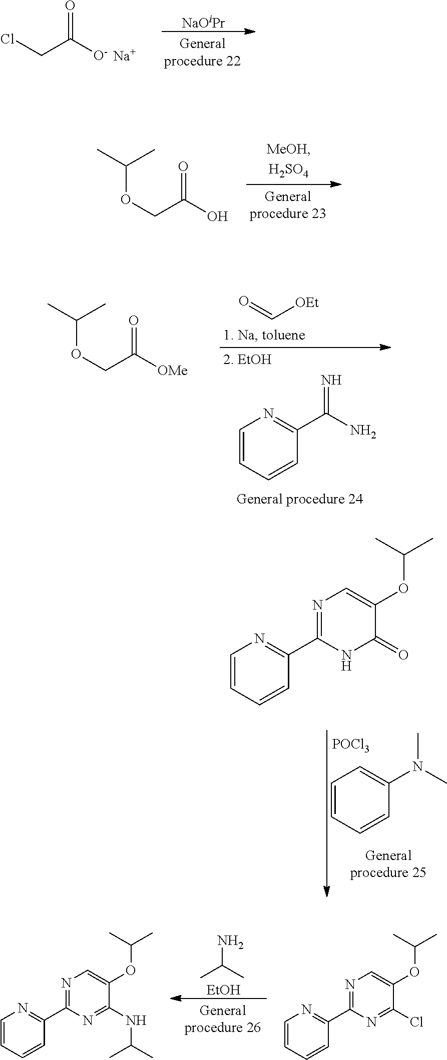

### General Procedure 22:

### Isopropoxy-Acetic Acid

The sodium salt of chloroacetic acid (20 g, 171 mmol) was added portionwise at 80° C. to sodium isopropoxide solution (prepared from 5.92 g of sodium and 60 ml of iso-propanol). The reaction mixture was heated under reflux for 4 h. After cooling, the mixture was concentrated in vacuo. The residue was diluted with water (80 ml) and acidified to pH 2-3 with 1N HCl. The aqueous phase was extracted with EtOAc (×6). The combined organic phases were dried (Na₂SO₄) and concentrated in vacuo to give the title compound (18 g, 89%), which was used without purification.

### General Procedure 23:

### Isopropoxy-Acetic Acid Methyl Ester

Thionyl chloride (22.2 ml, 303 mmol) was added dropwise to a solution of isopropoxy-acetic acid (17.9 g, 179 mmol) in MeOH (70 ml) at −5° C. The reaction mixture was heated under reflux for 9 h. After cooling, the mixture was concentrated in vacuo. The residue was diluted with saturated aqueous NaHCO₃ solution (100 ml) and extracted with Et₂O (×2). The combined organic phases were washed with brine, dried (Na₂SO₄) and concentrated in vacuo to give the title compound as yellow oil (15.5 g, 78%), which was used without purification.

### General Procedure 24:

### 5-Isopropoxy-2-pyridin-2-yl-3H-pyrimidin-4-one

Isopropoxy-acetic acid methyl ester (1.0 g, 7.5 mmol) and ethyl formate (0.56 g, 7.5 mmol) were added simultaneously to stirring suspension of sodium (0.18 g, 7.5 mmol) in toluene (20 ml) and the mixture was stirred at room temperature for 12 h. The toluene was decanted, the residue was diluted with EtOH (20 ml) and pyridine-2-carboxamidine (0.83 g, 5.3 mmol) was added followed by a solution of sodium ethoxide (prepared from Na 0.35 g, 15 mmol in 5 ml of EtOH). The reaction mixture was heated under reflux for 20 h. The mixture was filtered and the residue neutralized with 1N HCl (10 ml). The mixture was concentrated in vacuo and the crude residue was purified by column chromatography with DCM/1% NH₃ in MeOH (98:2) as the eluent to give the title compound (0.18 g, 11%).

### MW: 231.25

HPLCMS (Method D): [m/z]: 232

### General Procedure 25:

### 4-Chloro-5-isopropoxy-2-pyridin-2-yl-pyrimidine

A solution 5-isopropoxy-2-pyridin-2-yl-3H-pyrimidin-4-one (0.18 g, 0.78 mmol) and POCl₃ (0.76 ml, 7.8 mmol) in N,N-dimethyl aniline (0.22 ml) was heated under reflux for 1 h. The reaction mixture was poured into ice (50 ml) and basified to pH 8-9 with saturated aqueous NaHCO₃ solution. The aqueous phase was extracted with EtOAc (×3). The combined organic phases were dried (Na₂SO₄) and concentrated in vacuo.

The crude residue was purified by column chromatography with DCM/1% NH₃ in MeOH (98:2) as eluent to give the title compound (0.14 g, 72%).

### MW: 249.69

HPLCMS (Method D): [m/z]: 250

### General Procedure 26:

### Example 49

### (5-Isopropoxy-2-pyridin-2-yl-pyrimidin-4-yl)-isopropyl-amine

4-Chloro-5-isopropoxy-2-pyridin-2-yl-pyrimidine (0.13 g, 0.52 mmol), iso-propylamine (45 μl, 0.52 mmol) and DIPEA (0.18 ml, 1.04 mmol) were dissolved in EtOH (2 ml) and the mixture was stirred at 80° C. for 15 h. After cooling, the mixture was concentrated in vacuo. The residue was diluted with water (15 ml) and the aqueous phase was extracted with EtOAc (×3). The combined organic phases were dried (Na₂SO₄) and concentrated in vacuo. The crude residue was purified by column chromatography with DCM/1% NH₃ in MeOH (95:5) as the eluent to give the title compound (55 mg, 38%)

### MW: 272.34

HPLCMS (Method A): [m/z]: 273

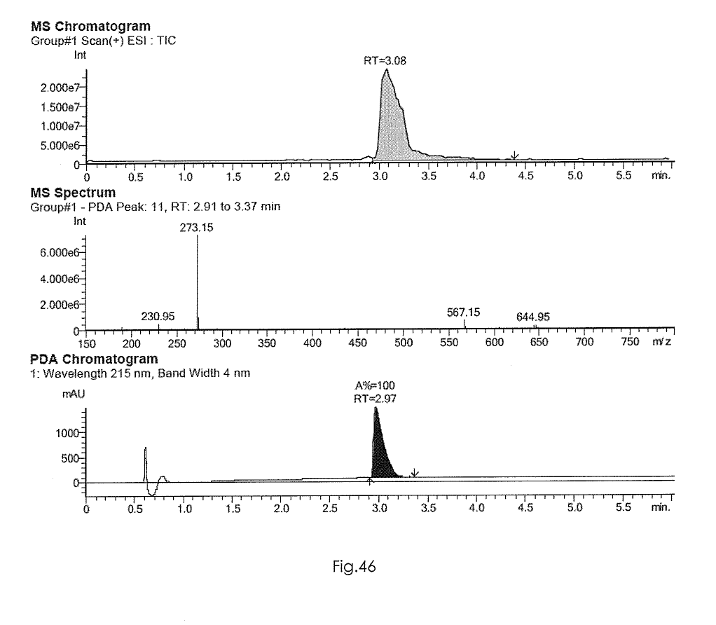

FIG. 46 shows the MS chromatogram, the MS spectrum and the PDA chromatogram of the compound of example 49.

### IC50 [μM]: >50.

### Route 12

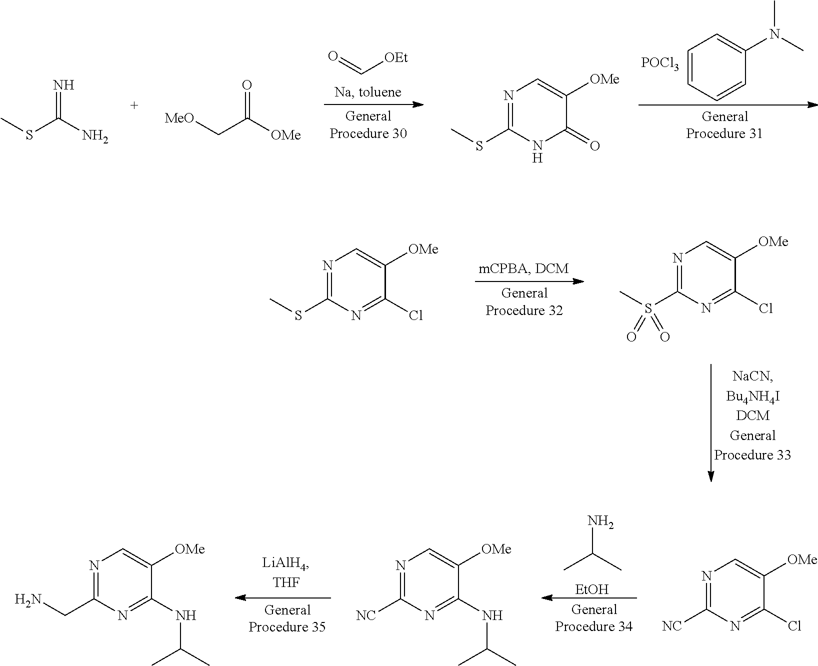

### General Procedure 30:

### 5-Methoxy-2-methylsulfanyl-3H-pyrimidin-4-one

Methyl methoxyacetate (2.0 g, 19.2 mmol) and ethyl formate (1.42 g, 19.2 mmol) were added simultaneously to a stirring suspension of sodium (0.44 g, 19.2 mmol) in toluene (20 ml) and the mixture stirred at room temperature for 12 h. The toluene was decanted, the crude residue was diluted with EtOH (20 ml) and S-methyl thiourea (1.3 g, 15 mmol) was added in one portion followed by a solution of sodium ethoxide (prepared from Na 0.35 g, 15 mmol and 5 ml of EtOH). The reaction mixture was heated under reflux for 15 h. The mixture was filtered and the residue was neutralized with 1N HCl (10 ml). The solvent was removed in vacuo. The crude residue was diluted with MeOH (20 ml), stirred for 0.25 h and filtered through celite. The filtrate was concentrated in vacuo to give the title compound (0.5 g, 21%).

### MW: 172.20

HPLCMS (Method D): [m/z]: 173

### General Procedure 31:

### 4-Chloro-5-methoxy-2-methylsulfanyl-pyrimidine

A solution of 5-methoxy-2-methylsulfanyl-3H-pyrimidin-4-one (0.77 g, 4.4 mmol) and POCl₃ (6.8 g, 44 mmol) in N,N-dimethyl aniline (0.4 ml) was heated under reflux for 1 h. The reaction mixture was poured into ice (50 ml) and basified to pH 8-9 with saturated aqueous NaHCO₃ and the aqueous phase was extracted with DCM (×3). The combined organic phases were dried (Na₂SO₄) and concentrated in vacuo. The crude residue was purified by column chromatography with EtOAc/hexane (1:9-4:6) as the eluent to give the title compound (0.2 g, 33%).

### MW: 190.65

HPLCMS (Method D): [m/z]: 191

### General Procedure 32:

### 4-Chloro-2-methanesulfonyl-5-methoxy-pyrimidine

A solution of 3-chloroperoxybenzoic acid (0.4 g, 2.3 mmol) in DCM (2 ml) was added dropwise to a solution of 4-chloro-5-methoxy-2-methylsulfanyl-pyrimidine (0.15 g, 0.78 mmol) in DCM (10 ml) and the mixture was stirred at room temperature for 12 h. Water (10 ml) was added, the aqueous phase was extracted with DCM and concentrated in vacuo. The crude residue was purified by column chromatography with DCM/1% NH₃ in MeOH (98:2) as the eluent to give the title compound (0.18 g, 100%).

### MW: 222.64

HPLCMS (Method D): [m/z]: 223

### General Procedure 33:

### 4-Chloro-5-methoxy-pyrimidine-2-carbonitrile

4-Chloro-2-methanesulfonyl-5-methoxy-pyrimidine (0.18 g, 0.8 mmol) was added to a solution of sodium cyanide, tetrabutyl ammonium iodide (16 mg, 0.04 mmol) in DCM (3 ml) and water (0.6 ml) and the mixture was stirred at room temperature for 16 h. Water (10 ml) was added and the mixture was extracted with DCM (×2), the combined organic phases were dried (Na₂SO₄) and concentrated in vacuo. The crude residue was purified by column chromatography with EtOAc/hexane (1:9-4:6) as the eluent to give the title compound (65 mg, 50%).

### MW: 169.56

HPLCMS (Method D): [m/z]: 170

### General Procedure 34:

### 4-Isopropylamino-5-methoxy-pyrimidine-2-carbonitrile

4-Chloro-5-methoxy-pyrimidine-2-carbonitrile (65 mg, 0.38 mmol), iso-propylamine (34 μl, 0.42 mmol) and DIPEA (75 μl, 0.46 mmol) were dissolved in EtOH (2 ml) and the mixture was stirred at room temperature for 15 h. The mixture was concentrated in vacuo. The residue was diluted with water (15 ml) and the reaction mixture extracted with ethyl acetate (×3). The combined organic phases were dried (Na₂SO₄) and concentrated in vacuo. The crude residue was triturated from pentane to give the title compound (30 mg, 40%).

### MW: 192.21

HPLCMS (Method D): [m/z]: 193

### Example 50

### General Procedure 35:

### (2-Aminomethyl-5-methoxy-pyrimidin-4-yl)-isopropyl-amine

4-Isopropylamino-5-methoxy-pyrimidine-2-carbonitrile (30 mg, 0.13 mmol) in THF (3 ml) was added dropwise to a solution of lithium aluminium hydride (19 mg, 0.52 mmol) in THF (2 ml) at 0° C. and the mixture was stirred at room temperature for 0.75 h. The residue was diluted with 1N NaOH solution (5 ml) and the mixture was concentrated in vacuo. The crude residue was purified by column chromatography with DCM/MeOH (98:2) as the eluent to give the title compound (29 mg, 96%).

### MW: 196.27

HPLCMS (Method D): [m/z]: 197

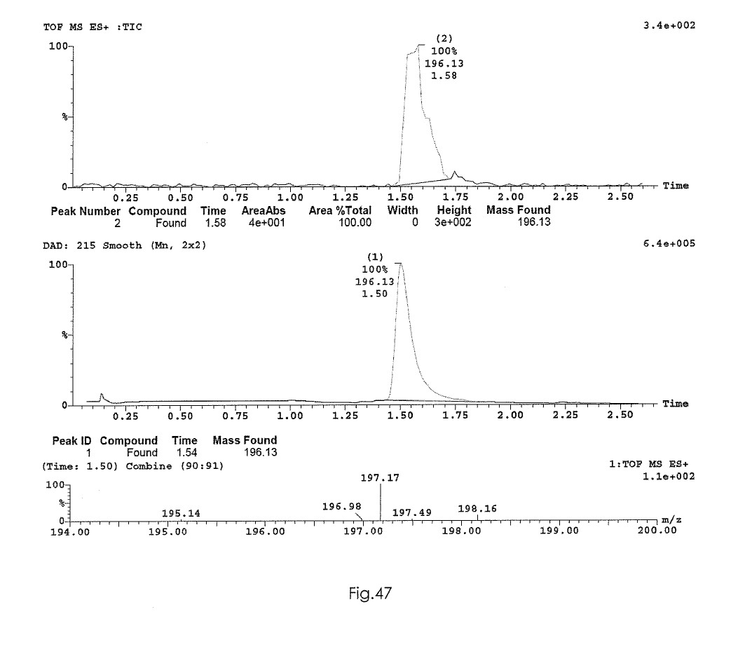

FIG. 47 shows the spectra/chromatograms of the compound of example 50. IC50 [μM]: <50.

### Route 13

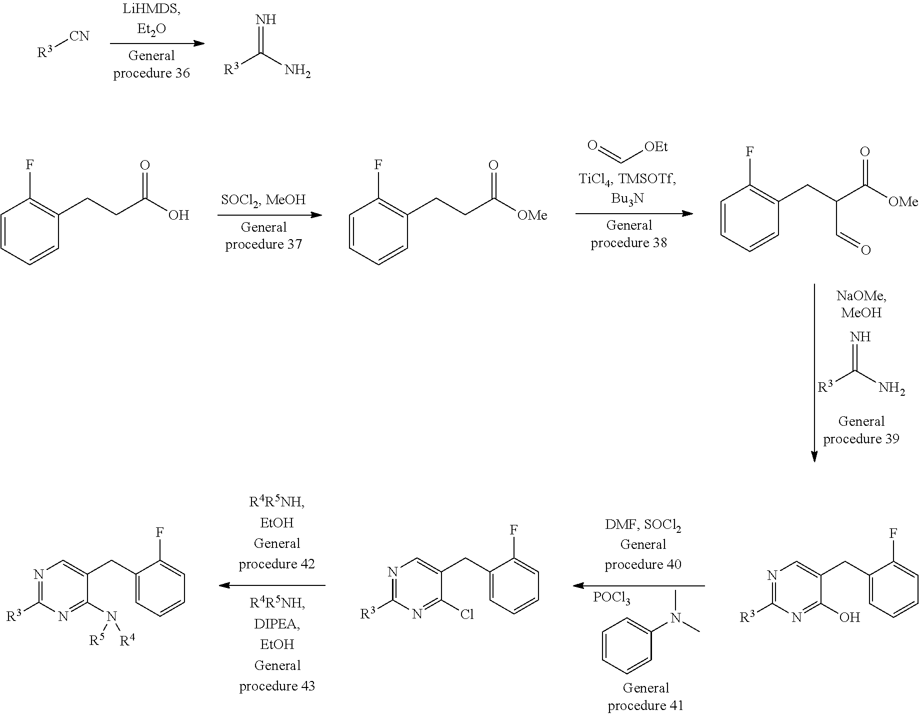

### General Procedure 36:

### Pyridine-2-carboxamidine

Lithium hexamethyl disilazide (1M solution in THF, 60.5 ml, 60.5 mmol) was added to a solution of pyridine-2-carbonitrile (3.0 g, 28.8 mmol) in Et₂O (30 ml) at 0° C. The reaction was allowed to warm to room temperature overnight. The reaction was cooled to 0° C. and 3 M HCl (54 ml) was added and the reaction was stirred for 30 min. Water (135 ml) was added and the organic phase was separated and discarded. The aqueous layer was basified to pH 14 with saturated aqueous NaOH and extracted with DCM (×3). The combined organic extracts were dried (Na₂SO₄) and concentrated in vacuo to give the title compound (1.70 g, 49%).

### MW: 121.14

HPLCMS (Method B): [m/z]: 122

### Nicotinamidine

In a similar fashion using route 13 general procedure 36, lithium hexamethyl disilazide (1M solution in THF, 40.4 ml, 40.4 mmol), nicotinonitrile (2.0 g, 19.2 mmol) in Et₂O (30 ml) gave the title compound (0.95 g, 41%).

### MW: 121.14

HPLCMS (Method B): [m/z]: 122

### General Procedure 37:

### 3-(2-Fluoro-phenyl)-propionic acid methyl ester

Thionyl chloride (0.65 ml, 9.82 mmol) was added dropwise to a solution of 3-(2-fluoro-phenyl)-propionic acid (1.0 g, 5.95 mmol) in MeOH (10 ml) at 0° C. The mixture was allowed to warm to room temperature and was heated under reflux for 2 h. The reaction mixture was concentrated in vacuo, diluted with saturated aqueous NaHCO₃ solution (10 ml) and extracted with Et₂O (×3). The combined organic phases were washed with brine, dried (Na₂SO₄) and concentrated in vacuo to give the title compound (1.0 g, 93%).

### MW: 182.20

HPLCMS (Method B): [m/z]: 183

### General Procedure 38:

### 2-(2-Fluoro-benzyl)-3-oxo-propionic acid methyl ester

Titanium(IV) chloride (0.91 ml, 8.24 mmol), trimethylsilyl trifluoromethanesulfonate (25 μl, 0.14 mmol) followed by tri-n-butylamine (2.9 ml, 12.35 mmol) were added dropwise to a solution of 3-(2-fluoro-phenyl)-propionic acid methyl ester (0.5 g, 2.74 mmol) and ethyl formate (0.33 ml, 4.11 mmol) in toluene (20 ml). The mixture was stirred at room temperature for 18 h. Water (20 ml) was added and the aqueous phase was extracted with EtOAc (×2). The combined organic phases were washed with brine, dried (Na₂SO₄) and concentrated in vacuo. Partial purification by column chromatography with EtOAc/heptane (8:92) as eluent gave the title compound (200 mg, 35%) in impure form. The product was used in the next step without further purification. The compound could not be detected by HPLCMS therefore structure was confirmed by 1H-NMR.

### Example 51

### General Procedure 39:

### 5-(2-Fluoro-benzyl)-2-pyridin-2-yl-pyrimidin-4-ol

NaOMe (133 mg, 2.48 mmol) was added to a solution of 2-(2-fluoro-benzyl)-3-oxo-propionic acid methyl ester (500 mg, 2.38 mmol) and pyridine-2-carboxamidine 33 (200 mg, 1.65 mmol) in MeOH (10 ml). The reaction was stirred at room temperature for 65 h. The reaction was concentrated in vacuo and purified by column chromatography with MeOH/DCM (5:95) as the eluent. The resulting solid was triturated from Et₂O to give the title compound (262 mg, 45%).

### MW: 281.28

HPLCMS (method A): [m/z]: 282

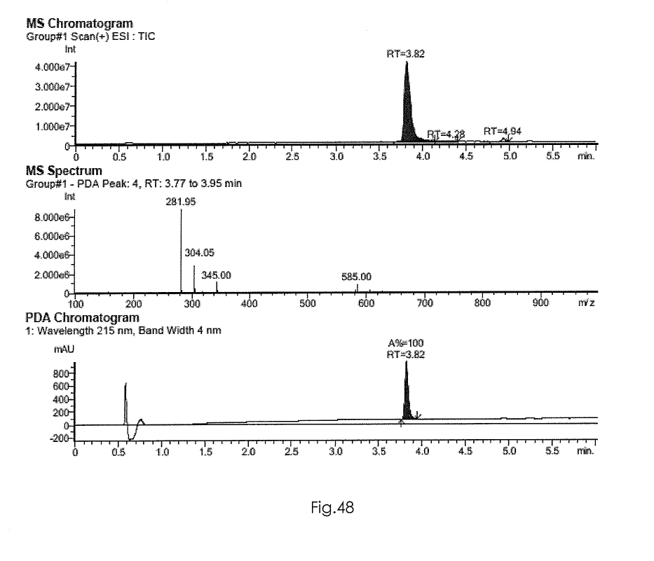

FIG. 48 shows the MS chromatogram, the MS spectrum and the PDA chromatogram of the compound of example 51.

### IC50 [μM]: <50.

### Example 52

### 5-(2-Fluoro-benzyl)-2-pyridin-3-yl-pyrimidin-4-ol

In a similar fashion using route 13 general procedure 39, NaOMe (167 mg, 3.10 mmol), 2-(2-fluoro-benzyl)-3-oxo-propionic acid methyl ester (650 mg, 3.10 mmol) and nicotinamidine 73 (250 mg, 2.06 mmol) gave the title compound (279 mg, 37%) after purification by column chromatography with DCM/MeOH (97:3) as the eluent followed by trituration from Et₂O.

### MW: 281.28

HPLCMS (Method A): [m/z]: 282

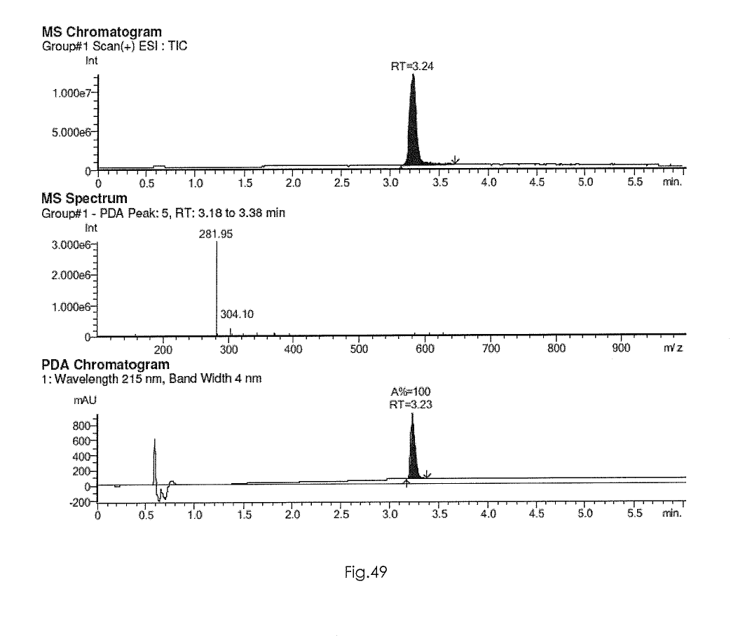

FIG. 49 shows the MS chromatogram, the MS spectrum and the PDA chromatogram of the compound of example 52.

### IC50 [μM]: >50.

### Example 53

### General Procedure 40:

### 4-Chloro-5-(2-fluoro-benzyl)-2-pyridin-2-yl-pyrimidine

DMF (cat) was added to a solution of 5-(2-fluoro-benzyl)-2-pyridin-2-yl-pyrimidin-4-ol (100 mg, 0.35 mmol) in thionyl chloride (1 ml) and the mixture was heated at 80° C. for 1 h. After cooling, the reaction mixture was concentrated in vacuo. The residue was basified with saturated aqueous NaHCO₃ solution (10 ml) and extracted with DCM (×3). The combined organic phases were dried (Na₂SO₄) and concentrated in vacuo to give the title compound (107 mg, 100%).

### MW: 299.73

HPLCMS (method A): [m/z]: 300

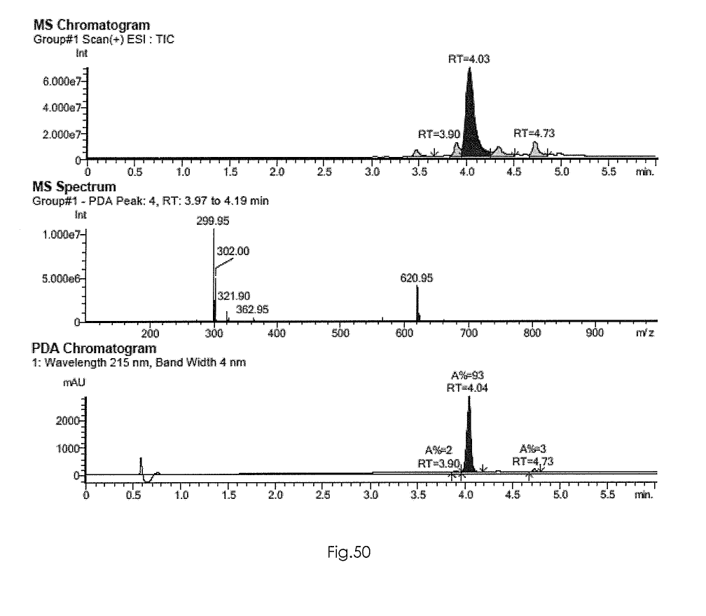

FIG. 50 shows the MS chromatogram, the MS spectrum and the PDA chromatogram of the compound of example 53.

### IC50 [μM]: <50.

### Example 54

### 4-Chloro-5-(2-fluoro-benzyl)-2-pyridin-3-yl-pyrimidine

In a similar fashion using route 13 general procedure 40, DMF (cat), 5-(2-fluoro-benzyl)-2-pyridin-3-yl-pyrimidin-4-ol (100 mg, 0.36 mmol) and thionyl chloride (1 ml) gave the title compound (107 mg, 100%) after aqueous work up.

### MW: 299.74

HPLCMS (Method A): [m/z]: 300

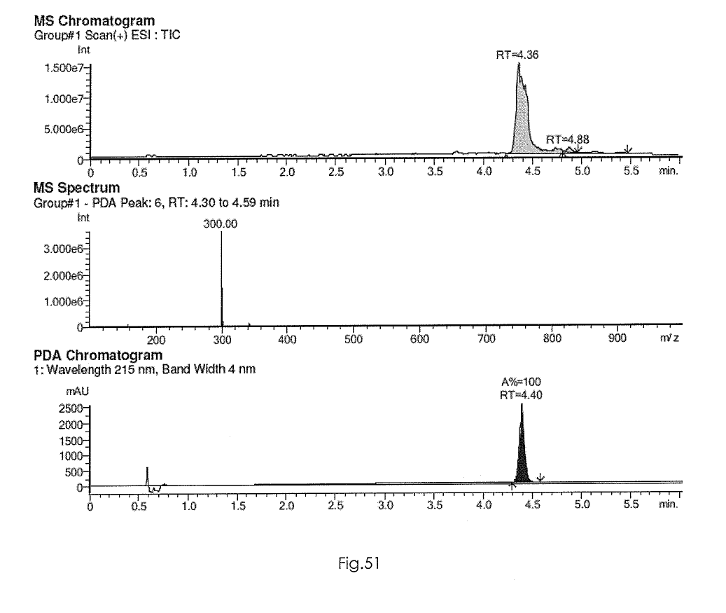

FIG. 51 shows the MS chromatogram, the MS spectrum and the PDA chromatogram of the compound of example 54.

### IC50 [μM]: >50.

### General Procedure 41:

### 4-Chloro-5-(2-fluoro-benzyl)-2-pyridin-2-yl-pyrimidine

5-(2-Fluorobenzyl)-2-(pyridin-2-yl)pyrimidin-4-ol (70 mg, 0.25 mmol) and POCl₃ (0.39 g, 2.5 mmol) in N,N-dimethyl aniline (0.07 ml) were heated under reflux for 1 h. The reaction mixture was poured into ice (50 ml) and basified to pH 8-9 with saturated aqueous NaHCO₃ solution. The aqueous phase was extracted with DCM (×3). The combined organic phases were dried (Na₂SO₄) and concentrated in vacuo. The crude residue was purified by column chromatography with DOM as the eluent to give the title compound (25 mg, 33%).

### MW: 299.74

HPLCMS (method D) [m/z]: 300

### Example 55

### General Procedure 42:

### [5-(2-Fluoro-benzyl)-2-pyridin-2-yl-pyrimidin-4-yl]-isopropyl-amine

Diisopropylamine (69 μl, 0.80 mmol) was added to a solution of 4-chloro-5-(2-fluoro-benzyl)-2-pyridin-2-yl-pyrimidine (107 mg, 0.36 mmol) in EtOH (1.1 ml) and the mixture was heated under reflux for 18 h. After cooling, the reaction mixture was concentrated in vacuo. The residue was basified with saturated aqueous NaHCO₃ solution (1 ml) and extracted with DCM (×3). The organic phase was washed with water (×2), dried (Na₂SO₄) and concentrated in vacuo to give the title compound (92 mg, 78%).

### MW: 322.39

HPLCMS (Method A): [m/z]: 323

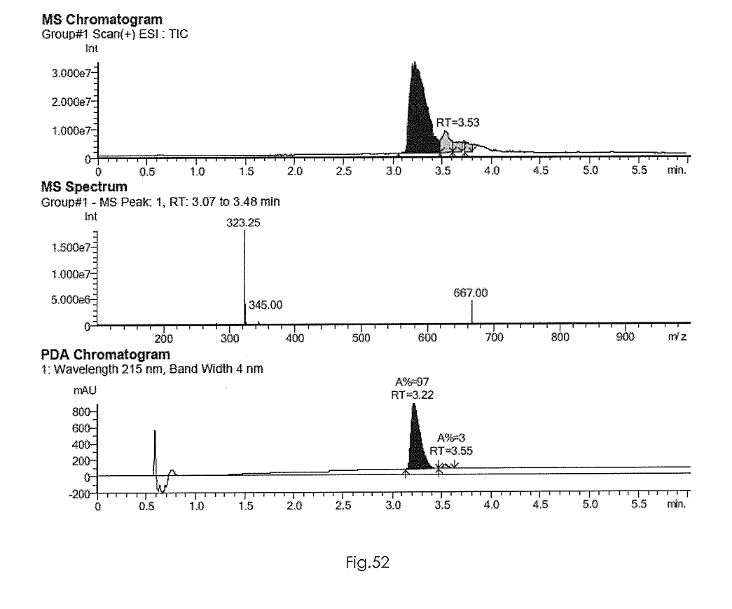

FIG. 52 shows the MS chromatogram, the MS spectrum and the PDA chromatogram of the compound of example 55.

### IC50 [μM]: <50.

### Example 56

### [5-(2-Fluoro-benzyl)-2-pyridin-2-yl-pyrimidin-4-yl]-methyl-amine

In a similar fashion using route 13 general procedure 42, 4-chloro-5-(2-fluoro-benzyl)-2-pyridin-2-yl-pyrimidine (103 mg, 0.34 mmol), methylamine (2M in THF, 0.75 ml, 1.53 mmol) in EtOH (1 ml) to give the title compound (57 mg, 57%) after purification by preparative HPLC (acidic conditions).

### MW: 294.32

HPLCMS (Method A): [m/z]: 295

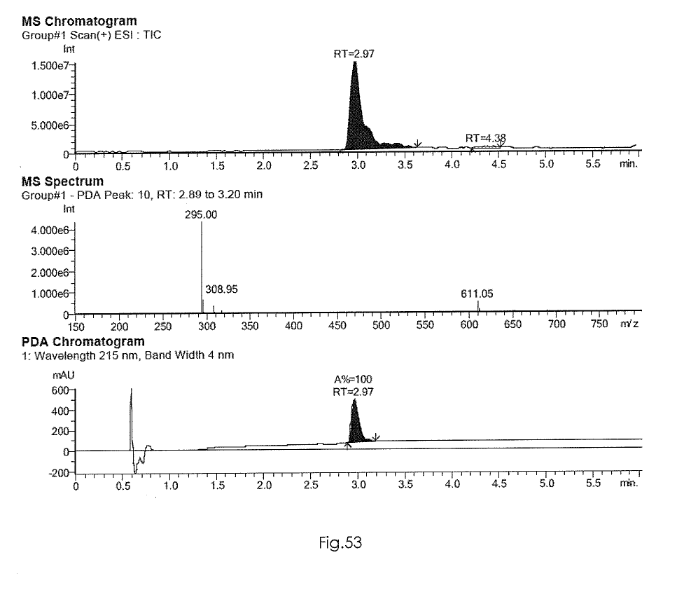

FIG. 53 shows the MS chromatogram, the MS spectrum and the PDA chromatogram of the compound of example 56.

### IC50 [μM]: >50.

### Example 57

### Diethyl-[5-(2-fluoro-benzyl)-2-pyridin-2-yl-pyrimidin-4-yl]-amine

In a similar fashion using route 13 general procedure 42, 4-chloro-5-(2-fluoro-benzyl)-2-pyridin-2-yl-pyrimidine (103 mg, 0.34 mmol), diethylamine (0.16 ml, 1.53 mmol) in EtOH (1 ml) to give the title compound (88 mg, 77%) after basic work up without further purification.

### MW: 336.41

HPLCMS (Method A): [m/z]: 337

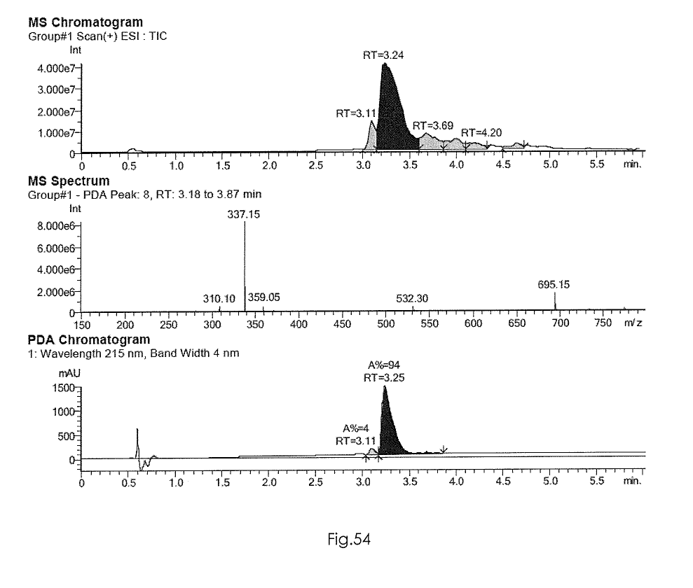

FIG. 54 shows the MS chromatogram, the MS spectrum and the PDA chromatogram of the compound of example 57.

### IC50 [μM]: <50.

### Example 58

### Cyclohexylmethyl-[5-(2-fluoro-benzyl)-2-pyridin-2-yl-pyrimidin-4-yl]-amine

In a similar fashion using route 13 general procedure 42, 4-chloro-5-(2-fluoro-benzyl)-2-pyridin-2-yl-pyrimidine (103 mg, 0.34 mmol), cyclohexanemethylamine (0.20 ml, 1.53 mmol) in EtOH (1 ml) to give the title compound (80 mg, 63%) purification by preparative HPLC (acidic conditions).

### MW: 376.47

HPLCMS (Method A): [m/z]: 377

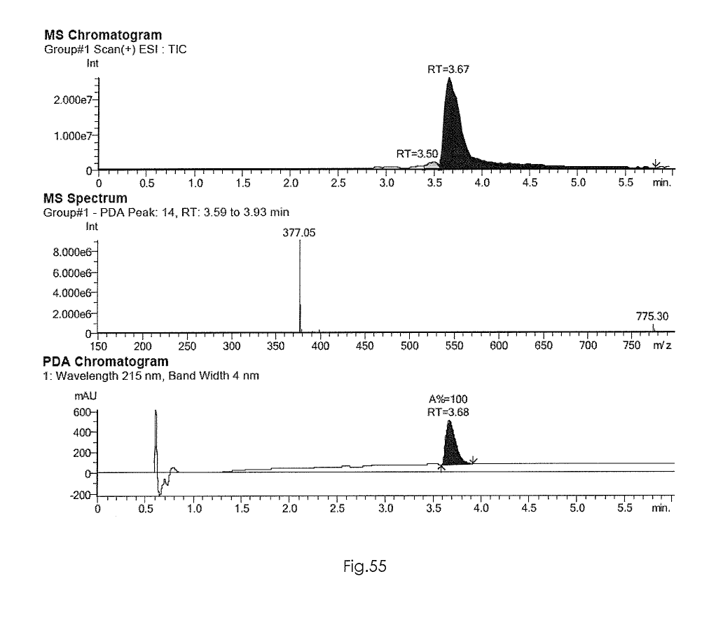

FIG. 55 shows the MS chromatogram, the MS spectrum and the PDA chromatogram of the compound of example 58.

### IC50 [μM]: >50.

### Example 59

### 4-[5-(2-Fluoro-benzyl)-2-pyridin-2-yl-pyrimidin-4-yl]-morpholine

In a similar fashion using route 13 general procedure 42, 4-chloro-5-(2-fluoro-benzyl)-2-pyridin-2-yl-pyrimidine (103 mg, 0.34 mmol), morpholine (0.13 ml, 1.53 mmol) in EtOH (1 ml) to give the title compound (82 mg, 69%) after basic work up without further purification.

### MW: 350.39

HPLCMS (Method A): [m/z]: 351

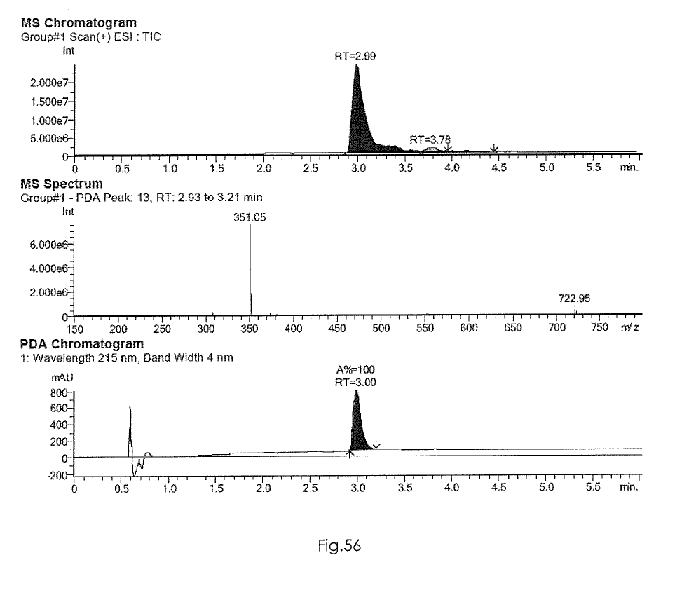

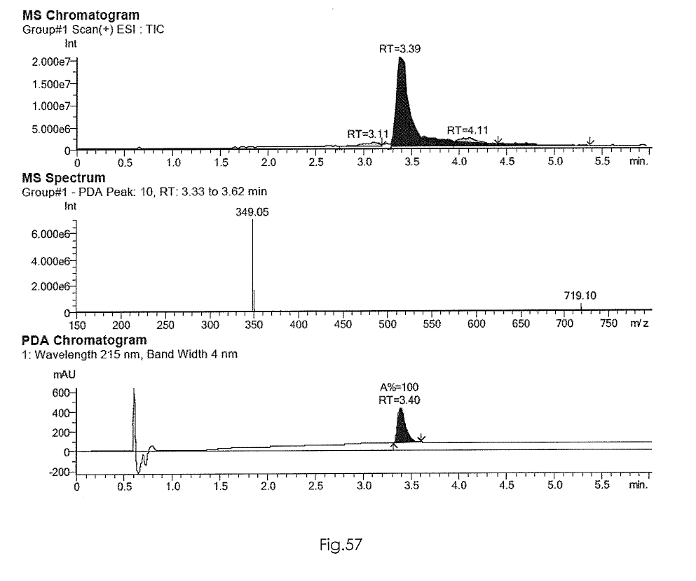

FIG. 56 shows the MS chromatogram, the MS spectrum and the PDA chromatogram of the compound of example 59.

### IC50 [μM]: <50.

### Example 60

### 5-(2-Fluoro-benzyl)-4-piperidin-1-yl-2-pyridin-2-yl-pyrimidine

In a similar fashion using route 13 general procedure 42, 4-chloro-5-(2-fluoro-benzyl)-2-pyridin-2-yl-pyrimidine (103 mg, 0.34 mmol), piperidine (0.15 ml, 1.53 mmol) in EtOH (1 ml) to give the title compound (88 mg, 74%) after basic work up without further purification.

### MW: 348.42

HPLCMS (Method A): [m/z]: 349

FIG. 57 shows the MS chromatogram, the MS spectrum and the PDA chromatogram of the compound of example 60.

### IC50 [μM]: <50.

### Example 61

### Benzyl-[5-(2-fluoro-benzyl)-2-pyridin-2-yl-pyrimidin-4-yl]-methyl-amine

In a similar fashion using route 13 general procedure 42, 4-chloro-5-(2-fluoro-benzyl)-2-pyridin-2-yl-pyrimidine (103 mg, 0.34 mmol), N-methylbenzylamine (0.20 ml, 1.53 mmol) in EtOH (1 ml) to give the title compound (97 mg, 74%) purification by preparative HPLC (acidic conditions).

### MW: 384.45

HPLCMS (Method A): [m/z]: 385

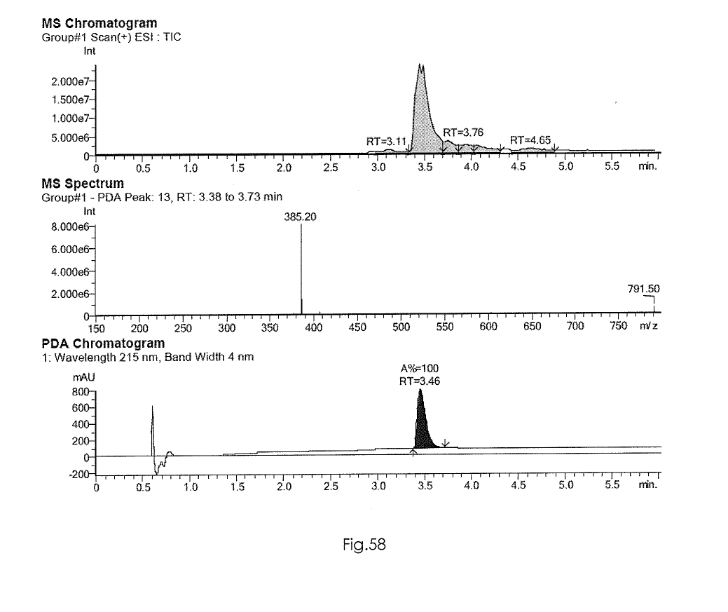

FIG. 58 shows the MS chromatogram, the MS spectrum and the PDA chromatogram of the compound of example 61.

### IC50 [μM]: >50.

### Example 62

### [5-(2-Fluoro-benzyl)-2-pyridin-3-yl-pyrimidin-4-yl]-isopropyl-amine

In a similar fashion using route 13 general procedure 42, diisopropylamine (126 μl, 1.49 mmol) and 4-chloro-5-(2-fluoro-benzyl)-2-pyridin-3-yl-pyrimidine (98 mg, 0.33 mmol) gave the title compound (83 mg, 79%) after aqueous work up.

### MW: 322.39

HPLCMS (Method A): [m/z]: 323

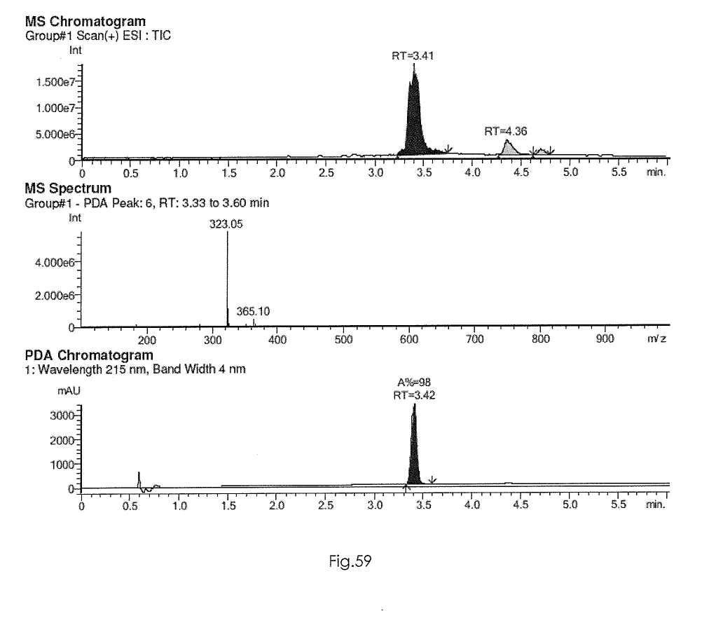

FIG. 59 shows the MS chromatogram, the MS spectrum and the PDA chromatogram of the compound of example 62.

### IC50 [μM]: >50.

### Example 63

### General Procedure 43:

### 5-(2-Fluoro-benzyl)-4-(4-methyl-piperazin-1-yl)-2-pyridin-2-yl-pyrimidine

4-Chloro-5-(2-fluoro-benzyl)-2-pyridin-2-yl-pyrimidine (25 mg, 0.08 mmol), 1-methyl piperazine (0.01 ml, 0.1 mmol) and DIPEA (17 μl, 0.1 mmol) were dissolved in EtOH (2 ml) and the mixture was stirred at room temperature for 15 h. The mixture was concentrated in vacuo. The residue was diluted with water (15 ml) and the reaction mixture extracted with ethyl acetate (×3). The combined organic phases were dried (Na₂SO₄) and concentrated in vacuo. The crude residue was purified by column chromatography with DCM/1% NH₃ in MeOH (96:4) as the eluent to give the title compound (14 mg, 46%).

### MW: 364.44

HPLCMS (method A) [m/z]: 364

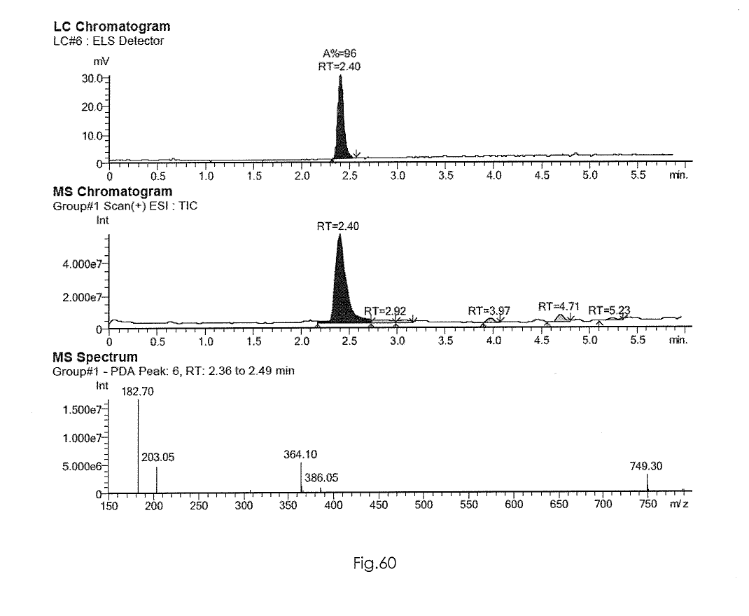

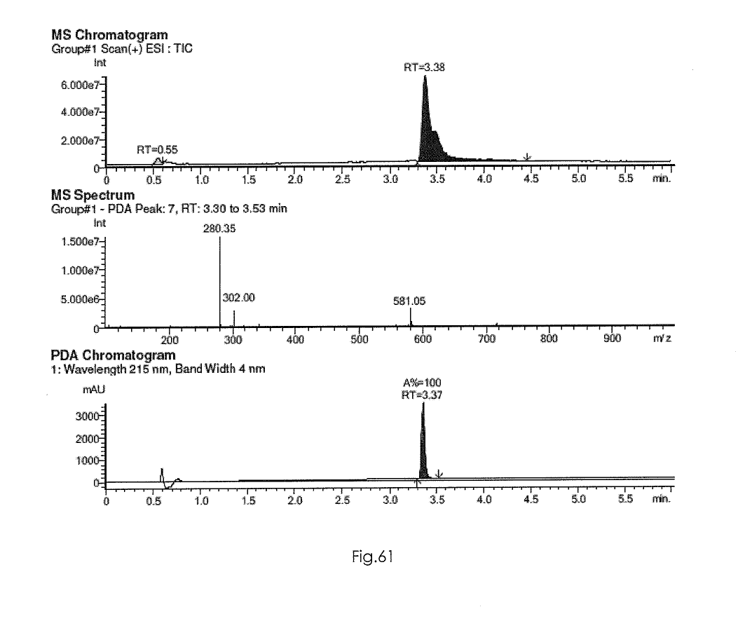

FIG. 60 shows the MS chromatogram, the MS spectrum and the PDA chromatogram of the compound of example 63.

### IC50 [μM]: <50.

### Route 14

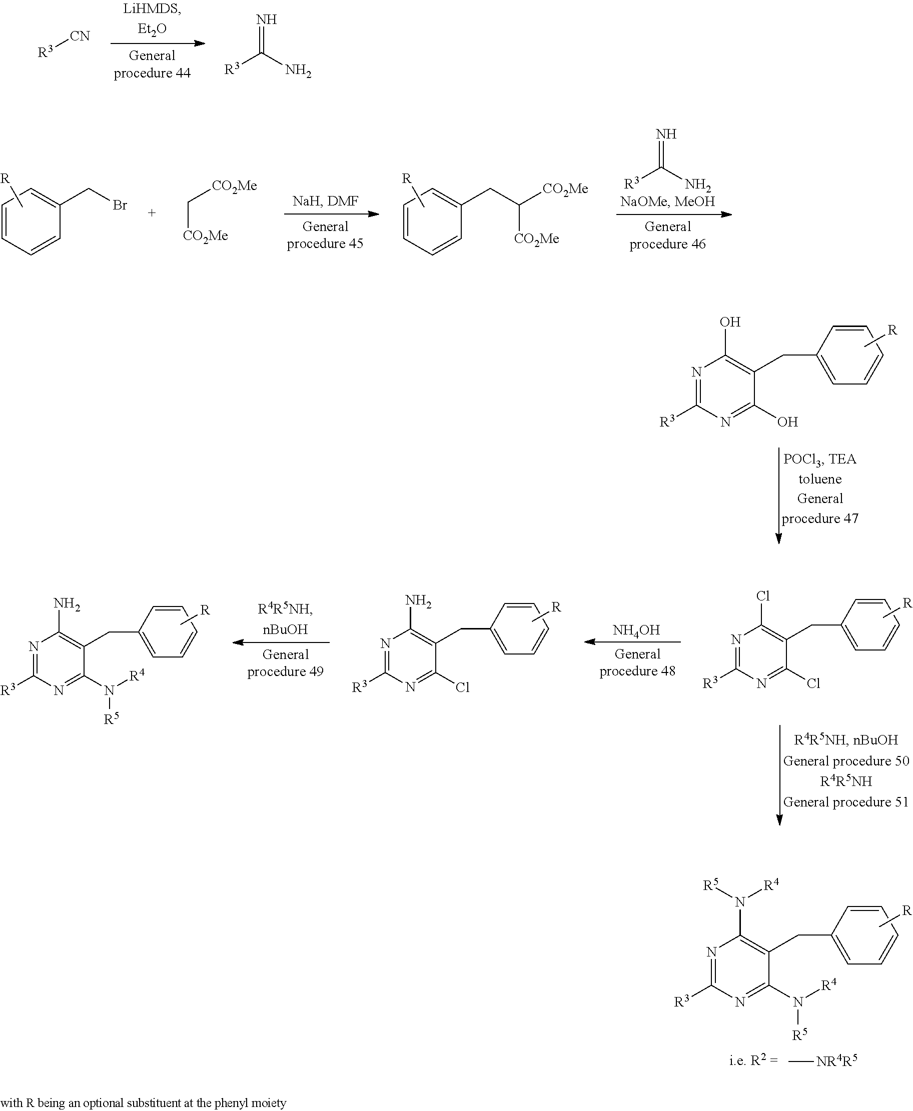

### General Procedure 44:

### 5-Fluoro-pyridine-2-carboxamidine

Trimethyl aluminum (3.54 g, 49.14 mmol) was added dropwise to a vigorously stirred solution of NH₄Cl (2.63 g, 49.14 mmol) in dry toluene (20 ml) at 0° C. The mixture was warmed to room temperature and was stirred for 15 min. A solution of 5-fluoropyridine-2-carbonitrile (2.00 g, 16.38 mmol) in toluene (20 ml) was added dropwise. The reaction mixture was heated at 80° C. for 18 h. After cooling, the mixture was transferred to a vigorously stirred and cooled (0° C.) slurry of silica (20.0 g) in chloroform (150 ml) and was stirred for 10 min. The mixture was filtered and the filter cake was washed with MeOH (×3). The filtrate was concentrated in vacuo. The residue was dissolved in 1M HCl (150 ml) and Et₂O (70 ml). The organic phase was separated and discarded. The aqueous phase was basified with saturated aqueous NaOH and extracted with chloroform (×2). The combined organic extracts were dried (Na₂SO₄) and concentrated in vacuo to give the title compound (394 mg, 17%). The compound could not be detected by HPLCMS therefore structure was confirmed by 1H-NMR.

### General Procedure 45:

### 2-Benzyl-malonic acid dimethyl ester

Malonic acid dimethyl ester (369 μl, 3.22 mmol) was added dropwise to a suspension of NaH (60% dispersion in mineral oil, 140 mg, 3.51 mmol) in DMF (5 ml) at 0° C. The reaction mixture was stirred at room temperature for 30 min. The reaction mixture was cooled to 0° C. and benzyl bromide (350 μl, 2.92 mmol) was added dropwise. The reaction mixture was allowed to warm to room temperature overnight. EtOAc (10 ml) was added followed by saturated aqueous NH₄Cl solution (10 ml). The phases were separated and the organic phase was washed with water, dried (Na₂SO₄) and concentrated in vacuo. The crude residue was purified by column chromatography with EtOAc/heptane (5:95) as the eluent to give the title compound (325 mg, 25%).

### MW: 222.24

HPLCMS (Method B): [m/z]: 223

### 2-(2-Fluoro-benzyl)-malonic acid dimethyl ester

In a similar fashion using route 14 general procedure 45, malonic acid dimethyl ester (2.0 ml, 17.46 mmol), NaH (60% dispersion in mineral oil, 0.76 g, 19.05 mmol), 2-fluorobenzyl bromide (2.1 ml, 19.05 mmol) in THF (60 ml) gave the title compound (1.80 g, 47%).

### MW: 240.23

HPLCMS (Method B): [m/z]: 241

### Example 64

### General Procedure 46:

### 5-Benzyl-2-pyridin-2-yl-pyrimidine-4,6-diol

NaOMe (316 mg, 5.85 mmol) was added to a solution of 2-benzyl-malonic acid dimethyl ester (650 mg, 2.92 mmol) and pyridine-2-carboxamidine (354 mg, 2.92 mmol) in MeOH (15 ml). The reaction mixture was stirred at room temperature for 40 min and then at 70° C. for 1 h. After cooling, the reaction mixture was concentrated in vacuo. The crude residue was purified by trituration from EtOAc to give the title compound (431 mg, 53%).

### MW: 279.29

HPLCMS (method A): [m/z]: 280

FIG. 61 shows the MS chromatogram, the MS spectrum and the PDA chromatogram of the compound of example 64.

### IC50 [μM]: >50.

### Example 65

### 5-(2-Fluoro-benzyl)-2-pyridin-2-yl-pyrimidine-4,6-diol

In a similar fashion using route 14 general procedure 46, NaOMe (111 mg, 2.06 mmol), 2-(2-fluoro-benzyl)-malonic acid dimethyl ester (496 mg, 2.06 mmol) and pyridine-2-carboxamidine (250 mg, 2.06 mmol) gave the title compound (361 mg, 59%).

### MW: 297.28

HPLCMS (Method A): [m/z]: 298

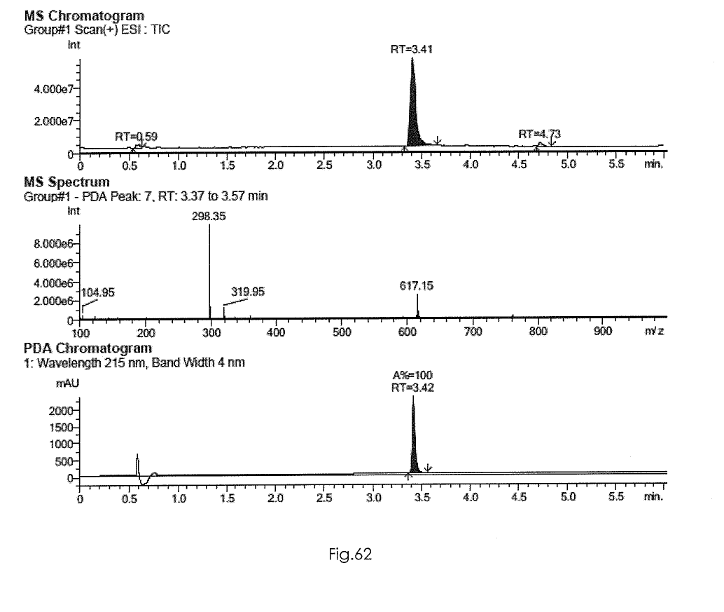

FIG. 62 shows the MS chromatogram, the MS spectrum and the PDA chromatogram of the compound of example 65.

### IC50 [μM]: >50.

### 5-(2-Fluoro-benzyl)-2-(5-fluoro-pyridin-2-yl)-pyrimidine-4,6-diol

In a similar fashion using route 14 general procedure 46, NaOMe (153 mg, 2.83 mmol), 2-(2-fluoro-benzyl)-malonic acid dimethyl ester (680 mg, 2.83 mmol) and 5-fluoro-pyridine-2-carboximidamide (394 mg, 2.83 mmol) gave the title compound (597 mg, 67%).

### MW: 315.27

HPLCMS (Method B): [m/z]: 316

### Example 66

### General Procedure 47:

### 5-Benzyl-4,6-dichloro-2-pyridin-2-yl-pyrimidine

A solution of POCl₃ (316 μl, 3.4 mmol) in toluene (3 ml) was added dropwise to a suspension of 5-benzyl-2-(pyridin-2-yl)pyrimidine-4,6-diol (430 mg, 1.54 mmol) and TEA (215 μl, 1.54 mmol) in toluene (5 ml) at 100° C. The reaction mixture was heated under reflux for 16 h. After cooling to room temperature and then to 0° C., water (3 ml) was added dropwise and the mixture was allowed to warm to room temperature. Attempted extraction with EtOAc failed therefore the mixture was concentrated in vacuo. The residue was basified with a saturated aqueous NaHCO₃ solution and extracted with DCM (×2) followed by chloroform (×2). The combined organic phases were dried (MgSO₄) and concentrated in vacuo to give the title compound (327 mg, 67%).

### MW: 316.19

HPLCMS (method A): [m/z]: 317

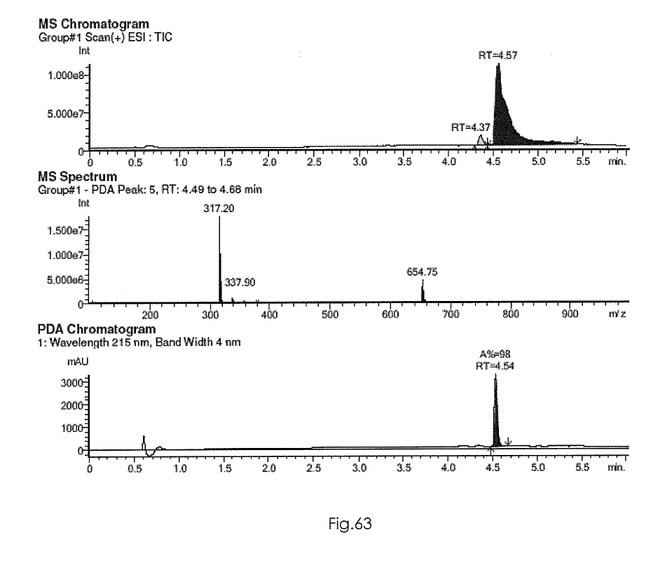

FIG. 63 shows the MS chromatogram, the MS spectrum and the PDA chromatogram of the compound of example 66.

### IC50 [μM]: >50.

### Example 67

### 4,6-Dichloro-5-(2-fluoro-benzyl)-2-pyridin-2-yl-pyrimidine

In a similar fashion using route 14 general procedure 47, POCl₃ (69 μl, 0.74 mmol), 5-(2-fluoro-benzyl)-2-pyridin-2-yl-pyrimidine-4,6-diol (100 mg, 0.34 mmol) and TEA (47 μl, 0.34 mmol) gave the title compound (112 mg, 77%).

### MW: 334.18

HPLCMS (Method A): [m/z]: 335

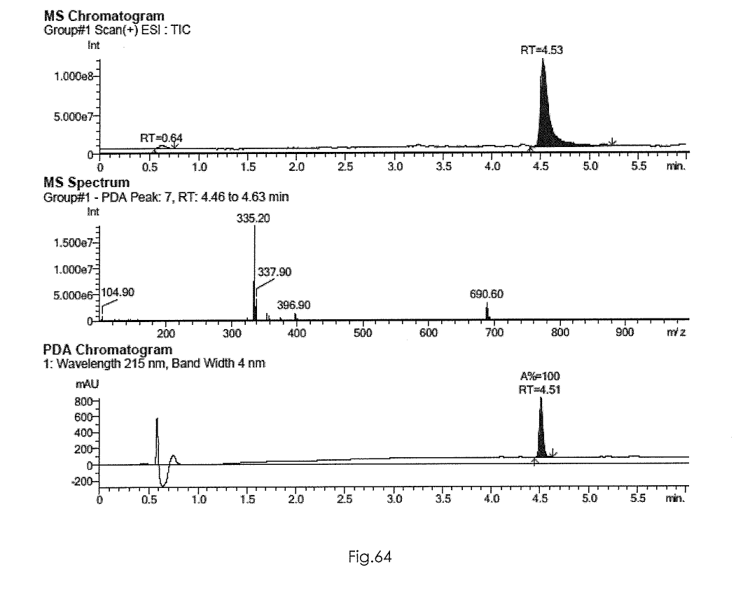

FIG. 64 shows the MS chromatogram, the MS spectrum and the PDA chromatogram of the compound of example 67.

### IC50 [μM]: >50.

### Example 68

### 4,6-Dichloro-5-(2-fluoro-benzyl)-2-(5-fluoro-pyridin-2-yl)-pyrimidine

In a similar fashion using route 14 general procedure 47, POCl₃ (384 μl, 4.12 mmol), 5-(2-fluoro-benzyl)-2-(5-fluoro-pyridin-2-yl)-pyrimidine-4,6-diol (590 mg, 1.87 mmol) and TEA (260 μl, 1.87 mmol) gave the title compound (496 mg, 75%).

### MW: 352.17

HPLCMS (method A): [m/z]: 352

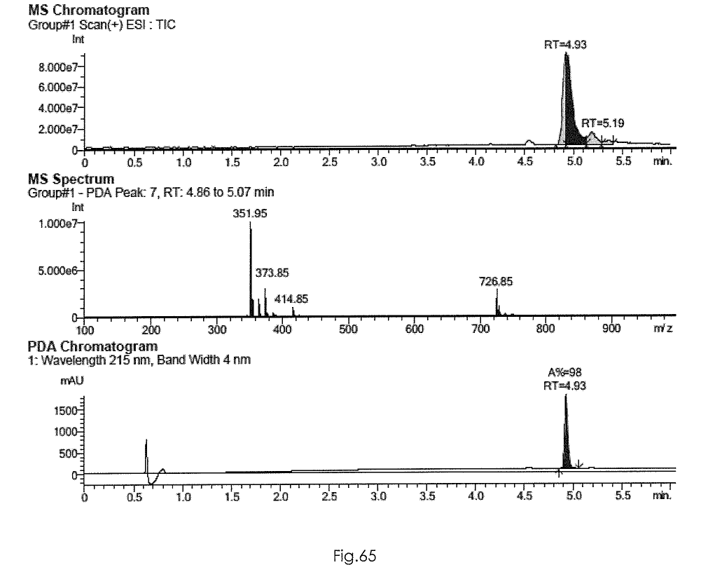

FIG. 65 shows the MS chromatogram, the MS spectrum and the PDA chromatogram of the compound of example 68.

### IC50 [μM]: >50.

### Example 69

### General Procedure 48:

### 5-Benzyl-6-chloro-2-pyridin-2-yl-pyrimidin-4-ylamine

A suspension of 5-benzyl-4,6-dichloro-2-(pyridin-2-yl)pyrimidine (50 mg, 0.16 mmol) in NH₄OH (35% solution in water, 1 ml, 9.3 mmol) in a microwave tube was heated at 100° C. for 30 min in the microwave. EtOH (1 ml) was added and the reaction heated at 100° C. for a further 30 min in the microwave. The resulting solid was collected by filtration, washed with EtOH (1 ml) and dried under vacuum to give the title compound (30 mg, 64%).

### MW: 296.75

HPLCMS (method A): [m/z]: 297

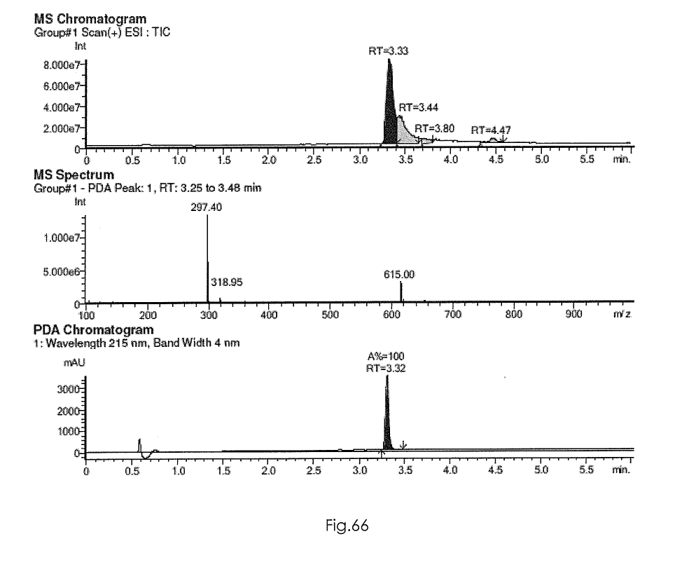

FIG. 66 shows the MS chromatogram, the MS spectrum and the PDA chromatogram of the compound of example 69.

### IC50 [μM]: >50.

### Example 70

### 6-Chloro-5-(2-fluoro-benzyl)-2-pyridin-2-yl-pyrimidin-4-ylamine

In a similar fashion using route 14 general procedure 48, 4,6-dichloro-5-(2-fluoro-benzyl)-2-pyridin-2-yl-pyrimidine (390 mg, 1.17 mmol) and NH₄OH (35% solution in water, 3.1 ml, 29.28 mmol) gave the title compound (334 mg, 91%).

### MW: 314.75

HPLCMS (method A): [m/z]: 315

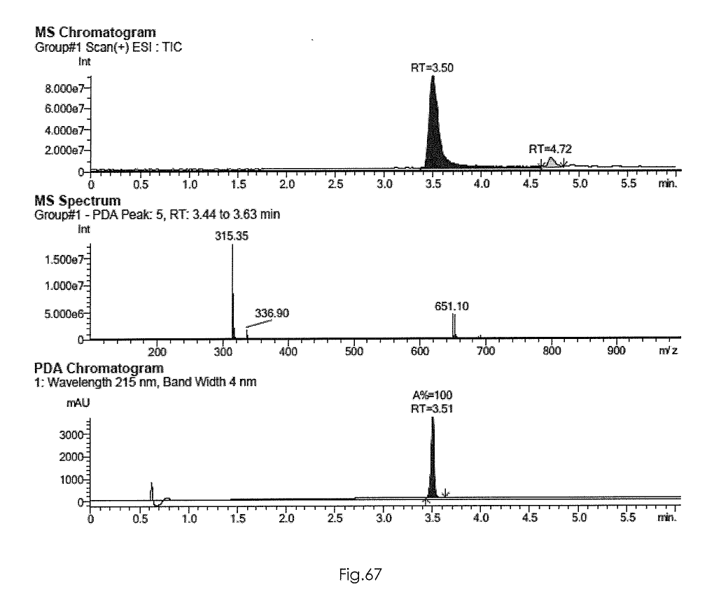

FIG. 67 shows the MS chromatogram, the MS spectrum and the PDA chromatogram of the compound of example 70.

### IC50 [μM]: >50.

### 6-Chloro-5-(2-fluoro-benzyl)-2-(5-fluoro-pyridin-2-yl)-pyrimidin-4-ylamine

In a similar fashion using route 14 general procedure 48, 4,6-dichloro-5-(2-fluoro-benzyl)-2-(5-fluoro-pyridin-2-yl)-pyrimidine (377 mg, 1.07 mmol) and NH₄OH (35% solution in water, 2.9 ml, 26.76 mmol) gave the title compound (307 mg, 86%).

### MW: 332.74

HPLCMS (method B): [m/z]: 333

### Example 71

### General Procedure 49:

### 5-Benzyl-N-isopropyl-2-pyridin-2-yl-pyrimidine-4,6-diamine

Isopropylamine (87 μl, 1.01 mmol) was added to a solution of 5-benzyl-6-chloro-2-(pyridin-2-yl)pyrimidin-4-amine (30 mg, 0.1 mmol) in n-BuOH (1 ml) in a microwave tube. The mixture was heated at 193° C. for 1 h in the microwave. Isopropylamine (1.0 ml, 11.61 mmol) was added and the mixture was heated at 193° C. for a further 150 min in the microwave. After cooling, water was added and the resulting precipitate was collected by filtration, washed with Et₂O and dried under vacuum to give the title compound (29 mg, 90%).

### MW: 319.40

HPLCMS (method A): [m/z]: 320.70

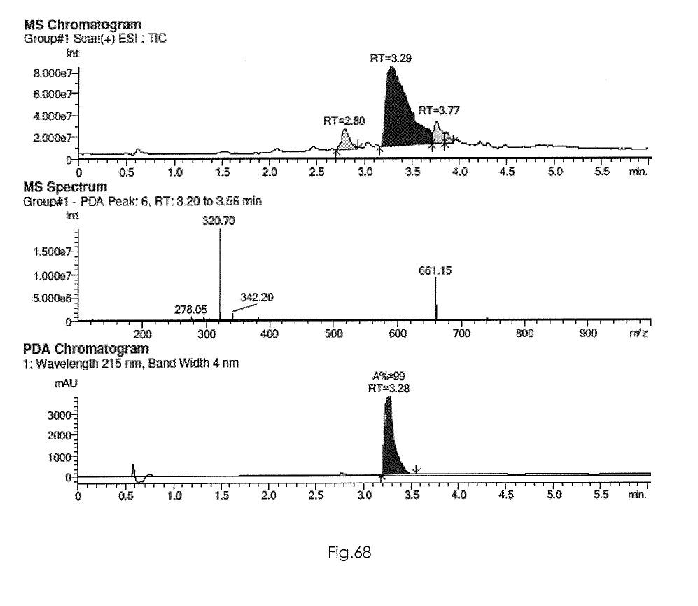

FIG. 68 shows the MS chromatogram, the MS spectrum and the PDA chromatogram of the compound of example 71.

### IC50 [μM]: <50.

### Example 72

### 5-(2-Fluoro-benzyl)-N-isopropyl-2-pyridin-2-yl-pyrimidine-4,6-diamine

In a similar fashion using route 14 general procedure 49, isopropylamine (273 μl, 3.18 mmol) and 6-chloro-5-(2-fluoro-benzyl)-2-pyridin-2-yl-pyrimidin-4-ylamine (100 mg, 0.32 mmol) gave the title compound (58 mg, 54%).

### MW: 337.40

HPLCMS (method A): [m/z]: 338

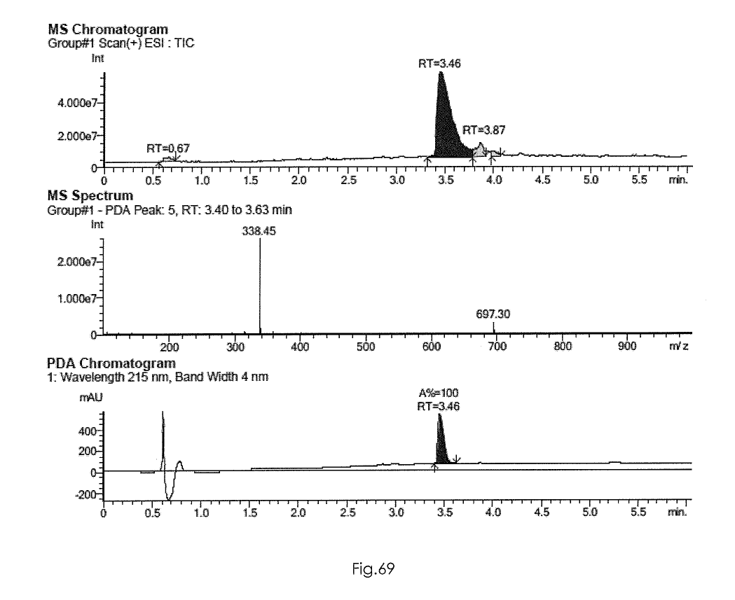

FIG. 69 shows the MS chromatogram, the MS spectrum and the PDA chromatogram of the compound of example 72.

### IC50 [μM]: <50.

### Example 73

### 5-Benzyl-6-morpholin-4-yl-2-pyridin-2-yl-pyrimidin-4-ylamine

In a similar fashion using route 14 general procedure 49, 5-benzyl-6-chloro-2-(pyridin-2-yl)pyrimidin-4-amine (30 mg, 0.1 mmol) and morpholine (1 ml) gave the title compound (35 mg, 100%).

### MW: 347.41

HPLCMS (method A): [m/z]: 348

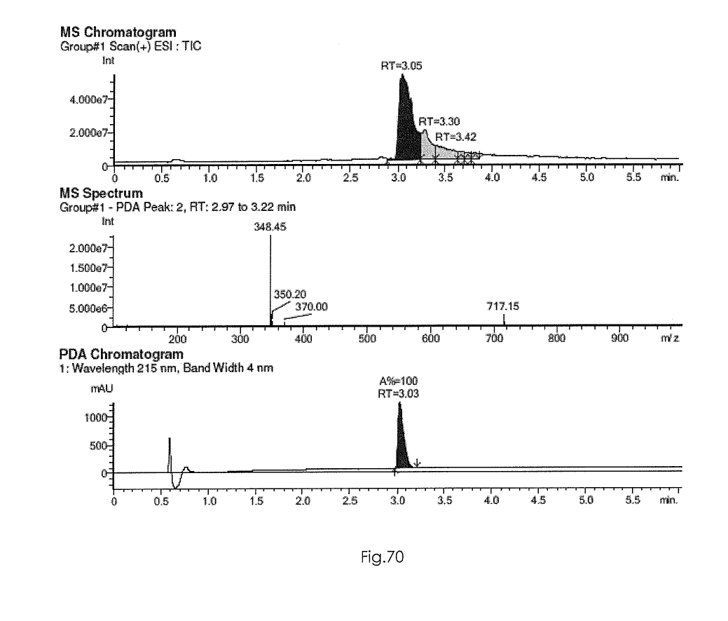

FIG. 70 shows the MS chromatogram, the MS spectrum and the PDA chromatogram of the compound of example 73.

### IC50 [μM]: <50.

### Example 74

### 5-(2-Fluoro-benzyl)-6-morpholin-4-yl-2-pyridin-2-yl-pyrimidin-4-ylamine

In a similar fashion using route 14 general procedure 49, 6-chloro-5-(2-fluoro-benzyl)-2-pyridin-2-yl-pyrimidin-4-ylamine (100 mg, 0.32 mmol) and morpholine (1 ml) gave the title compound (111 mg, 96%).

### MW: 365.40

HPLCMS (method A): [m/z]: 366

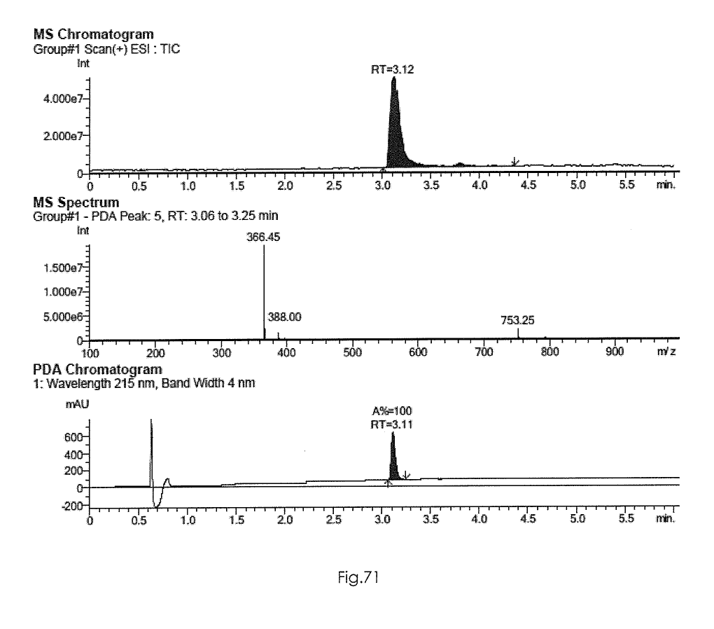

FIG. 71 shows the MS chromatogram, the MS spectrum and the PDA chromatogram of the compound of example 74.

### IC50 [μM]: <50.

### Example 75

### General Procedure 50:

### 5-(2-Fluoro-benzyl)-N,N′-diisopropyl-2-pyridin-2-yl-pyrimidine-4,6-diamine

Isopropylamine (257 μl, 2.99 mmol) was added to a solution of 4,6-dichloro-5-(2-fluoro-benzyl)-2-pyridin-2-yl-pyrimidine (100 mg, 0.30 mmol) in n-BuOH (1 ml) in a microwave tube. The mixture was heated at 200° C. for 5 h in the microwave. The reaction mixture was diluted with water (1 ml) and concentrated in vacuo. The residue was dissolved in EtOAc (2 ml) and was washed with saturated aqueous NaHCO₃ solution and water. The organic phase was dried (Na₂SO₄) and concentrated in vacuo to give the title compound (82 mg, 72%).

### MW: 379.48

HPLCMS (method A): [m/z]: 380

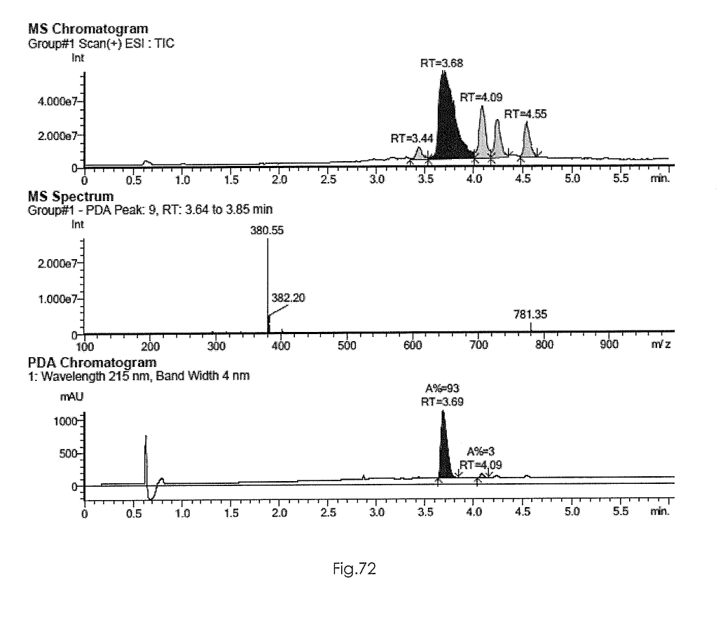

FIG. 72 shows the MS chromatogram, the MS spectrum and the PDA chromatogram of the compound of example 75.

### IC50 [μM]: >50.

### Example 76

### General Procedure 51:

### 4-[5-Benzyl-6-(morpholin-4-yl)-2-(pyridin-2-yl)pyrimidin-4-yl]morpholine

A solution of 5-benzyl-4,6-dichloro-2-(pyridin-2-yl)pyrimidine (65 mg, 0.21 mmol) in morpholine (1 ml) in a microwave tube was heated at 200° C. for 1 h in the microwave. The solution was diluted with water (3 ml) and extracted with DCM (×3). The combined organic phases were dried (MgSO₄) and concentrated in vacuo. The residue was dissolved in Et₂O (4 ml) and washed with water (×2) and brine. The organic phase was dried (MgSO₄) and concentrated in vacuo. The crude residue was purified by trituration from Et₂O to give the title compound (25 mg, 29%).

### MW: 417.5

HPLCMS (method A): [m/z]: 418

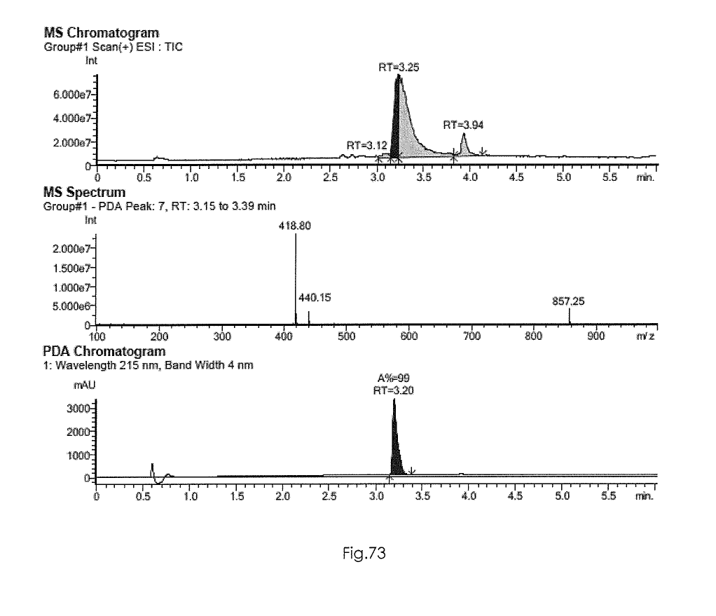

FIG. 73 shows the MS chromatogram, the MS spectrum and the PDA chromatogram of the compound of example 76.

### IC50 [μM]: >50.

### Example 77

### 4-{5-[(2-Fluorophenyl)methyl]-6-(morpholin-4-yl)-2-(pyridin-2-yl)pyrimidin-4-yl}morpholine

In a similar fashion using route 14 general procedure 51, 4,6-dichloro-5-(2-fluoro-benzyl)-2-(5-fluoro-pyridin-2-yl)-pyrimidine (100 mg, 0.30 mmol) and morpholine (1 ml) gave the title compound (60 mg, 46%).

### MW: 435.49

HPLCMS (method A): [m/z]: 436

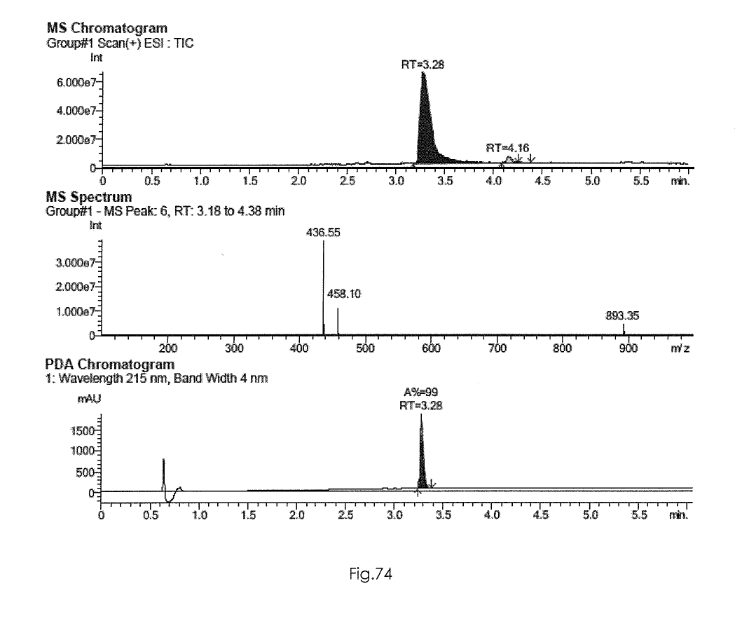

FIG. 74 shows the MS chromatogram, the MS spectrum and the PDA chromatogram of the compound of example 77.

### IC50 [μM]: >50.

### Route 15

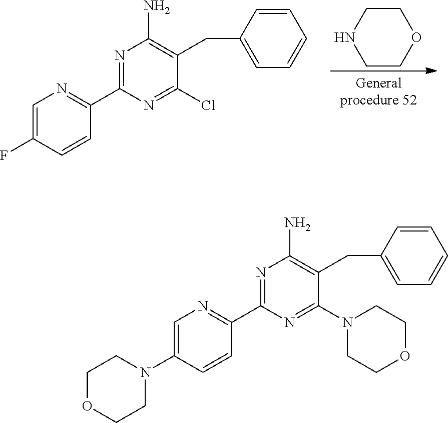

### General Procedure 52:

### Example 78

### 5-(2-Fluoro-benzyl)-6-morpholin-4-yl-2-(5-morpholin-4-yl-pyridin-2-yl)-pyrimidin-4-ylamine

A solution of 6-chloro-5-(2-fluoro-benzyl)-2-(5-fluoro-pyridin-2-yl)-pyrimidin-4-ylamine (100 mg, 0.30 mmol) in morpholine (1 ml) in a microwave tube was heated at 200° C. for 1 h in the microwave. Et₂O (0.5 ml) was added and the resulting precipitate was collected by filtration. The solid was dissolved in EtOAc (2 ml) and washed with saturated aqueous NaHCO₃ solution and water. The organic phase was dried (Na₂SO₄) and concentrated in vacuo. The crude residue was purified by trituration from Et₂O to give the title compound (100 mg, 74%).

### MW: 450.51.41

HPLCMS (method A): [m/z]: 451

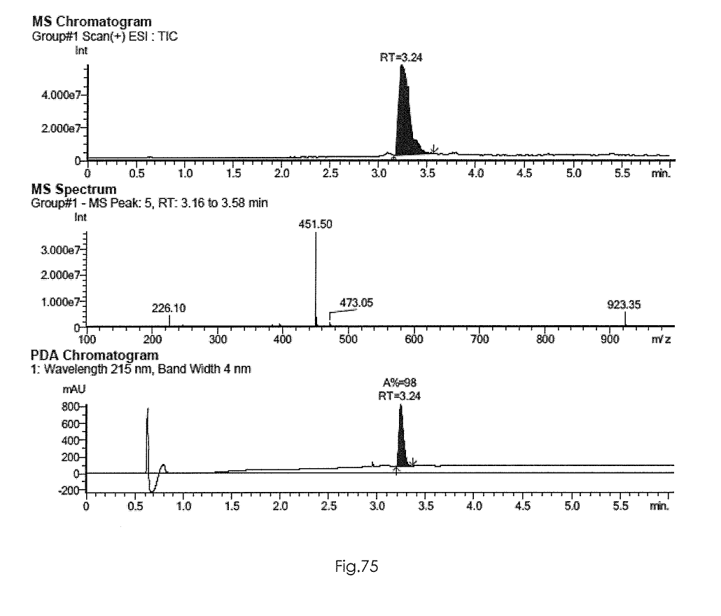

FIG. 75 shows the MS chromatogram, the MS spectrum and the PDA chromatogram of the compound of example 78.

### IC50 [μM]: >50.

### Example 79

### Route 16

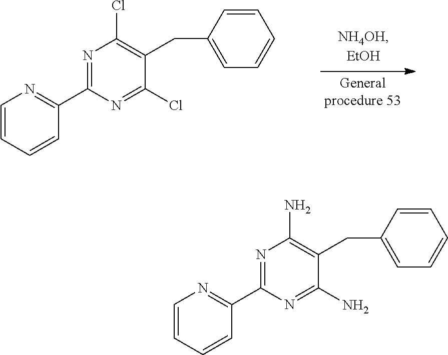

### General Procedure 53:

### 5-Benzyl-2-pyridin-2-yl-pyrimidine-4,6-diamine

A suspension of 5-benzyl-4,6-dichloro-2-(pyridin-2-yl)pyrimidine (50 mg, 0.16 mmol) in NH₄OH (1 ml, 9.3 mmol) and EtOH (1 ml) in a microwave tube was heated at 130° C. for 30 min in the microwave. The reaction was re-heated, in stages, at 150° C. for a total of 60.5 h. The reaction was diluted with water and the resulting solid was collected by filtration, washed with Et₂O and dried under vacuum to give the title compound (32 mg, 73%).

### MW: 277.32

HPLCMS (method A): [m/z]: 278

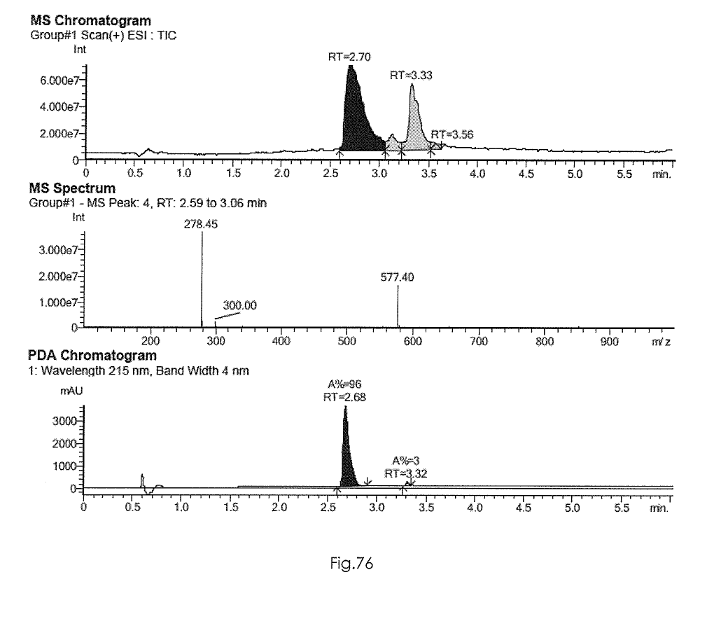

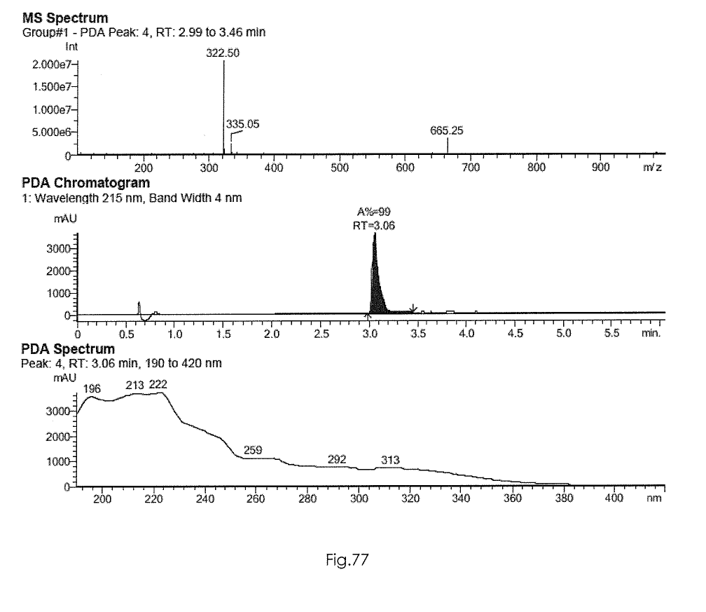

FIG. 76 shows the MS chromatogram, the MS spectrum and the PDA chromatogram of the compound of example 79.

### IC50 [μM]: <50.

### Route 17

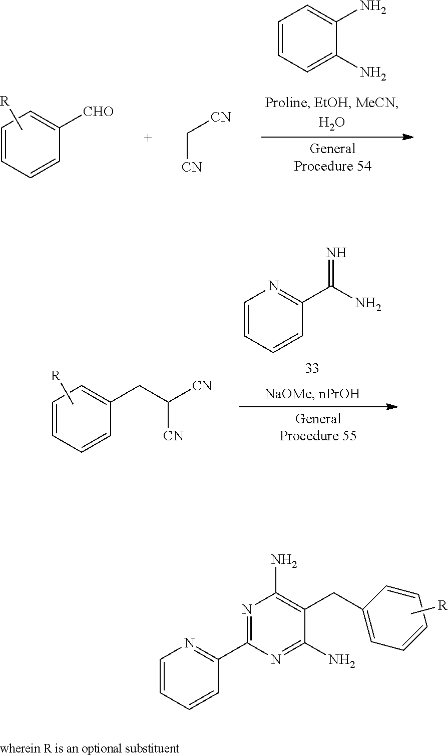

### General Procedure 54:

### 2-(2-Ethoxy-benzyl)-malononitrile

A solution of the 2-ethoxybenzaldehyde (736 mg, 4.9 mmol) in EtOH (3 ml) was treated with malononitrile (162 mg, 2.45 mmol) in EtOH (3 ml), benzene-1,2-diamine (265 mg, 2.45 mol) in MeCN (3 ml) and finally proline (56 mg, 0.5 mmol) in water (1 ml) and the solution was stirred at room temperature for 1 h. The mixture was concentrated in vacuo and the residue purified by column chromatography with DCM/heptane (50:50-100) as the eluent to the give title compound (407 mg, 42%). The compound could not be detected by HPLCMS therefore structure was confirmed by 1H-NMR.

### 2-(2-Methoxy-5-methyl-benzyl)-malononitrile

In a similar fashion using route 17 general procedure 54, 2-methoxy-5-benzaldehyde (736 mg, 4.9 mmol), malononitrile (162 mg, 2.45 mmol), benzene-1,2-diamine (265 mg, 2.45 mol) and proline (56 mg, 0.5 mmol) gave the title compound (474 mg, 48%) after purification by column chromatography with DCM/heptane (25:75-100) as the eluent. The compound could not be detected by HPLCMS therefore structure was confirmed by 1H-NMR.

### 2-(2,4-Dimethoxy-benzyl)-malononitrile

In a similar fashion using route 17 general procedure 54, 2,4-dimethoxybenzaldehyde (814 mg, 4.9 mmol), malononitrile (162 mg, 2.45 mmol), benzene-1,2-diamine (265 mg, 2.45 mol) and proline (56 mg, 0.5 mmol) gave the title compound (325 mg, 31%) after purification by column chromatography with DCM/heptane (25:75-100) as the eluent. The compound could not be detected by HPLCMS therefore structure was confirmed by 1H-NMR.

### 2-(3-Methoxy-benzyl)-malononitrile

In a similar fashion using route 17 general procedure 54, 3-methoxybenzaldehyde (2.26 g, 16.6 mmol), malononitrile (0.55 g, 8.30 mmol), benzene-1,2-diamine (0.90 g, 8.30 mol) and proline (0.19 g, 1.66 mmol) gave the title compound (481 mg, 31%) after purification by column chromatography with DCM/heptane (25:75-100) as the eluent. The compound could not be detected by HPLCMS, therefore structure was confirmed by 1H-NMR.

### 2-(2-Methyl-benzyl)-malononitrile

In a similar fashion using route 17 general procedure 54, 2-methylbenzaldehyde (1.99 g, 16.6 mmol), malononitrile (0.55 g, 8.30 mmol), benzene-1,2-diamine (0.90 g, 8.30 mol) and proline (0.19 g, 1.66 mmol) gave the title compound (670 mg, 47%) after purification by column chromatography with DCM/heptane (25:75-100) as the eluent. The compound could not be detected by HPLCMS, therefore structure was confirmed by 1H-NMR.

### 2-(3-Methyl-benzyl)-malononitrile

In a similar fashion using route 17 general procedure 54, 3-methylbenzaldehyde (1.99 g, 16.6 mmol), malononitrile (0.55 g, 8.30 mmol), benzene-1,2-diamine (0.90 g, 8.30 mol) and proline (0.19 g, 1.66 mmol) gave the title compound (862 mg, 61%) after purification by column chromatography with DCM/heptane (25:75-100) as the eluent. The compound could not be detected by HPLCMS, therefore structure was confirmed by 1H-NMR.

### 2-(3-Fluoro-benzyl)-malononitrile

In a similar fashion using route 17 general procedure 54, 3-fluorobenzaldehyde (2.06 g, 16.6 mmol), malononitrile (0.55 g, 8.30 mmol), benzene-1,2-diamine (0.90 g, 8.30 mol) and proline (0.19 g, 1.66 mmol) gave the title compound (410 mg, 63%) after purification by column chromatography with DCM/heptane (25:75-100) as the eluent. The compound could not be detected by HPLCMS, therefore structure was confirmed by 1H-NMR.

### 2-(4-Fluoro-benzyl)-malononitrile

In a similar fashion using route 17 general procedure 54, 4-fluorobenzaldehyde (2.06 g, 16.6 mmol), malononitrile (0.55 g, 8.30 mmol), benzene-1,2-diamine (0.90 g, 8.30 mol) and proline (0.19 g, 1.66 mmol) gave the title compound (1.34 g, 92%) after purification by column chromatography with DCM/heptane (25:75-100) as the eluent. The compound could not be detected by HPLCMS, therefore structure was confirmed by 1H-NMR.

### 2-(3-Chloro-benzyl)-malononitrile

In a similar fashion using route 17 general procedure 54, 3-chlorobenzaldehyde (2.33 g, 16.6 mmol), malononitrile (0.55 g, 8.30 mmol), benzene-1,2-diamine (0.90 g, 8.30 mol) and proline (0.19 g, 1.66 mmol) gave the title compound (784 mg, 50%) after purification by column chromatography with DCM/heptane (25:75-100) as the eluent. The compound could not be detected by HPLCMS, therefore structure was confirmed by 1H-NMR.

### 2-(2,5-Difluoro-benzyl)-malononitrile

In a similar fashion using route 17 general procedure 54, 2,5-difluoro-benzaldehyde (2.36 g, 16.6 mmol), malononitrile (0.55 g, 8.30 mmol), benzene-1,2-diamine (0.90 g, 8.30 mol) and proline (0.19 g, 1.66 mmol) gave the title compound (650 mg, 41%) after purification by column chromatography with DCM/heptane (25:75-100) as the eluent. The compound could not be detected by HPLCMS, therefore structure was confirmed by 1H-NMR.

### 2-(2-Fluoro-4-methoxy-benzyl)-malononitrile

In a similar fashion using route 17 general procedure 54, 2-fluoro-4-methoxybenzaldehyde (2.36 g, 16.6 mmol), malononitrile (0.55 g, 8.30 mmol), benzene-1,2-diamine (0.90 g, 8.30 mol) and proline (0.19 g, 1.66 mmol) gave the title compound (470 mg, 20%) after purification by column chromatography with DCM/heptane (25:75-100) as the eluent. The compound could not be detected by HPLCMS, therefore structure was confirmed by 1H-NMR.

### Example 80

### General Procedure 55:

### 5-(2-Ethoxy-benzyl)-2-pyridin-2-yl-pyrimidine-4,6-diamine

NaOMe (89 mg, 1.65 mmol) was added to a solution of 2-(2-ethoxybenzyl)-malononitrile 110 (174 mg, 0.87 mmol) and pyridine-2-carboximidamide (100 mg, 0.83 mmol) in n-PrOH (2 ml), in a microwave tube, under N₂ and the mixture was heated at 150° C. for 1 h in the microwave. The crude reaction mixture was diluted with water (8 ml). The cloudy solution was decanted off and the residual gum was triturated with Et₂O and MeCN (1:1, 2 ml) to give the title compound (26 mg, 10%).

### MW: 321.38

HPLCMS (method A): [m/z]: 322

FIG. 77 shows the MS chromatogram, the MS spectrum and the PDA chromatogram of the compound of example 80.

### IC50 [μM]: >50.

### Example 81

### 5-(2-Methoxy-5-methyl-benzyl)-2-pyridin-2-yl-pyrimidine-4,6-diamine

In a similar fashion using route 17 general procedure 55, pyridine-2-carboximidamide (100 mg, 0.83 mmol), 2-(2-methoxy-5-methyl-benzyl)-malononitrile (174 mg, 0.87 mmol) and NaOMe (89 mg, 1.65 mmol) gave the title compound (58 mg, 22%) after purification by trituration from EtOH.

### MW: 321.38

HPLCMS (method A): [m/z]: 322

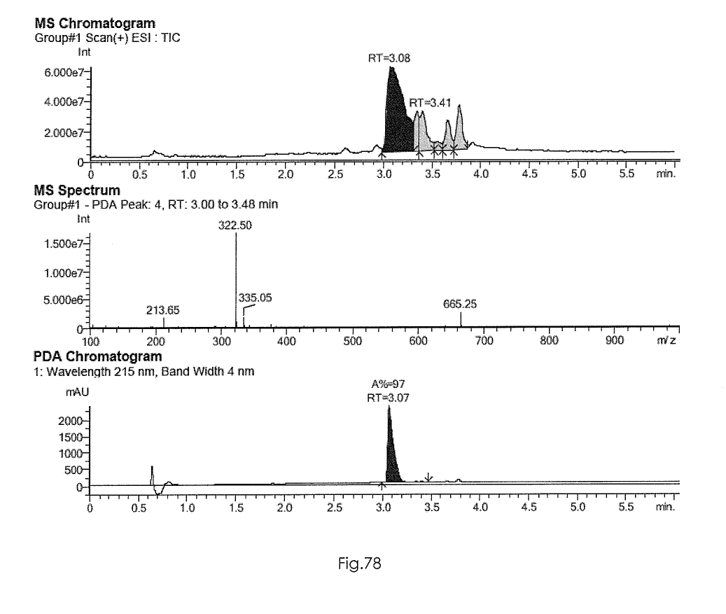

FIG. 78 shows the MS chromatogram, the MS spectrum and the PDA chromatogram of the compound of example 81.

### IC50 [μM]: >50.

### Example 82

### 5-(2,4-Dimethoxy-benzyl)-2-pyridin-2-yl-pyrimidine-4,6-diamine

In a similar fashion using route 17 general procedure 55, pyridine-2-carboximidamide (100 mg, 0.83 mmol), 2-(2,4-dimethoxy-benzyl)-malononitrile (188 mg, 0.87 mmol) and NaOMe (89 mg, 1.65 mmol) gave the title compound (11 mg, 4%) after purification by trituration from MeCN/Et₂O.

### MW: 337.38

HPLCMS (method A): [m/z]: 338

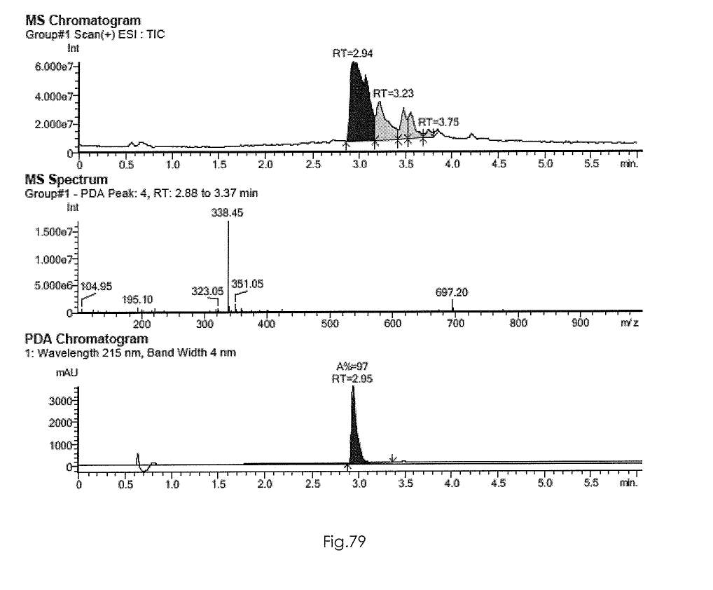

FIG. 79 shows the MS chromatogram, the MS spectrum and the PDA chromatogram of the compound of example 82.

### IC50 [μM]: <50.

### Example 83

### 5-(3-Methoxy-benzyl)-2-pyridin-2-yl-pyrimidine-4,6-diamine

In a similar fashion using route 17 general procedure 53, pyridine-2-carboximidamide 33 (100 mg, 0.83 mmol), 2-(3-methoxy-benzyl)-malononitrile (162 mg, 0.87 mmol) and NaOMe (89 mg, 1.65 mmol) gave the title compound (15 mg, 6%) after purification by trituration from MeCN/Et₂O.

### MW: 307.35

HPLCMS (method A): [m/z]: 308

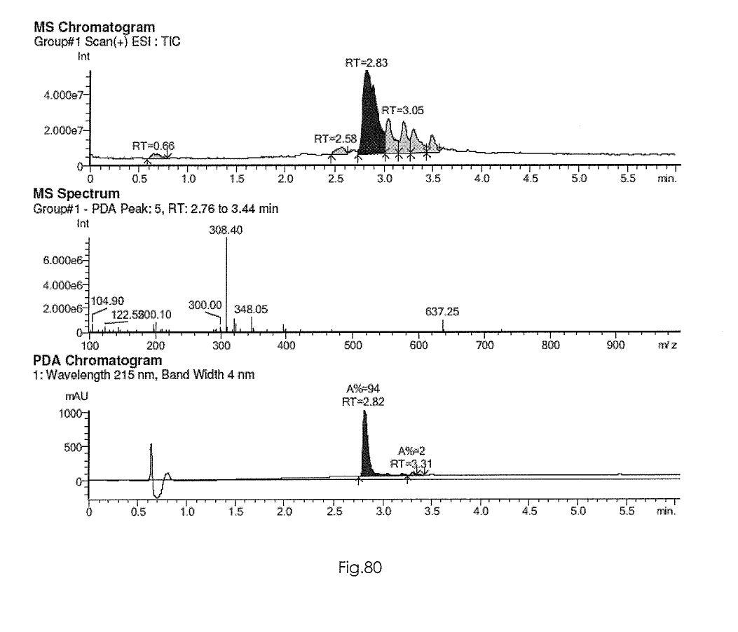

FIG. 80 shows the MS chromatogram, the MS spectrum and the PDA chromatogram of the compound of example 83.

### IC50 [μM]: <50.

### Example 84

### 5-(2-Methyl-benzyl)-2-pyridin-2-yl-pyrimidine-4,6-diamine

In a similar fashion using route 17 general procedure 55, pyridine-2-carboximidamide (100 mg, 0.83 mmol), 2-(2-methyl-benzyl)-malononitrile (148 mg, 0.87 mmol) and NaOMe (89 mg, 1.65 mmol) gave the title compound (8 mg, 3%) after purification by trituration from MeCN/Et₂O.

### MW: 291.35

HPLCMS (method A): [m/z]: 292

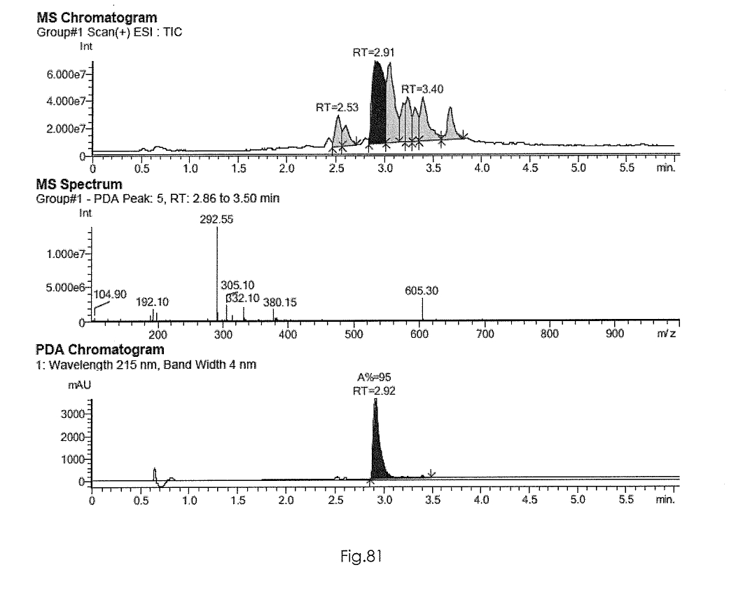

FIG. 81 shows the MS chromatogram, the MS spectrum and the PDA chromatogram of the compound of example 84.

### IC50 [μM]: <50.

### Example 85

### 5-(3-Methyl-benzyl)-2-pyridin-2-yl-pyrimidine-4,6-diamine

In a similar fashion using route 17 general procedure 55, pyridine-2-carboxamidine (100 mg, 0.83 mmol), 2-(3-methyl-benzyl)-malononitrile (155 mg, 0.91 mmol) and NaOMe (89 mg, 1.65 mmol) gave the title compound (40 mg, 15%).

### MW: 291.35

HPLCMS (Method A): [m/z]: 292

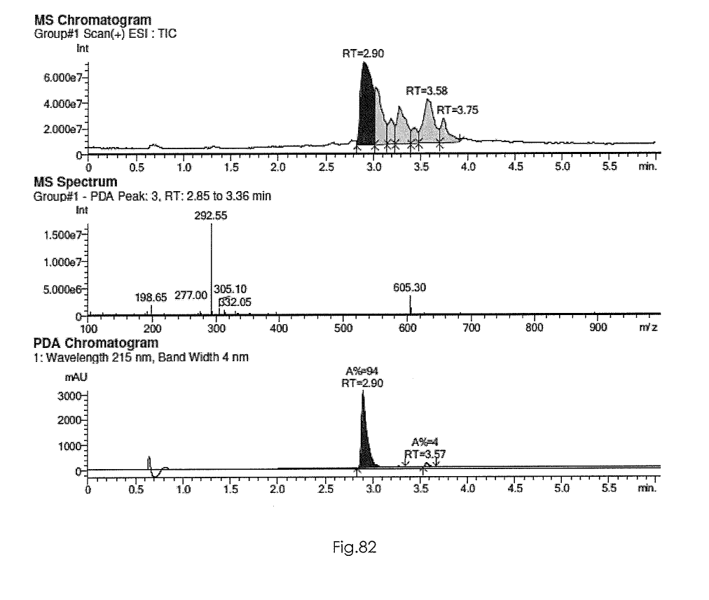

FIG. 82 shows the MS chromatogram, the MS spectrum and the PDA chromatogram of the compound of example 85.

### IC50 [μM]: <50.

### Example 86

### 5-(3-Fluoro-benzyl)-2-pyridin-2-yl-pyrimidine-4,6-diamine

In a similar fashion using route 17 general procedure 55, pyridine-2-carboxamidine (100 mg, 0.83 mmol), 2-(3-fluoro-benzyl)-malononitrile (158 mg, 0.91 mmol) and NaOMe (89 mg, 1.65 mmol) gave the title compound (49 mg, 18%) after purification by trituration from MeCN/Et₂O.

### MW: 295.31

HPLCMS (Method A): [m/z]: 296

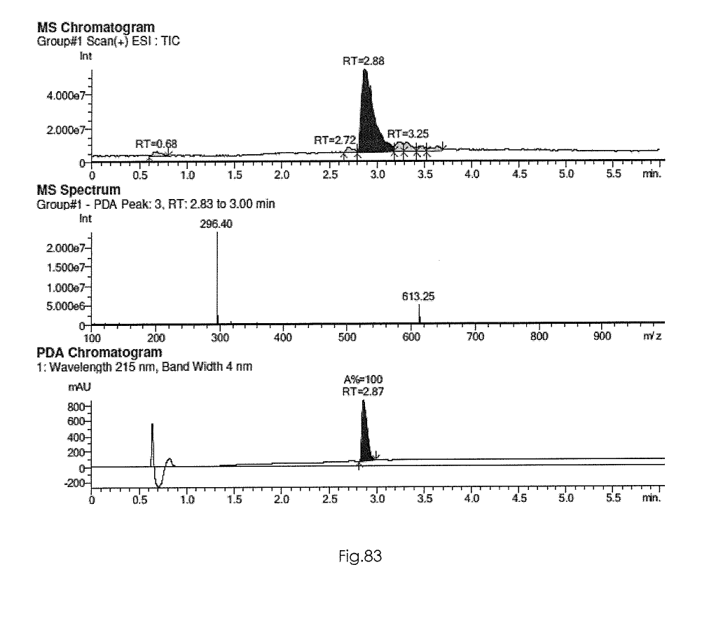

FIG. 83 shows the MS chromatogram, the MS spectrum and the PDA chromatogram of the compound of example 86.

### IC50 [μM]: <50.

### Example 87

### 5-(4-Fluoro-benzyl)-2-pyridin-2-yl-pyrimidine-4,6-diamine

In a similar fashion using route 17 general procedure 55, pyridine-2-carboxamidine (100 mg, 0.83 mmol), 2-(4-fluoro-benzyl)-malononitrile (158 mg, 0.91 mmol) and NaOMe (89 mg, 1.65 mmol) gave the title compound (23 mg, 9%) after purification by trituration from MeCN/Et₂O.

### MW: 295.31

HPLCMS (Method A): [m/z]: 296

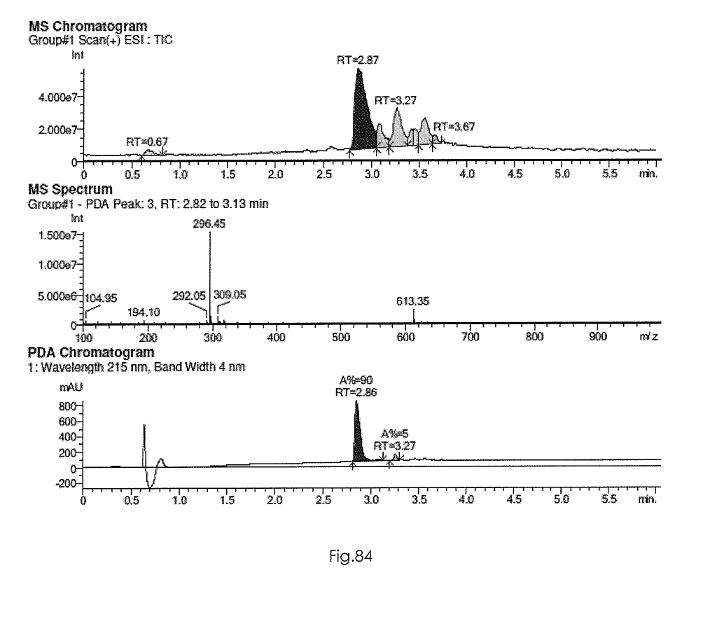

FIG. 84 shows the MS chromatogram, the MS spectrum and the PDA chromatogram of the compound of example 87.

### IC50 [μM]: <50.

### Example 88

### 5-(3-Chloro-benzyl)-2-pyridin-2-yl-pyrimidine-4,6-diamine

In a similar fashion using route 17 general procedure 55, pyridine-2-carboxamidine (100 mg, 0.83 mmol), 2-(3-chloro-benzyl)-malononitrile (173 mg, 0.91 mmol) and NaOMe (89 mg, 1.65 mmol) gave the title compound (23 mg, 9%) after purification by trituration from MeCN/Et₂O.

### MW: 311.77

HPLCMS (Method A): [m/z]: 313

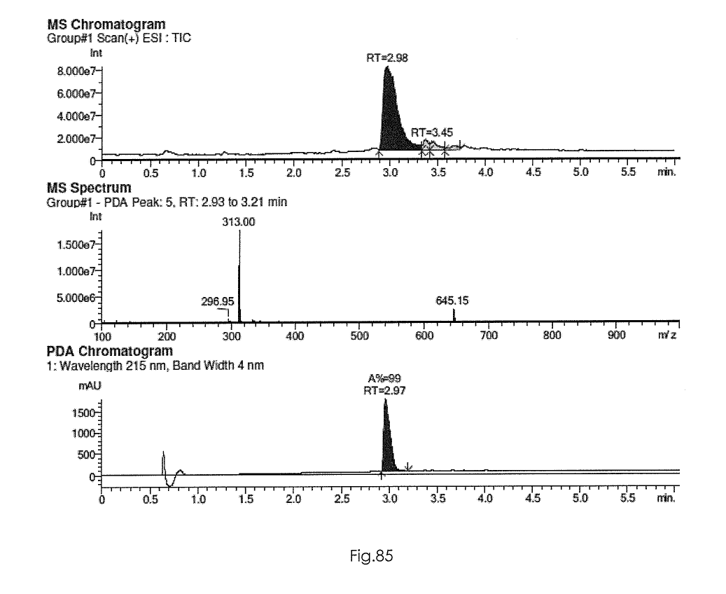

FIG. 85 shows the MS chromatogram, the MS spectrum and the PDA chromatogram of the compound of example 88.

### IC50 [μM]: <50.

### Example 89

### 5-(2,5-Difluoro-benzyl)-2-pyridin-2-yl-pyrimidine-4,6-diamine

In a similar fashion using route 17 general procedure 55, pyridine-2-carboxamidine (100 mg, 0.83 mmol), 2-(2,5-difluoro-benzyl)-malononitrile (175 mg, 0.91 mmol) and NaOMe (89 mg, 1.65 mmol) gave the title compound (21 mg, 7%) after purification by trituration from MeCN/Et₂O.

### MW: 313.30

HPLCMS (Method A): [m/z]: 314

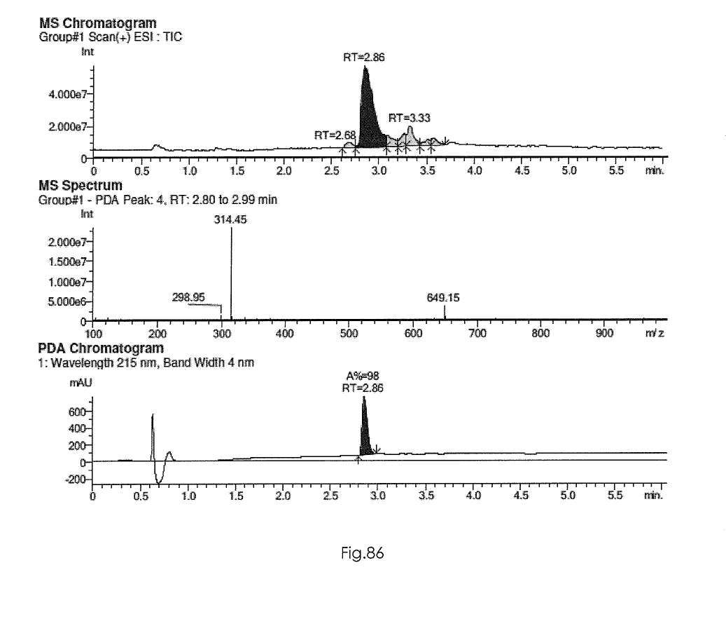

FIG. 86 shows the MS chromatogram, the MS spectrum and the PDA chromatogram of the compound of example 89.

### IC50 [μM]: <50.

### Example 90

### 5-(2-Fluoro-4-methoxy-benzyl)-2-pyridin-2-yl-pyrimidine-4,6-diamine

In a similar fashion using route 17 general procedure 55, pyridine-2-carboxamidine (100 mg, 0.83 mmol), 2-(2-fluoro-4-methoxy-benzyl)-malononitrile (186 mg, 0.91 mmol) and NaOMe (89 mg, 1.65 mmol) gave the title compound (14 mg, 5%) after purification by trituration from MeCN/Et₂O.

### MW: 325.34

HPLCMS (Method A): [m/z]: 326

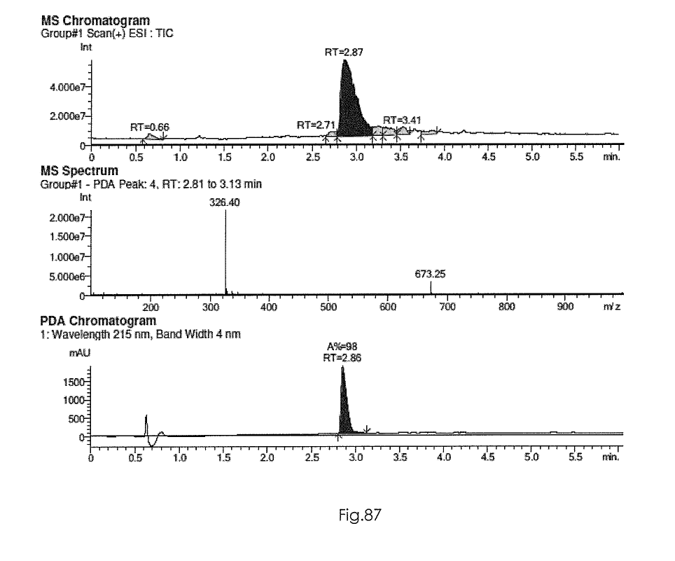

FIG. 87 shows the MS chromatogram, the MS spectrum and the PDA chromatogram of the compound of example 90.

### IC50 [μM]: <50.

### Example 91

### 5-(4-Methoxy-benzyl)-2-pyridin-2-yl-pyrimidine-4,6-diamine

In a similar fashion using route 17 general procedure 53, pyridine-2-carboxamidine (100 mg, 0.83 mmol), 2-(4-methoxy-benzyl)-malononitrile (186 mg, 0.91 mmol) and NaOMe (89 mg, 1.65 mmol) in MeOH (2 ml) gave the title compound (72 mg, 28%) after purification by trituration from MeCN/Et₂O.

### MW: 307.35

HPLCMS (method A): [m/z]: 308

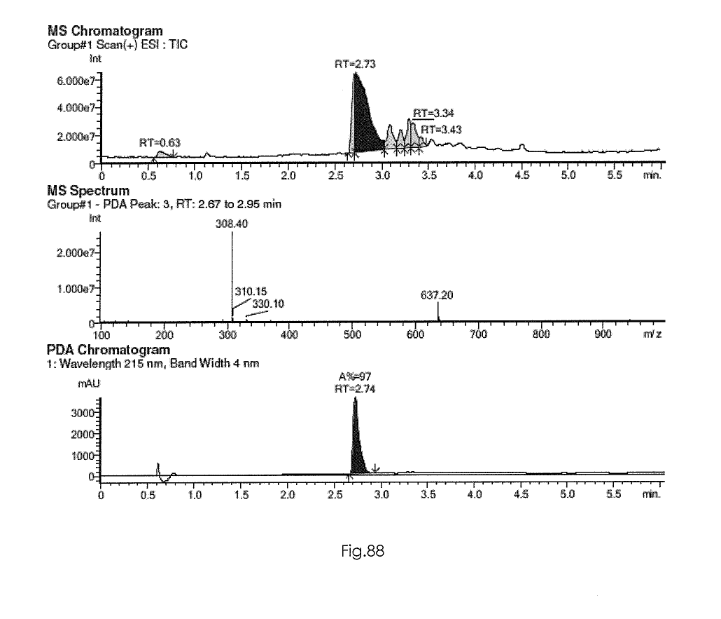

FIG. 88 shows the MS chromatogram, the MS spectrum and the PDA chromatogram of the compound of example 91.

### IC50 [μM]: >50.

### Example 92

### Route 18

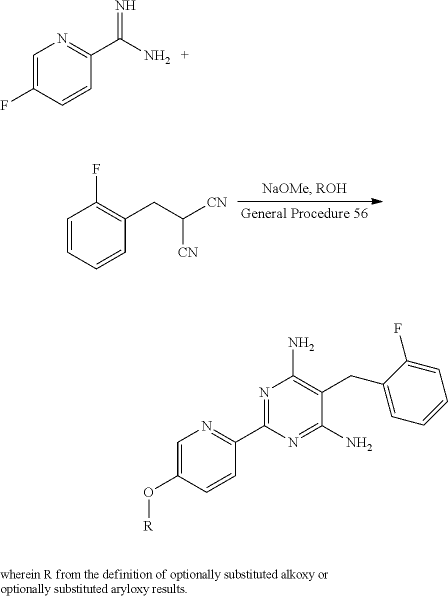

### General Procedure 56:

### 5-(2-Fluoro-benzyl)-2-(5-methoxy-pyridin-2-yl)-pyrimidine-4,6-diamine

NaOMe (89 mg, 1.65 mmol) was added to a solution of 2-(2-fluoro-benzyl)-malononitrile (138 mg, 0.72 mmol) and 5-fluoro-pyridine-2-carboxamidine (100 mg, 0.72 mmol) in MeOH (2 ml), in a microwave tube, under N₂ and the mixture was heated at 150° C. for 1 h in the microwave. The crude reaction mixture was diluted with water (8 ml). The cloudy solution was decanted off and the residual gum was triturated with Et₂O and MeCN (1:1, 2 ml) to give the title compound (33 mg, 14%).

### MW: 325.35

HPLCMS (Method A): [m/z]: 326

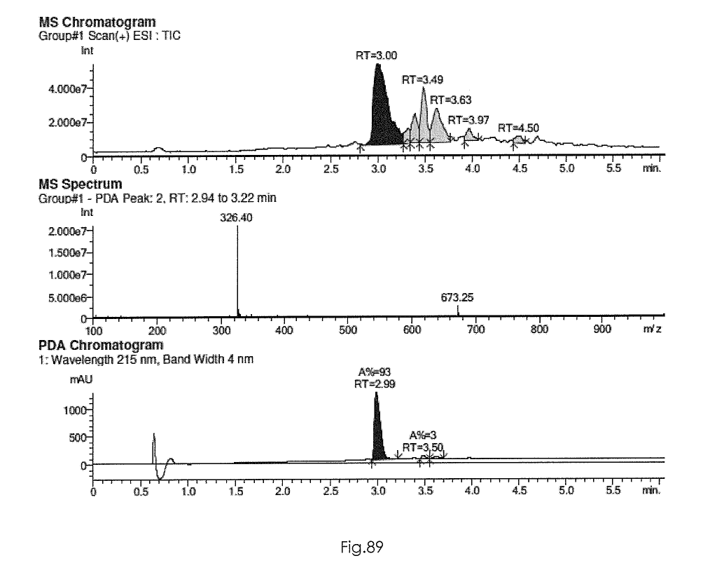

FIG. 89 shows the MS chromatogram, the MS spectrum and the PDA chromatogram of the compound of example 92.

### IC50 [μM]: <50.

### Example 93

### 5-(2-Fluoro-benzyl)-2-(5-propoxy-pyridin-2-yl)-pyrimidine-4,6-diamine

In a similar fashion using route 18 general procedure 56, 5-fluoro-pyridine-2-carboxamidine (100 mg, 0.72 mmol), 2-(2-fluoro-benzyl)-malononitrile (138 mg, 0.72 mmol) and NaOMe (89 mg, 1.65 mmol) in n-PrOH (2 ml) gave the title compound (9 mg, 4%) after purification by preparative HPLC (basic conditions).

### MW: 353.40

HPLCMS (Method A): [m/z]: 355

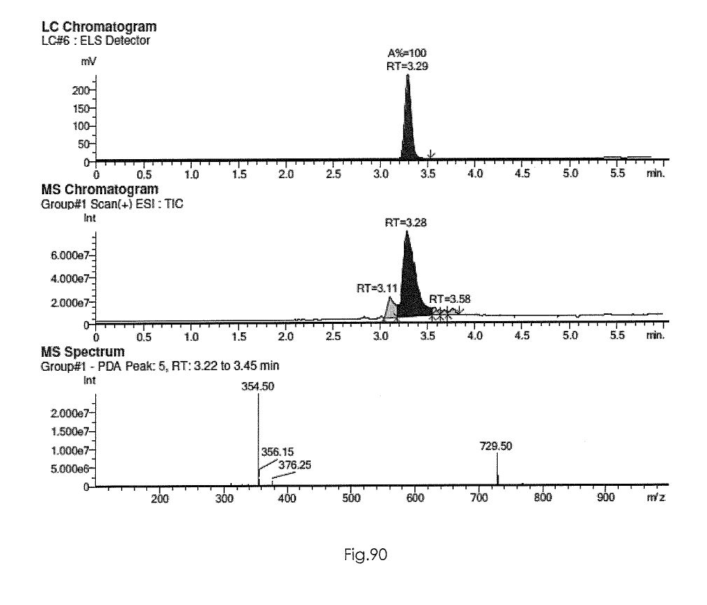

FIG. 90 shows the LC chromatogram, the MS spectrum and the MS chromatogram of the compound of example 93.

### IC50 [μM]: <50.

### Route 19

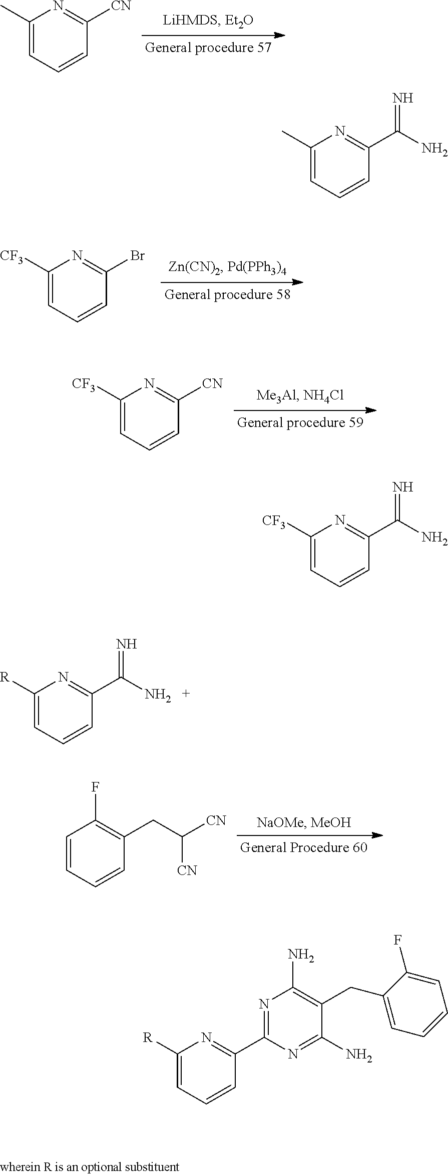

### General Procedure 57:

### 6-Methyl-pyridine-2-carboxamidine

Lithium hexamethyl disilazide (1M solution in THF, 36.0 ml, 36.0 mmol) was added to a solution of 6-methyl-2-pyridine carbonitrile (2.0 g, 16.9 mmol) in Et₂O (30 ml) at 0° C. The reaction was allowed to warm to room temperature overnight. The reaction was cooled to 0° C. and 3 M HCl (54 ml) was added and the reaction was stirred for 30 min. Water (135 ml) was added and the organic phase was separated and discarded. The aqueous phase was basified to pH 14 with saturated aqueous NaOH and extracted with DCM (×3). The combined organic extracts were dried (Na₂SO₄) and concentrated in vacuo to give the title compound (1.55 g, 66%). The compound could not be detected by HPLCMS therefore structure was confirmed by 1H-NMR.

### General Procedure 58:

### 6-Trifluoromethyl-pyridine-2-carbonitrile

Tetrakis (triphenylphosphine)palladium (0) (3.20 g, 2.77 mmol) was added to a solution of 2-bromo-6-trifluoromethylpyridine (3.13 g, 13.85 mmol) and Zn(CN)₂ (1.63 g, 13.85 mmol) in DMF under N₂. The reaction mixture was heated at 85° C. overnight. After cooling, the mixture was diluted with water (200 ml) and extracted with EtOAc (×2). The combined organic phases were dried (Na₂SO₄) and concentrated in vacuo. The crude residue was purified by column chromatography with EtOAc/heptane (4:1-1:1) as the eluent, to give the title compound (1.34 g, 56%). The compound could not be detected by HPLCMS therefore structure was confirmed by ¹H-NMR.

### General Procedure 59:

### 6-Trifluoromethyl-pyridine-2-carboxamidine

Trimethyl aluminum (2.10 g, 29.11 mmol) was added dropwise to a vigorously stirred solution of NH₄Cl (1.56 g, 29.11 mmol) in dry toluene (15 ml) at 0° C. The mixture was warmed room temperature and was stirred for 15 min. A solution of 6-trifluoromethyl-pyridine-2-carbonitrile (1.67 g, 9.703 mmol) in toluene (15 ml) was added dropwise. The reaction mixture was heated at 80° C. for 18 h. After cooling, the mixture was transferred to a vigorously stirred and cooled (0° C.) slurry of silica (20.0 g) in chloroform (150 ml) and was stirred for 10 min. The mixture was filtered and the filter cake was washed with MeOH (×3). The filtrate was concentrated in vacuo. The residue was dissolved in 1M HCl (150 ml) and Et₂O (70 ml). The organic phase was separated and discarded. The aqueous phase was basified with saturated aqueous NaOH and extracted with chloroform (×2). The combined organic extracts were dried (Na₂SO₄) and concentrated in vacuo to give the title compound (980 mg, 53%). The compound could not be detected by HPLCMS, therefore structure was confirmed by NMR.

### Example 94

### General Procedure 60:

### 5-(2-Fluoro-benzyl)-2-(6-methyl-pyridin-2-yl)-pyrimidine-4,6-diamine

NaOMe (200 mg, 3.70 mmol) was added to a solution of 2-(2-fluorobenzyl)-malononitrile (387 mg, 2.22 mmol) and 6-methyl-pyridine-2-carboximidamide (200 mg, 1.48 mmol) in MeOH (4 ml), in a microwave tube, under N₂ and the mixture was heated at 150° C. for 1 h in the microwave. After cooling, the mixture was diluted with water (8 ml) and sonicated, the resulting precipitate was removed by filtration. The filtrate was concentrated in vacuo, the residue was triturated from EtOAc and dried under vacuum to give the title compound (24 mg, 5%).

### MW: 309.34

HPLCMS (Method A): [m/z]: 310

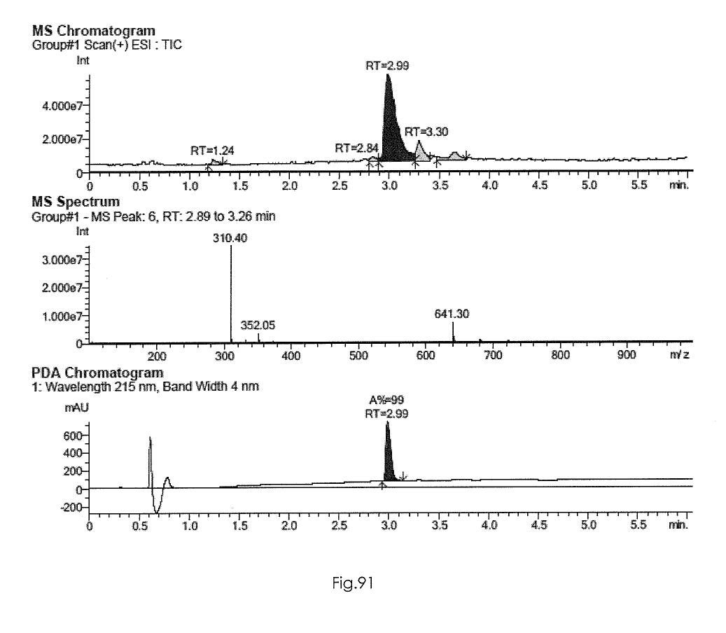

FIG. 91 shows the MS chromatogram, the MS spectrum and the PDA chromatogram of the compound of example 94.

### IC50 [μM]: >50.

### Example 95

### 5-(2-Fluoro-benzyl)-2-(6-trifluoromethyl-pyridin-2-yl)-pyrimidine-4,6-diamine

In a similar fashion using route 19 general procedure 60, 2-(2-fluorobenzyl)-malononitrile (101 mg, 0.58 mmol), 6-trifluoromethyl-pyridine-2-carboximidamide (100 mg, 0.53 mmol) and NaOMe (57 mg, 1.06 mmol) in MeOH (2 ml) gave the title compound (31 mg, 16%) after purification by trituration from Et₂O/MeCN.

### MW: 363.31

HPLCMS (Method A): [m/z]: 364

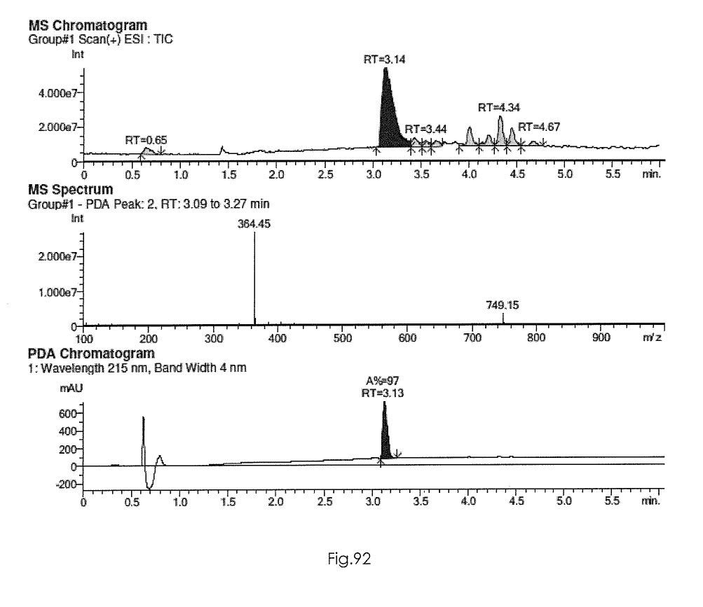

FIG. 92 shows the MS chromatogram, the MS spectrum and the PDA chromatogram of the compound of example 95.

### IC50 [μM]: >50.

### Route 20

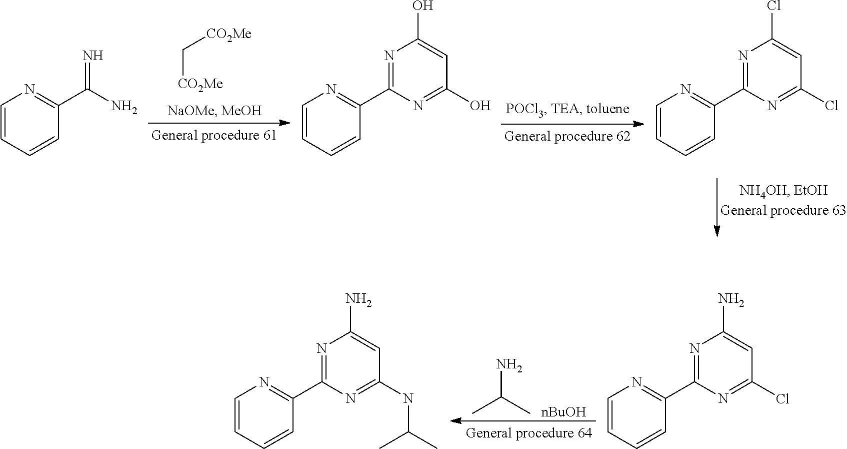

### Example 96

### General Procedure 61:

### 2-Pyridin-2-yl-pyrimidine-4,6-diol

NaOMe (0.22 g, 4.13 mmol) was added to a solution of malonic acid dimethyl ester (0.55 g, 4.13 mmol) and pyridine-2-carboxamidine (0.5 g, 84.13 mmol) in MeOH (5 ml). The reaction mixture was heated under reflux for 40 min resulting in the formation of a precipitate. The reaction mixture was diluted with MeOH (2 ml) and EtOAc (2 ml) and the precipitate was triturated and collected by filtration to give the title compound (0.54 g, 69%).

### MW: 189.17

HPLCMS (Method A): [m/z]: 190

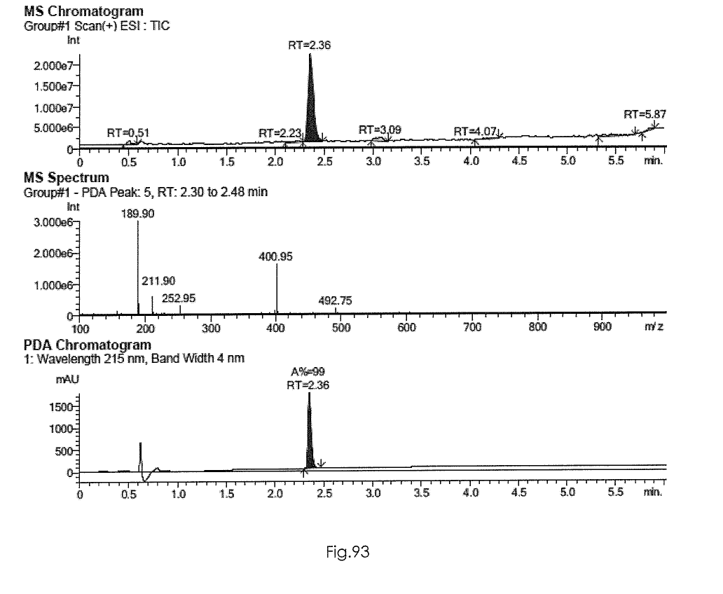

FIG. 93 shows the MS chromatogram, the MS spectrum and the PDA chromatogram of the compound of example 96.

### IC50 [μM]: >50.

### General Procedure 62:

### 4,6-Dichloro-2-pyridin-2-yl-pyrimidine

POCl₃ (2.7 ml, 28.97 mmol) was added dropwise to a solution of 2-pyridin-2-yl-pyrimidine-4,6-diol 140 (532 mg, 2.81 mmol) in toluene (3.7 ml) at 0° C. TEA (1.57 ml, 11.25 mmol) was added dropwise and the mixture was allowed to warm to room temperature before being heated at 110° C. for 1 h. The reaction mixture was concentrated in vacuo and the residue was quenched by the addition of ice/water (10 ml). The aqueous phase was extracted with EtOAc (×3). The combined organic phases were washed with NaHCO₃ and water, dried (Na₂SO₄) and concentrated in vacuo to give the title compound (310 mg, 49%).

### MW: 226.06

HPLCMS (Method B): [m/z]: 226

### Example 97

### General Procedure 63:

### 6-Chloro-2-pyridin-2-yl-pyrimidin-4-ylamine

NH₄OH (35% solution in water, 2.0 ml, 18.58 mmol) was added to a solution of 4,6-dichloro-2-pyridin-2-yl-pyrimidine (210 mg, 0.93 mmol) in EtOH (2 ml) in a microwave tube and the mixture was heated at 100° C. for 30 min in the microwave. The reaction mixture was concentrated in vacuo and the resulting residue was purified by trituration from iso-propyl alcohol to give the title compound (135 mg, 70%).

### MW: 206.63

HPLCMS (Method A): [m/z]: 207

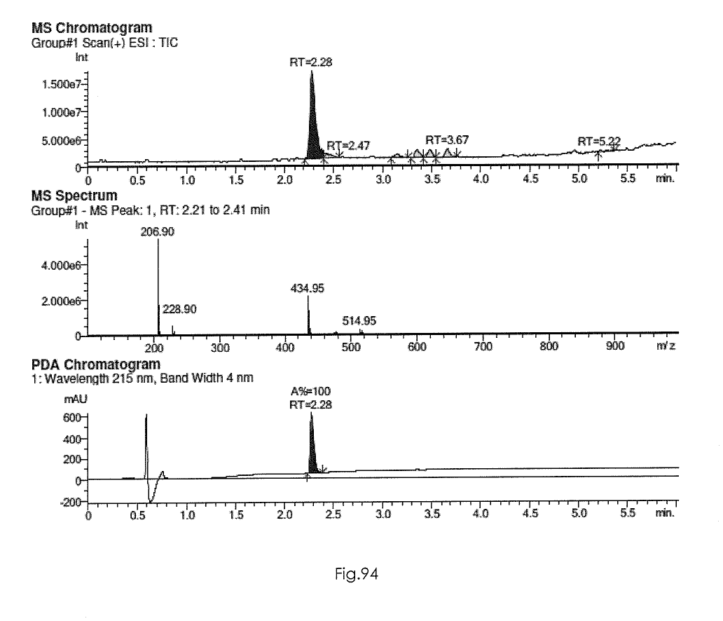

FIG. 94 shows the MS chromatogram, the MS spectrum and the PDA chromatogram of the compound of example 97.

### IC50 [μM]: >50.

### Example 98

### General Procedure 64:

### N-Isopropyl-2-pyridin-2-yl-pyrimidine-4,6-diamine

Isopropylamine (181 μl, 2.42 mmol) was added to a solution of 6-chloro-2-pyridin-2-yl-pyrimidin-4-ylamine (100 mg, 0.48 mmol) in n-BuOH (1 ml) in a microwave tube and the mixture was heated at 180° C. for 1 h in the microwave. Isopropylamine (181 μl, 2.42 mmol) was added and the mixture was heated at 180° C. for a further 7 h in the microwave. The reaction mixture was diluted with water (1 ml) and concentrated in vacuo. The residue was dissolved in EtOAc (2 ml) and washed with saturated aqueous NaHCO₃ solution (2 ml) and water (2 ml). The organic phase was dried (Na₂SO₄) and concentrated in vacuo. The crude residue was purified by trituration from Et₂O to give the title compound (32 mg, 29%).

### MW: 229.28

HPLCMS (Method A): [m/z]: 230

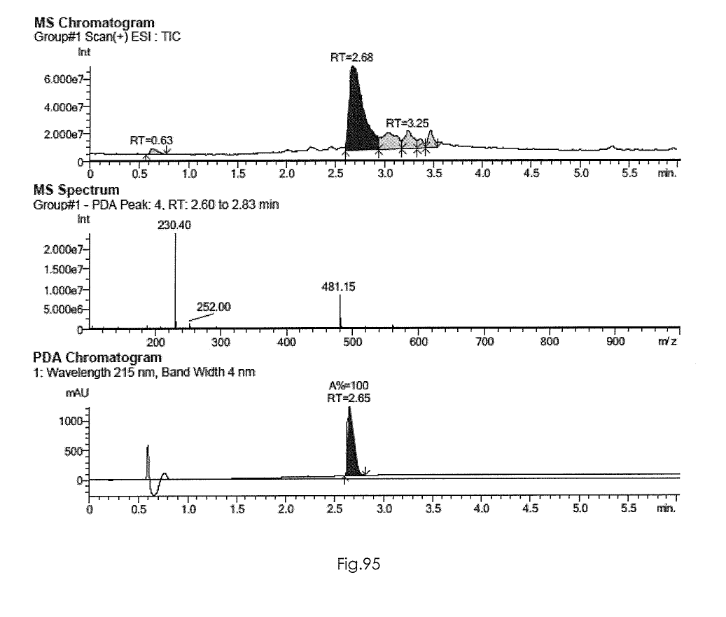

FIG. 95 shows the MS chromatogram, the MS spectrum and the PDA chromatogram of the compound of example 98.

### IC50 [μM]: <50.

### Example 99

### 5-Methoxy-2-pyridin-2-yl-4-pyrrolidin-1-yl-pyrimidine

### MW: 256.30

### Manufacturer: Key Organics

HPLCMS (Method A): [m/z]: 256.95

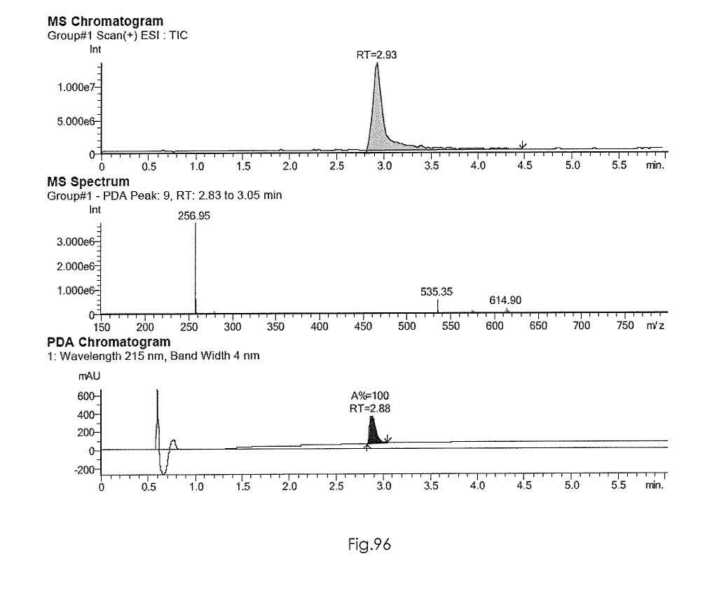

FIG. 96 shows the MS chromatogram, the MS spectrum and the PDA chromatogram of the compound of example 99.

### IC50 [μM]: <50.

### Example 100

### 5-Methoxy-4-(4-methyl-piperazin-1-yl)-2-pyridin-2-yl-pyrimidine

### MW: 285.34

### Manufacturer: Key Organics

HPLCMS (Method E): [m/z]: 286

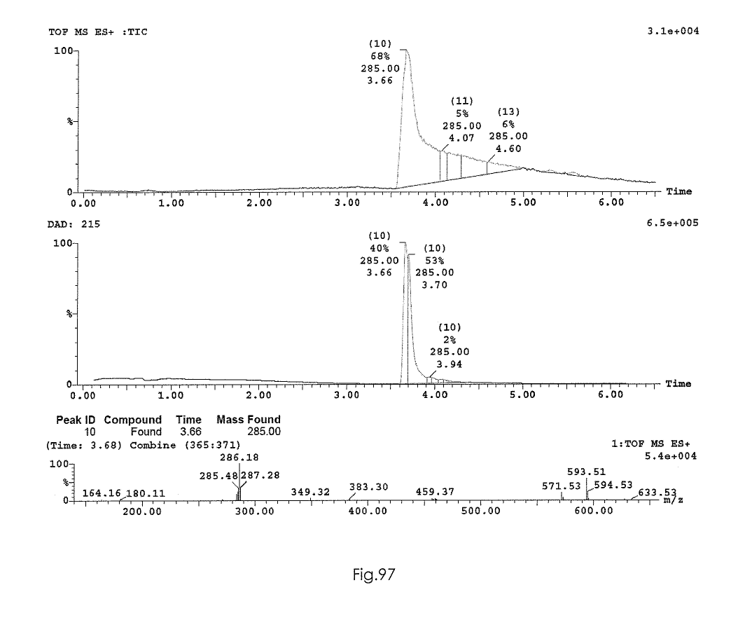

FIG. 97 shows the spectra/chromatograms of the compound of example 100. IC50 [μM]: >50.

### Example 101

### (5-Methoxy-2-pyridin-2-yl-pyrimidin-4-yl)-methyl-phenyl-amine

### MW: 292.36

### Manufacturer: Key Organics

HPLCMS (Method A): [m/z]: 293

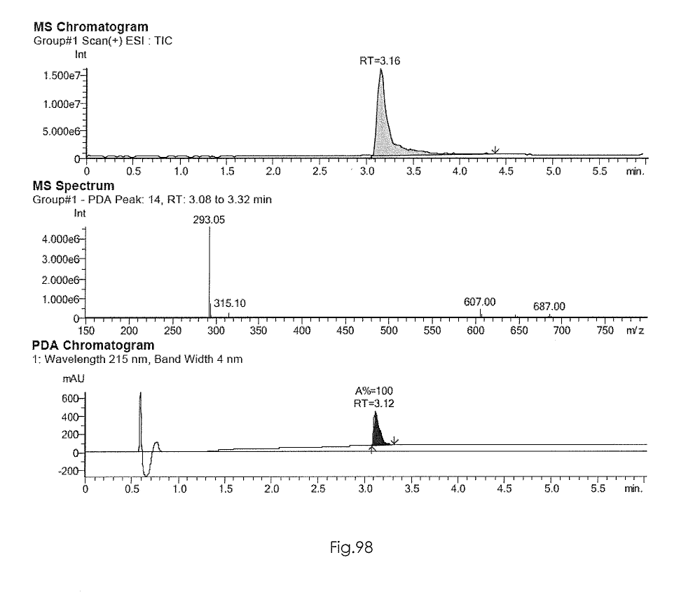

FIG. 98 shows the MS chromatogram, the MS spectrum and the PDA chromatogram of the compound of example 101.

### IC50 [μM]: <50.

### Example 102

### 5-Methoxy-4-phenoxy-2-pyridin-2-yl-pyrimidine

### MW: 279.29

### Manufacturer: Key Organics

HPLCMS (Method A): [m/z]: 280

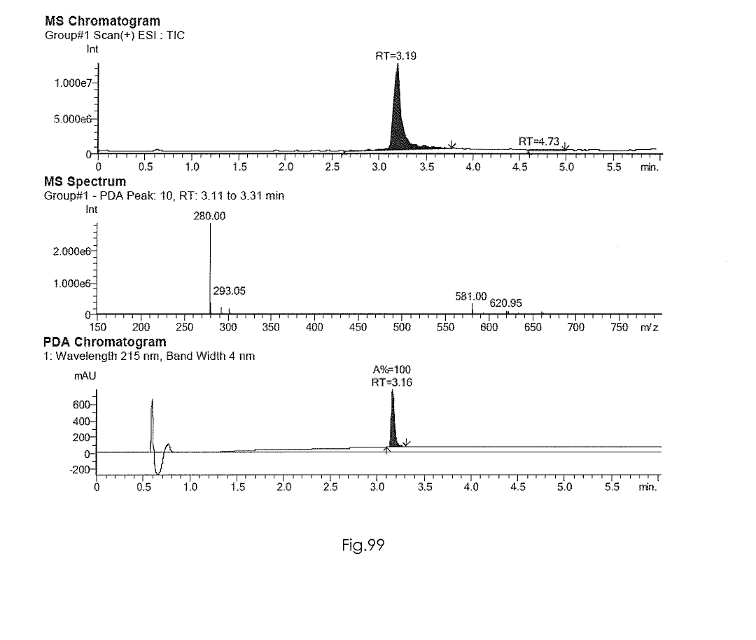

FIG. 99 shows the MS chromatogram, the MS spectrum and the PDA chromatogram of the compound of example 102.

### IC50 [μM]: >50.

### Example 103

### 5-(2-Methoxy-benzyl)-2-pyridin-2-yl-pyrimidine-4,6-diamine

### MW: 307.35

### Manufacturer: Key Organics

HPLCMS (Method A): [m/z]: 308

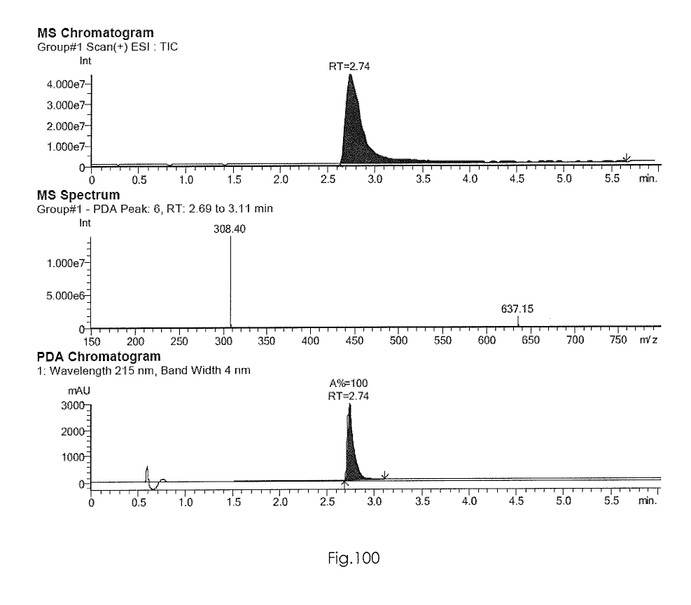

FIG. 100 shows the MS chromatogram, the MS spectrum and the PDA chromatogram of the compound of example 103.

### IC50 [μM]: <50.

### Example 104

### 5-(2,4-Dichloro-benzyl)-2-pyridin-2-yl-pyrimidine-4,6-diamine

### MW: 346.21

### Manufacturer: Key Organics

HPLCMS (Method A): [m/z]: 347

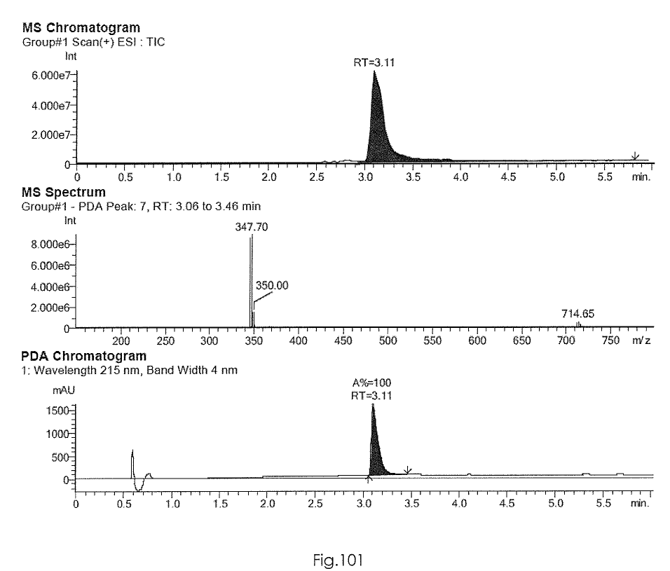

FIG. 101 shows the MS chromatogram, the MS spectrum and the PDA chromatogram of the compound of example 104.

### IC50 [μM]: <50.

### Examples 105-112

In the following examples the subsequently described analytical methods etc. were used:

### Analytical HPLC-MS Method A

Column: Waters Atlantis dC18 (2.1×100 mm, 3 mm column)
Flow rate: 0.6 ml/min
Solvent A: 0.1% Formic acid/water
Solvent B: 0.1% Formic acid/acetonitrile

### Injection Volume: 3 μl

Column temperature: 40° C.
UV Detection wavelength: 215 nm
Eluent: 0 mins to 5 mins, constant gradient from 95% solvent A+5% solvent B to 100% solvent B; 5 mins to 5.4 mins, 100% solvent B; 5.4 mins to 5.42 mins, constant gradient from 100% solvent B to 95% solvent A+5% solvent B; 5.42 mins to 7.00 mins, 95% solvent A+5% solvent B

### Method B

Column: Waters Atlantis dC18 (2.1×50 mm, 3 mm)
Solvent A: 0.1% Formic acid/water
Solvent B: 0.1% Formic acid/acetonitrile
Flow rate 1 ml/min
Injection volume 3 ml
UV Detection wavelength: 215 nm
Eluent: 0 to 2.5 minutes, constant gradient from 95% solvent A+5% solvent B to 100% solvent B; 2.5 minutes to 2.7 minutes, 100% solvent B; 2.71 to 3.0 minutes, 95% solvent A+5% solvent B.

### Method C

Column: Waters Atlantis dC18 (2.1×30 mm, 3 mm column)
Flow rate: 1 ml/min
Solvent A: 0.1% Formic acid/water
Solvent B: 0.1% Formic acid/acetonitrile
Injection volume: 3 ml
UV Detection wavelength: 215 nm
Eluent: 0 mins to 1.5 mins, constant gradient from 95% solvent A+5% solvent B to 100% solvent B; 1.5 mins to 1.6 mins, 100% solvent B; 1.60 min to 1.61 mins, constant gradient from 100% solvent B to 95% solvent A+5% solvent B; 1.61 mins to 2.00 min, 95% solvent A+5% solvent B.
MS detection using Waters LCT or LCT Premier, or ZQ or ZMD UV detection using Waters 2996 photodiode array or Waters 2787 UV or Waters 2788 UV

### Preparative HPLC—Neutral Conditions

### Column: Waters SunFire Prep C18 OBD (5 mm 19×100 mm)

Flow rate: 20 ml/min

### Solvent A: Water

### Solvent B: Acetonitrile

### Injection Volume: 1000 μl

Column Temperature: room temperature
Detection: UV directed
Eluent: 0 min to 2 min, 5% solvent B+95% solvent A; 2 min to 2.5 min constant gradient to 10% solvent B+90% solvent A, 2.5 min to 14.5 min constant gradient to 100% solvent B; 14.5 min to 16.5 min 100% solvent B; 16.5 to 16.7 min constant gradient to 5% B+95% A; 16.7 min to 17.2 min 5% solvent B+95% solvent A. Gilson semi-prep HPLC modules with 119 UV detector and 5.11 Unipoint control software
Waters 515 ancillary pumps
Waters 2487 UV detector
Gilson 215 autosampler and fraction collector

Flash silica gel chromatography was carried out on silica gel 230-400 mesh or on pre-packed silica cartridges.

Microwave reactions were carried out using a CEM Discover or Explorer focussed microwaves apparatus.

### Compound Naming

Some compounds are isolated as TFA or HCl salts, which are not reflected by the chemical name. Within the meaning of the present invention the chemical name represents the compound in neutral form as well as its TFA salt or any other salt, especially pharmaceutically acceptable salt, if applicable.

### Abbreviations

AcOH Acetic acid
n-BuOH n-Butanol
Cat. Catalytic
d Day(s)
DCE 1,2-Dichloroethane
DCM Dichloromethane
DIPEA N,N-diisoproylethylamine
DMAP 4-Dimethylaminopyridine
EDC.HCl N-[3-(dimethylamino)propyl]-N′-ethylcarbodiimide hydrochloride
Et₂O Diethyl ether
EtOAc Ethyl acetate
EtOH Ethanol
h Hour(s)
HPLC High Performance Liquid Chromatography
MeOH Methanol
min Minute(s)
MW Molecular Weight
i-PrOH iso-propanol
STAB Sodium triacetoxyborohydride
TEA Triethylamine
TFA Trifluoroacetic acid
THF Tetrahydrofuran
p-TSA para-toluenesulfonic acid

### Route 1

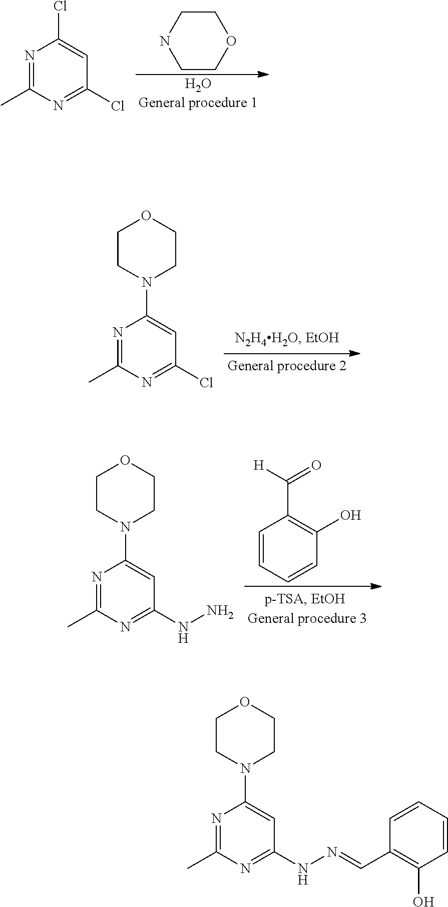

### General Procedure 1:

### 4-(6-Chloro-2-methyl-pyrimidin-4-yl)-morpholine

A mixture of morpholine (2.36 ml, 27.0 mmol) and 4,6-dichloro-2-methyl-pyrimidine (2.0 g, 12.3 mmol) in water (20 ml) was heated at 100° C. for 2 h. The reaction was allowed to cool to room temperature and was diluted with water (20 ml). The resulting precipitate was collected by filtration to give the title compound (1.90 g, 72% yield).

### MW: 213.67

HPLCMS (Method B): [m/z]: 214

### General Procedure 2:

### (2-Methyl-6-morpholin-4-yl-pyrimidin-4-yl)-hydrazine

A mixture of hydrazine monohydrate (150 ml, 3.09 mmol) and 4-(6-chloro-2-methyl-pyrimidin-4-yl)-morpholine (300 mg, 1.40 mmol) in EtOH (3 ml) was heated under reflux overnight. Additional hydrazine monohydrate (200 ml, 4.20 mmol) was added and the reaction was heated under reflux for a further 24 h. The reaction was allowed to cool to room temperature. The resulting precipitate was collected by filtration to give the title compound (246 mg, 84% yield).

### MW: 209.25

HPLCMS (Method B): [m/z]: 210

### Example 105

### General Procedure 3:

### 2-[(2-Methyl-6-morpholin-4-yl-pyrimidin-4-yl)-hydrazonomethyl]-phenol

2-Hydroxy-benzaldehyde (15 ml, 0.14 mmol) and p-toluenesulfonic acid monohydrate (cat) were added to a solution of (2-methyl-6-morpholin-4-yl-pyrimidin-4-yl)-hydrazine (30 mg, 0.14 mmol) in EtOH (0.6 ml). The reaction was stirred at room temperature for 20 min. The resulting precipitate was collected by filtration. The crude residue was purified by column chromatography with EtOAc/heptane (55%) as the eluent to give the title compound (24 mg, 55% yield).

### MW: 313.36

Title compound was not stable to HPLCMS conditions—structure confirmed by NMR.

### Route 2

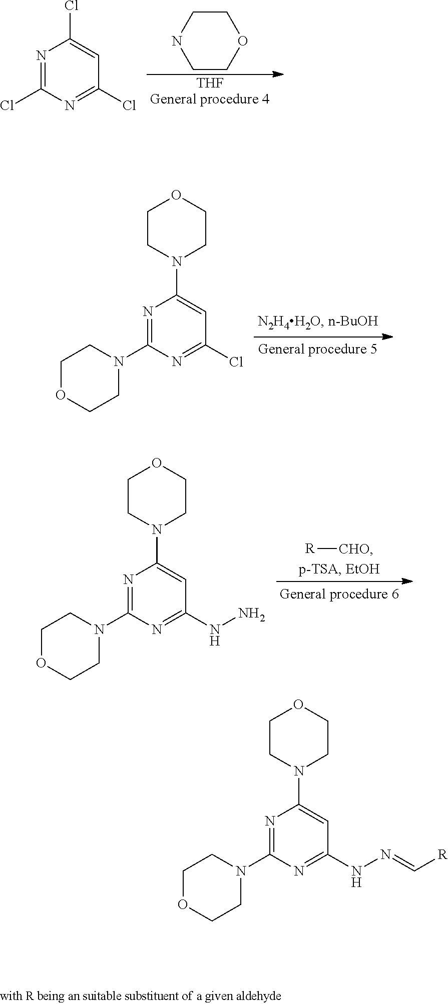

### General Procedure 4:

### 2,6-Di-morpholinyl-4-chloro-pyrimidine

Morpholine (4.74 ml, 54.52 mmol) was added dropwise to a solution of 2,4,6-trichloro-pyrimidine (2.0 g 10.90 mmol) in THF (30 ml) at 0° C. The reaction was allowed to warm to room temperature and was heated at 50° C. for 16 h. The reaction was cooled to room temperature, diluted with water (60 ml) and extracted with Et₂O (×3). The combined organic phases were dried (Na₂SO₄) and concentrated in vacuo. The crude residue was purified by column chromatography with EtOAc/heptane (20-30% gradient) as the eluent to give the title compound (2.52 g, 82% yield).

### MW: 284.75

HPLCMS (Method B): [m/z]: 285

### General Procedure 5:

### (2,6-Di-morpholin-4-yl-pyrimidin-4-yl)-hydrazine

Hydrazine monohydrate (256 ml, 5.27 mmol) was added dropwise to a solution of 2,6-di-morpholinyl-4-chloro-pyrimidine (300 mg, 1.05 mmol) in n-BuOH (1.2 ml). The reaction was heated under reflux for 16 h. The reaction was concentrated in vacuo. The crude residue was triturated with EtOH to give the title compound (276 mg, 94% yield).

### MW: 280.33

HPLCMS (Method B): [m/z]: 281

### Example 106

### General Procedure 6:

### N-Benzylidene-N′-(2,6-di-morpholin-4-yl-pyrimidin-4-yl)-hydrazine

p-Toluenesulfonic acid monohydrate (cat) was added to a solution of (2,6-di-morpholin-4-yl-pyrimidin-4-yl)-hydrazine (50 mg, 0.18 mmol) and benzaldehyde (18.2 ml, 0.18 mmol) in EtOH (2 ml). The resulting precipitate was collected by filtration and was triturated with a solution of Et₂O, MeOH and DCM (1:1:1) to give the title compound (13 mg, 18% yield).

### MW: 368.44

HPLCMS (Method A): [m/z]: 369

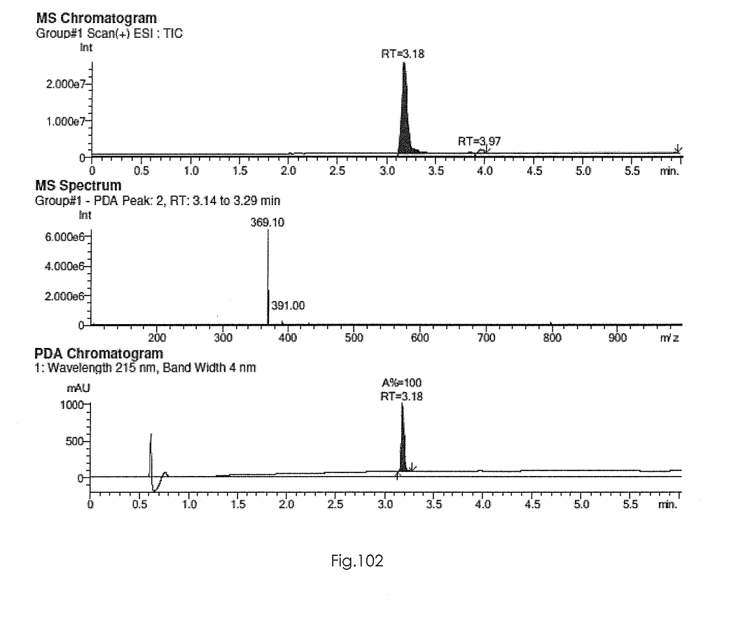

FIG. 102 shows the MS chromatogram, the MS spectrum and the PDA chromatogram of the compound of example 106.

### IC50 [μM]: >50.

### Example 107

### 4-[(2,6-Di-morpholin-4-yl-pyrimidin-4-yl)-hydrazonomethyl]-benzene-1,3-diol

In a similar fashion using route 2, general procedure 6, p-toluenesulfonic acid monohydrate (cat), (2,6-di-morpholin-4-yl-pyrimidin-4-yl)-hydrazine (50 mg, 0.18 mmol) and 2,4-dihydroxy-benzaldehyde (24.6 mg, 0.18 mmol) in EtOH (2 ml) gave the title compound (34 mg, 51% yield) after purification by trituration from EtOH.

### MW: 400.44

HPLCMS (Method A): [m/z]: 401

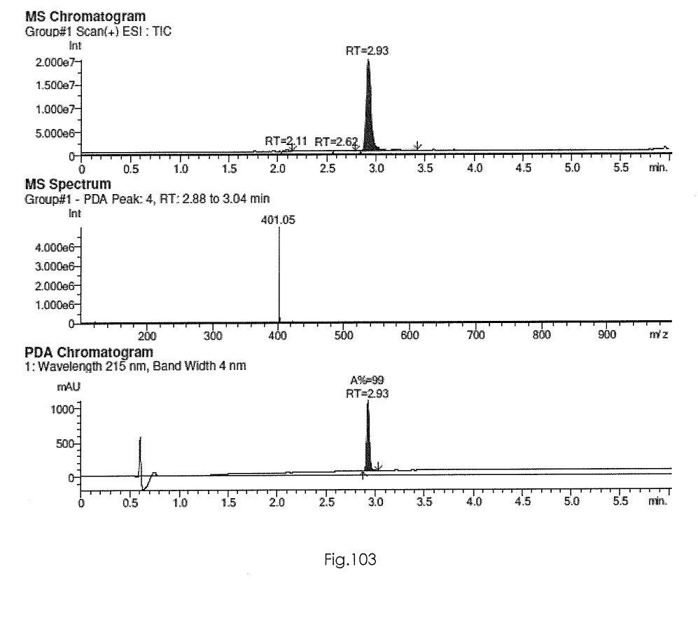

FIG. 103 shows the MS chromatogram, the MS spectrum and the PDA chromatogram of the compound of example 107.

### IC50 [μM]: >50.

### Example 108

### Route 3

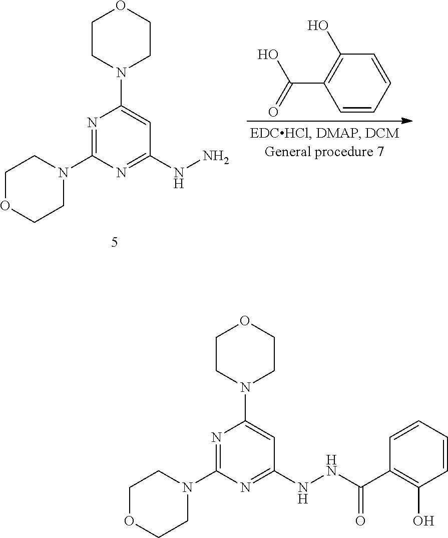

### General Procedure 7:

### 2-Hydroxy-benzoic acid N′-(2,6-di-morpholin-4-yl-pyrimidin-4-yl)-hydrazide

EDC.HCl (97 mg, 0.49 mmol) was added to a solution of (2,6-di-morpholin-4-yl-pyrimidin-4-yl)-hydrazine (127 mg, 0.48 mmol), 2-hydroxy-benzoic acid (63 mg, 0.48 mmol) and DMAP (cat) in DCM and the mixture was stirred for 16 h at room temperature. The reaction mixture was concentrated in vacuo. The crude residue was purified by preparative HPLC (neutral conditions) followed by trituration from Et₂O/EtOAc to give the title compound (8 mg, 4% yield).

### MW: 400.44

HPLCMS (Method A): [m/z]: 401

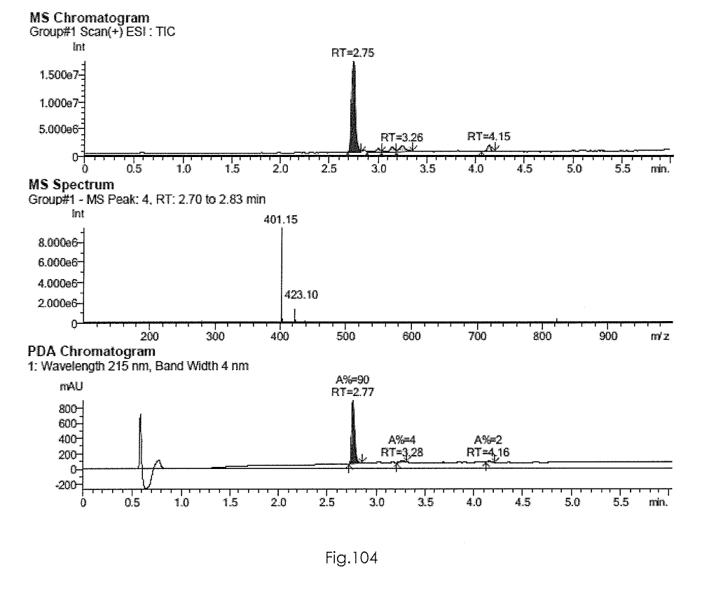

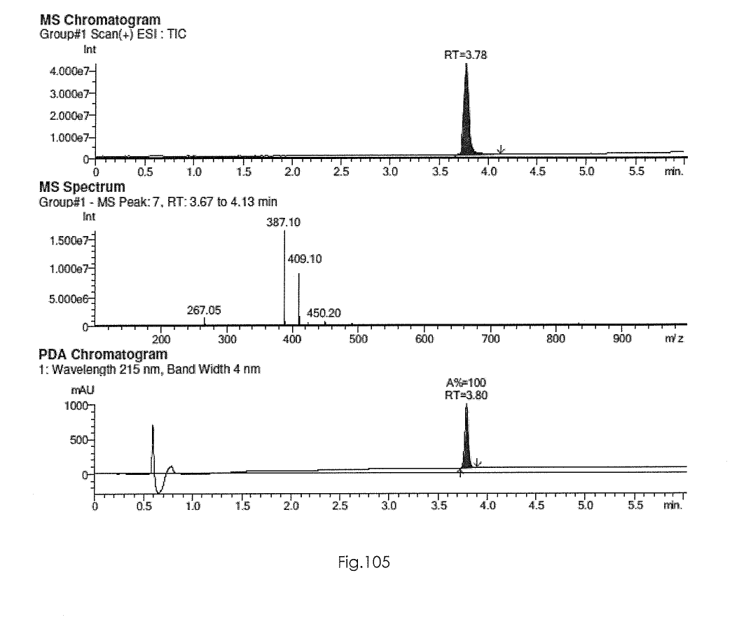

FIG. 104 shows the MS chromatogram, the MS spectrum and the PDA chromatogram of the compound of example 108.

### IC50 [μM]: >50.

### Example 109

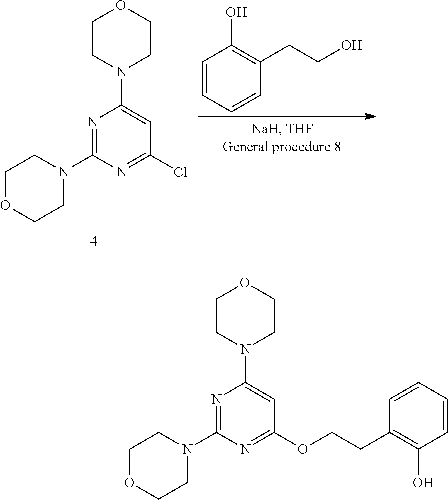

### General Procedure 8:

### 2-[2-(2,6-Di-morpholin-4-yl-pyrimidin-4-yloxy)-ethyl]-phenol

Sodium hydride (60% dispersion in mineral oil, 14 mg, 0.35 mmol) was added to a solution of 2,6-di-morpholinyl-4-chloro-pyrimidine (50 mg, 0.18 mmol) and 2-(2-hydroxy-ethyl)-phenol (24 mg, 0.18 mmol) in THF (1 ml) at 0° C. under N₂ in a microwave tube. The reaction was allowed to warm to room temperature and was then stirred at room temperature for 1 h. The microwave tube was then flushed with N₂, sealed and heated at 120° C. in the microwave for 11 h. The reaction was diluted with water (1 ml) and neutralised by the dropwise addition of 0.1M aqueous HCl. The resulting solution was extracted with EtOAc (×3). The combined organic phases were dried (Na₂SO₄) and concentrated in vacuo. The crude residue was purified by column chromatography with EtOAc/heptane (30-45% gradient) as the eluent to give the title compound (17 mg, 25% yield).

### MW: 386.45

HPLCMS (Method A): [m/z]: 387

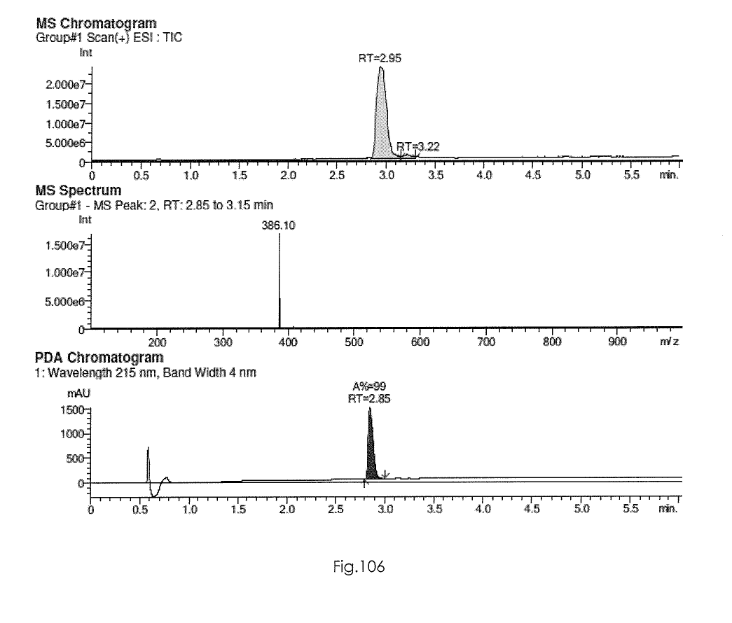

FIG. 105 shows the MS chromatogram, the MS spectrum and the PDA chromatogram of the compound of example 109.

### IC50 [μM]: >50.

### Route 5

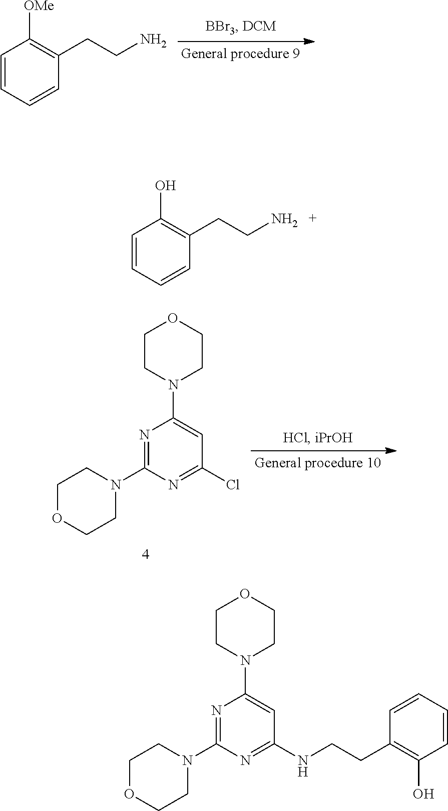

### General Procedure 9:

### 2-(2-Amino-ethyl)-phenol

Boron tribromide (1.0 M solution in DCM, 33.1 ml, 33.1 mmol) was added dropwise to a solution of 2-(2-methoxy-phenyl)-ethylamine (2.0 g, 13.2 mmol) in DCM (20 ml) at −78° C. The reaction was allowed to warm to room temperature overnight. The reaction was quenched by the addition of MeOH (20 ml) at −78° C. The reaction was allowed to warm to room temperature and was then stirred for 1 h. The resulting solution was concentrated in vacuo, diluted with saturated aqueous NaHCO₃ solution (100 ml) and extracted with i-PrOH/CHCl₃ (1:1, ×3). The combined organic phases were dried (Na₂SO₄) and concentrated in vacuo to give the title compound (0.34 g, 19% yield).

### MW: 137.18

HPLCMS (Method B): [m/z]: 138

### Example 110

### General Procedure 10:

### 2-[2-(2,6-Di-morpholin-4-yl-pyrimidin-4-ylamino)-ethyl]-phenol

Concentrated HCl (2 drops) was added to a solution of 2,6-di-morpholinyl-4-chloro-pyrimidine (180 mg, 0.63 mmol) and 2-(2-amino-ethyl)-phenol 10 (130 mg, 0.95 mmol) in i-PrOH (3.5 ml). The reaction was heated at 170° C. in the microwave for 1 h. The reaction was basified with saturated aqueous NaHCO₃ solution and extracted with DCM (×3). The combined organic phases were dried (Na₂SO₄) and concentrated in vacuo. The crude residue was purified by column chromatography with EtOAc/heptane (75%) as the eluent, to give the title compound (30 mg, 12% yield).

### MW: 385.47

HPLCMS (Method A): [m/z]: 386

FIG. 106 shows the MS chromatogram, the MS spectrum and the PDA chromatogram of the compound of example 110.

### IC50 [μM]: >50.

### Route 6

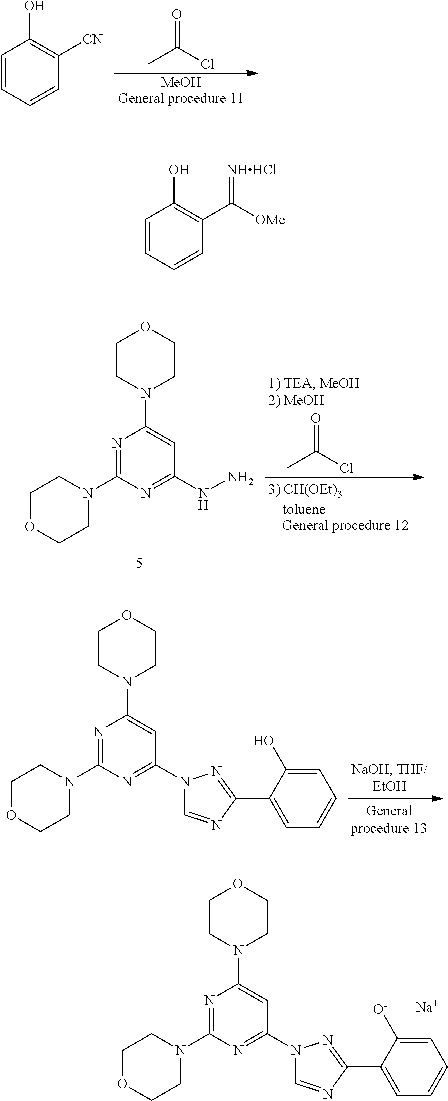

### General Procedure 11:

### 2-Hydroxy-benzimidic acid methyl ester

Acetyl chloride (5.9 ml, 83.95 mmol) was added dropwise to MeOH (11 ml) at room temperature under N₂. The reaction was stirred for 2 h and 2-hydroxy-benzonitrile (2.0 g, 16.79 mmol) was added. After 48 h the reaction was concentrated in vacuo. The residue was dissolved in DCM (5 ml) and Et₂O was added dropwise to form a precipitate. The precipitate was collected by filtration to give the title compound as the HCl salt (0.59 g, 19% yield).

### MW: 151.17

HPLCMS (Method B): [m/z]: 152

### General Procedure 12:

### 2-[1-(2,6-Di-morpholin-4-yl-pyrimidin-4-yl)-1H-[1,2,4]triazol-3-yl]-phenol

TEA (148 ml, 1.07 mmol) was added to a solution of 2-hydroxy-benzimidic acid methyl ester HCl (167 mg, 0.89 mmol) in MeOH (3.5 ml). After 30 min (2,6-di-morpholin-4-yl-pyrimidin-4-yl)-hydrazine (275 mg, 0.98 mmol) was added and the reaction was heated under reflux for 6 h. In a separate flask acetyl chloride (69 ml, 0.98 mmol) was added dropwise to MeOH (3.5 ml) and stirred at room temperature for 30 min. This was added to the main reaction mixture at 0° C. The reaction was stirred at room temperature for 10 min before being concentrated in vacuo. The residue was dissolved in toluene (5 ml) and Methyl orthoformate (5 ml) was added. The reaction was heated at 100° C. for 30 min. After cooling to 85° C., EtOH (3 ml) was added and the reaction was maintained at 85° C. for 30 min. After cooling to room temperature, the mixture was basified with saturated aqueous NaHCO₃ solution. The phases were separated and the aqueous phase was extracted with DCM (×3). The combined organic phases were dried (Na₂SO₄) and concentrated in vacuo. The crude residue was purified by column chromatography with EtOAc/heptane (25%) as the eluent.

The resulting solid was triturated in MeOH to give the title compound (40 mg, 11% yield).

### MW: 409.45

HPLCMS (Method A): [m/z]: 410

### Example 111

### General Procedure 13:

Sodium 2-[1-(2,6-Di-morpholin-4-yl-pyrimidin-4-yl)-1H-[1,2,4]triazol-3-yl]-phenoxide NaOH (0.1M solution in water, 0.5 ml, 48.8 mmol) was added to a suspension of 2-[1-(2,6-di-morpholin-4-yl-pyrimidin-4-yl)-1H-[1,2,4]triazol-3-yl]-phenol (20 mg, 48.8 mmol) in EtOH/THF (1:20, 5.25 ml). The reaction mixture was concentrated in vacuo to give the title compound (21 mg, 100% yield).

MW: 408.44 (anion)
HPLCMS (Method A): [m/z]: 410

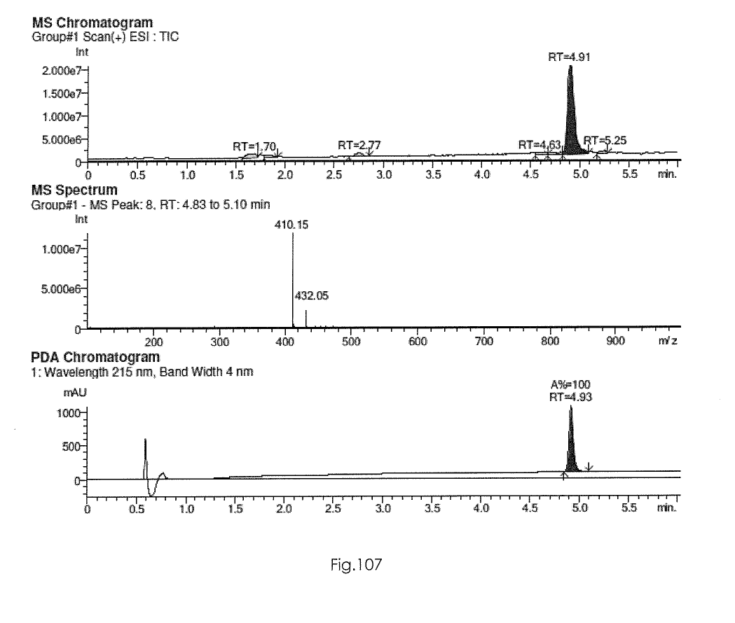

FIG. 107 shows the MS chromatogram, the MS spectrum and the PDA chromatogram of the compound of example 111.

### IC50 [μM]: >50.

### Route 7

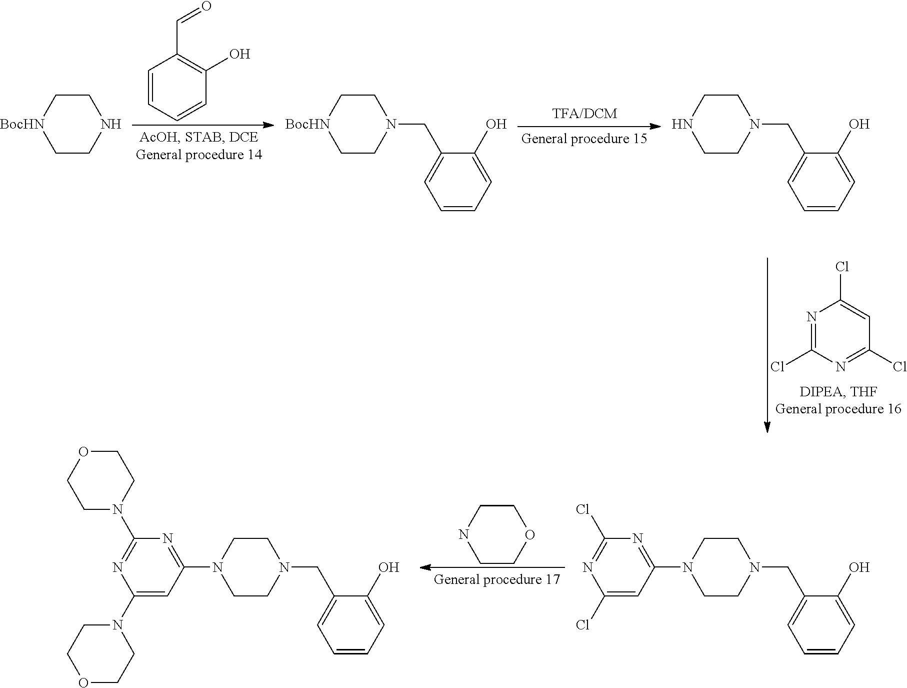

### General Procedure 14:

### 4-(2-Hydroxy-benzyl)-piperazine-1-carboxylic acid tert-butyl ester

Acetic acid (308 ml, 5.37 mmol) was added to a solution of piperazine-1-carboxylic acid Cert-butyl ester (1.0 g, 5.37 mmol) and 2-hydroxy-benzaldehyde (570 ml, 5.37 mmol) in DCE over 4μ molecular sieves. The reaction was stirred for 1 h at room temperature and then sodium triacetoxyborohydride (2.28 g, 10.74 mmol) was added. After stirring for a further 16 h the reaction was quenched with MeOH (10 ml). After stirring for 30 min the mixture was filtered and the filtrate was concentrated in vacuo. The crude residue was purified by column chromatography with EtOAc/heptane (25%) as the eluent to give the title compound (0.69 g, 44% yield).

### MW: 292.38

HPLCMS (Method B): [m/z]: 293

### General Procedure 15:

### 2-piperazin-1-ylmethyl-phenol

4-(2-Hydroxy-benzyl)-piperazine-1-carboxylic acid tert-butyl ester (0.69 g, 2.36 mmol) was dissolved in TFA/DCM (1:3, 7 ml) and the mixture was stirred at room temperature for 18 h. The reaction mixture was concentrated in vacuo to give the title compound as the TFA salt (0.99 g, 100% yield).

### MW: 192.26

HPLCMS (Method B): [m/z]: 193

### General Procedure 16:

### 2-[4-(2,6-Dichloro-pyrimidin-4-yl)-piperazin-1-ylmethyl]-phenol

DIPEA (0.5 ml, 2.85 mmol) was added to a solution of 2-piperazin-1-ylmethyl-phenol trifluoroacetic acid salt (400 mg, 0.95 mmol) in THF (5 ml) and stirred for 30 min at room temperature. The resulting solution was added dropwise to a stirred solution of 2,4,6-trichloro-pyrimidine (109 ml, 0.95 mmol) in THF (1 ml) at 0° C. and the reaction was stirred for 18 h at room temperature. The reaction mixture was diluted with water (6 ml) and was extracted with EtOAc (×3). The combined organic phases were dried (Na₂SO₄) and concentrated in vacuo. The crude residue was purified by column chromatography with EtOAc/heptane (25%) as the eluent to give the title compound (145 mg, 35% yield).

### MW: 339.23

HPLCMS (Method B): [m/z]: 339

### Example 112

### General Procedure 17:

### 2-[4-(2,6-Di-morpholin-4-yl-pyrimidin-4-yl)-piperazin-1-ylmethyl]-phenol

A solution of 2-[4-(2,6-dichloro-pyrimidin-4-yl)-piperazin-1-ylmethyl]-phenol (128 mg, 0.38 mmol) in morpholine (4 ml) was heated under reflux for 18 h. The mixture was concentrated in vacuo and the residue was dissolved in EtOAc (10 ml). The organic phase was washed with saturated aqueous NaHCO₃ solution (10 ml). The organic phase was dried (Na₂SO₄) and concentrated in vacuo. The crude residue was triturated in EtOAc to give the title compound (84 mg, 50% yield).

### MW: 440.55

HPLCMS (Method A): [m/z]: 441

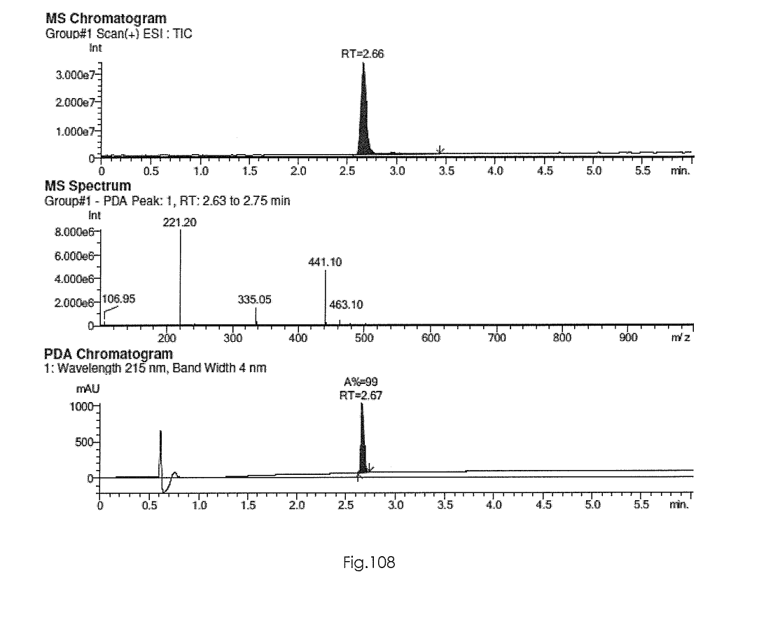

FIG. 108 shows the MS chromatogram, the MS spectrum and the PDA chromatogram of the compound of example 112.

### IC50 [μM]: >50.

### Examples 113-117

In the following examples the subsequently described analytical methods etc. were used:

### Analytical HPLC-MS

### Method A

Column: Waters Atlantis dC18 (2.1×100 mm, 3 μm column)
Flow rate: 0.6 ml/min
Solvent A: 0.1% Formic acid/water
Solvent B: 0.1% Formic acid/acetonitrile

### Injection Volume: 3 μl

Column temperature: 40° C.
UV Detection wavelength: 215 nm
Eluent: 0 mins to 5 mins, constant gradient from 95% solvent A+5% solvent B to 100% solvent B; 5 mins to 5.4 mins, 100% solvent B; 5.4 mins to 5.42 mins, constant gradient from 100% solvent B to 95% solvent A+5% solvent B; 5.42 mins to 7.00 mins, 95% solvent A+5% solvent B

### Method B

Column: Waters Atlantis dC18 (2.1×50 mm, 3 μm)
Solvent A: 0.1% Formic acid/water
Solvent B: 0.1% Formic acid/acetonitrile
Flow rate 1 ml/min
Injection volume 3 μl
UV Detection wavelength: 215 nm
Eluent: 0 to 2.5 minutes, constant gradient from 95% solvent A+5% solvent B to 100% solvent B; 2.5 minutes to 2.7 minutes, 100% solvent B; 2.71 to 3.0 minutes, 95% solvent A+5% solvent B.
MS detection using Waters LCT or LCT Premier, or ZQ or ZMD
UV detection using Waters 2996 photodiode array or Waters 2787 UV or Waters 2788 UV

Flash silica gel chromatography was carried out on silica gel 230-400 mesh or on pre-packed silica cartridges.

### ABBREVIATIONS

d Day(s)
DCM Dichloromethane
DIPEA N,N-disopropylethylamine
EtOAc Ethyl acetate
EtOH Ethanol
h Hour(s)
HPLC High Performance Liquid Chromatography
min Minutes
MW Molecular weight
p-TSA para-toluenesulfonic acid
TFA Trifluoroacetic acid
THF Tetrahydrofuran

### Route 1

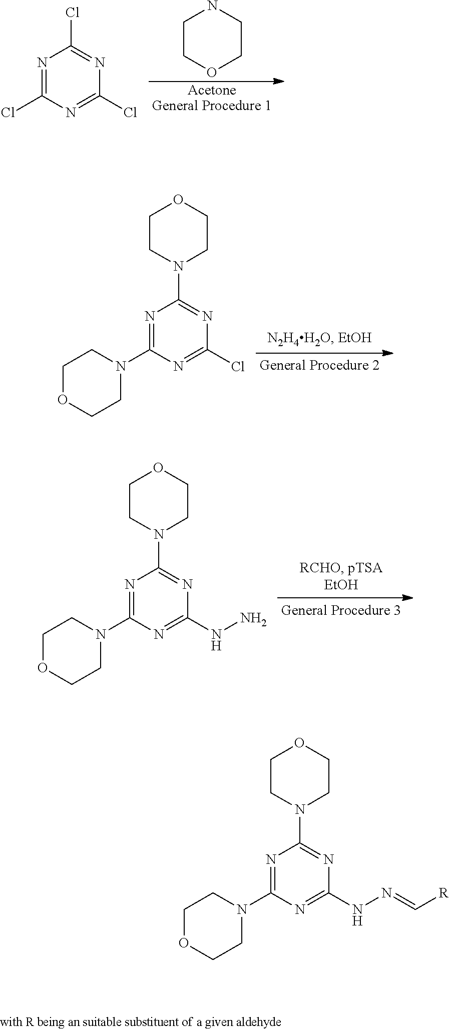

### General Procedure 1:

### 2-Chloro-4,6-di-morpholin-4-yl-[1,3,5]triazine

A solution of morpholine (4.0 ml, 45.8 mmol) in water (2 ml) was added to a solution of cyanuric chloride (2.0 g, 10.9 mmol) in acetone (30 ml) at 0° C. and the mixture was stirred at 0° C. for 1.75 h. Water (50 ml) was added and the resulting precipitate was collected by filtration, washed with water and dried at 40° C. under vacuum to give the title compound (2.84 g, 91%).

### MW: 285.74

HPLCMS (Method B): [m/z]: 286

### General Procedure 2:

### (4,6-Di-morpholin-4-yl-[1,3,5]triazin-2-yl)-hydrazine

Hydrazine hydrate (0.88 ml, 1.75 mmol) was added to a solution of 2-chloro-4,6-di-morpholin-4-yl-[1,3,5]triazine 1 (100 mg, 0.35 mmol) in EtOH (1 ml) and the mixture was heated under reflux for 1.5 h. After cooling, the resulting solid was collected by filtration and washed with EtOH to give the title compound (85 mg, 86%).

### MW: 281.32

HPLCMS (Method B): [m/z]: 282

### Example 113

### General Procedure 3:

### 2-[(4,6-Di-morpholin-4-yl-[1,3,5]triazin-2-yl)-hydrazonomethyl]-phenol

2-Hydroxybenzaldehyde (15 μl, 0.14 mmol) and p-toluenesulfonic acid (2 mg, 0.01 mmol) were added to a solution of (4,6-di-morpholin-4-yl-[1,3,5]triazin-2-yl)-hydrazine (40 mg, 0.14 mmol) in EtOH (0.5 ml) at 0° C. and the mixture was stirred for 1.25 h. Additional 2-hydroxybenzaldehyde (3 μl) was added and stirring continued at 0° C. for 30 min and at room temperature for 18 h. Finally the mixture was heated at 50° C. for 3 h. After cooling, the resulting precipitate was collected by filtration and washed with EtOH. The crude solid was purified by column chromatography with MeOH/DCM (2%) as the eluent to give the title compound (25 mg, 46%).

### MW: 385.43

HPLCMS (Method A): [m/z]: 386

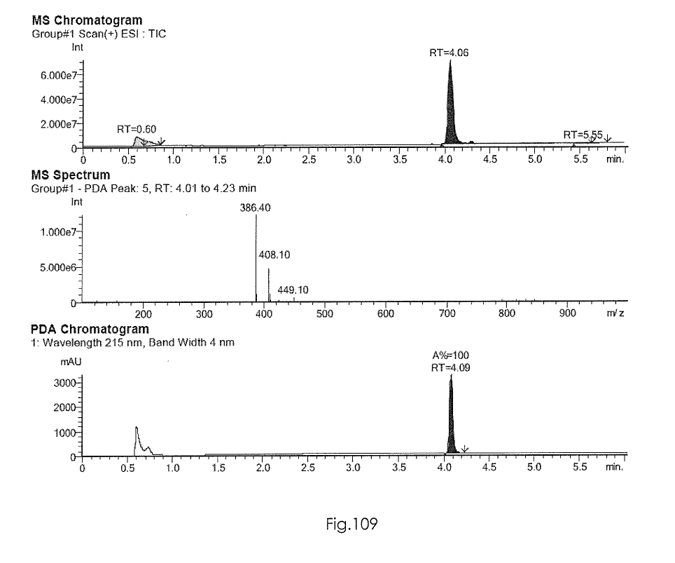

FIG. 109 shows the MS chromatogram, the MS spectrum and the PDA chromatogram of the compound of example 113.

### IC50 [μM]: >50.

### Example 114

### 4-[(4,6-Di-morpholin-4-yl-[1,3,5]triazin-2-yl)-hydrazonomethyl]-benzene-1,3-diol

In a similar fashion using route 1 general procedure 3, (4,6-di-morpholin-4-yl-[1,3,5]triazin-2-yl)-hydrazine (40 mg, 0.14 mmol), 2,4-dihydroxybenzaldehyde (19 mg, 0.14 mmol) and p-toluenesulfonic acid (2 mg, 0.08 mmol) gave the title compound (20 mg, 36%) after purification by column chromatography with MeOH/DCM (0-3% gradient) as the eluent.

### MW: 401.43

HPLCMS (Method A): [m/z]: 402

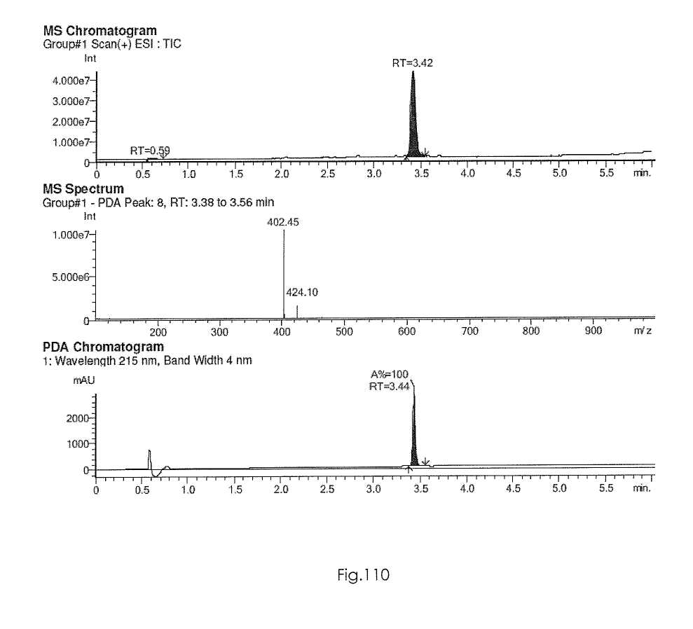

FIG. 110 shows the MS chromatogram, the MS spectrum and the PDA chromatogram of the compound of example 114.

### IC50 [μM]: >50.

### Route 2

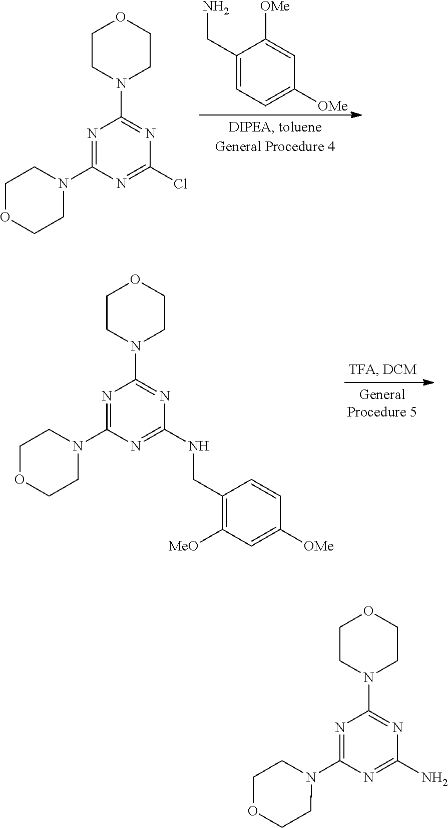

### General Procedure 4:

### (2,4-Dimethoxy-benzyl)-(4,6-di-morpholin-4-yl-[1,3,5]triazin-2-yl)-amine (covered by the invention)

2,4-Dimethoxybenzylamine (0.79 ml, 5.25 mmol) was added to a solution of 2-chloro-4,6-di-morpholin-4-yl-[1,3,5]triazine (0.5 g, 1.75 mmol) in toluene (10 ml) followed by DIPEA (0.61 ml, 3.50 mmol) and the mixture was heated at 90° C. for 18 h. After cooling, the resulting suspension was filtered through celite and washed with toluene. The filtrate was concentrated in vacuo and the residue was dissolved in DCM. The organic phase was washed with water (×2) and brine, dried (Mg504) and concentrated in vacuo. The crude residue was purified by column chromatography with MeOH/DCM (0-5% gradient) as the eluent to give the title compound (0.64 g, 88%)

### MW: 416.48

HPLCMS (Method B): [m/z]: 417

### Example 115

### General Procedure 5: 4,6-Di-morpholin-4-yl-[1,3,5]triazin-2-ylamine

TFA (2.5 ml) was added to a solution of (2,4-dimethoxy-benzyl)-(4,6-di-morpholin-4-yl-[1,3,5]triazin-2-yl)-amine (0.50 g, 1.20 mmol) in DCM (5 ml) and the mixture was stirred at room temperature for 18 h. Water was added and the mixture was stirred for 1 h.

FIG. 110 shows the MS chromatogram, the MS spectrum and the PDA chromatogram of the compound of example 114.

### IC50 [μM]: >50.

### Route 2

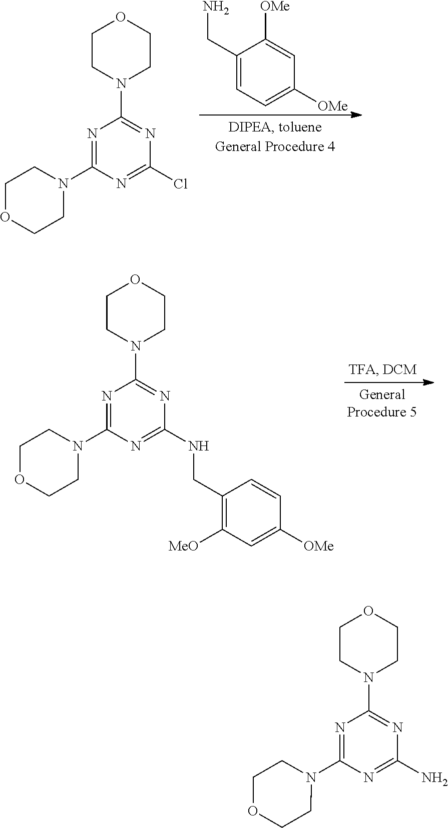

### General Procedure 4:

### (2,4-Dimethoxy-benzyl)-(4,6-di-morpholin-4-yl-[1,3,5]triazin-2-yl)-amine (covered by the invention)

2,4-Dimethoxybenzylamine (0.79 ml, 5.25 mmol) was added to a solution of 2-chloro-4,6-di-morpholin-4-yl-[1,3,5]triazine (0.5 g, 1.75 mmol) in toluene (10 ml) followed by DIPEA (0.61 ml, 3.50 mmol) and the mixture was heated at 90° C. for 18 h. After cooling, the resulting suspension was filtered through celite and washed with toluene. The filtrate was concentrated in vacuo and the residue was dissolved in DCM. The organic phase was washed with water (×2) and brine, dried (MgSO₄) and concentrated in vacuo. The crude residue was purified by column chromatography with MeOH/DCM (0-5% gradient) as the eluent to give the title compound (0.64 g, 88%)

### MW: 416.48

HPLCMS (Method B): [m/z]: 417

### Example 115

### General Procedure 5: 4,6-Di-morpholin-4-yl-[1,3,5]triazin-2-ylamine

TFA (2.5 ml) was added to a solution of (2,4-dimethoxy-benzyl)-(4,6-di-morpholin-4-yl-[1,3,5]triazin-2-yl)-amine (0.50 g, 1.20 mmol) in DCM (5 ml) and the mixture was stirred at room temperature for 18 h. Water was added and the mixture was stirred for 1 h. The phases were separated and the organic phase dried (MgSO₄) and concentrated in vacuo. The residue was dissolved in EtOAc and Na₂CO₃ (aq) and the resulting mixture was stirred for 30 min. The phases were separated and the aqueous phase was extracted with EtOAc. The combined organic phases were dried (MgSO₄) and concentrated in vacuo. A quarter of the crude residue (60 mg) was purified by column chromatography with MeOH/DCM as the eluent to give the title compound (54 mg, ˜68% overall).

### MW: 266.31

HPLCMS (Method A): [m/z]: 267

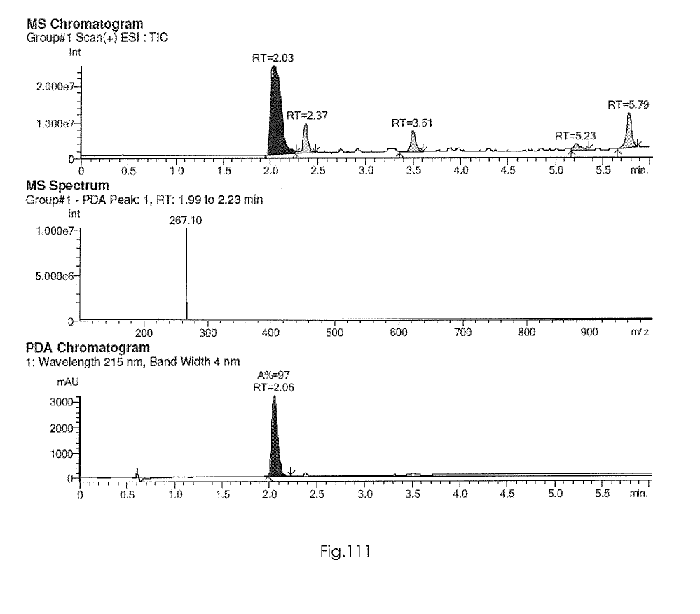

FIG. 111 shows the MS chromatogram, the MS spectrum and the PDA chromatogram of the compound of example 115.

### IC50 [μM]: >50.

### Example 116

### Route 4 (Route 3 See Example 12).

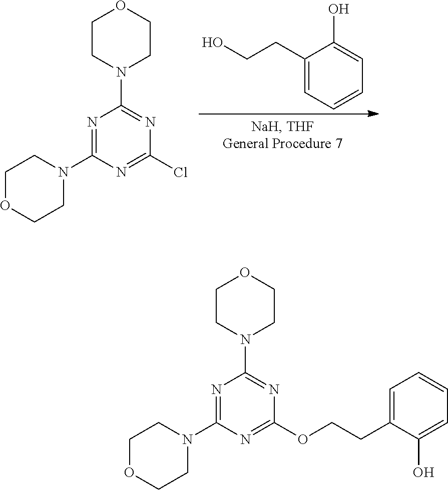

### 2-[2-(4,6-Di-morpholin-4-yl-[1,3,5]triazin-2-yloxy)-ethyl]-phenol

2-(2-Hydroxy-ethyl)-phenol (73 mg, 0.53 mmol) was added to a solution of 2-chloro-4,6-di-morpholin-4-yl-[1,3,5]triazine (151 mg, 0.53 mmol) in THF (3 ml) followed by sodium hydride (60% suspension in mineral oil; 14 mg, 1.06 mmol) and the mixture was stirred at room temperature for 1 h and heated at 70° C. for 18 h. After cooling, the mixture was partitioned between water and EtOAc and the aqueous phase was extracted with EtOAc. The combined organic phases were dried (MgSO₄) and concentrated in vacuo. The crude residue was purified by column chromatography with EtOAc/heptane (0-40% gradient) as the eluent followed by trituration from DCM/heptane to give the title compound (30 mg, 15%).

### MW: 387.44

HPLCMS (Method A): [m/z]: 388

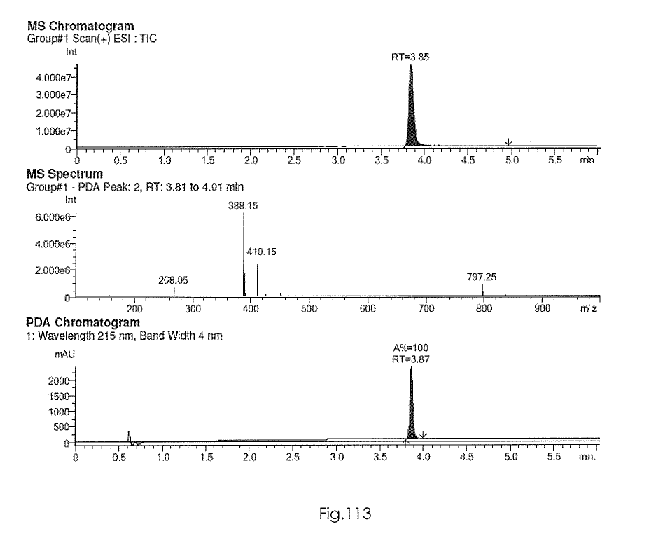

FIG. 113 shows the MS chromatogram, the MS spectrum and the PDA chromatogram of the compound of example 116.

### IC50 [μM]: >50.

### Route 5

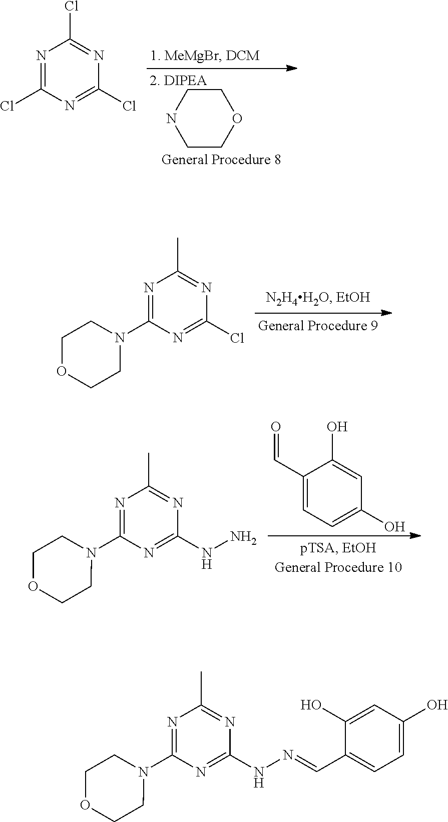

### General Procedure 8:

### 2-Chloro-4-methyl-6-morpholin-4-yl-[1,3,5]triazine

Methyl magnesium bromide (3M in ether; 3.4 ml, 10.4 mmol) was added to a solution of cyanuric chloride (2.0 g, 10.9 mmol) in anhydrous DCM (40 ml) at 0° C. After complete addition the mixture was allowed to warm to room temperature over 2 h. The mixture was cooled to 0° C. and morpholine (0.96 ml, 10.9 mmol) was added dropwise followed by DIPEA (1.9 ml, 10.9 mmol) and the reaction was allowed to warm to room temperature over 1 h. Water was added and the resulting mixture was filtered through celite. The organic phase was washed with water and brine, dried (MgSO₄) and concentrated in vacuo. The crude residue was purified by column chromatography with MeOH/DCM (0-10% gradient) as the eluent to give the title compound (0.54 g, 23%).

### MW: 214.66

HPLCMS (Method B): [m/z]: 215

### General Procedure 9:

### (4-Methyl-6-morpholin-4-yl-[1,3,5]triazin-2-yl)-hydrazine

Hydrazine hydrate (0.18 ml, 2.35 mmol) was added to a solution of 2-chloro-4-methyl-6-morpholin-4-yl-[1,3,5]triazine (100 mg, 0.47 mmol) in EtOH (1 ml) and the mixture was heated under reflux for 1.5 h. After cooling to 0° C., the resulting solid was collected by filtration and washed with EtOH to give the title compound (64 mg, 65%).

### MW: 210.24

HPLCMS (Method B): [m/z]: 211

### Example 117

### General Procedure 10:

### 4-[(4-Methyl-6-morpholin-4-yl-[1,3,5]triazin-2-yl)-hydrazonomethyl]-benzene-1,3-diol

2,4-Dihydroxybenzaldehyde (40 mg, 0.29 mmol) and p-toluenesulfontc acid (3,5 mg. 0.02 mmol) were added to a solution of (4-methyl-6-morpholin-4-yl-[1,3,5]triazin-2-yl)-hydrazine (60 mg, 0.29 mmol) in EtOH (1 ml) at 0° C. and the mixture was stirred for 1.5 h. The resulting precipitate was collected by filtration and washed with EtOH. The combined solid and filtrate were purified by column chromatography with 2M ammonia in MeOH/DCM (0-7% gradient) as the eluent followed by trituration from iso-propyl alcohol to give the title compound (13.5 mg, 14%).

### MW: 330.35

HPLCMS (Method A):[m/z]: 331

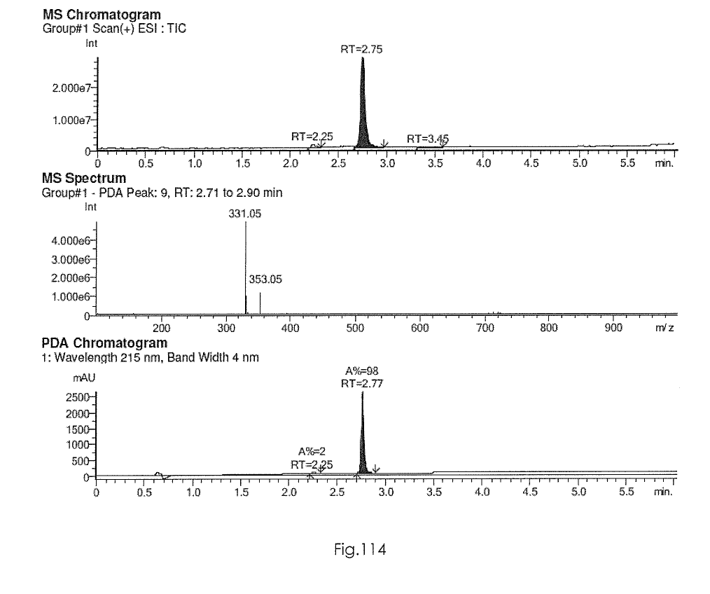

FIG. 114 shows the MS chromatogram, the MS spectrum and the PDA chromatogram of the compound of example 117.

IC50 [μM]:>50.

1. A method of treating iron metabolism disorders, comprising, administering to a patient in need, a preparation including compounds of general formula (I) 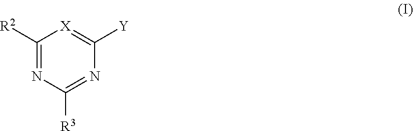
wherein
X is selected from the group consisting of N or C—R¹, wherein
R¹ is selected from the group consisting of:
hydrogen,
hydroxyl,
halogen
carboxyl,
sulfonic acid residue (—SO₃H),
optionally substituted aminocarbonyl,
optionally substituted aminosulfonyl,
optionally substituted amino,
optionally substituted alkyl,
optionally substituted acyl,
optionally substituted alkoxycarbonyl,
optionally substituted acyloxy,
optionally substituted alkoxy,
optionally substituted alkenyl,
optionally substituted alkynyl,
optionally substituted aryl,
optionally substituted heterocyclyl;
R² and R³ are the same or different and are each selected from the group consisting of:
hydrogen,
hydroxyl,
halogen
carboxyl,
sulfonic acid residue (—SO₃H),
optionally substituted aminocarbonyl,
optionally substituted aminosulfonyl,
optionally substituted amino,
optionally substituted alkyl,
optionally substituted acyl,
optionally substituted alkoxycarbonyl,
optionally substituted acyloxy,
optionally substituted alkoxy,
optionally substituted alkenyl,
optionally substituted alkynyl,
optionally substituted aryl,
optionally substituted heterocyclyl;
Y is selected from the group consisting of
hydrogen
hydroxyl,
halogen,
optionally substituted aryloxy, and 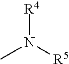
wherein
R⁴ and R⁵ are the same or different and are each selected from the group consisting of:
hydrogen,
optionally substituted amino,
optionally substituted aminocarbonyl,
optionally substituted alkyl-, aryl- or heterocyclylsulfonyl,
optionally substituted alkyl,
optionally substituted alkenyl,
optionally substituted alkynyl,
optionally substituted acyl,
optionally substituted aryl,
optionally substituted heterocyclyl or
wherein R⁴ and R⁵, together with the nitrogen atom, to which they are bound, form a saturated or unsaturated, optionally substituted 3- to 8-membered ring, which can optionally contain further heteroatoms;
or pharmaceutically acceptable salts thereof.

2. The method according to claim 1, wherein the compound of general formula (I) has the formula (I′) 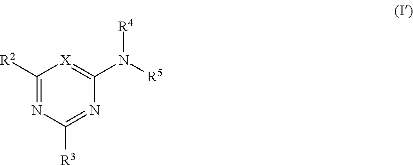
wherein
X is selected from the group consisting of N or C—R′, wherein
R¹ is selected from the group consisting of:
hydrogen,
hydroxyl,
halogen,
carboxyl,
sulfonic acid residue (—SO₃H),
optionally substituted aminocarbonyl,
optionally substituted aminosulfonyl,
optionally substituted amino,
optionally substituted alkyl,
optionally substituted acyl,
optionally substituted alkoxycarbonyl,
optionally substituted acyloxy,
optionally substituted alkoxy,
optionally substituted alkenyl,
optionally substituted alkynyl,
optionally substituted aryl,
optionally substituted heterocyclyl;
R² and R³ are the same or different and are each selected from the group consisting of:
hydrogen,
hydroxyl,
halogen,
carboxyl,
sulfonic acid residue (—SO₃H),
optionally substituted aminocarbonyl,
optionally substituted aminosulfonyl,
optionally substituted amino,
optionally substituted alkyl,
optionally substituted acyl,
optionally substituted alkoxycarbonyl,
optionally substituted acyloxy,
optionally substituted alkoxy,
optionally substituted alkenyl,
optionally substituted alkynyl,
optionally substituted aryl,
optionally substituted heterocyclyl;
R⁴ and R⁵ are the same or different and are each selected from the group consisting of:
hydrogen,
optionally substituted amino,
optionally substituted alkyl-, aryl- or heterocyclylsulfonyl,
optionally substituted alkyl,
optionally substituted alkenyl,
optionally substituted alkynyl,
optionally substituted acyl,
optionally substituted aryl,
optionally substituted heterocyclyl or
wherein R⁴ and R⁵ together with the nitrogen atom, to which they are bound, form a saturated or unsaturated, optionally substituted 3- to 8-membered ring, which can optionally contain further heteroatoms;
or pharmaceutically acceptable salts thereof.

3. The method according to claim 1, wherein
X has the meaning N or C—R¹, wherein
R¹ is selected from the group consisting of:
hydrogen,
halogen,
optionally substituted amino,
optionally substituted alkyl,
optionally substituted alkoxy,
optionally substituted aryl,
optionally substituted heterocyclyl;
R² and R³ are the same or different and are each selected from the group consisting of:
hydrogen,
halogen,
hydroxy,
optionally substituted amino,
optionally substituted aminocarbonyl,
optionally substituted alkyl,
optionally substituted alkoxy,
optionally substituted aryl,
optionally substituted heterocyclyl;
R⁴ and R⁵ are the same or different and are each selected from the group consisting of:
hydrogen,
optionally substituted amino,
optionally substituted alkyl,
optionally substituted aryl,
optionally substituted heterocyclyl or
wherein R⁴ and R⁵ together with the nitrogen atom, to which they are bound, form a saturated or unsaturated, optionally substituted 5- to 6-membered ring, which can optionally contain further heteroatoms;
or pharmaceutically acceptable salts thereof.

4. The method of claim 1, wherein
X has the meaning N or C—R¹, wherein
R¹ is selected from the group consisting of:
hydrogen,
halogen,
optionally substituted alkyl,
optionally substituted alkoxy,
optionally substituted aryl,
optionally substituted heterocyclyl;
R² and R³ are the same or different and are each selected from the group consisting of:
hydrogen,
halogen,
hydroxy,
optionally substituted amino,
optionally substituted aminocarbonyl,
optionally substituted alkoxy,
optionally substituted alkyl,
optionally substituted aryl,
optionally substituted heterocyclyl;
R⁴ and R⁵ are the same or different and are each selected from the group consisting of:
hydrogen,
optionally substituted amino,
optionally substituted alkyl,
optionally substituted aryl,
optionally substituted heterocyclyl or
wherein R⁴ and R⁵ together with the nitrogen atom, to which they are bound, form a saturated or unsaturated, optionally substituted 5- to 6-membered ring, which can optionally contain one to two further heteroatoms;
or pharmaceutically acceptable salts thereof.

5. The method of claim 1, wherein
X has the meaning N or C—R¹, wherein
R¹ is selected from the group consisting of:
hydrogen,
halogen,
optionally substituted alkyl,
optionally substituted alkoxy,
R² and R³ are the same or different and are each selected from the group consisting of
hydrogen,
halogen,
hydroxy,
optionally substituted amino,
optionally substituted aminocarbonyl,
optionally substituted alkoxy,
optionally substituted alkyl,
optionally substituted heterocyclyl,
R⁴ and R⁵ are the same or different and are each selected from the group consisting of:
hydrogen,
optionally substituted amino,
optionally substituted alkyl;
optionally substituted heterocyclyl; or
R⁴ and R⁵ together with the nitrogen atom, to which they are bound, form a saturated or unsaturated, optionally substituted 5- to 6-membered ring, which can optionally contain one to two further heteroatoms;
or pharmaceutically acceptable salts thereof.

6. The method of claim 1, wherein X has the meaning of N,
or pharmaceutically acceptable salts thereof.

7. The method of claim 1, wherein
X has the meaning C—R¹, wherein
R¹ is selected from the group consisting of:
hydrogen,
halogen, or
optionally substituted alkyl,
optionally substituted alkoxy,
or pharmaceutically acceptable salts thereof.

8. The method according to claim 1, wherein
R² and R³ are the same or different and are each selected from the group consisting of:
hydrogen,
halogen,
hydroxy,
optionally substituted amino,
optionally substituted aminocarbonyl,
optionally substituted alkoxy,
optionally substituted alkyl,
optionally substituted heterocyclyl,
or pharmaceutically acceptable salts thereof.

9. The method of claim 1, wherein
R⁴ and R⁵ are the same or different and are each selected from the group consisting of:
hydrogen,
optionally substituted amino;
optionally substituted alkyl;
optionally substituted heterocyclyl; or
R⁴ and R⁵ together with the nitrogen atom, to which they are bound, form a saturated or unsaturated, optionally substituted 5- to 6-membered ring, which can optionally contain one to two further heteroatoms.
or pharmaceutically acceptable salts thereof.

10. The method of claim 1, wherein the compound having formula (I) is selected from the group consisting of: 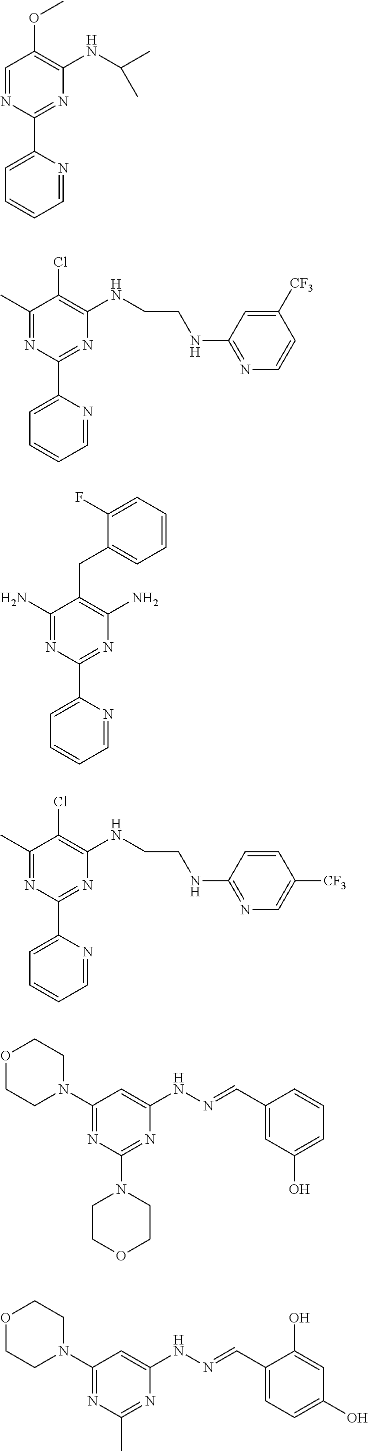 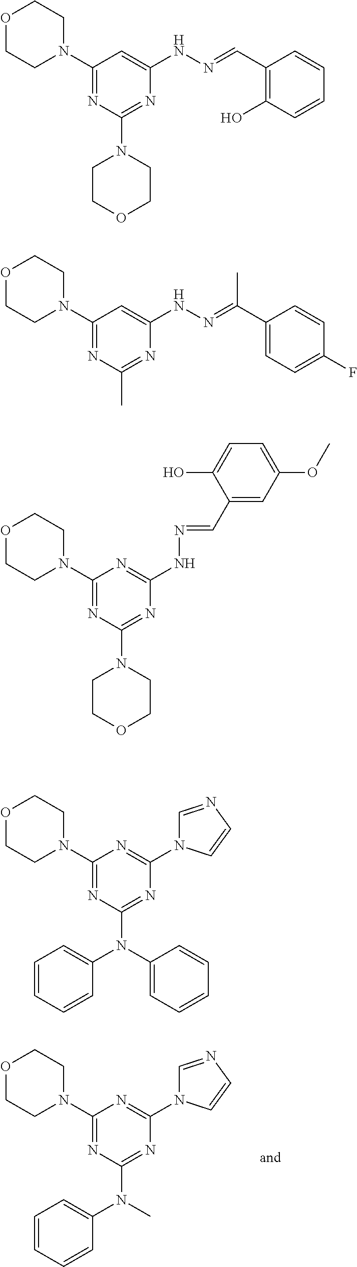 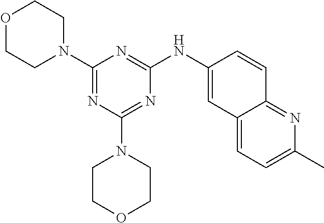
or pharmaceutically acceptable salts thereof, and selected from 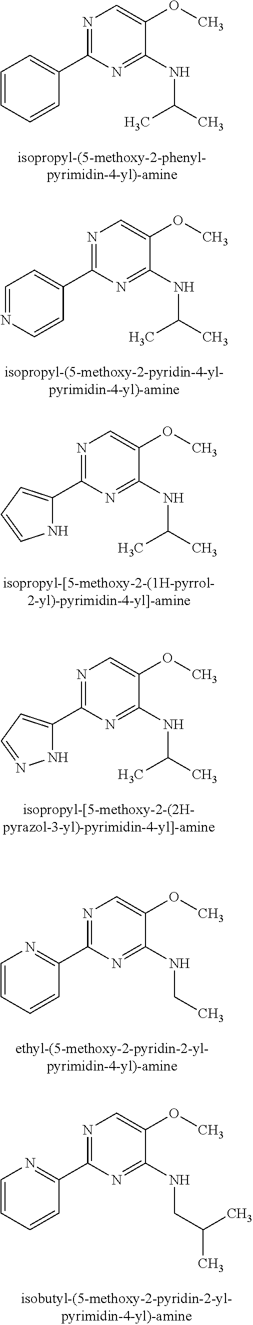 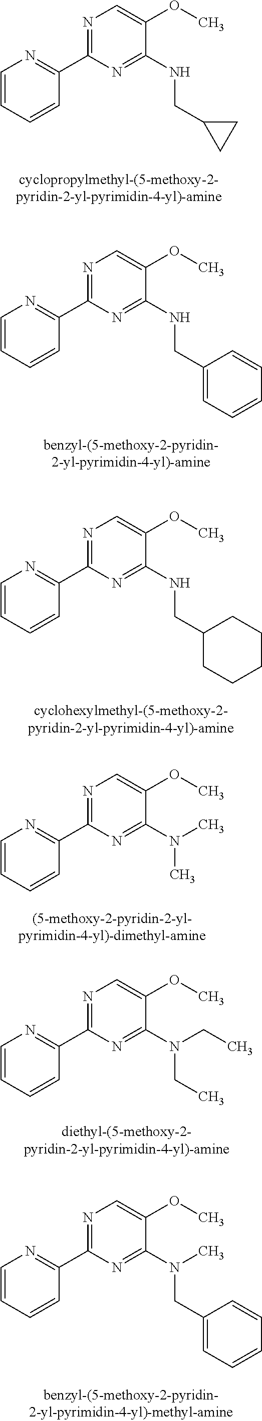 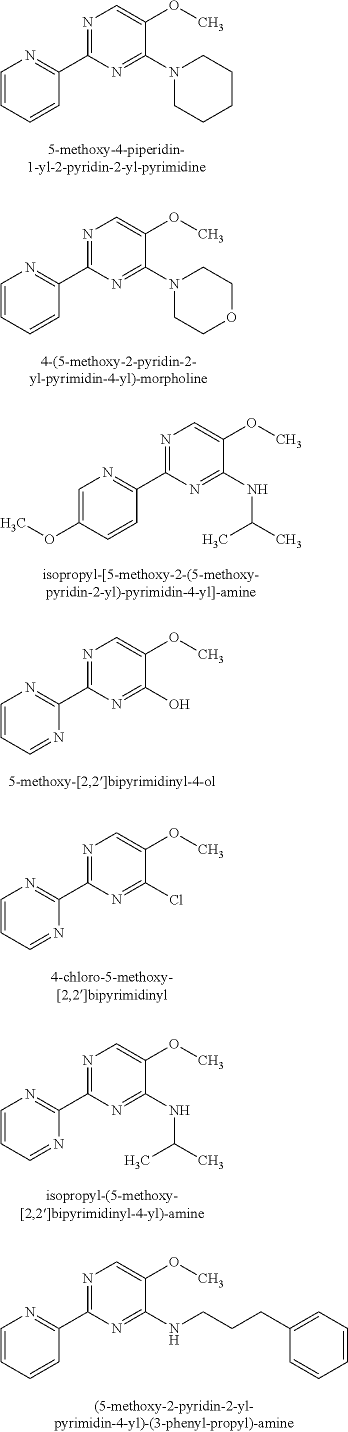 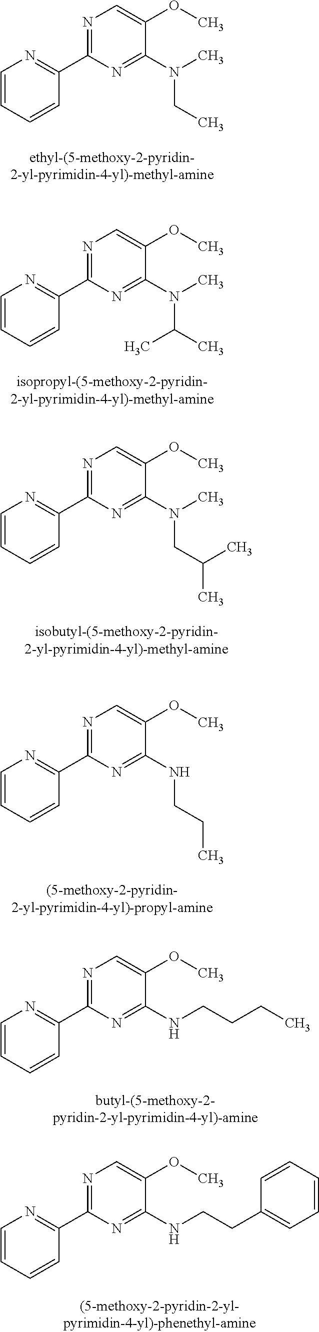 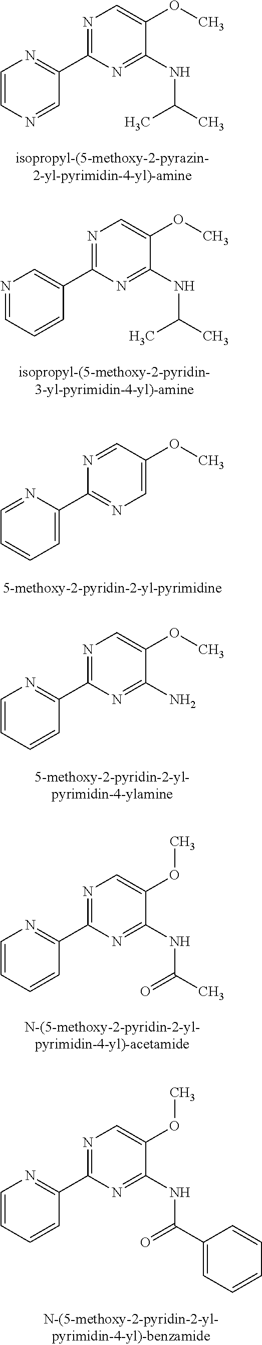 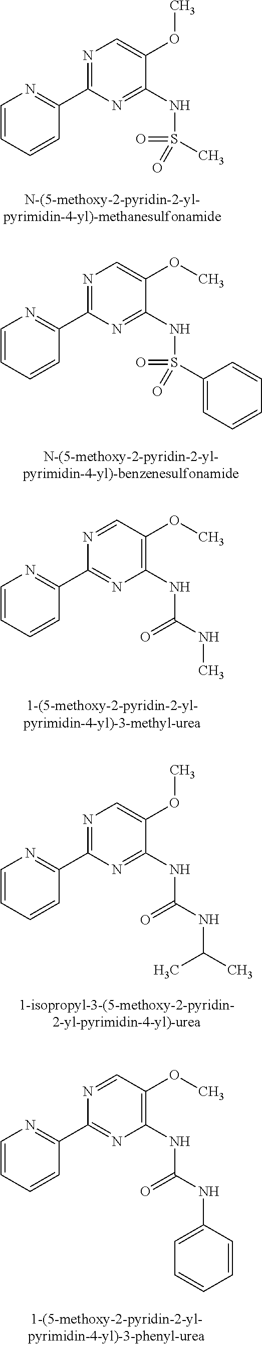 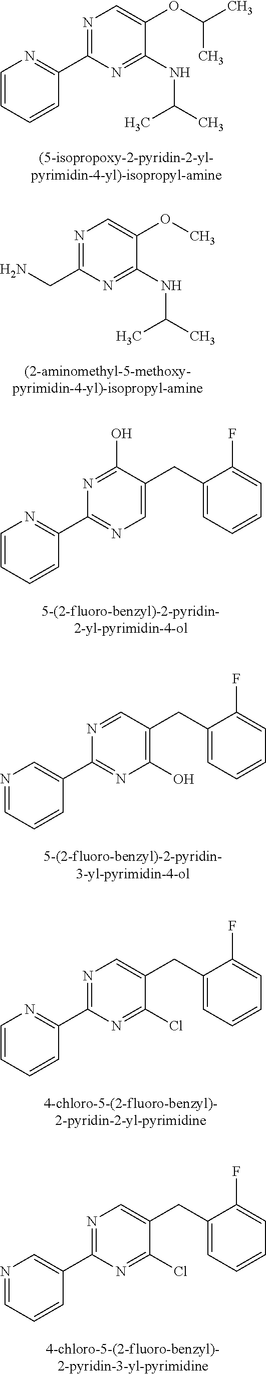 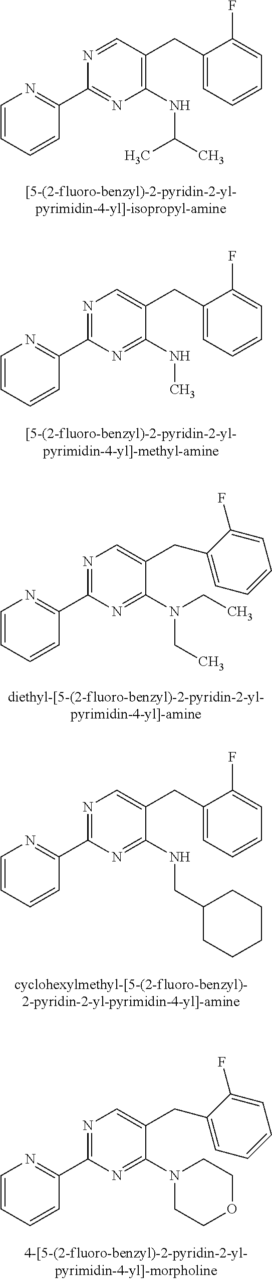 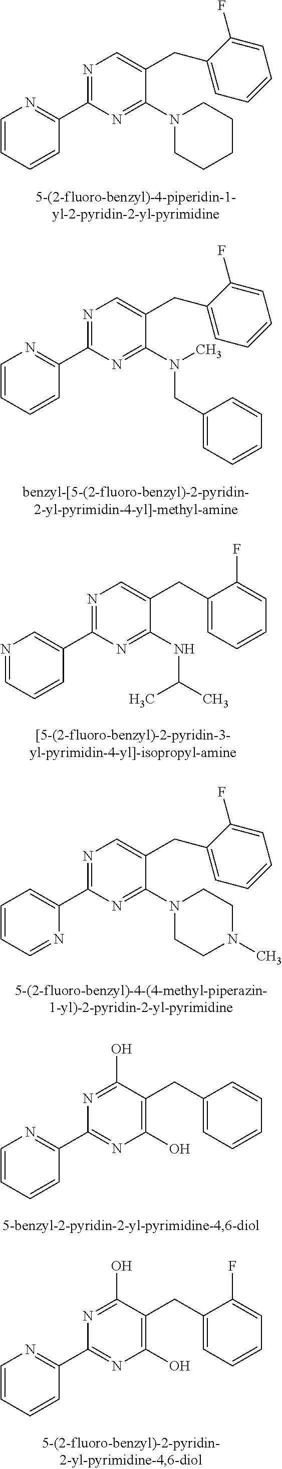 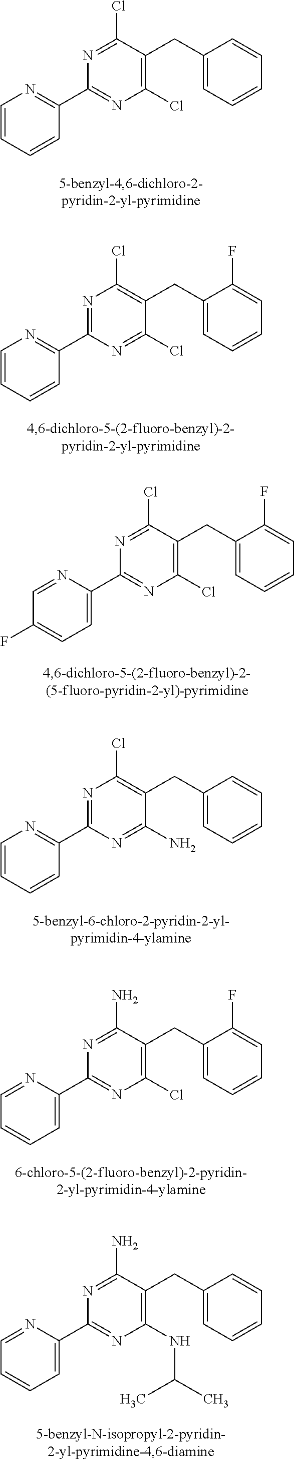 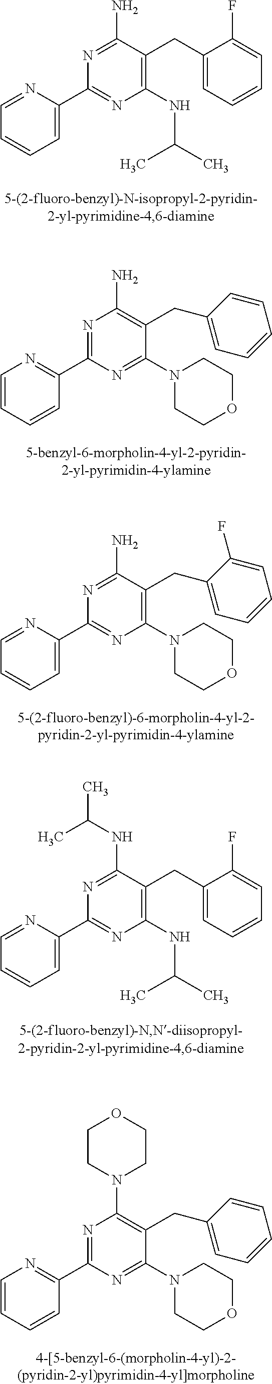 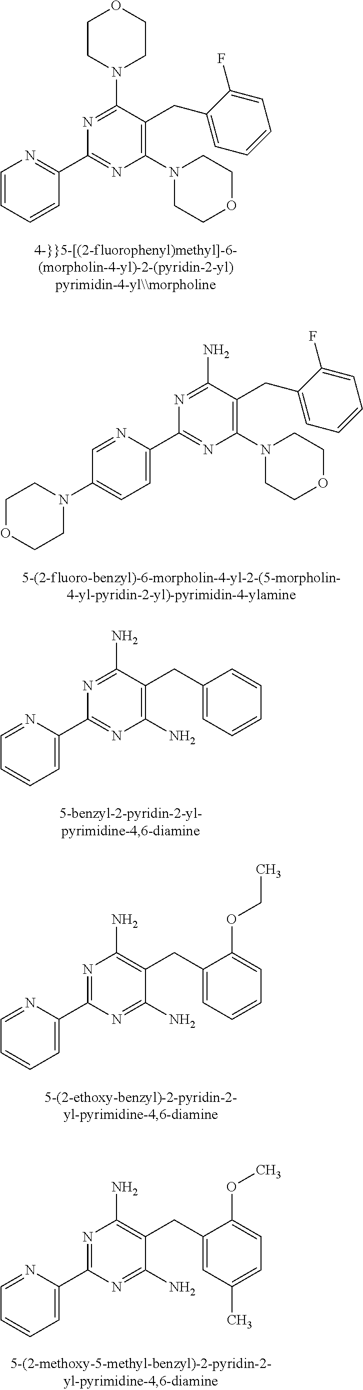 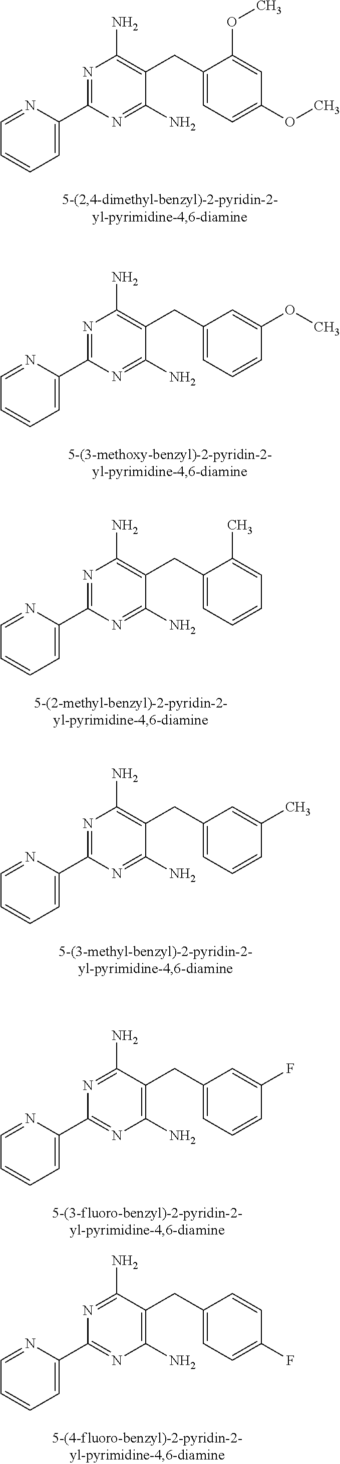 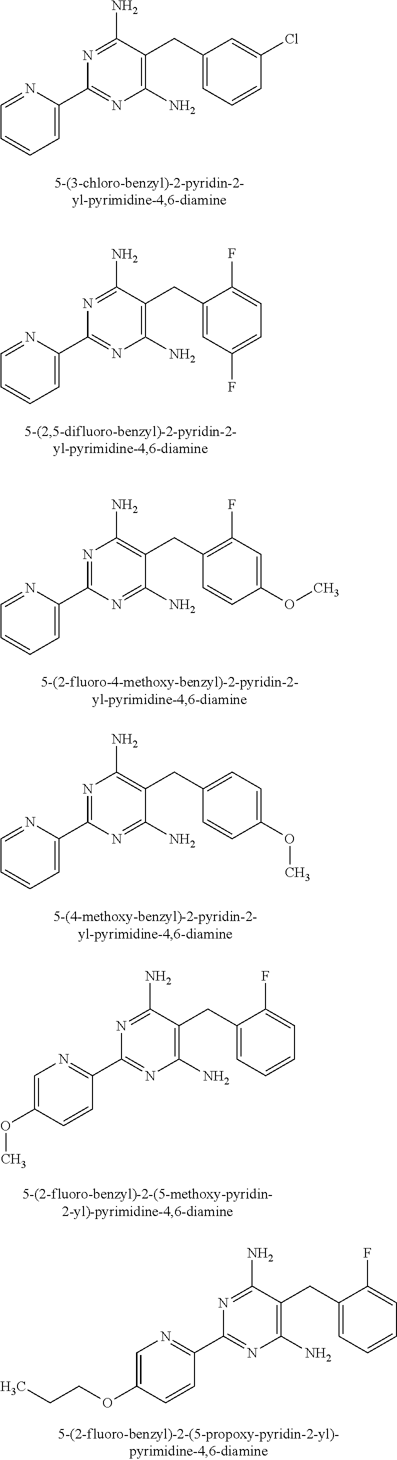 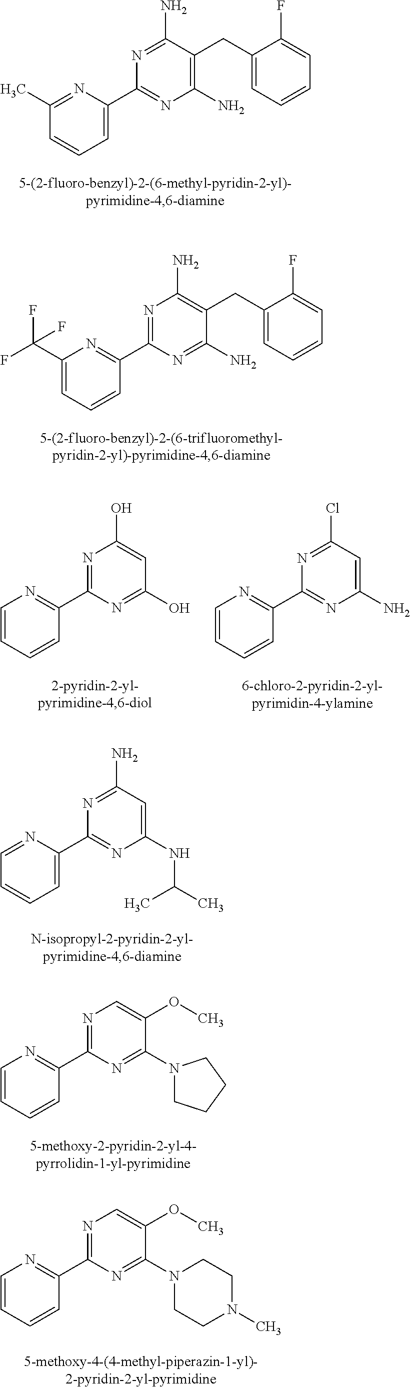 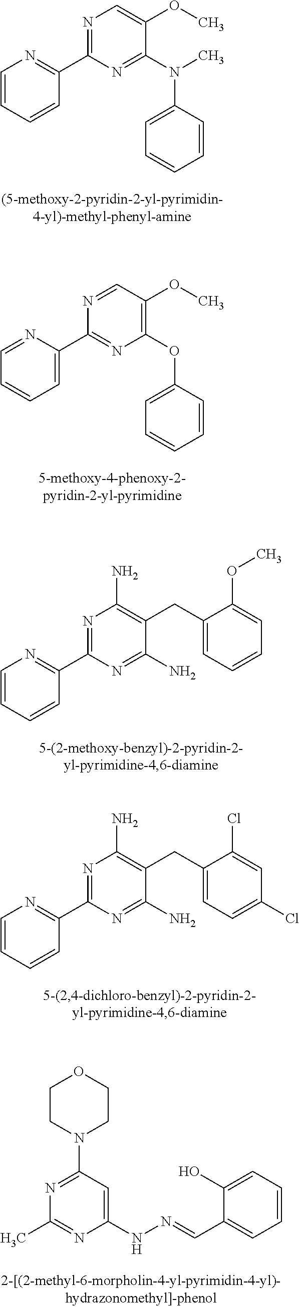 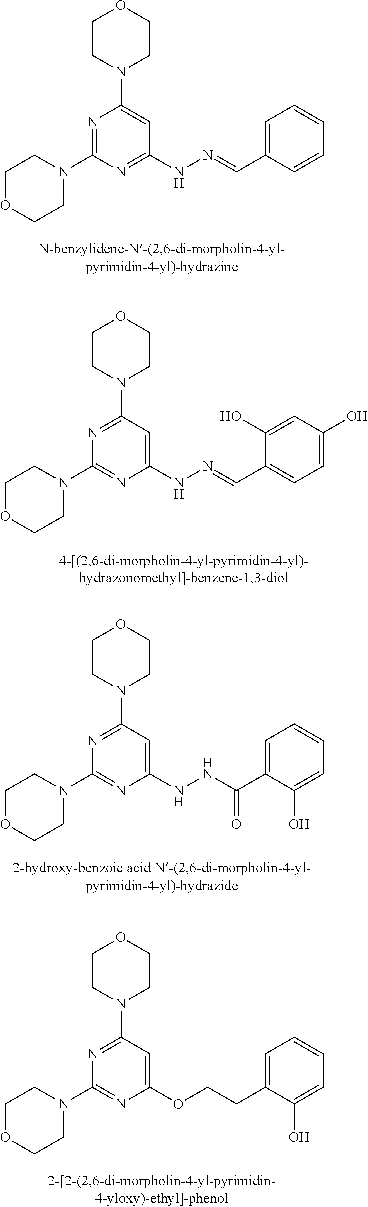 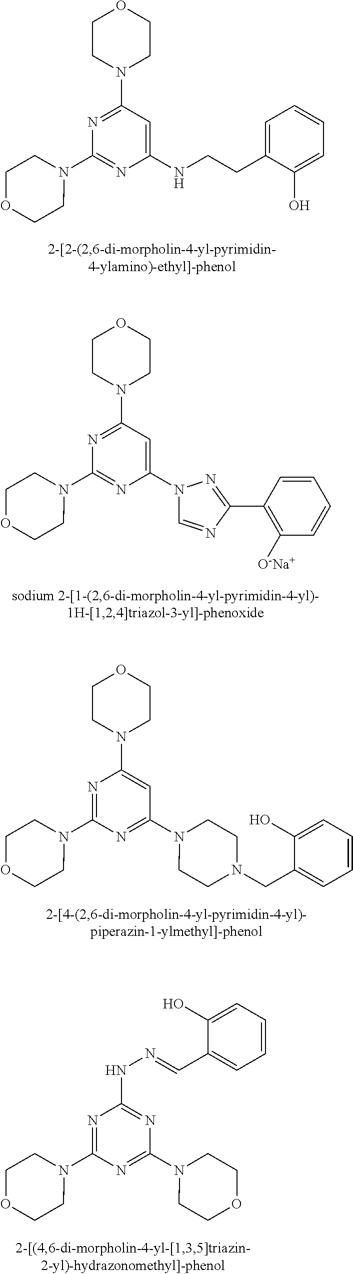 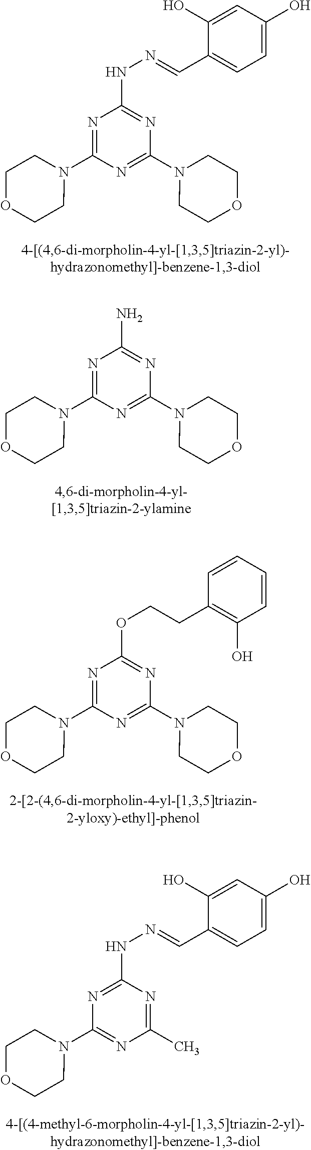
or pharmaceutically acceptable salts thereof.

11-12. (canceled)

13. The method according to claim 1, wherein the iron metabolism disorder is selected from the group consisting of iron deficiency diseases, anaemia, anaemia in cancer, anaemia triggered by chemotherapy, anaemia triggered by inflammation, anaemia in congestive heart failure, anaemia in chronic kidney disease stage 3-5, anaemia trigged by chronic inflammation (AC-D-), anaemia in rheumatoid arthritis, anaemia in systemic lupus erythematosus and anaemia in inflammatory bowel disease.

14. The method according to claim 1, wherein the preparation further comprises at least one of pharmaceutical carriers auxiliaries and solvents.

15. The method of claim 1, wherein the preparation further comprises at least one further pharmaceutically active compound, wherein the pharmaceutically active compound is a compound for the treatment of iron metabolism disorders and the associated symptoms, wherein said pharmaceutically active compound is an iron-containing compound.

16. (canceled)

17. The method of claim 1, wherein the iron metabolism disorders are selected from iron deficiency diseases and anaemia.

18. The method of claim 2, wherein the iron metabolism disorders are selected from iron deficiency diseases and anaemia.

19. The method of claim 3, wherein the iron metabolism disorders are selected from iron deficiency diseases and anaemia.

20. The method of claim 4, wherein the iron metabolism disorders are selected from iron deficiency diseases and anaemia.

21. The method of claim 5, wherein the iron metabolism disorders are selected from iron deficiency diseases and anaemia.

22. The method of claim 6, wherein the iron metabolism disorders are selected from iron deficiency diseases and anaemia.

23. The method of claim 7, wherein the iron metabolism disorders are selected from iron deficiency diseases and anaemia.
